(19) United States
(12) Patent Application Publication
(10) Pub. No.: US 2022/0248744 A1
(43) Pub. Date: Aug. 11, 2022

(54) NICOTINE MATERIALS, METHODS OF MAKING SAME, AND USES THEREOF

(71) Applicant: The Research Foundation for the State University of New York, Amherest, NY (US)

(72) Inventors: Jason B. BENEDICT, Buffalo, NY (US); Devin James ANGEVINE, Kenmore, NY (US)

(21) Appl. No.: 17/627,012

(22) PCT Filed: Jul. 15, 2020
(86) PCT No.: PCT/US20/42190

§ 371 Date: Jan. 13, 2022

Related U.S. Application Data
(60) Provisional application No. 62/874,449, filed on Jul. 15, 2019.

(51) Int. Cl. A24B 15/167; A24B 15/16; A24B 13/00; C07D 401/04

(52) U.S. Cl. CPC A24B 15/167; C07D 401/04; A24B 13/00; A24B 15/16

(57) ABSTRACT

Nicotine materials, compositions including one or more nicotine material(s), and articles of manufacture comprising the nicotine material(s) and/or the composition(s), and uses of the nicotine materials, compositions, and articles of manufacture. The nicotine materials may be nicotine co-crystals, which may include one or more coformer(s), nicotine salts, or the like. The nicotine materials may be made by evaporation of one or more coformer(s), which independently may be solvent(s), and/or solvent(s), if present, from a nicotine material-forming solution. A nicotine material and/or a composition may be used in a nicotine delivery method.

### CROSS REFERENCE TO RELATED APPLICATIONS

This application claims priority to U.S. Provisional Application No. 62/874,449, filed on Jul. 15, 2019, the disclosure of which is hereby incorporated by reference.

### BACKGROUND OF THE DISCLOSURE

Cigarettes, cigars and pipes are popular smoking articles that employ tobacco in various forms. Such smoking articles are used by heating or burning tobacco, and aerosol (e.g. smoke) is inhaled by the smoker. Electronic smoking articles are a further type of tobacco product which comprise a reservoir and heating system for the delivery of aerosolizable materials. Tobacco also may be enjoyed in a so-called “smokeless” form. Particularly popular smokeless tobacco products are employed by inserting some form of processed tobacco or tobacco-containing formulation into the mouth the user.

Certain types of smoking articles, smokeless tobacco products, and electronic smoking articles comprise a tobacco extract, which in some products may be purified such that the extract is comprised primarily of nicotine. However, tobacco extracts comprising a high percentage of nicotine (including extracts comprising at least about 90%, at least about 95%, and at least about 99% nicotine by weight) are typically in oil form. As such, nicotine extracts can be difficult to store, handle, and incorporate into certain tobacco products.

Nicotine salts have been created; however, these flaws are generally constructed within these salts. The flaws can be highly scattered. The body of work thus far lacks any sort of criteria upon which coformers are chosen. In fact, multiple selected coformers are not recognized as even being safe for human consumption. In addition to this, the synthesized salts thus far have the same issue as nicotinium benzoate—a lack of degradation by design.

### SUMMARY OF THE DISCLOSURE

The present disclosure provides nicotine materials, compositions comprising one or more nicotine material(s), and articles of manufacture comprising nicotine material(s) and/or composition(s) comprising one or more nicotine material(s). The present disclosure also provides uses of the nicotine materials, compositions, and articles of manufacture. The present disclosure describes engineering of solid state single crystalline nicotine salts and co-crystals, which may possess more desirable properties including, but not limited to, higher or lower melting point, increased photostability, greater temperature stability, improved vaping and pharmaceutical safety, or a combination thereof, over, for example, pure nicotine and other prior art nicotine salts.

In an aspect, the present disclosure provides methods of making nicotine materials. Non-limiting examples of nicotine materials are described herein. The methods are based on evaporation of at least a portion of the solvent(s), which may be coformer(s), from a solution comprising one or more nicotine source(s) and one or more coformer(s), which may be one or more solvent(s), and, optionally, one or more solvent(s). The nicotine material may be a salt or a mixture of salts, a co-crystal or a mixture of co-crystals, or a combination thereof, comprising one or more polymorph(s), one or more phase(s), and the like, or a combination thereof.

In an aspect, the present disclosure provides nicotine materials and compositions comprising nicotine materials. A nicotine material may comprise one or more nicotine co-crystal(s) and/or one or more nicotine salt(s). A nicotine composition material may comprise one or more nicotine material(s). One or more nicotine material(s) and/or one or more composition(s), each composition comprising one or more nicotine material(s), may be present in an article of manufacture. A nicotine material may be made by a method of the present disclosure. The nicotine materials can exist in various polymorphic and pseudopolymorphic forms. The nicotine materials may comprise materials present in polymorphs exhibiting various Bravais lattice symmetries and corresponding space groups.

A composition may be a vaping composition. A tobacco product (e.g., smoking articles, smokeless tobacco products, and electronic smoking articles) may comprise one or more nicotine material(s) and/or one or more composition(s), each composition comprising one or more nicotine material(s). A composition may further comprise various other substances, such as, for example, one or more excipient(s). A composition may comprise nicotine that is present in a form other than a nicotine material of the present disclosure. A composition may be a pharmaceutical composition, which may include one or more standard pharmaceutically acceptable carrier(s).

In an aspect, the present discloses articles of manufacture. An article of manufacture may comprise one or more nicotine material(s) and/or one or more composition(s). Non-limiting examples of articles of manufacture include transdermal delivery devices, oral delivery devices, a solid state vaporization tablet or capsule, a dissolvable formulation tablet, and the like.

In an aspect, the present disclosure provide methods of using the nicotine materials. The nicotine material may be used in various methods. In various examples, one or more nicotine material(s) and/or one or more composition(s) are used in nicotine storage methods, in nicotine delivery methods (e.g., in nicotine delivery methods, such as, for example, vaping methods, nicotine-based treatment methods, in nicotine addiction treatment methods, and the like), or in nicotine product formulation, or the like. Non-limiting examples of uses of nicotine materials and compositions comprising nicotine materials of the present disclosure are provided herein.

In an aspect, the present disclosure provide kits. A kit may comprise pharmaceutical preparations containing one or more nicotine material and/or one or more nicotine composition of the present disclosure. In various examples, a kit comprises a package (e.g., a closed or sealed package) that contains one or more nicotine material(s) and/or one or more nicotine composition(s), such as, for example, one or more closed or sealed vial(s), bottle(s), blister (bubble) pack(s), or any other suitable packaging for the sale, distribution, or use of the nicotine compounds and compositions comprising them.

### BRIEF DESCRIPTION OF THE FIGURES

For a fuller understanding of the nature and objects of the disclosure, reference should be made to the following detailed description taken in conjunction with the accompanying figures.

FIG. 1 shows a single crystal of orthorhombic P2₁2₁2₁ (S)-nicotinium L-malate.

FIG. 2 shows a vial of crystals of orthorhombic P2₁2₁2₁ (S)-nicotinium L-malate.

FIG. 3 shows a CuKα radiation source simulated powder X-ray diffraction pattern of orthorhombic P2₁2₁2₁ (S)-nicotinium L-malate.

FIG. 4 shows a symmetric unit of orthorhombic P2₁2₁2₁ (S)-nicotinium L-malate.

FIG. 5 shows a down crystallographic a-axis of orthorhombic P2₁2₁2₁ (S)-nicotinium L-malate with b and c normal to the plane.

FIG. 6 shows a down crystallographic b-axis of orthorhombic P2₁2₁2₁ (S)-nicotinium L-malate with a and c normal to the plane.

FIG. 7 shows a single crystal of monoclinic P2₁ (S)-nicotinium L-malate.

FIG. 8 shows a vial of crystals of monoclinic P2₁ (S)-nicotinium L-malate.

FIG. 9 shows a CuKα radiation source simulated powder X-ray diffraction pattern of monoclinic P2₁ (S)-nicotinium L-malate.

FIG. 10 shows an asymmetric unit of monoclinic P2₁ (S)-nicotinium L-malate.

FIG. 11 shows a view down crystallographic a-axis of monoclinic P2₁ (S)-nicotinium L-malate with b normal to the plane and c non-normalized with respect to the plane.

FIG. 12 shows a view down crystallographic b-axis of monoclinic P2₁ (S)-nicotinium L-malate with a and c normal to the plane.

FIG. 13 shows a single crystal of orthorhombic P2₁2₁2₁ (S)-nicotinium D-malate.

FIG. 14 shows a vial of crystals of orthorhombic P2₁2₁2₁ (S)-nicotinium D-malate.

FIG. 15 shows a simulated powder X-ray diffraction pattern of orthorhombic P2₁2₁2₁ (S)-nicotinium D-malate.

FIG. 16 shows an asymmetric unit of orthorhombic P2₁2₁2₁ (S)-nicotinium D-malate.

FIG. 17 show an asymmetric unit of orthorhombic P2₁2₁2₁ (S)-nicotinium D-malate.

FIG. 18 shows a view down crystallographic a-axis of orthorhombic P2₁2₁2₁ (S)-nicotinium D-malate with b and c normal to the plane.

FIG. 19 shows a view down crystallographic b-axis of orthorhombic P2₁2₁2₁ (S)-nicotinium D-malate with a and c normal to the plane.

FIG. 20 shows a single crystal of orthorhombic P2₁2₁2₁ (S)-nicotinium DL-malate.

FIG. 21 shows a vial of crystals of orthorhombic P2₁2₁2₁ (S)-nicotinium DL-malate.

FIG. 22 shows a simulated powder X-ray diffraction pattern of orthorhombic P2₁2₁2₁ (S)-nicotinium DL-malate.

FIG. 23 shows a diagram of occupancy within an orthorhombic P2₁2₁2₁ (S)-nicotinium DL-malate crystal grown from a racemic mixture.

FIG. 24 shows a view down crystallographic a-axis of orthorhombic P2₁2₁2₁ (S)-nicotinium DL-malate with b and c normal to the plane.

FIG. 25 shows a view down crystallographic b-axis of orthorhombic P2₁2₁2₁ (S)-nicotinium DL-malate with a and c normal to the plane.

FIG. 26 shows a single crystal of monoclinic P2₁ (S)-nicotinium salicylate.

FIG. 27 shows a vial of crystals of monoclinic P2₁ (S)-nicotinium salicylate.

FIG. 28 shows a simulated powder X-ray diffraction pattern of P2₁ (S)-nicotinium salicylate.

FIG. 29 shows an asymmetric unit of monoclinic P2₁ (S)-nicotinium salicylate.

FIG. 30 shows a view down crystallographic a-axis of monoclinic P2₁ (S)-nicotinium salicylate with b normal to the plane and c non-normalized with respect to the plane.

FIG. 31 shows a view down crystallographic b-axis of monoclinic P2₁ (S)-nicotinium salicylate with a and c normal to the plane.

FIG. 32 shows a view down crystallographic c-axis of monoclinic P2₁ (S)-nicotinium salicylate with the b axis normal to the plane and a non-normalized with respect to the plane.

FIG. 33 shows a single crystal of monoclinic P2₁ (S)-nicotinium 2,6-dihydroxybenzoate.

FIG. 34 shows a vial of crystals of monoclinic P2₁ (S)-nicotinium 2,6-dihydroxybenzoate.

FIG. 35 shows a simulated powder X-ray diffraction pattern of monoclinic P2₁ (S)-nicotinium 2,6-dihydroxybenzoate at room temperature.

FIG. 36 shows an asymmetric unit of monoclinic P2₁ (S)-nicotinium 2,6-dihydroxybenzoate at room temperature.

FIG. 37 shows a view down crystallographic a-axis of monoclinic P2₁ (S)-nicotinium 2,6-dihydroxybenzoate at room temperature with b normal to the plane and c non-normalized with respect to the plane.

FIG. 38 shows a view down crystallographic b-axis of monoclinic P2₁ (S)-nicotinium 2,6-dihydroxybenzoate at room temperature with a and c normal to the plane.

FIG. 39 shows a view down crystallographic c-axis of monoclinic P2₁ (S)-nicotinium 2,6-dihydroxybenzoate at room temperature with the b axis normal to the plane and a non-normalized with respect to the plane.

FIG. 40 shows a simulated powder X-ray diffraction pattern of monoclinic P2₁ (S)-nicotinium 2,6-dihydroxybenzoate at 90 K.

FIG. 41 shows an asymmetric unit of monoclinic P2₁ (S)-nicotinium 2,6-dihydroxybenzoate at 90 K.

FIG. 42 shows a view of monoclinic P2₁ (S)-nicotinium 2,6-dihydroxybenzoate at 90 K.

FIG. 43 shows a view of monoclinic P2₁ (S)-nicotinium 2,6-dihydroxybenzoate at 90 K along [1 0 1], with b normal to the plane and a and c non-normalized with respect to the plane.

FIG. 44 shows a view down crystallographic a-axis of monoclinic P2₁ (S)-nicotinium 2,6-dihydroxybenzoate at 90 K with b normal to the plane and c non-normalized with respect to the plane.

FIG. 45 shows a view down crystallographic b-axis of monoclinic P2₁ (S)-nicotinium 2,6-dihydroxybenzoate at 90 K with a and c normal to the plane.

FIG. 46 shows a view down crystallographic c-axis of monoclinic P2₁ (S)-nicotinium 2,6-dihydroxybenzoate at 90 K with the b axis normal to the plane and a non-normalized with respect to the plane.

FIG. 47 shows a graph of monoclinic P2₁ (S)-nicotinium 2,6-dihydroxybenzoate unit cell axes lengths and the unique monoclinic system angle β as a function of the temperature.

FIG. 48 shows a graph of monoclinic P2₁ (S)-nicotinium 2,6-dihydroxybenzoate unit cell axes lengths and the unit cell volume as a function of the temperature.

FIG. 49 shows a single crystal of orthorhombic P222₁ (S)-nicotinium orotate monohydrate.

FIG. 50 shows vials of crystals of orthorhombic P222₁ (S)-nicotinium orotate monohydrate.

FIG. 51 shows a simulated powder X-ray diffraction pattern of P222₁ (S)-nicotinium orotate monohydrate.

FIG. 52 shows an asymmetric unit of orthorhombic P222₁ (S)-nicotinium orotate monohydrate.

FIG. 53 shows a diagram of orthorhombic P222₁ (S)-nicotinium orotate monohydrate, depicting the H-bonds that are present between the acid coformer.

FIG. 54 shows a view down crystallographic a-axis of (S)-nicotinium P222₁ orotate monohydrate with b and c normal to the plane.

FIG. 55 shows a view down crystallographic b-axis of orthorhombic P222₁ (S)-nicotinium orotate monohydrate with a and c normal to the plane.

FIG. 56 shows a view of a crystal of monoclinic P2₁ (S)-nicotinium 2,5-dihydroxyterephthalate.

FIG. 57 shows a vial of crystals of monoclinic P2₁ (S)-nicotinium 2,5-dihydroxyterephthalate.

FIG. 58 shows a simulated powder X-ray diffraction pattern of monoclinic P2₁ (S)-nicotinium 2,5-dihydroxyterephthalate.

FIG. 59 shows an asymmetric unit of monoclinic P2₁ (S)-nicotinium 2,5-dihydroxyterephthalate.

FIG. 60 shows a view down crystallographic a-axis of monoclinic P2₁ (S)-nicotinium 2,5-dihydroxyterephthalate with b normal to the plane and c non-normalized with respect to the plane.

FIG. 61 shows a view down crystallographic b-axis of monoclinic P2₁ (S)-nicotinium 2,5-dihydroxyterephthalate with a and c normal to the plane.

FIG. 62 shows a view down crystallographic c-axis of monoclinic P2₁ (S)-nicotinium 2,5-dihydroxyterephthalate with the b axis normal to the plane and a non-normalized with respect to the plane.

FIG. 63 shows a crystal of monoclinic P2₁ (S)-nicotinium bi-L-(+)-tartrate dihydrate.

FIG. 64 shows a vial of crystals of monoclinic P2₁ (S)-nicotinium bi-L-(+)-tartrate dihydrate.

FIG. 65 shows a simulated powder X-ray diffraction pattern of monoclinic P2₁ (S)-nicotinium bi-L-(+)-tartrate dehydrate.

FIG. 66 shows an asymmetric unit of monoclinic P2₁ (S)-nicotinium bi-L-(+)-tartrate dehydrate.

FIG. 67 shows a view down crystallographic a-axis of monoclinic P2₁ (S)-nicotinium bi-L-(+)-tartrate dihydrate with b normal to the plane and c non-normalized with respect to the plane.

FIG. 68 shows a view down crystallographic b-axis of monoclinic P2₁ (S)-nicotinium bi-L-(+)-tartrate dihydrate with a and c normal to the plane.

FIG. 69 shows a view along [1 1 0] of monoclinic P2₁ (S)-nicotinium bi-L-(+)-tartrate dihydrate with c normal to the plane and a and b non-normalized with respect to the plane.

FIG. 70 shows a depiction of the occupancy of the N-methylpyrrolidine ring found within the ring puckering conformation of monoclinic P2₁ (S)-nicotinium bi-L-(+)-tartrate dihydrate.

FIG. 71 shows an ¹H NMR spectrum detailing amorphous (S)-nicotinium 2,4-dihydroxybenzoate formation in solution.

FIG. 72 shows a diagram of lattice view down crystallographic b-axis of monoclinic P2₁ (S)-nicotinium L-malate, detailing the nicotine's packing.

FIG. 73 shows a diagram of lattice view down crystallographic b-axis of orthorhombic P2₁2₁2₁ (S)-nicotinium L-malate and orthorhombic P2₁2₁2₁ (S)-nicotinium D-malate, detailing the nicotine's packing.

FIG. 74 shows a diagram of lattice view down crystallographic a-axis of salt orthorhombic P222₁ (S)-nicotinium orotate monohydrate, showing the be plane with water on special positions due to the C₂ rotation axes going through the oxygen molecule of each water.

FIG. 75 shows an overlay of the enantiomerically pure nicotinium malate salts DSC data detailing the observed endothermic curves of each (S)-nicotinium malate salt.

FIG. 76 shows an overlay of the DSC spectrum acquired for eight of the acquired crystalline compounds.

FIG. 77 shows (S)-nicotine photodegradation.

FIG. 78 shows an (S)-nicotine photodegradation.

FIG. 79 shows an ¹H NMR spectrum detailing (S)-nicotine degradation after 24 hours of UV irradiation.

FIG. 80 shows (upper) an ¹H NMR spectrum of L-malic acid prior to UV irradiation. (lower) an ¹H NMR spectrum of the same L-malic acid after 24 hours of UV irradiation. The d₄-methanol peaks were also observed.

FIG. 81 shows (upper) an ¹H NMR spectrum of D-malic acid prior to UV irradiation. (lower) an ¹H NMR spectrum of the same D-malic acid after 24 hours of UV irradiation. The d₄-methanol peaks were also observed.

FIG. 82 shows (upper) an ¹H NMR spectrum of DL-malic acid prior to UV irradiation. (lower) an ¹H NMR spectrum of the same DL-malic acid after 24 hours of UV irradiation. The d₃-methanol peak was also observed, along with water.

FIG. 83 shows an ¹H NMR spectrum detailing no orotic acid degradation after 24 hours of UV irradiation.

FIG. 84 shows (upper) an ¹H NMR spectrum of salicylic acid prior to UV irradiation. (lower) an ¹H NMR spectrum of the same salicylic acid after 24 hours of UV irradiation. The d₄-methanol peaks are also observed.

FIG. 85 shows (upper) an ¹H NMR spectrum of 2,6-dihydroxybenzoic acid prior to UV irradiation. (lower) an ¹H NMR spectrum of the same 2,6-dihydroxybenzoic acid after 24 hours of UV irradiation The d₄-methanol peaks were also observed.

FIG. 86 shows an ¹H NMR spectrum detailing no 2,5-dihydroxyterephthalic acid degradation after 24 hours of UV irradiation.

FIG. 87 shows an ¹H NMR spectrum detailing no degradation of orthorhombic P2₁2₁2₁ (S)-nicotinium L-malate after 24 hours of UV irradiation.

FIG. 88 shows an ¹H NMR spectrum detailing no degradation of monoclinic P2₁ (S)-nicotinium L-malate after 24 hours of UV irradiation.

FIG. 89 shows an ¹H NMR spectrum detailing no degradation of orthorhombic P2₁2₁2₁ (S)-nicotinium D-malate after 24 hours of UV irradiation.

FIG. 90 shows an ¹H NMR spectrum detailing no degradation of orthorhombic P2₁2₁2₁ (S)-nicotinium DL-malate after 24 hours of UV irradiation.

FIG. 91 shows an ¹H NMR spectrum detailing no degradation of orthorhombic P222₁ (S)-nicotinium orotate monohydrate after 24 hours of UV irradiation.

FIG. 92 shows an ¹H NMR spectrum detailing no degradation of monoclinic P2₁ (S)-nicotinium salicylate after 24 hours of UV irradiation.

FIG. 93 shows an ¹H NMR spectrum detailing no degradation of monoclinic P2₁ (S)-nicotinium 2,6-dihydroxybenzoate after 24 hours of UV irradiation.

FIG. 94 shows an ¹H NMR spectrum detailing no degradation of monoclinic P2₁ (S)-nicotinium 2,5-dihydroxyterephthalate after 24 hours of UV irradiation.

FIG. 95 shows examples of the experimental setup for sample vaporization.

FIG. 96 shows the top down view of Sai atomizer mouthpiece with crystals collected after vaping.

FIG. 97 shows the crystals that formed in the Sai atomizer mouthpiece after vaporization.

FIG. 98 shows the nanocrystalline material that formed in the analogous syringe lung.

FIG. 99 shows a diagram depicting the structure and occupancy of single crystals of a nicotine material of the present disclosure, which were recovered from both the mouthpiece and inside of the syringe.

FIG. 100 shows an ¹H NMR spectrum detailing the fate of orthorhombic P2₁2₁2₁ (S)-nicotinium L-malate after vaporization.

FIG. 101 shows and ¹H NMR spectrum detailing the fate of monoclinic P2₁ (S)-nicotinium L-malate after vaporization.

FIG. 102 shows an ¹H NMR spectrum detailing the fate of orthorhombic P2₁2₁2₁ (S)-nicotinium D-malate after vaporization.

FIG. 103 shows ¹H NMR spectrum detailing the fate of orthorhombic P2₁2₁2₁ (S)-nicotinium DL-malate after vaporization.

FIG. 104 shows ¹H NMR spectrum detailing the fate of P222₁ (S)-nicotinium orotate monohydrate after vaporization.

FIG. 105 shows ¹H NMR spectrum detailing the fate of monoclinic P2₁ (S)-nicotinium salicylate after vaporization.

FIG. 106 shows ¹H NMR spectrum detailing the fate of monoclinic P2₁ (S)-nicotinium 2,6-dihydroxybenzoate after vaporization.

FIG. 107 shows ¹H NMR spectrum detailing the fate of monoclinic P2₁ (S)-nicotinium 2,5-dihydroxyterephthalate after vaporization.

### DETAILED DESCRIPTION OF THE DISCLOSURE

Although claimed subject matter is described in terms of certain examples, other examples, including examples that do not necessarily provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include all values to the magnitude of the smallest value (either the lower limit value or the upper limit value) and ranges between the values of the stated range.

The present disclosure provides nicotine materials, compositions comprising one or more nicotine material(s), and articles of manufacture comprising nicotine material(s) and/or composition(s) comprising one or more nicotine material(s). The present disclosure also provides uses of the nicotine materials, compositions, and articles of manufacture.

Safety with regard to use of nicotine can be improved by engineering nicotine materials (e.g., nicotine co-crystals, nicotine salts, and the like) specifically for degradation. Crystal engineering methods were used with nicotine and various coformers (e.g., food safe coformers, flavor additive coformers, generally recognized as safe (GRAS) coformers, and the like, and combinations thereof. The present disclosure describes engineering of solid state single crystalline nicotine salts and co-crystals, which may possess more desirable properties including, but not limited to, higher or lower melting point, increased photostability, greater temperature stability, improved vaping and pharmaceutical safety, or a combination thereof, over, for example, pure nicotine and other prior art nicotine salts. Various compounds may potentially be suitable for use as a coformer to produce a nicotine salt or coformer. Non-limiting examples of these potential coformers may be found in the Food and Drug Administration's (FDA's) GRAS database and notices, the Canadian database of food constituents (FooDB), as well as in the Complex Systems Lab's (CoSyLab's) comprehensive database of flavor molecules (FlavorDB). Also, the compounds in Table 1 in Example 1 provide non-limiting examples of coformers considered to be safe that may be used in the tuning of these solid state nicotine salts and co-crystals. Any enantiomer, conformation, or hydrate/solvate or salt of these coformers, or a combination thereof, may be used to form desired salts and co-crystals, and polymorphs thereof.

In an aspect, the present disclosure provides methods of making nicotine materials. Non-limiting examples of nicotine materials are described herein.

The methods are based on evaporation of at least a portion of the solvent from a solution comprising one or more nicotine source(s) and one or more coformer(s), which may be one or more solvent(s), and, optionally, one or more solvent(s) (i.e., solvent(s) that are not coformer(s)). Non-limiting examples of methods of the present disclosure are described herein.

Nicotine can be obtained (e.g., purchased, isolated, derived, and the like) from various sources. Suitable nicotine sources are commercially available or are known in the art. The nicotine may be derived from some form of a plant of the Nicotiana species. The nicotine may be in the form of a highly purified tobacco extract. Various methods are known for the isolation and purification of nicotine from tobacco (including, but not limited to, extraction from tobacco with water; extraction from tobacco with organic solvents; steam distillation from tobacco; or pyrolytic degradation of tobacco and distillation of nicotine therefrom).

In various examples, a method of making a nicotine material of the present disclosure comprises providing a nicotine material-forming solution comprising nicotine, a coformer, which may be a combination of two or more coformers, nicotine, and, optionally, a solvent, which may be a mixture of solvents; and removing (e.g., evaporating) at least a portion (e.g., substantially all or all) of the solvent(s), which may be conformer(s), from the nicotine material-forming solution, where the nicotine material is formed. In various examples, a method comprises evaporating at least a portion of the solvent(s), if the resulting nicotine material(s) contain a solvent that is a coformer, and/or solvent(s), if present, from the nicotine material-forming solution.

The nicotine material may be a salt or a mixture of salts, a co-crystal or a mixture of co-crystals, or a combination thereof, comprising one or more polymorph(s), one or more phase(s), and the like, or a combination thereof. In various examples, the nicotine mixture comprises two or more polymorphs or a mixture of two or more different co-crystals. This nicotine material may be made with a single coformer having two different phases.

A nicotine material-forming solution may comprise various coformers and/or solvents. Combinations of coformers may be used. Non-limiting examples of coformers are described herein. A coformer may be a solvent, such as, for example, a solvent described herein.

The nicotine source(s) and coformer(s) may be present in the solution in various concentrations. Illustrative, non-limiting examples of nicotine source and coformer concentrations are provided herein.

A portion of or all of the solvent(s), which may be one or more coformer(s), may be removed in various ways. By “substantially all of the solvent(s) of the nicotine material-forming solution” it is meant that solvent is removed until at least solid nicotine material formation is observed. A portion of or all of the solvent(s) may be removed in active and/or passive ways. Non-limiting, illustrative examples of active removal of the solvent(s) include using vacuum, a stream of gas, heating, or a combination thereof. An illustrative, non-limiting example of passive removal of the solvent(s) is allowing the solvent to evaporate under ambient (e.g., room temperature and ambient pressure, etc.) conditions (e.g., without the use of, for example, vacuum, a stream of gas, heating, or a combination thereof). In various examples, the evaporating is carried out by heating the solution to a temperature below the decomposition temperature of nicotine, which may be above the boiling point of one or more or all the solvent(s), exposing at least a portion of a surface of the solution to a dynamic atmosphere of gas, which may be an inert gas, or a sub-ambient pressure (e.g., a pressure of 0.01 to 759 torr, including all 0.01 torr values and ranges therebetween), or a combination thereof. It is desirable that the nicotine is completely soluble in the solvent(s). In various examples, the method (or at least the evaporating) is carried out in the dark (e.g., in the absence of visible light wavelengths (e.g., 400-700 nm) or by blocking 90% or more, 95% or more, 99% or more of the visible light wavelengths of ambient visible light (e.g., sunlight, incandescent light(s), fluorescent light(s), or a combination thereof).

Without intending to be bound by any particular theory, it is considered that the nicotine material is formed by spontaneous nucleation, crystallization, or precipitation. In various examples, solvent removal (e.g., the evaporating) is carried out without adding seed crystals to the nicotine material-forming solution. In various examples, the nicotine is not precipitated. In various examples, the nicotine material-forming solution does not comprise a nicotine non-solvent (e.g., a solvent in which nicotine has little, such as, for example, 5% by weight or less or no measurable, for example, measurable by gravimetric methods, spectrophotometric methods, spectrometry methods, or the like). In various examples, a method comprises one or more or all of these examples.

A nicotine material may be subjected to various post-formation processes. In various examples, a nicotine-material is isolated, dried, or isolated and dried. A nicotine material may be formed into a tablet or other solid form or a liquid comprising the nicotine material formed.

A nicotine material may have various structural features. A nicotine material may be an individual solid particle or a plurality of solid particles, which are independently, in the case of a plurality of solid particles, amorphous, polycrystalline, single crystalline, or a combination thereof. At least a portion of or all of the nicotine material may exhibit a specific symmetry or a combination of specific symmetries. Examples of specific symmetries are provided herein.

In an aspect, the present disclosure provides nicotine materials and compositions comprising nicotine materials. A nicotine material may comprise one or more nicotine co-crystal(s) and/or one or more nicotine salt(s). The nicotine materials may be crystalline nicotine materials. A nicotine composition material may comprise one or more nicotine material(s). One or more nicotine material(s) and/or one or more composition(s), each composition comprising one or more nicotine material(s), may be present in an article of manufacture. Non-limiting examples of nicotine materials, compositions comprising nicotine materials, and articles of manufacture of the present disclosure are provided herein. A nicotine material may be made by a method of the present disclosure. In various examples, a nicotine material is made by a method of the present disclosure.

By isolating nicotine materials in the solid state, the need for a liquid carrier for nicotine products, such as, for example, a vaporization delivery product, a nicotine addiction treatment product, a pill or capsule product, a nicotine storage product, or the like, can be eliminated. These nicotine materials may also be used for nicotine formulation products. In various examples, a nicotine material or composition comprising one or more nicotine material(s) does not comprise a liquid carrier.

A nicotine co-crystal may comprise nicotine and one or more coformer(s). Without intending to be bound by any particular theory, it is considered that a nicotine co-crystal comprises nicotine and one or more coformer(s), where there has been no proton transfer between the nicotine and coformer(s). Without intending to be bound by any particular theory, it is considered that a nicotine salt comprises nicotinium cations and anions formed from one or more coformer(s) and/or neutral/uncharged nicotine and one or more cation/anion pair(s) formed from at least two coformers, where there has been proton transfer between the nicotine and coformer(s) or between at least two coformers, respectively.

A composition may be a vaping composition. A tobacco product (e.g., smoking articles, smokeless tobacco products, and electronic smoking articles) may comprise one or more nicotine material(s) and/or one or more composition(s), each composition comprising one or more nicotine material(s).

In various examples, a nicotine material, composition, or article of manufacture of the present disclosure is an easier to handle form than the original source nicotine (e.g., a solid or semi-solid form) and/or is provided in a higher purity form than the original source nicotine. In various examples, a nicotine material, composition, or article of manufacture of the present disclosure exhibit greater thermodynamic and/or physical, and/or chemical stability (e.g., a higher resistance to oxidation, reduced risk of hydrate formation, and/or a longer shelf life) when compared with the original source nicotine.

The stoichiometry of the nicotine material can vary. For example, the nicotine: coformer stoichiometry can range from about 5:1 to about 1:5 nicotine:coformer, including all 0.1 ratio values and ranges therebetween. Where more than one coformer is used to form a nicotine salt, co-crystal, or salt co-crystal, the ratios of the coformers with respect to both the nicotine and to one another can also vary. A nicotine material may have substantially a single stoichiometry.

The nicotine materials can exist in various polymorphic and pseudopolymorphic forms. The nicotine materials may comprise materials present in polymorphs exhibiting various Bravais lattice symmetries and corresponding space groups.

Certain coformers as described herein contain one or more chiral center(s), which may be (R) or (S) configuration, or mixture of such coformers may be used. In such cases, where various nicotine sources may be used, various enantiomeric and diastereomeric nicotine materials may be provided according to the present disclosure. In various examples, the nicotine materials include such enantiomers and diastereomers, either individually, or admixed in any proportions. Certain coformers as described herein may be geometric isomers, including, but not limited to, cis and trans isomers across a double bond. The nicotine materials may be provided in the form of pure geometric isomers or various geometric isomers in admixture with other geometric isomers.

Coformers may be selected (e.g., coformers disclosed herein) in order to improve nicotine's properties, such as nicotine's melting point, vapor phase stability, solid state stability, UV sensitivity, moisture and/or air sensitivity, temperature sensitivity, and dissolution rate. By improving these properties, solid-state nicotine materials that offer desirable shelf stability, improved vaporization, sublimation, dissolution properties, or a combination thereof can be created.

The nicotine material may be tunable to a desired melting point, vaporization phase stability, dissolution rate, or a combination thereof dependent upon the coformer(s) used. The resulting nicotine material that is formed may exhibit improved properties over pure nicotine, as well as over forms other than the nicotine materials.

Nicotine is known to also have a bitter taste. To at least partially or completely address this, nicotine materials may be selectively created, such as, for example, salts and/or co-crystals, which possess a unique and desirable flavor profile. This may be done by identifying a molecule or molecules that possess(es) desirable flavoring profile(s) and using the molecule(s) as the coformer(s).

A nicotine material (e.g., a specific nicotine co-crystal or a specific nicotine salt) may exhibit one or more specific x-ray-crystallographic feature(s). The form of the nicotine materials may be characterized by an x-ray diffraction pattern (e.g., a single crystal or powder diffraction pattern) having one or more peak(s) at one or more (including all) of the 2-theta diffraction peak(s) for a nicotine material. For example, a specific nicotine co-crystal or a specific nicotine salt exhibits one or more specific single-crystal and/or powder diffraction 2-theta diffraction peak(s). In various examples, the form of a nicotine material is characterized by 1-10 (e.g., 1, 2, 3, 4, 5, 6, 7, 8, 9, or 10) of the 10 highest intensity 2-theta peaks in the x-ray diffraction pattern. The powder pattern may be a measured or simulated (from single-crystal x-ray diffraction data).

A person skilled in the art of X-ray powder diffraction is able to judge the substantial identity of X-ray powder diffraction patterns. Generally, a measurement error of a diffraction angle in an X-ray powder diffractogram is about 2-theta=0.5° or less (more suitably, about 2-theta=0.2° or less) and such degree of a measurement error should be taken into account when considering the X-ray powder diffraction pattern in the figures provided herewith and/or the peak values provided herein. In various examples, the 2-theta peaks (e.g., the 2-theta peaks in the figures) include values +/−0.5° or +/−0.2° from the specific peak value.

A composition may further comprise various other substances. Non-limiting examples of other substances include excipients, such as, for example, fillers or carriers for active ingredients (e.g., calcium polycarbophil, microcrystalline cellulose, hydroxypropylcellulose, sodium carboxymethylcellulose, cornstarch, silicon dioxide, calcium carbonate, lactose, and starches including potato starch, maize starch, and the like, and combinations thereof), thickeners, film formers and binders (e.g., hydroxypropyl cellulose, hydroxypropyl methylcellulose, acacia, sodium alginate, gum arabic, lecithin, xanthan gum and gelatin, and the like, and combinations thereof), antiadherents (e.g., talc, and the like), glidants (e.g., colloidal silica and the like), humectants (e.g., glycerin, and the like), preservatives and antioxidants (e.g., sodium benzoate, ascorbyl palmitate, and the like, and combinations thereof), surfactants (e.g., polysorbate 80, and the like), dyes or pigments (e.g., titanium dioxide, D&C Yellow No. 10, and the like, and combinations thereof), and lubricants or processing aids (e.g., calcium stearate, magnesium stearate, and the like, and combinations thereof), and the like. A composition may comprise nicotine that is present in a form other than a nicotine material of the present disclosure.

A composition may be a pharmaceutical composition. The compositions described herein may include one or more standard pharmaceutically acceptable carrier(s). Pharmaceutically acceptable carriers may be determined in part by the particular composition being administered, as well as by the particular method used to administer the composition. Accordingly, there are a wide variety of suitable formulations of pharmaceutical compositions of the present disclosure. The compounds may be freely suspended in a pharmaceutically acceptable carrier or the compounds may be encapsulated in liposomes and then suspended in a pharmaceutically acceptable carrier. Examples of carriers include, but are not limited to, solutions, suspensions, emulsions, solid injectable compositions that are dissolved or suspended in a solvent before use, and the like. The injections may be prepared by dissolving, suspending or emulsifying one or more of the active ingredient(s) in a diluent. Examples of diluents, include, but are not limited to distilled water for injection, physiological saline, vegetable oil, alcohol, dimethyl sulfoxide, and a combination thereof. Further, the injections may contain stabilizers, solubilizers, suspending agents, emulsifiers, soothing agents, buffers, preservatives, etc. The injections may be sterilized in the final formulation step or prepared by sterile procedure. The composition of the disclosure may also be formulated into a sterile solid preparation, for example, by freeze-drying, and can be used after sterilized or dissolved in sterile injectable water or other sterile diluent(s) immediately before use. Additional examples of pharmaceutically include, but are not limited to, sugars, such as lactose, glucose, and sucrose; starches, such as corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2005) 21st Edition, Philadelphia, Pa. Lippincott Williams & Wilkins. Effective formulations include, but are not limited to, oral and nasal formulations, formulations for parenteral administration, and compositions formulated for with extended release. Parenteral administration includes infusions such as, for example, intramuscular, intravenous, intraarterial, intraperitoneal, subcutaneous administration, and the like.

A nicotine material may be incorporated in a known pharmaceutical product or tobacco product. In various examples, a nicotine material or composition is used as a replacement for, or in addition to, the nicotine in nicotine-containing pharmaceutical products or tobacco products.

In an aspect, the present discloses articles of manufacture. An article of manufacture may comprise one or more nicotine material(s) and/or one or more composition(s).

In various examples, the article of manufacture is a transdermal delivery device. A transdermal delivery device may be referred to as a transdermal nicotine patch or nicotine patch. Non-limiting examples of transdermal delivery devices include transdermal nicotine patches, nicotine patches, and the like. In various examples, an article of manufacture is an oral delivery device. Non-limiting examples of oral delivery devices include pills, capsules, and the like. In various examples, an article of manufacture is a solid state vaporization tablet or capsule, a dissolvable formulation tablet, or the like.

In an aspect, the present disclosure provide methods of using the nicotine materials. The nicotine material may be used in various methods. In various examples, one or more nicotine material(s) and/or one or more composition(s) are used in nicotine storage methods, in nicotine delivery methods (e.g., in nicotine delivery methods, such as, for example, vaping methods, nicotine-based treatment methods, in nicotine addiction treatment methods, and the like), or in nicotine product formulation, or the like. Non-limiting examples of uses of nicotine materials and compositions comprising nicotine materials of the present disclosure are provided herein.

In various examples, a method of forming vapor phase or aerosol phase nicotine comprises: vaporizing (e.g., thermally vaporizing) or aerosolizing one or more nicotine material(s), such that the vapor phase or aerosol phase of the nicotine is formed. The nicotine material may be heated using a resistive heater. The vaporizing may be carried out in the absence of a liquid carrier (e.g., PEG, liquid acid(s), glycerin, and the like, and combinations thereof). The vaporizing may be carried out at a temperature of 100 to 350° C., including all 0.1° C. values and ranges therebetween. The vaporizing or aerosolizing may be carried out in a device (which may be an electronic device). In a method, 90 to 100% (e.g., 90% or more, 95% or more, 99% or more, or 100%) of the nicotine of the nicotine material may be vaporized or aerosolized (e.g., as nicotine, as one or more nicotine(s) or coformer degradation product(s), as one or more nicotine material(s) (e.g., salt or co-crystal(s)) or a combination thereof).

Nicotine and/or one or more nicotine degradation product(s) may be delivered to an individual (e.g., a human or a non-human animal) using a nicotine material, composition, or article of manufacture of the present disclosure. A method of delivering nicotine or a nicotine degradation product to an individual may comprise administration of a nicotine material, a composition, or an article of manufacture of the present disclosure to an individual. The individual may be in need of treatment as described herein.

The nicotine materials and/or compositions of present disclosure may be administered to the subject in a variety of ways including but not limited to injection by the intravenous, intraarterial, intraperitoneal, intramuscular, intradermal, intrapulmonary, intranasal or oral, sublingual, dermal, subcutaneous routes, or any other route.

The nicotine materials and/or compositions may be administered as a single administration or as multiple administrations or may be introduced in a continuous manner over a period of time. For example, the administration(s) can be a pre-specified number of administrations or daily, weekly or monthly administrations, which may be continuous or intermittent, as may be clinically needed and/or therapeutically indicated.

A pharmaceutical composition comprising one or more nicotine product(s) and/or one or more composition(s) of the present disclosure may be used for treatment of a wide variety of conditions, diseases, and disorders responsive to stimulation of one or more type(s) of nicotinic acetylcholinergic receptors (nAChRs). The pharmaceutical compositions may be used to treat those types of conditions, diseases, and disorders that have been reported to be treatable through the use or administration of nicotine as an agonist of nAChRs (e.g., various CNS conditions, diseases, and disorders. Non-limiting examples of diseases or disorders that can be treated include cognitive disorders, such as, for example, Alzheimer's disease and attention deficit disorder, schizophrenia, Parkinson's disease, Tourette's syndrome, ulcerative colitis, dry eye disease, hypertension, depression, overactive bladder, obesity, seven year itch/scabies, hemorrhoids, and the like. A pharmaceutical composition comprising one or more nicotine product(s) and/or one or more composition(s) of the present disclosure may be used for treatment to reduce stress or pain and/or as a smoking cessation aid.

The combined amount of nicotine present (including nicotine present as the nicotine material(s) and, optionally, any one or more other form(s) of nicotine may be the amount effective to treat some symptoms of, or prevent occurrence of the symptoms of, a condition, disease, or disorder from which the subject or patient suffers.

In an aspect, the present disclosure provide kits. A kit may comprise pharmaceutical preparations containing one or more nicotine material(s) and/or one or more nicotine composition of the present disclosure(s). In various examples, a kit comprises a package (e.g., a closed or sealed package) that contains one or more nicotine material(s) and/or one or more nicotine composition(s), such as, for example, one or more closed or sealed vial(s), bottle(s), blister (bubble) pack(s), or any other suitable packaging for the sale, distribution, or use of the nicotine compounds and compositions comprising them.

In various examples, the printed material includes, but not limited to, printed information. The printed information may be provided on a label, or on a paper insert, or printed on the packaging material itself. The printed information may include information that, for example, identifies the composition in the package, the amounts and types of other active and/or inactive ingredients, and instructions for taking the composition, such as, for example, the number of doses to take over a given period of time, and/or information directed to a pharmacist and/or another health care provider, such as a physician, or a patient. The printed material may include, for example, an indication that the nicotine material and/or any other agent provided with it is for treatment of an individual with a condition, disease, or disorders described herein. In an example, the product includes a label describing the contents of the container and providing indications and/or instructions regarding use of the contents of the container to treat an individual with a disease characterized by stimulation of one or more types of nAChR(s) or use as an agonist of nAChRs.

The steps of the method described in the various examples disclosed herein are sufficient to carry out the methods of the present disclosure. Thus, in an example, a method consists essentially of a combination of steps of the methods disclosed herein. In another example, a method consists of such steps.

The following Statements describe examples of nicotine materials, compositions comprising one or more nicotine material(s), articles of manufacture comprising nicotine material(s) and/or compositions comprising one or more nicotine material(s), and uses of the nicotine materials, the compositions, and the articles of manufacture.

Statement 1. A method of making a nicotine material of the present disclosure, which may be a method comprising: providing a nicotine material-forming solution comprising nicotine, a coformer, which may be a combination of two or more coformers and one or more of which may be solvent(s), and, optionally, a solvent, which may be a mixture of solvents; and evaporating, which may be carried out without adding seed crystals to the nicotine material-forming solution, at least a portion (e.g., substantially all or all) of the solvent(s), which may be one or more coformer(s), from the nicotine material-forming solution, where the nicotine material, which may be a salt or a mixture of salts, a co-crystal or a mixture of co-crystals, or a combination thereof, comprising one or more polymorphs, one or more phases, and the like, or a combination thereof and/or may be formed by spontaneous nucleation, crystallization, or precipitation, is formed). The method (or at least the evaporating) may be carried out in the dark (e.g., in the absence of visible light wavelengths (e.g., 400-700 nm) or by blocking 90% or more, 95% or more, 99% or more of the visible light wavelengths of ambient visible light (e.g., sunlight, incandescent light(s), fluorescent light(s), or a combination thereof).
Statement 2. A method according to Statement 1, further comprising isolating the nicotine material. E.g., at least a portion of, substantially all, or all of the nicotine material is collected by filtration, which may be vacuum filtration.
Statement 3. A method according to Statement 1 or 2, where further comprising rinsing the nicotine material. E.g., contacting the nicotine material with a solvent, which may be a mixture of solvents, and, optionally, isolating the rinsed nicotine material from the rinsing solvent(s). The rinsing may remove at least a portion of or all of the solvent(s) used in the nicotine material-forming reaction.
Statement 4. A method according to Statement 3, where the nicotine material is rinsed (e.g., washed) with a solvent chosen from hydrocarbon solvents (e.g., alkanes such as, for example, n-heptane, hexane, pentane and the like), alcohols (such as, for example, methanol, ethanol, butanol, and the like), ketone solvents (e.g., acetone, butanone, and the like), ester solvents (e.g., ethyl acetate, ethyl lactate, triacetin, and the like), halogenated solvents (e.g., halogenated alkenes, such as, for example, dichloromethane, chloromethane, chloroform, carbon tetrachloride, and the like), and the like, and combinations thereof.
Statement 5. A method according to any one of the preceding Statements, further comprising drying the nicotine material (e.g., the unrinsed or rinsed nicotine material).
Statement 6. A method according to Statement 5, where the drying is carried out under vacuum (e.g., 0.01 to 800 torr, including all 0.01 torr values and ranges therebetween) and/or at a temperature below the melting and/or decomposition temperature of the nicotine material (e.g., 0 to 100° C., including all 0.1° C. values and ranges therebetween).
Statement 7. A method according to any one of the preceding Statements, where the solvent of the nicotine material-forming solution is chosen from organic solvents, water, and combinations thereof. A solvent may be a coformer or solvent(s) may be coformer(s). E.g., water is present in the nicotine material-forming solution at 5% by weight or less (e.g., 5 to 0.001% by weight, including all 0.001% by weight values and ranges therebetween) based on the total weight of the nicotine material-forming solution.
Statement 8. A method according to Statement 7, where the organic solvents are chosen from alcohols (e.g., C₁ to C₄ alcohols, such as, for example, methanol, ethanol, propanols, and butanols, and the like), cyclic ethers (e.g., tetrahydrofuran, tetrahydropyran, ethylene oxide, furans, and the like), polar protic solvents (e.g., formic acid, ammonia, alcohols, and the like), polar aprotic solvents (e.g., dimethylformamide, dimethyl sulfoxide, acetonitrile, ethyl acetate, and the like), halogenated alkanes (e.g., C₁ to C₆ halogenated alkanes comprising 1, 2, 3, 4, 5, 6, 7, 8, 9, 10, 11, 12, 13, or 14 halogens (Cl, Br, I, F, or a combination thereof)), such as, for example, chloroform, methylene chloride, chloromethane, tetrafluoroethane, carbon tetrachloride, and the like), which may be perhalogenated alkenes, halogenated aryl solvents (e.g., halogenated benzenes and benzene derivatives comprising 1, 2, 3, 4, 5, or 6 halogens (—Cl, —Br, —I, —F, or a combination thereof), such as, for example, chlorobenzene, dichlorobenzene, trifluorotoluene, and the like, and the like, and combinations thereof. One or more of the solvent(s) may also be a coformer, e.g., as the coformer and solvent (which may provide a solvate material). Non-limiting examples of such solvents include alcohols, such as, for example, methanol, ethanol, tert-butanol, and the like, hydrocarbon solvents, such as, for example, n-heptane, hexane, pentane and the like, ketone solvents, such as, for example, isophorone, acetone, butanone, and the like, ester solvents, such as, for example, ethyl acetate, ethyl lactate, triacetin, and the like, halogenated solvents, such as, for example, dichloromethane, chloromethane, chloroform and the like, and the like.
Statement 9. A method according to any one of the preceding Statements, where the nicotine is S-nicotine, R-nicotine, or a combination thereof (e.g., an enantiomerically high form or pure form of nicotine or racemic mixture thereof).
Statement 10. A method according to any one of the preceding Statements, where the nicotine is present in the nicotine material-forming solution at its solubility limit in the solvent(s) or at 90% or more or 95% or more of its solubility limit in the solvent(s) or at a concentration of at least 0.01 M (e.g., 0.01 to 2 M).
Statement 11. A method according to any one of the preceding Statements, where the coformer(s) is/are chosen from organic compounds, mineral acids (e.g., sulfuric acid, hydrochloric acid, nitric acid, boric acid, hydrofluoric acid, hydrobromic acid, phosphoric acid, hydroiodic acid, perchloric acid, and the like, and combinations thereof), and combinations thereof. The organic compound(s) may be acidic organic compounds (e.g., organic compounds that can undergo proton transfer with nicotine).
A coformer may form a nicotine material and/or may provide additional functionality (e.g., provide a nicotine material with one or more of a desirable melting point, vapor point, nicotine material vaporization component(s) (e.g., nicotine and/or coformer(s) vaporization product(s)).
Statement 12. A method according to Statement 11, where the organic compounds are chosen from carboxylic acids (e.g., C₁ to C₆ carboxylic acids, which may be aliphatic carboxylic acids or aryl carboxylic acids, (such as, for example, benzoic acid, orotic acid, vanillic acid, succinic acid, aminosalicylic acid, cinnamic acid and the like), hydroxycarboxylic acids, which may be aliphatic carboxylic acids or aryl carboxylic acids, (such as, for example, 2,6-dihydroxybenzoic acid, 2,4,6-trihydroxybenzoic acid, gallic acid, malic acid, salicylic acid, dihydroxyterephthalic acid, vanillylmandelic acid, galactaric acid, galacturonic acid, lactic acid, galactonic acid, tartronic acid, and the like), alcohols (such as, for example, menthol, phenol, cannabidiol, raspberry ketone/frambinone, and the like), polyacids (e.g., aliphatic and aryl polyacids, such as, for example, polyacrylic acid, polysialic acid, polyglutamic acid, metaphosphoric acid, and the like), sweetening agents (such as, for example, aspartame, saccharin, steviol, sucralose, sucrose, erythritol, xylitol, sorbitol, maltitol, and the like), amino acids (such as, for example, aspartic acid, glutamic acid, serine, proline, cysteine, histidine, isoleucine, leucine, lysine, methionine, phenylalanine, threonine, tryptophan, valine, carnitine, betaine, and the like), and the like, and combinations thereof. Non-limiting examples of organic compounds are GRAS (Generally Stared as Safe) compounds (examples of which may be found at the FDA website—https://www.fda.gov/food/food-ingredients-packaging/generally-recognized-safe-gras), the Canadian database of food constituents (FooDB), and Complex System's Lab's (CoSyLab's) comprehensive database of flavor molecules (FlavorDB). The individual organic compounds(s) may be one or more particular stereoisomer(s) or a combination of two or more stereoisomers.
Statement 13. A method according to any one of the preceding Statements, where the coformer(s) is/are present in the nicotine material-forming solution at 0.01 to 2 M (e.g., 0.1 to 1 M), including all 0.01 M values and ranges therebetween.
Statement 14. A method according to anyone of the preceding Statements, where the nicotine material is (e.g., individual nicotine material solid particles are) amorphous, polycrystalline, single crystalline, or a combination thereof.
Statement 15. A method according to anyone of the preceding Statements, where at least a portion of or all of the nicotine material exhibits orthorhombic symmetry (which may correspond to a space group, such as, for example, a P222, P222₁, P2₁2₁2, P2₁2₁2₁, C222₁, C222, F222, I222, or I2₁2₁2₁ space group), monoclinic symmetry (which may correspond to a space group, such as for example, a P2₁, P2, or C2 space group), hexagonal symmetry (which may correspond to a space group, such as, for example, a P6, P6₁, P6₂, P6₃, P6₄, P6₅, P622, P6₁22, P6₂22, P6₃22, P6₄22, or P6₅22 space group), trigonal symmetry (which may correspond to a space group, such as, for example, a P3, P3₁, P3₂, R3, P312, P321, P3₁12, P3₁21, P3₂12, P3₂21, or R32 space group), triclinic symmetry (which may correspond to a space group, such as, for example, a P1 space group), tetragonal symmetry (which may correspond to a space group such as, for example, a P4, P4₁, P4₂, P4₃, I4, I4₁, P 422, P42₁2, P4₁22, P4₁2₁2, P4₂22, P4₂2₁2, P4₃22, P4₃2₁2, I422, or I4₁22 space group), or cubic symmetry (which may correspond to a space group such as, for example, aP23, F23, I23, P2₁3, I2₁3, P432, P4₂32, F432, F4₁32, I432, P4₃32, P4₁32, I4₁32 space group).
Statement 16. A method according to any one of the preceding Statements, further comprising forming the nicotine material into a tablet or forming a liquid comprising the nicotine material.
Statement 17. A nicotine material comprising nicotine and one or more coformer. The nicotine materials may be crystalline nicotine materials. E.g., a nicotine material comprising nicotine (e.g., S-nicotine, R-nicotine, S-nicotinium, R-nicotinium, or a combination thereof, such as, for example, a racemic mixture thereof) and 1, 2, 3, 4, 6, or 7 structurally distinct coformers. E.g., the nicotine material is not a solvate crystal.
Statement 18. A nicotine material according to Statement 17, where the nicotine material is (e.g., individual nicotine material solid particles are) amorphous, polycrystalline, single crystalline, or a combination thereof.
Statement 19. A nicotine material according to Statement 17 or 18, where the coformer is chosen from organic compounds, mineral acids (e.g., sulfuric acid, hydrochloric acid, nitric acid, boric acid, hydrofluoric acid, hydrobromic acid, phosphoric acid, hydroiodic acid, perchloric acid, and the like, and combinations thereof), and combinations thereof. The organic compound(s) may be acidic organic compounds (e.g., organic compounds that can undergo proton transfer with nicotine). A coformer may form a nicotine material and/or may provide additional functionality (e.g., provide a nicotine material with one or more of a desirable melting point, vapor point, nicotine material vaporization component(s) (e.g., nicotine and/or coformer(s) vaporization product(s))
Statement 20. A nicotine material according to Statement 19, where the organic compounds are chosen from carboxylic acids (e.g., C₁ to C₆ carboxylic acids, which may be aliphatic carboxylic acids or aryl carboxylic acids, (such as, for example, benzoic acid, orotic acid, vanillic acid, succinic acid, aminosalicylic acid, cinnamic acid and the like), hydroxycarboxylic acids, which may be aliphatic carboxylic acids or aryl carboxylic acids, (such as, for example, 2,6-dihydroxybenzoic acid, 2,4,6-trihydroxybenzoic acid, gallic acid, malic acid, salicylic acid, dihydroxyterephthalic acid, vanillylmandelic acid, galactaric acid, galacturonic acid, lactic acid, galactonic acid, tartronic acid, and the like), alcohols (such as, for example, menthol, phenol, cannabidiol, raspberry ketone/frambinone, and the like), polyacids (e.g., aliphatic and aryl polyacids, such as, for example, polyacrylic acid, polysialic acid, polyglutamic acid, metaphosphoric acid, and the like), sweetening agents (such as, for example, aspartame, saccharin, steviol, sucralose, sucrose, erythritol, xylitol, sorbitol, maltitol, and the like), amino acids (such as, for example, aspartic acid, glutamic acid, serine, proline, cysteine, histidine, isoleucine, leucine, lysine, methionine, phenylalanine, threonine, tryptophan, valine, carnitine, betaine, and the like), and the like, and combinations thereof. Non-limiting examples of organic compounds are GRAS (Generally Stared as Safe) compounds (examples of which may be found at the FDA website—https://www.fda.gov/food/food-ingredients-packaging/generally-recognized-safe-gras), the Canadian database of food constituents (FooDB), and Complex System's Lab's (CoSyLab's) comprehensive database of flavor molecules (FlavorDB). The individual organic compounds(s) may be one or more particular stereoisomer(s) or a combination of two or more stereoisomers.
Statement 21. A nicotine material according to any one of Statements 17-20, where at least a portion of or all of the nicotine material exhibits orthorhombic symmetry (which may correspond to a space group, such as, for example, a P222, P222₁, P2₁2₁2, P2₁2₁2₁, C222₁, C222, F222, I222, or I2₁2₁2₁ space group), monoclinic symmetry (which may correspond to a space group, such as for example, a P2₁, P2, or C2 space group), hexagonal symmetry (which may correspond to a space group, such as, for example, a P6, P6₁, P6₂, P6₃, P6₄, P6₅, P622, P6₁22, P6₂22, P6₃22, P6₄22, or P6₅22 space group), trigonal symmetry (which may correspond to a space group, such as, for example, a P3, P3₁, P3₂, R3, P312, P321, P3₁12, P3₁21, P3₂12, P3₂21, or R32 space group), triclinic symmetry (which may correspond to a space group, such as, for example, a P1 space group), tetragonal symmetry (which may correspond to a space group such as, for example, a P4, P4₁, P4₂, P4₃, I4, I4₁, P 422, P42₁2, P4₁22, P4₁2₁2, P4₂22, P4₂2₁2, P4₃22, P4₃2₁2, I422, or I4₁22 space group), or cubic symmetry (which may correspond to a space group such as, for example, aP23, F23, I23, P2₁3, I2₁3, P432, P4₂32, F432, F4₁32, I432, P4₃32, P4₁32, I4₁32 space group).
Statement 22. A nicotine material according to Statement 17-21, where the ratio of nicotine molecules to coformer molecule(s) is 1:5 to 5:1, including all 0.1 ratio values and ranges therebetween, e.g. 1:2, 2:1, or 1:1.
Statement 23. A nicotine material according to any one of Statements 17-22, where the coformer is chosen from L-malic acid, D-malic acid, DL-malic acid, and 2,6-dihydroxybenzoic acid and at least a portion of or all of the nicotine material is a certain polymorph (e.g., monoclinic P2₁ (S)-nicotinium L-malate, orthorhombic P2₁2₁2₁ (S)-nicotinium D-malate, orthorhombic P2₁2₁2₁ (S)-nicotinium DL-malate, monoclinic P2₁ (S)-nicotinium 2,6-dihydroxybenzoate).
Statement 24. A nicotine material according to any one of Statements 17-22, where the coformer is chosen from L-malic acid, D-malic acid, DL-malic acid, and 2,6-dihydroxybenzoic acid and at least a portion of or all of the nicotine material may exhibit an orthorhombic structure, a monoclinic structure, or the like, or a combination thereof.
Statement 25. A nicotine material according to any one of Statements 17-24, where the nicotine material exhibits substantially no degradation (e.g., 5% by weight or less, 2% by weight or less, 1% by weight or less, 0.1% by weight or less, or 0.01% by weight or less of the nicotine is degraded, for example, as determined by one or more spectroscopic and/or spectrometric methods) after 6 hours, 12 hours, or 24 hours under the UV photodegradation conditions described herein.
Statement 26. A nicotine material according to any one of Statements 17-25, where the nicotine material exhibits a desired melting point, vaporization phase stability, dissolution rate, or a combination thereof dependent upon the coformer(s) present in the material. The resulting nicotine material that is formed may exhibit one or more improved properties over pure nicotine or other forms of nicotine.
Statement 27. A composition comprising one or more nicotine material(s) of the present disclosure (e.g., one or more nicotine material(s) of Statement 17-26, one or more nicotine material(s) made by a method of Statement 1-16, or a combination thereof) and one or more additive(s). E.g., the composition is an edible composition.
Statement 28. A composition according to Statement 27, where the additive(s) is/are chosen from flavoring agents, excipients, sweeteners, binding agents, and the like, and combinations thereof.
Statement 29. A composition according to Statement 27 or 28, where the composition is in a solid form (e.g., a powder, a tablet, lozenge, film, strip, or the like), a liquid form (e.g., in nicotine delivery liquid (e.g., PEG, liquid acid)), a solution, for example, with a pharmaceutically acceptable carrier, or the like), or a vapor or aerosol form. E.g., a composition is a vaping composition (which may further comprise a carrier, such as, for example, glycerin, water, and a flavorant). E.g., a composition is a smokeless tobacco product (e.g., loose moist snuff, loose dry snuff, chewing tobacco, pelletized tobacco pieces; extruded or formed tobacco strips, pieces, rods, cylinders or sticks, finely divided ground powders; finely divided or milled agglomerates of powdered pieces and components; flake-like pieces; molded tobacco pieces; gums; rolls of tape-like films; readily water-dissolvable or water-dispersible films or strips, meltable compositions, lozenges, pastilles, and the like. E.g., a composition is a pharmaceutical product (e.g., a pill, tablet, lozenge, capsule, caplet, pouch, gum, inhaler, solution, cream or the like).
Statement 30. An article of manufacture comprising one or more nicotine material(s) of the present disclosure (e.g., one or more nicotine material(s) according to any one of Statements 17-26, one or more nicotine material(s) made by a method according to any one of Statements 1-16, or a combination thereof).
Statement 31. An article of manufacture according to Statement 30, where the article of manufacture is a transdermal delivery device (which may be referred to as a transdermal nicotine patch or nicotine patch), an oral delivery device (such as a pill, capsule or the like), a solid state vaporization tablet or capsule, a dissolvable formulation tablet, or the like.
Statement 32. A method of forming vapor phase or aerosol phase nicotine comprising: vaporizing (e.g., thermally vaporizing) or aerosolizing one or more nicotine material of the present disclosure (e.g., one or more nicotine material(s) according to any one of Statements 17-26, one or more nicotine material(s) made by a method according to any one of Statements 1-16, or a combination thereof), such that the vapor phase or aerosol phase of the nicotine is formed. E.g., the nicotine material is heated using a resistive heater. E.g., the vaporizing is carried out in the absence of a liquid carrier (e.g., PEG, liquid acid(s), glycerin and the like).
Statement 33. A method of forming vapor phase or aerosol phase nicotine according to Statement 32, where the vaporizing is carried out at a temperature of 100 to 350° C., including all 0.1° C. values and ranges therebetween.
Statement 34. A method of forming vapor phase or aerosol phase nicotine according to Statement 32 or 33, where the vaporizing or aerosolizing is carried out in a device (which may be an electronic device).
Statement 35. A method of forming vapor phase or aerosol phase nicotine according to any one of Statements 32-34, where 90 to 100% (e.g., 90% or more, 95% or more, 99% or more, or 100%) of the nicotine of the nicotine material is vaporized or aerosolized (e.g., as nicotine, as one or more nicotine(s) or coformer degradation product(s), as one or more nicotine material(s) (e.g., salt or co-crystal(s)) or a combination thereof).
Statement 36. Use of one or more nicotine material(s) of the present disclosure (e.g., one or more nicotine material(s) according to any one of Statements 17-26, one or more nicotine material made by a method according to any one of Statements 1-16, or a combination thereof): in nicotine storage methods, in nicotine delivery methods (e.g., in nicotine delivery methods, such as, for example, vaping methods, nicotine-based treatment methods, in nicotine addiction treatment methods, and the like) in nicotine product formulation, or the like.

The following example is presented to illustrate the present disclosure. It is not intended to be in any way limiting.

### Example

This example provides a description of nicotine salts of the present disclosure, characterization of same, methods of making same, and uses thereof.

Multiple polymorphs of (S)-nicotinium L-malate were synthesized, along with one morphology of (S)-nicotinium D-malate, as well as (S)-nicotinium DL-malate, (S)-nicotinium salicylate, (S)-nicotinium 2,6-dihydroxybenzoate, and (S)-nicotinium orotate monohydrate. All synthesized salts included (S)-nicotine, the naturally occurring enantiomer, complexed with a generally recognized as safe (GRAS) coformer. GRAS listed coformers are being utilized to help improve the safety of nicotine usage. This is being done to provide a safer alternative, not only to nicotine combustion as a delivery source, but also to previously known heavily halogenated toxic nicotine-coformer salts. Halogenated nicotine salts may have desirable properties but they are not safe for usage as nicotine salts for vaping or nicotine products.

TABLE 1
Safer Potential Coformers for Solid State Nicotine Formulation

| Potential Coformer | pKa (Strongest Acidic) |
|---|---|
| 2,6-Dihydroxybenzoic Acid | 1.51 |
| 2,4,6-Trihydroxybenzoic Acid | 1.83 |
| Vanillylmandelic Acid | 3.11 |
| DL-Malic Acid | 3.20 |
| D-Malic Acid | 3.20 |
| L-Malic Acid | 3.20 |
| Galactaric Acid/Mucic acid | 2.83 |
| Mandelic Acid | 3.75 |
| Gallic Acid | 3.94 |
| 2,5-Dihydroxyterepthalic Acid | 2.75 |
| Galacturonic Acid | 3.24 |
| Menthol | 19.55 |
| Frambinone (Raspberry Ketone) | 9.51 |
| Aminomalonic Acid | 0.45 |
| Proline | 1.99 |
| Serine | 2.21 |
| Glutamic Acid | 1.88 |
| Para-Vanillic Acid | 4.16 |
| Ortho-Vanillic Acid | 2.55 |
| 4-Methoxysalicylic Acid | 3.09 |
| 5-Methoxysalicylic acid | 2.52 |
| 2,4-Dihydroxybenzoic Acid | 3.10 |
| Pyruvic Acid | 2.93 |
| Maleic Acid | 3.05 |
| Caffeine | 10.40 |
| Ortho-Anisic Acid | 3.73 |
| Para-Anisic Acid | 4.37 |
| Meta-Anisic Acid | 3.84 |
| Gentisic Acid | 2.53 |
| Fumaric Acid | 3.55 |
| Benzoic Acid | 4.08 |
| Galactonic Acid | 3.39 |
| Salicylic Acid | 2.79 |
| Aspartame | 3.53 |
| Tartronic Acid | 2.42 |
| Tartaric Acid | 2.72 |
| Succinic Acid | 3.55 |
| 3-Aminosalicylic Acid | 2.90 |
| Lactic Acid | 3.78 |
| Hydroxycaffeic Acid | 3.38 |
| 5,5′-Methylenedisalicylic Acid | 2.94 |
| Caftaric Acid | 2.96 |
| Betaine | 2.26 |
| Carnitine | 4.20 |
| Garcinia Acid (Hydroxycitric Acid) | 2.90 |
| Cannabidiol (CBD) | 9.13 |
| Saccharin | 1.60 |
| Steviol | 4.70 |
| Sucralose | 1.91 |
| Sucrose | 11.84 |
| Erythritol | 13.04 |
| Xylitol | 12.76 |
| Sorbitol | 13.6 |
| α, β, or γ cyclodextrin | 11.5 |
| Maltitol | 12.1 |
| Cysteine | 1.96 |
| Oxalic Acid | 1.27 |
| Aspartic Acid | 1.88 |
| Phenol | 9.88 |
| Cinnamic acid | 4.01 |
| Glutaric acid | 3.76 |
| Citramalic Acid | 3.35 |
| Citric Acid | 3.13 |
| α, β, or γ Hydroxy Acids | N/A |

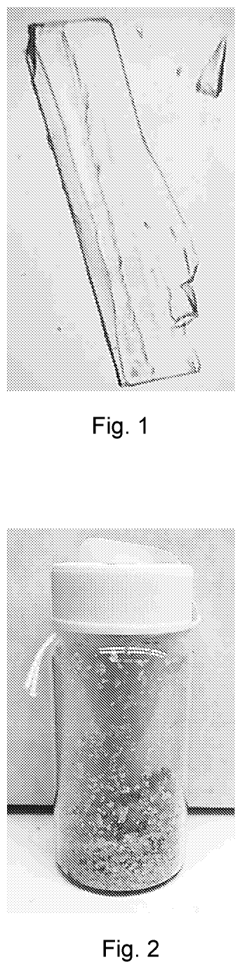

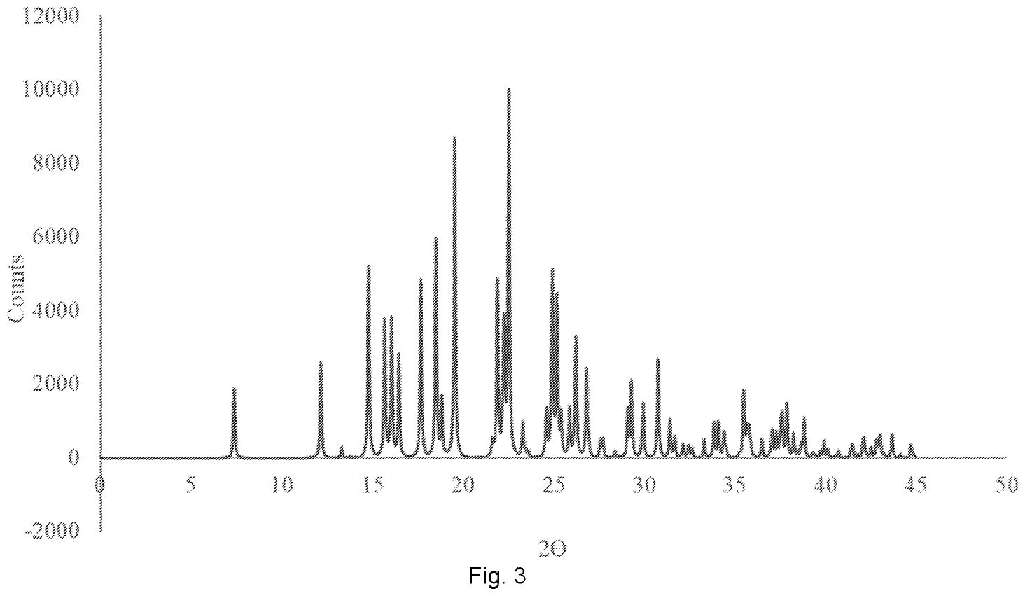

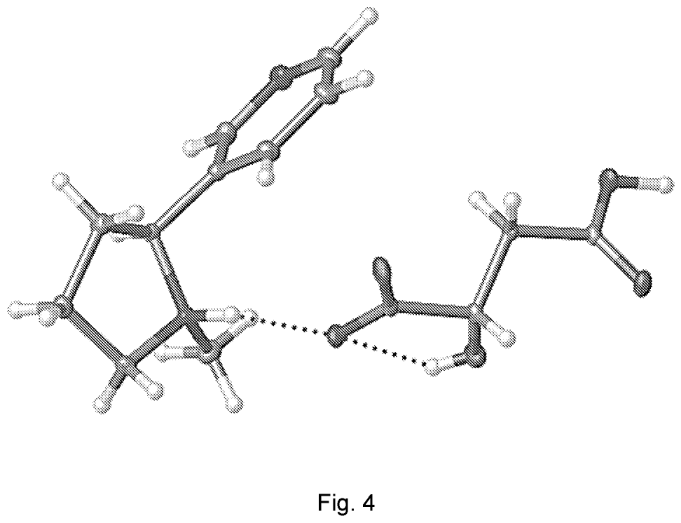

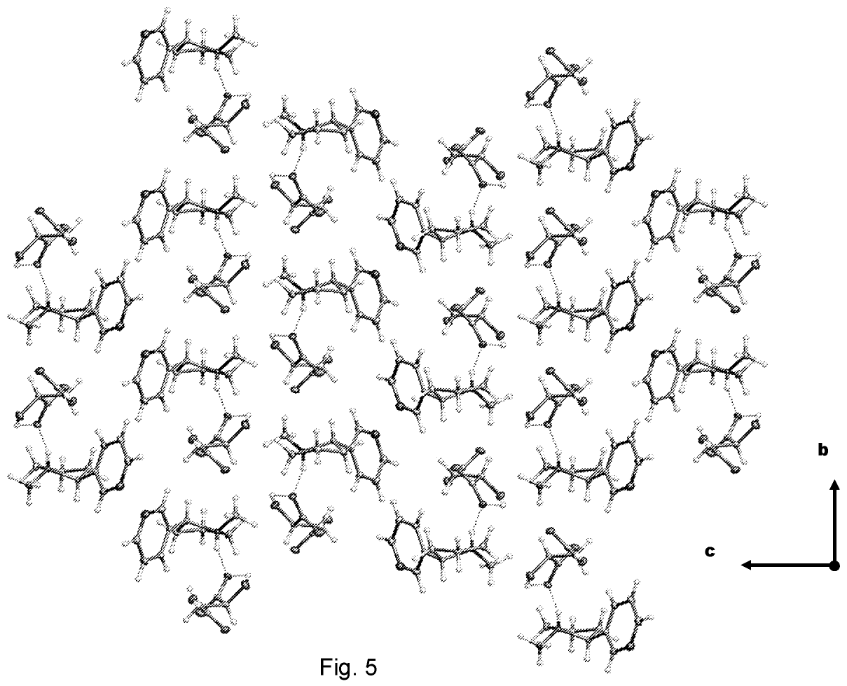

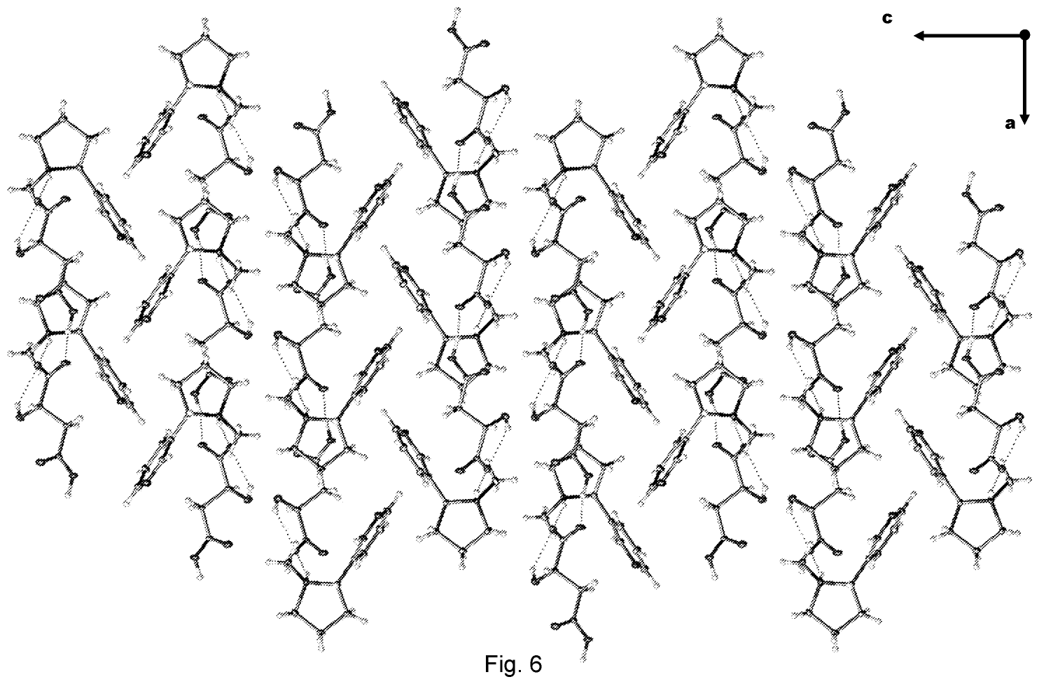

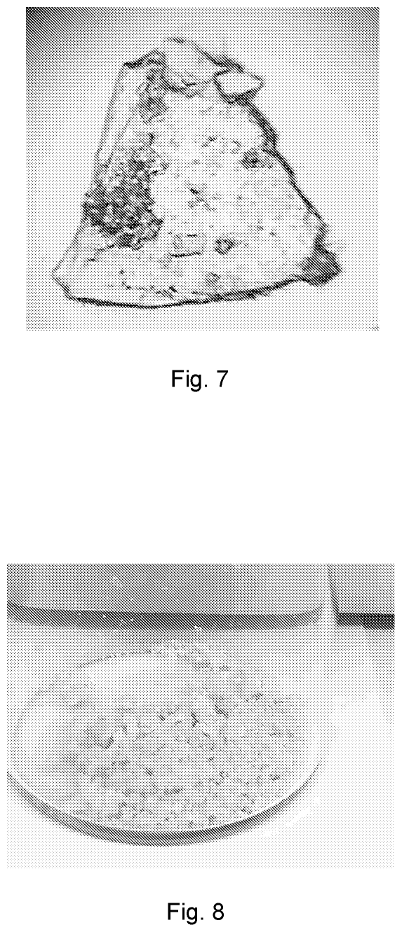

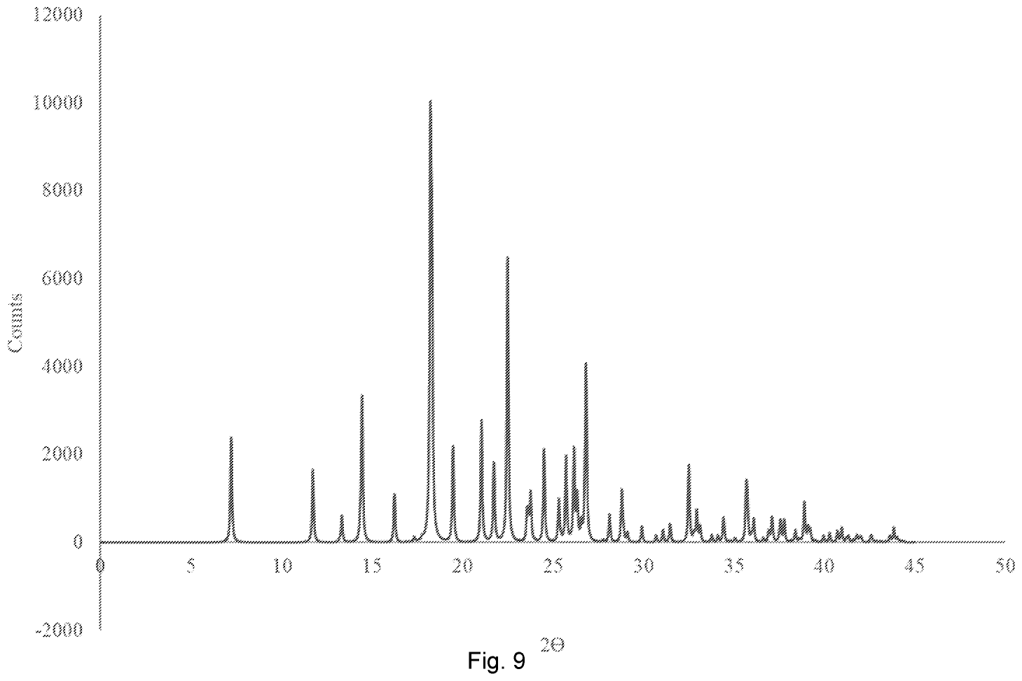

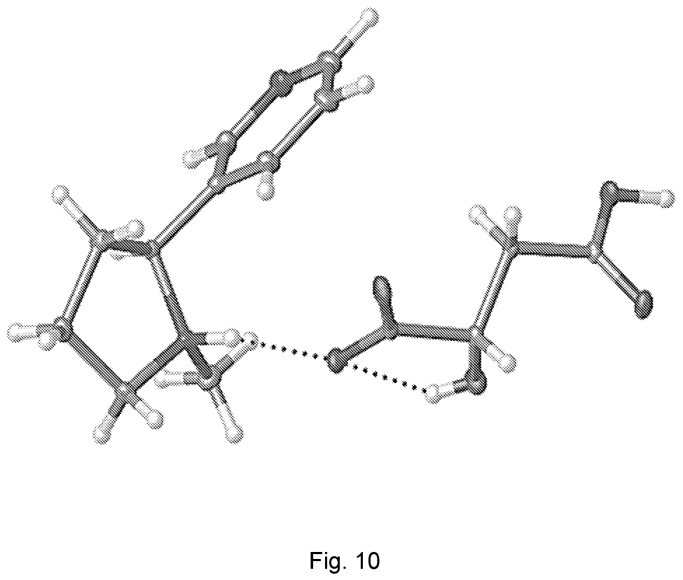

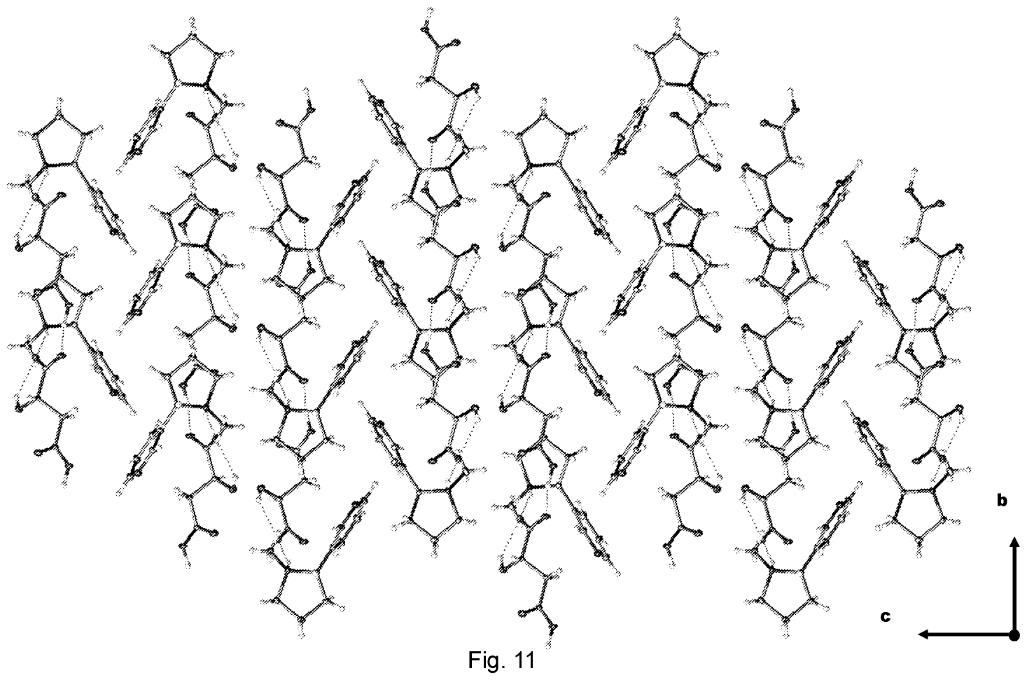

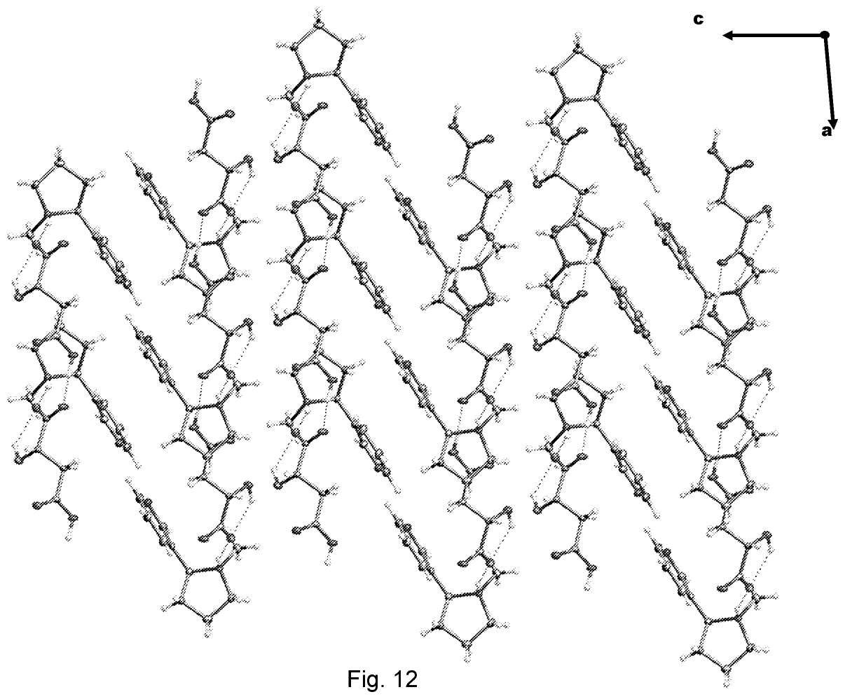

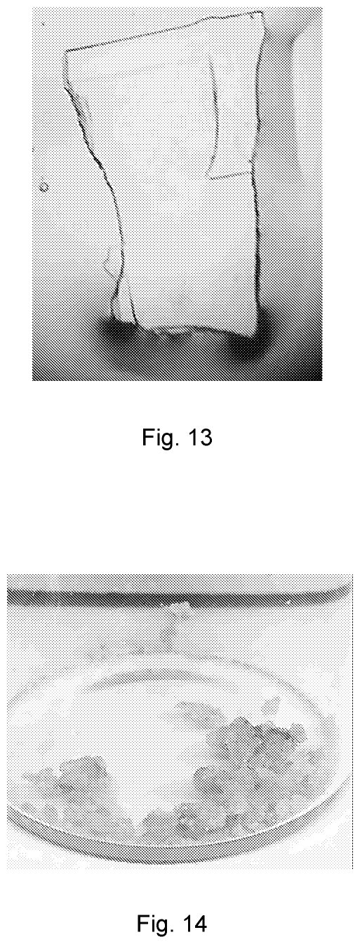

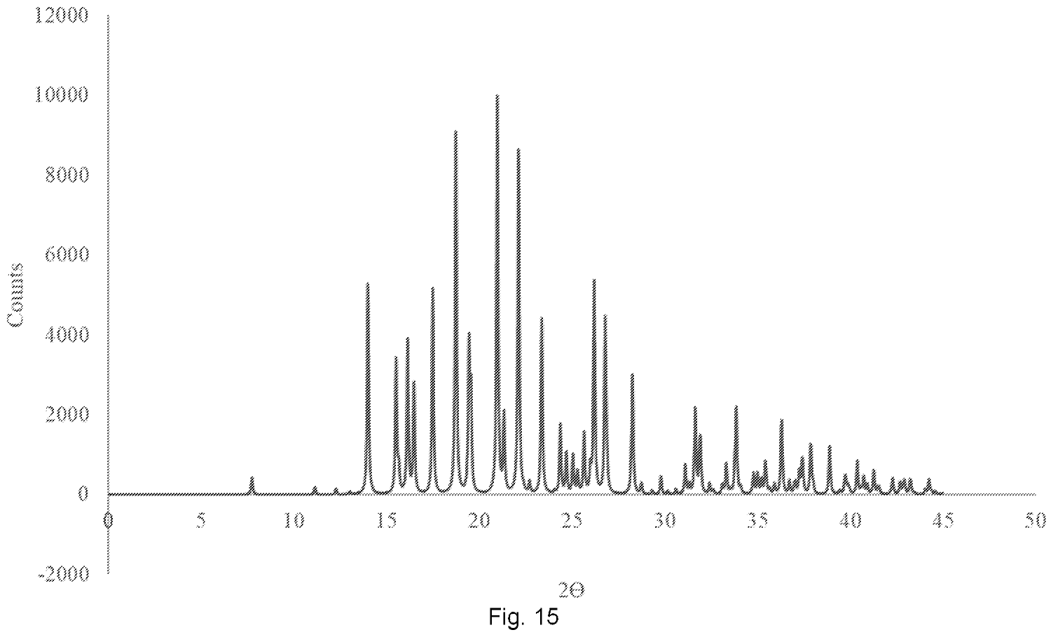

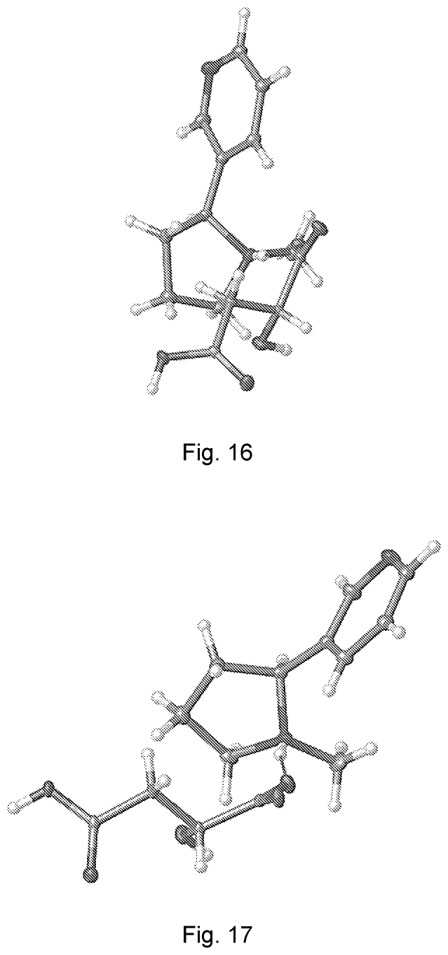

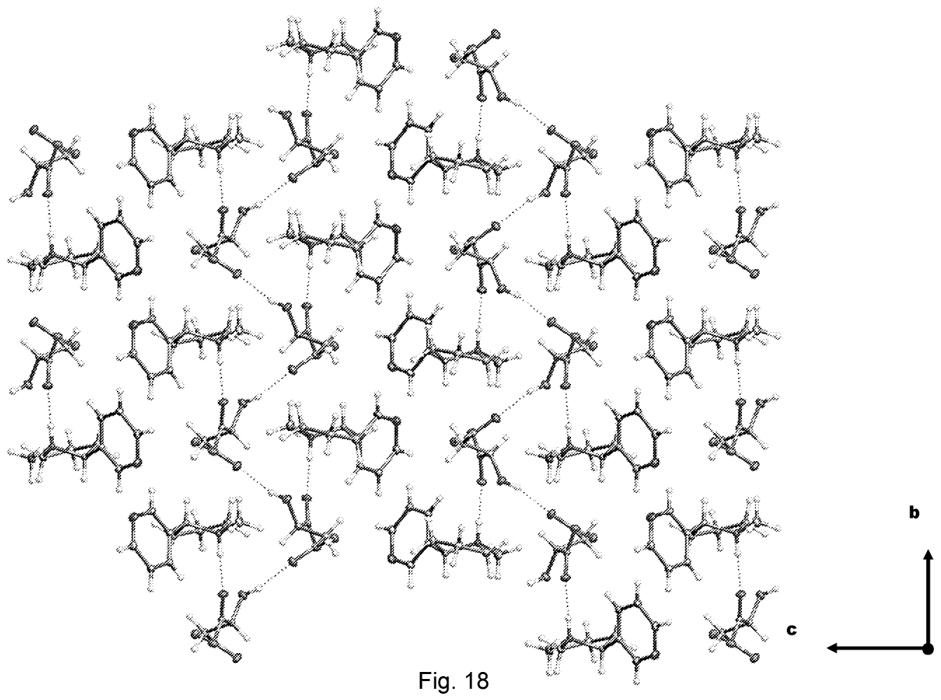

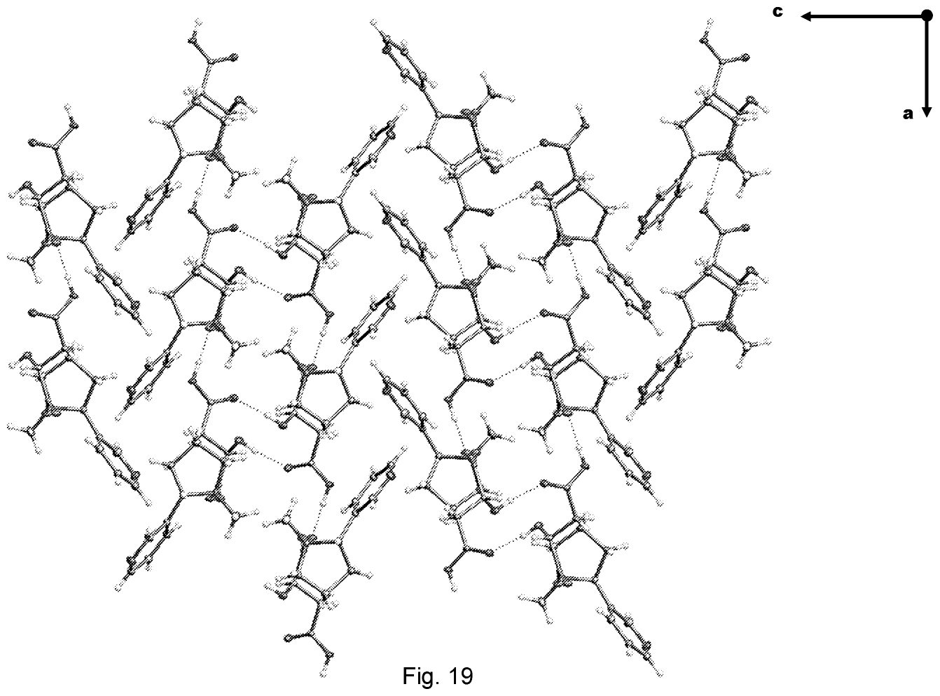

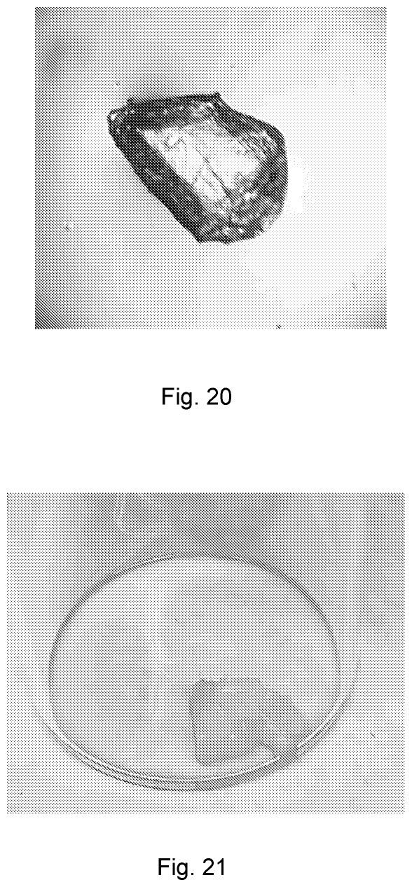

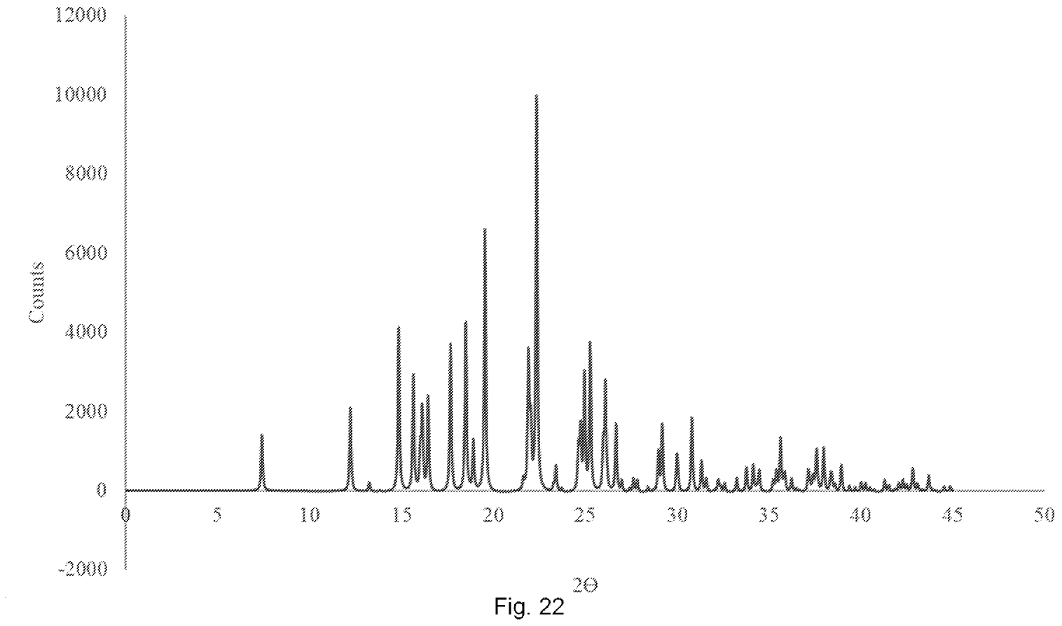

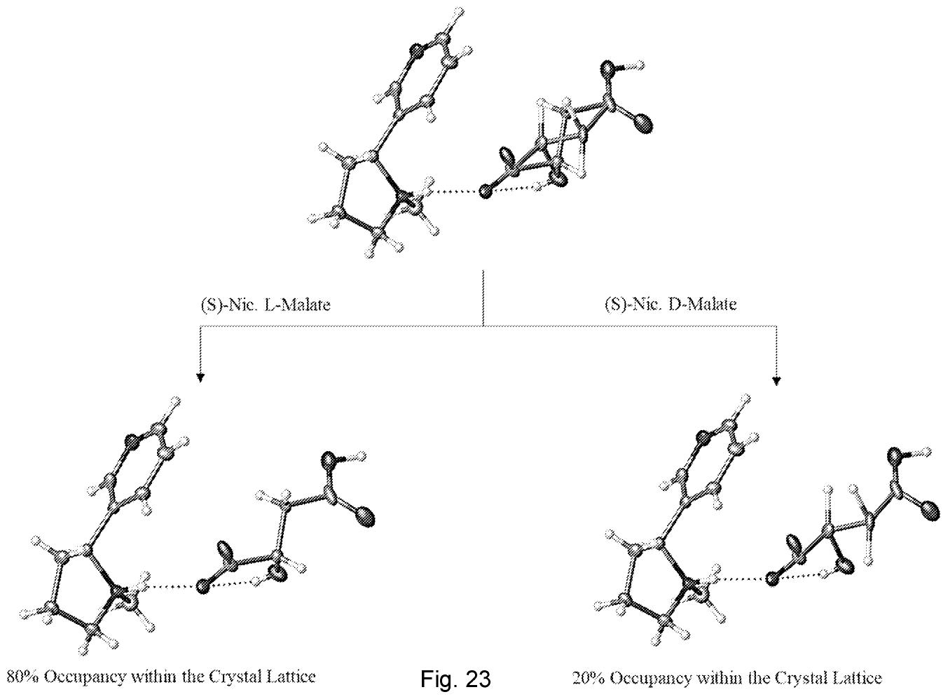

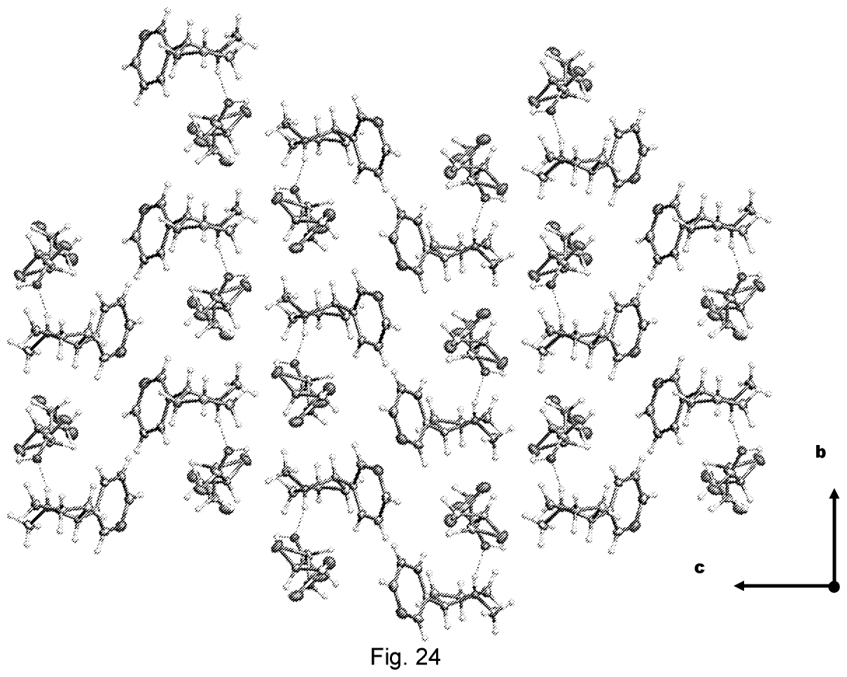

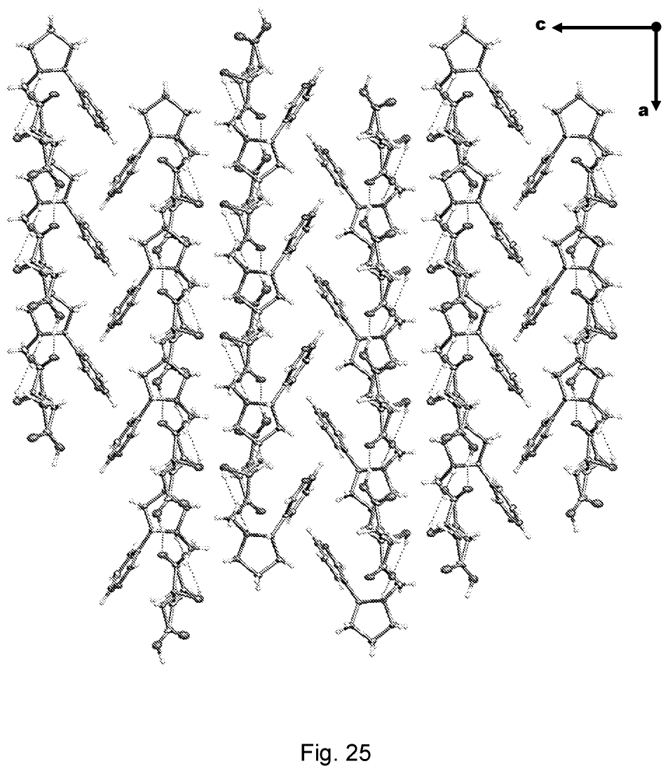

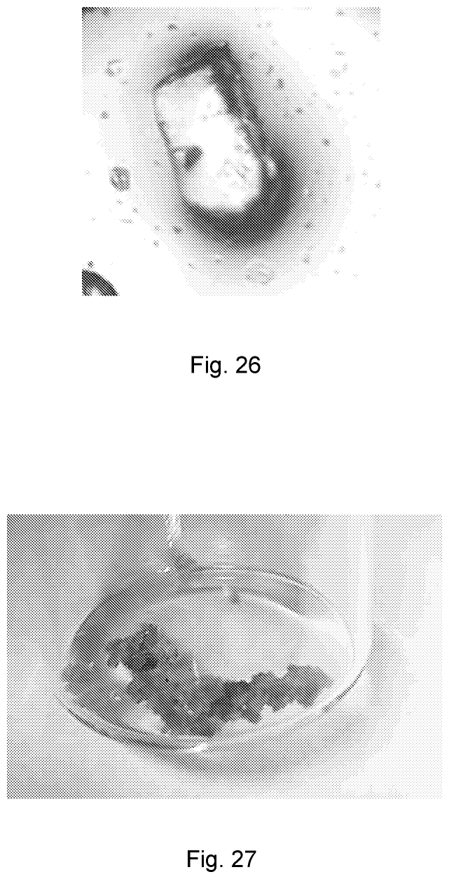

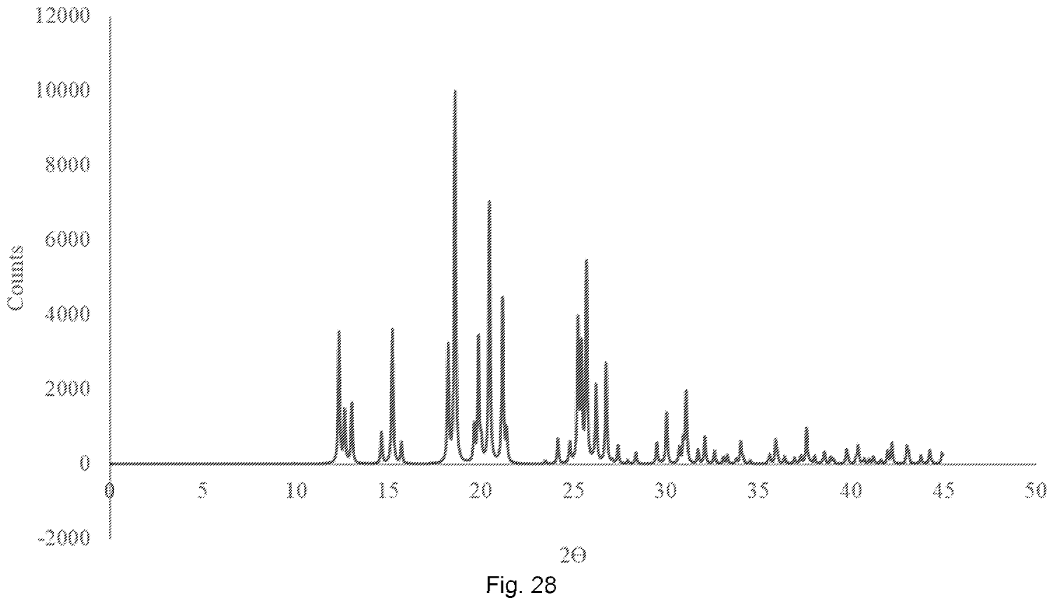

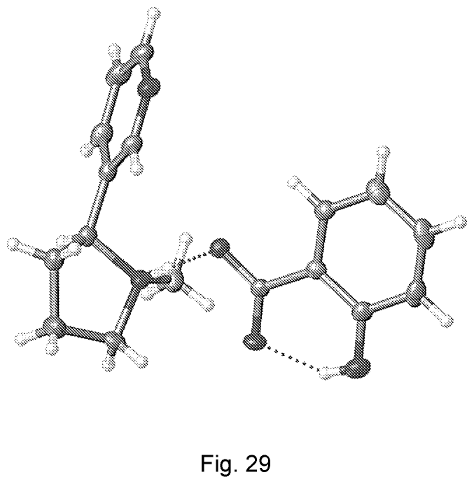

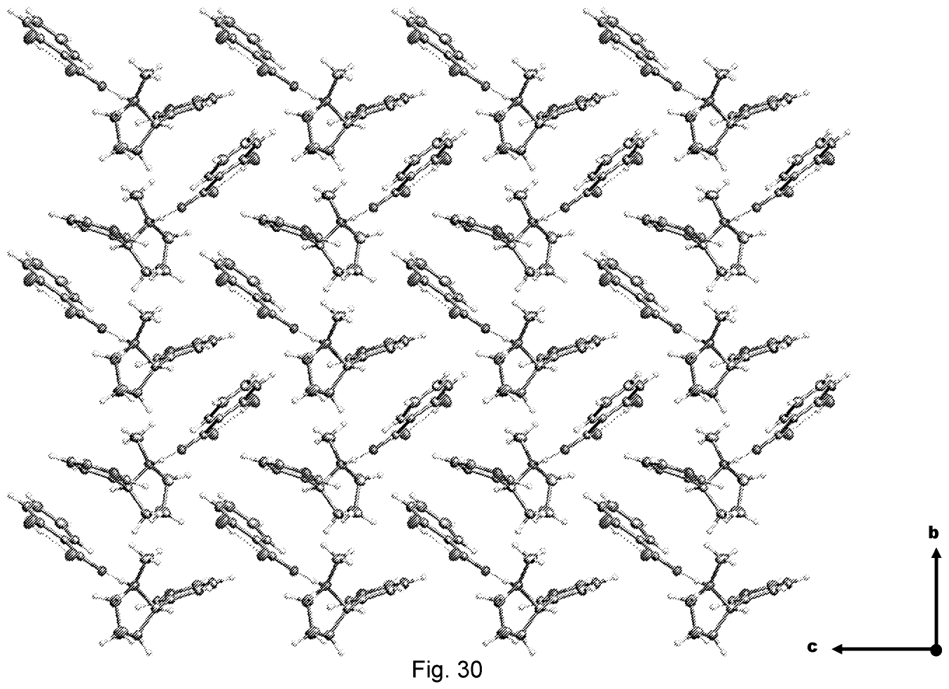

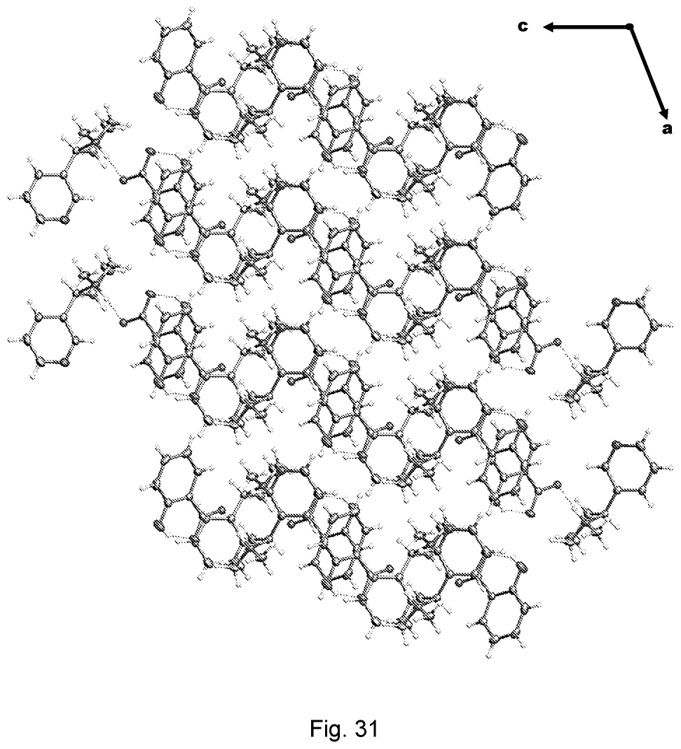

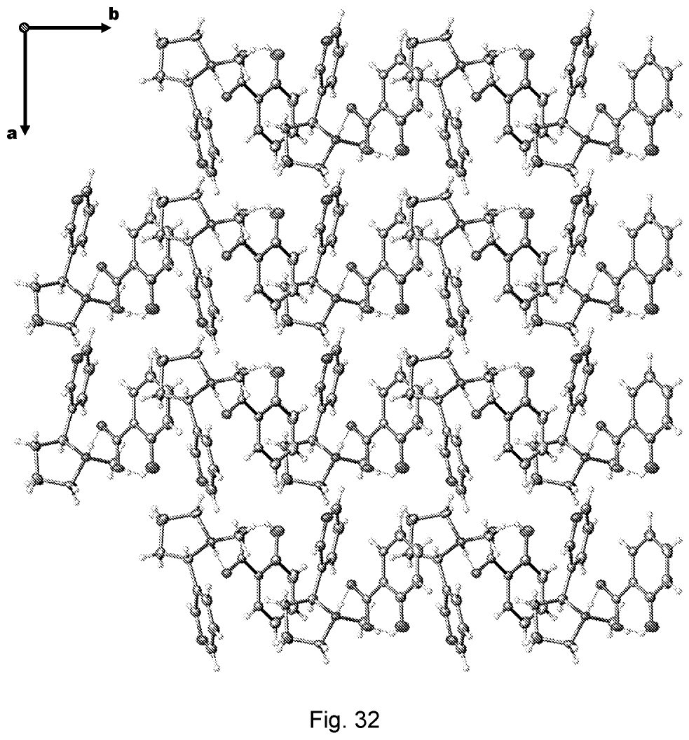

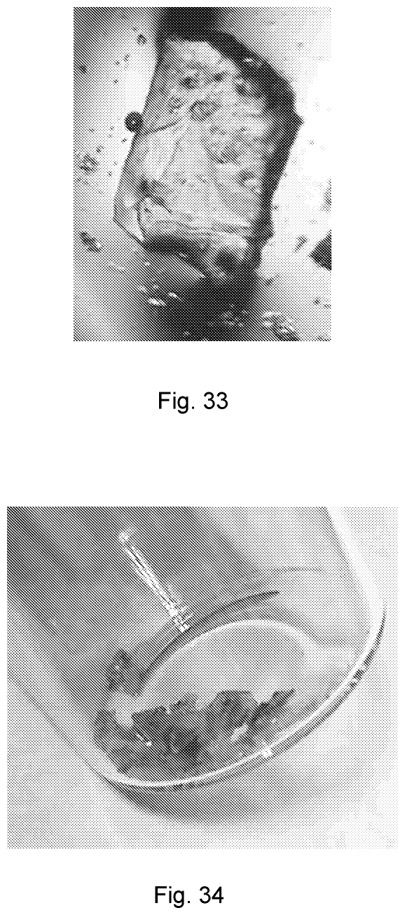

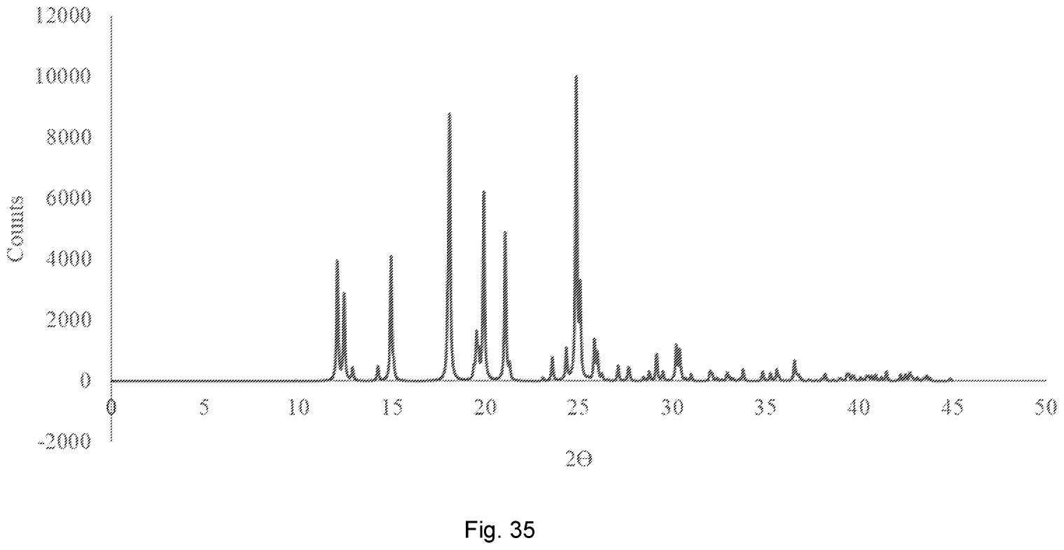

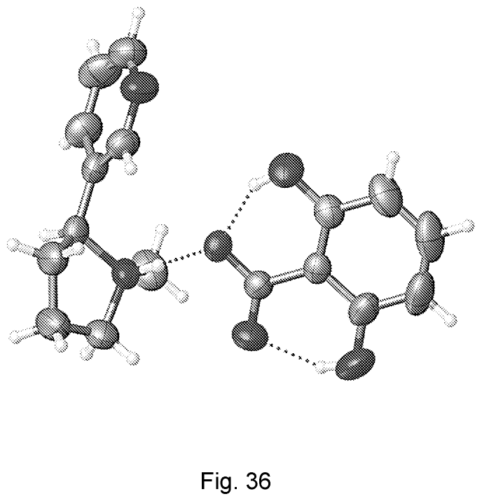

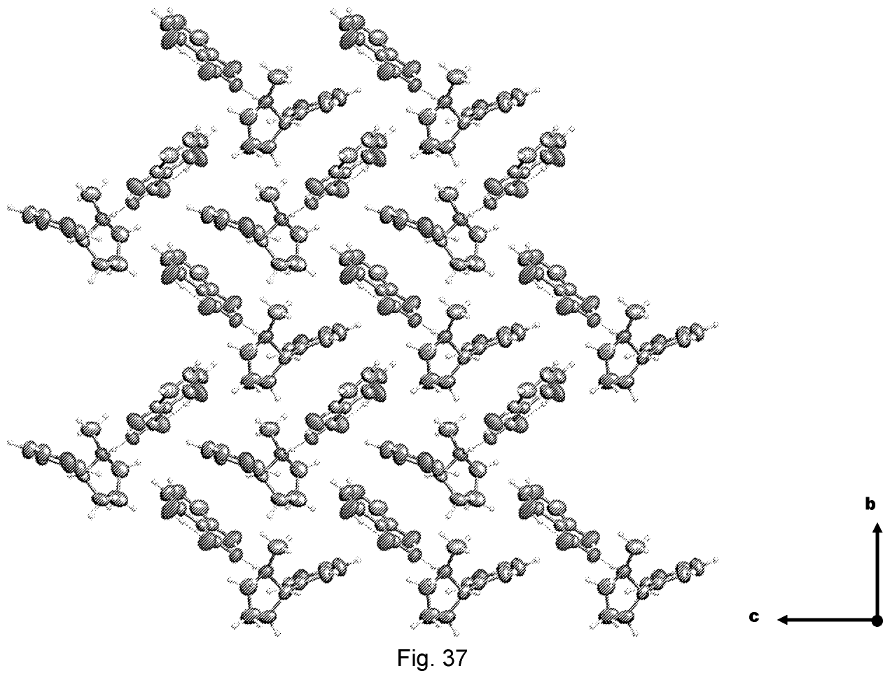

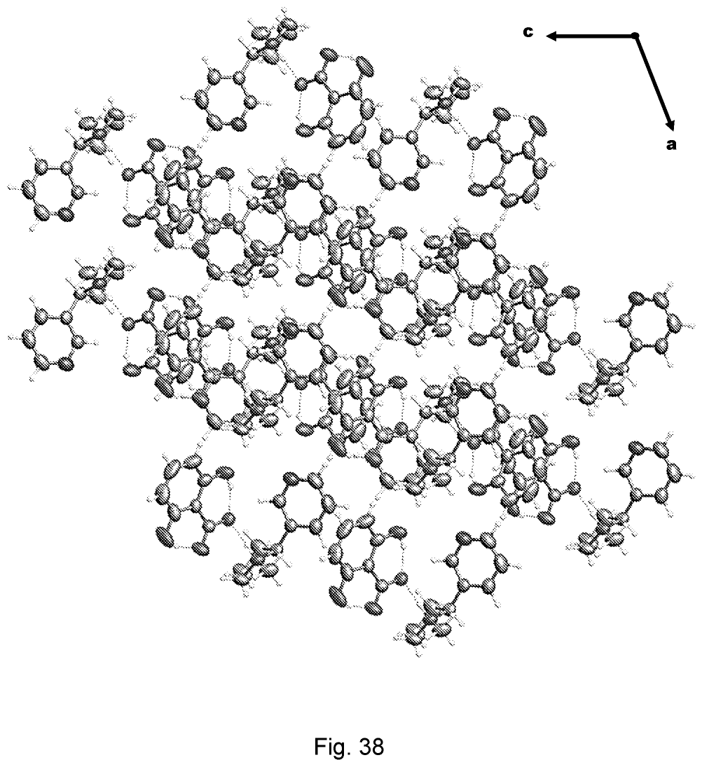

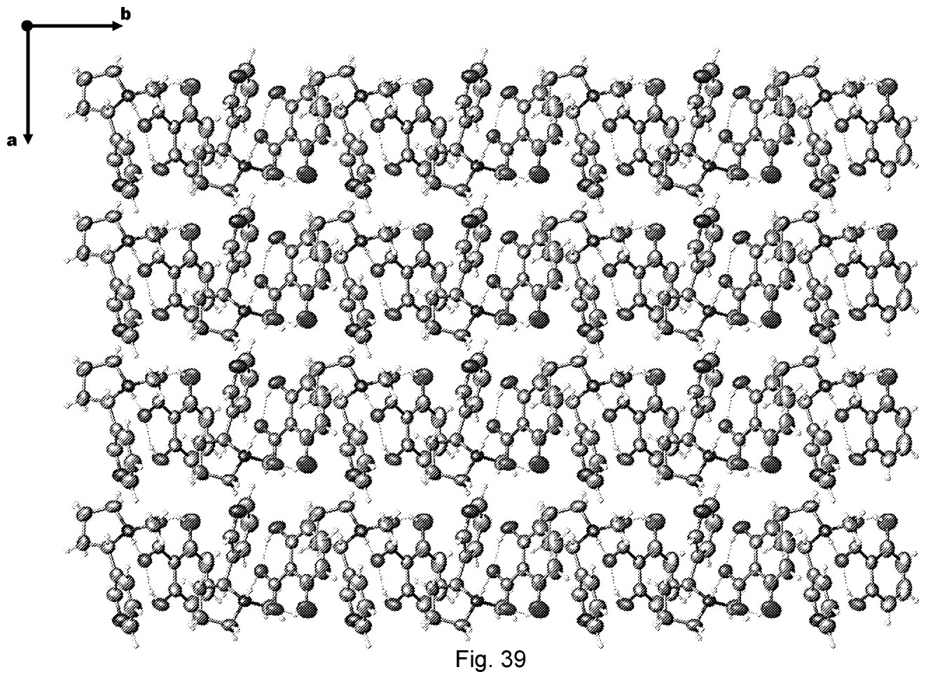

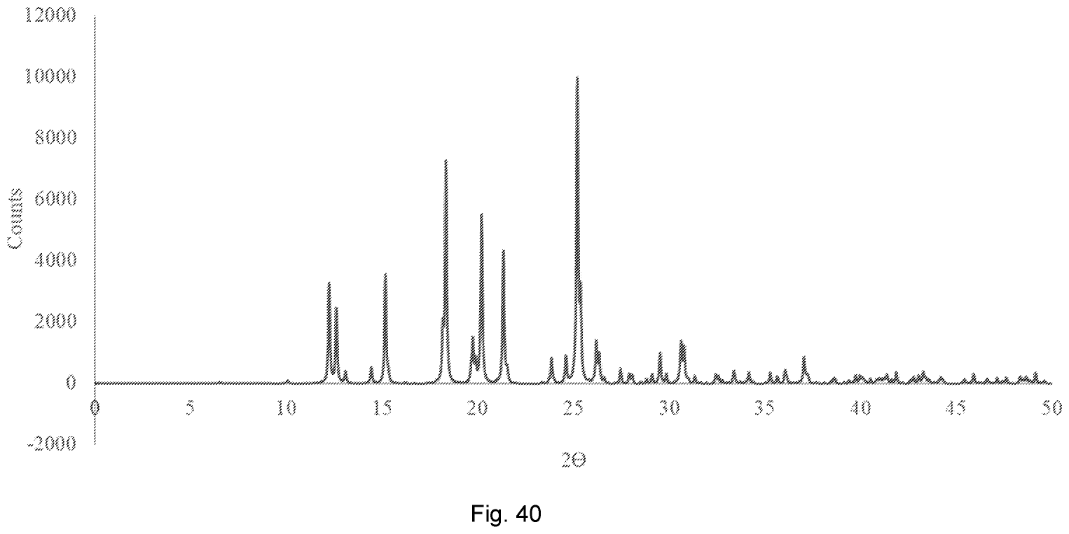

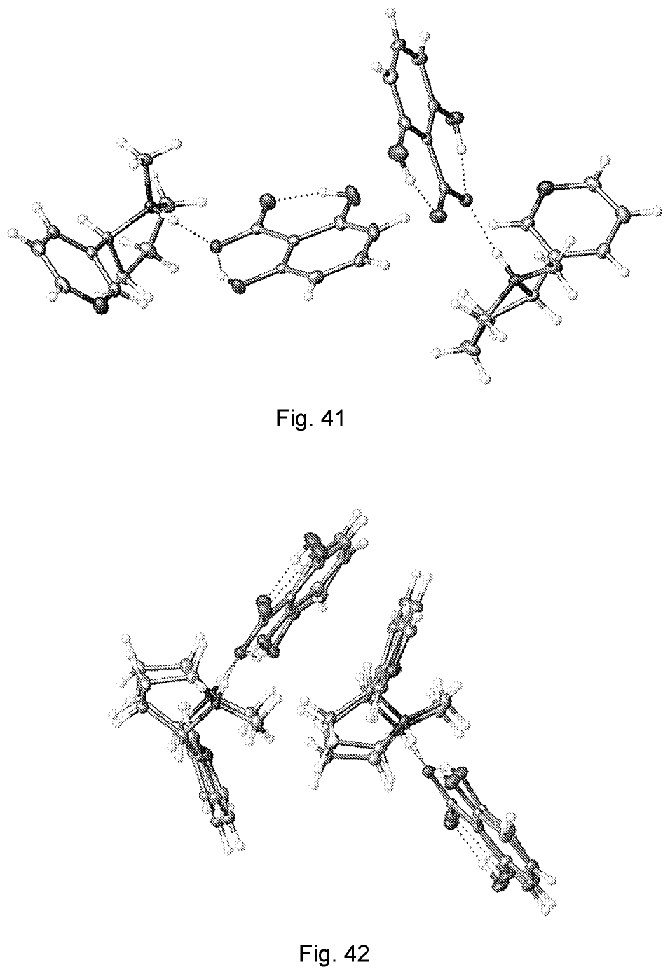

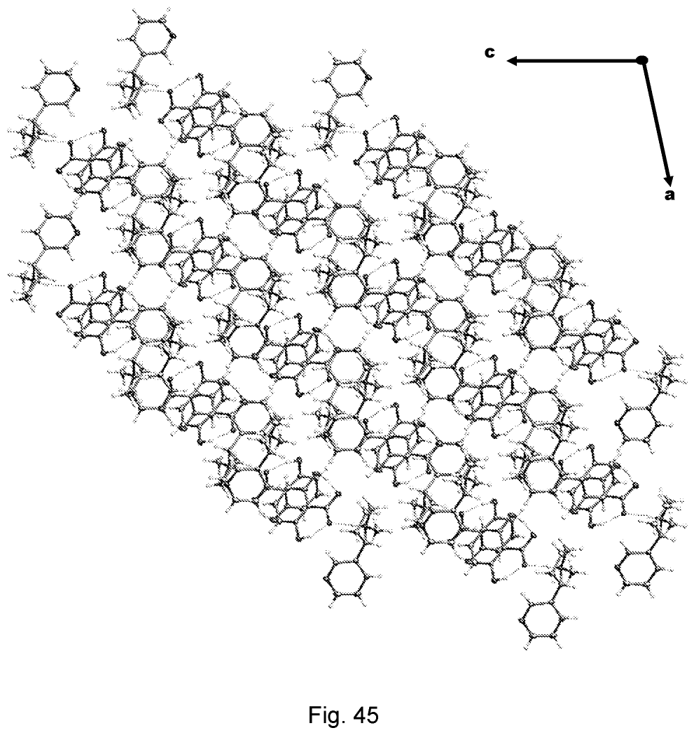

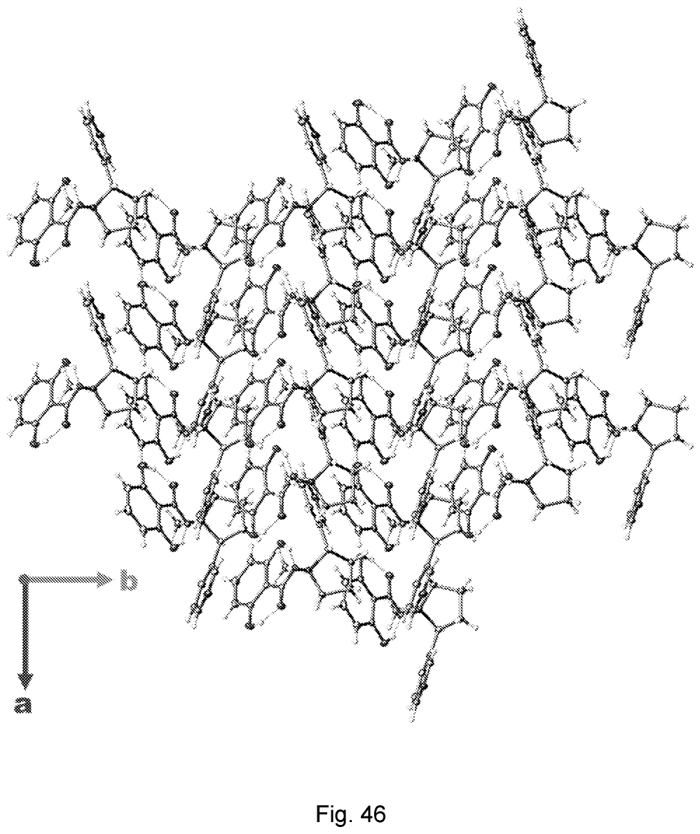

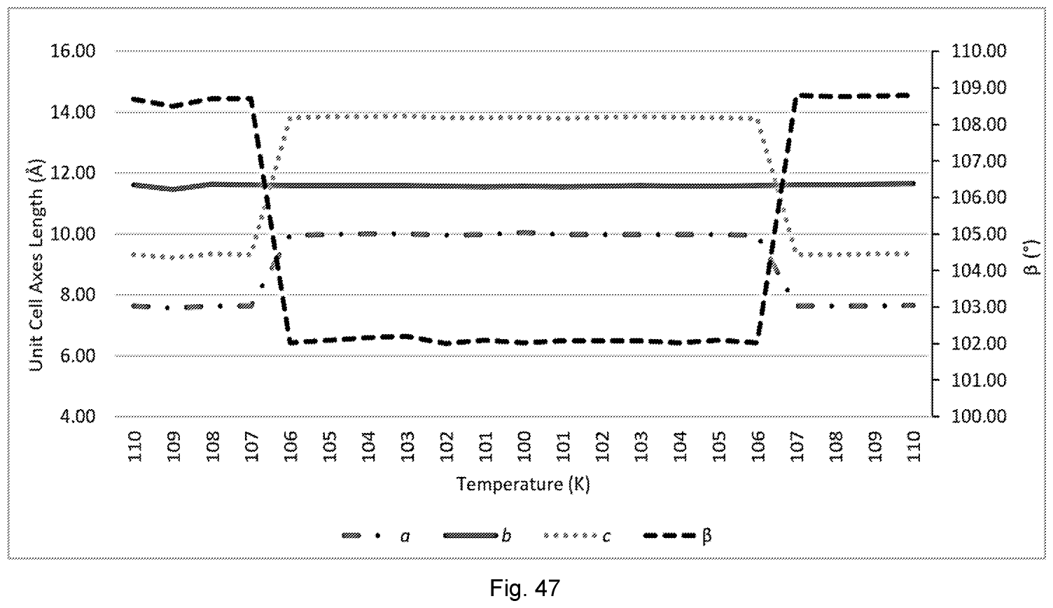

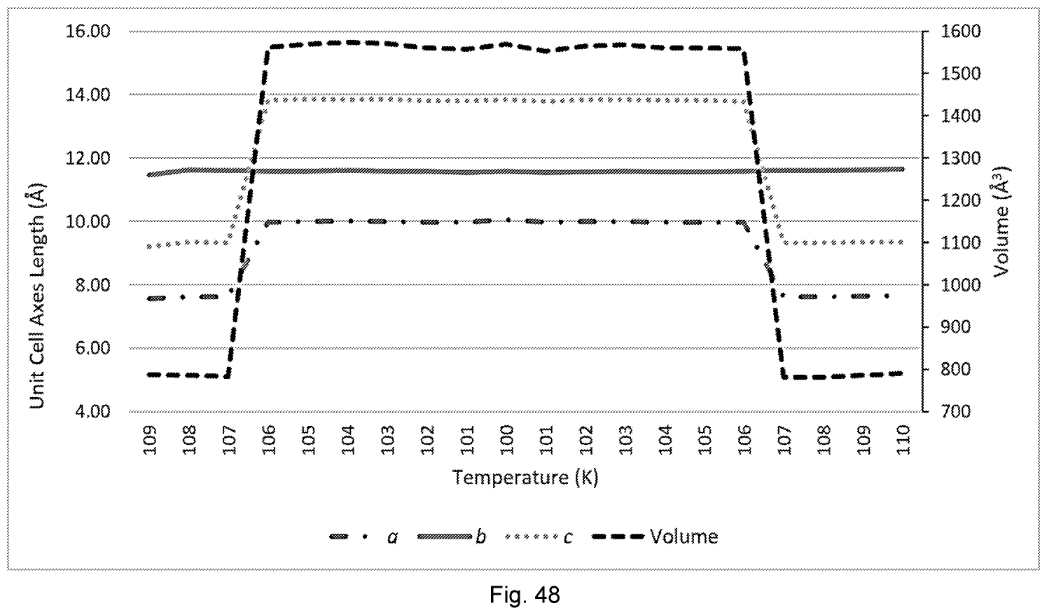

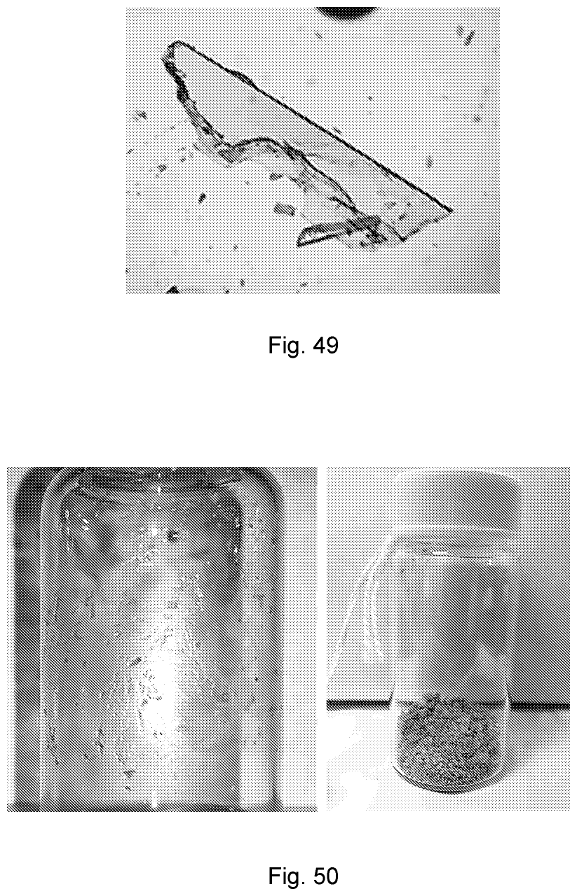

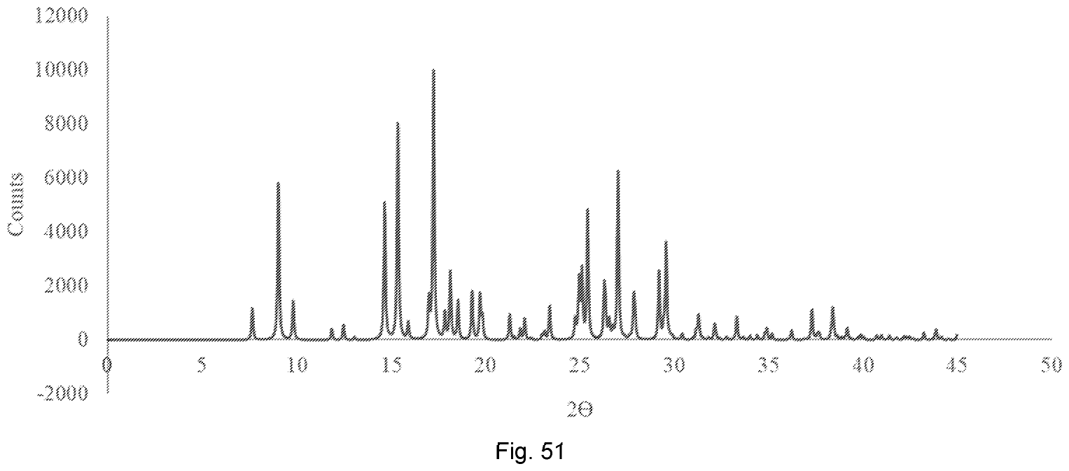

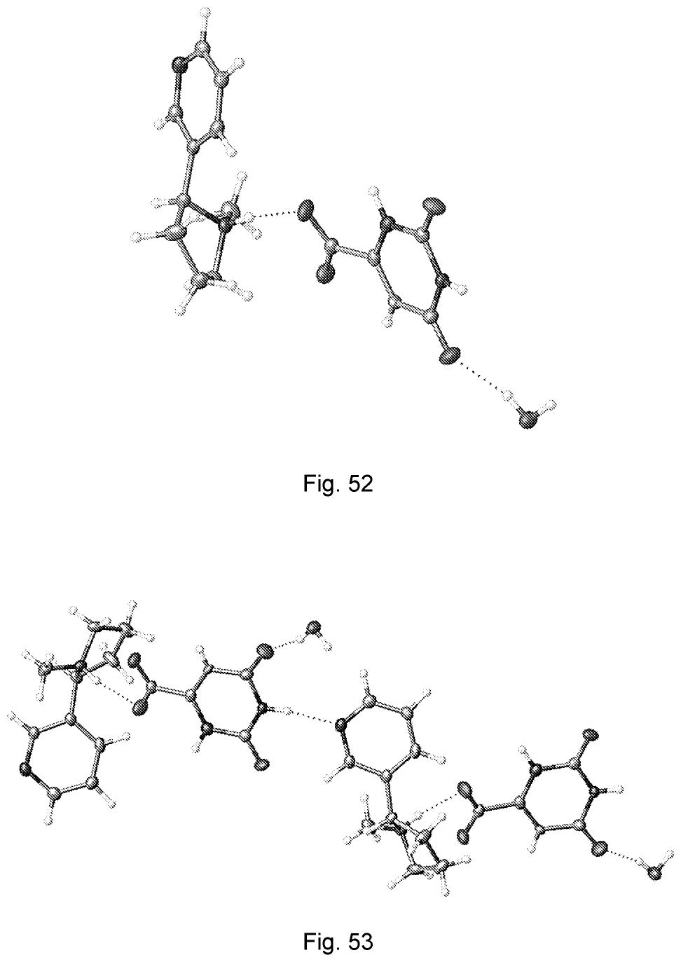

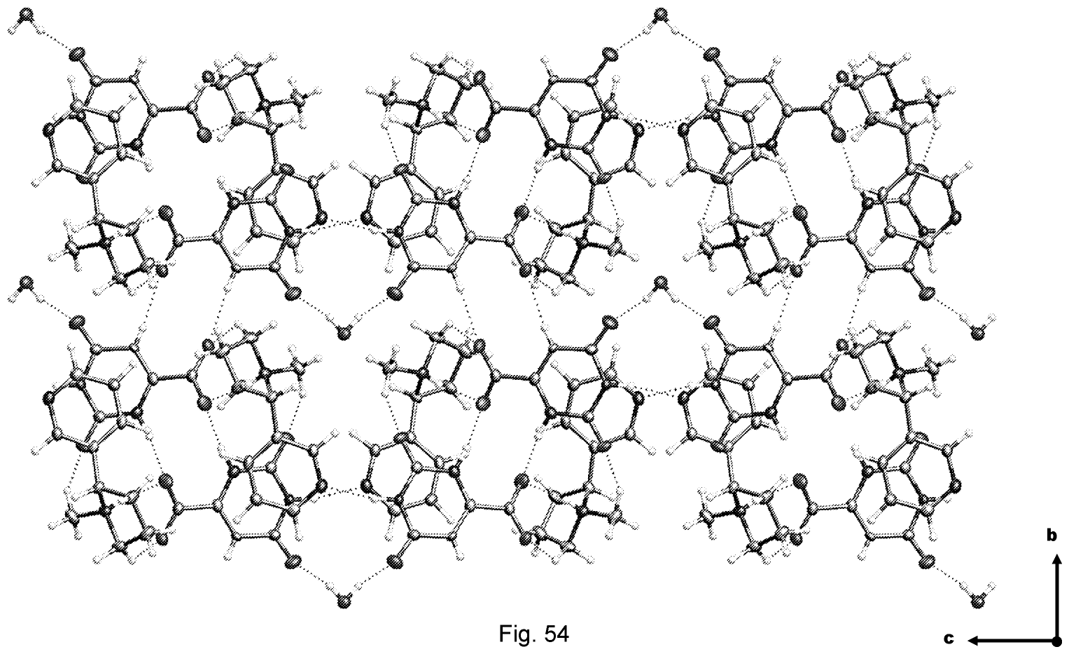

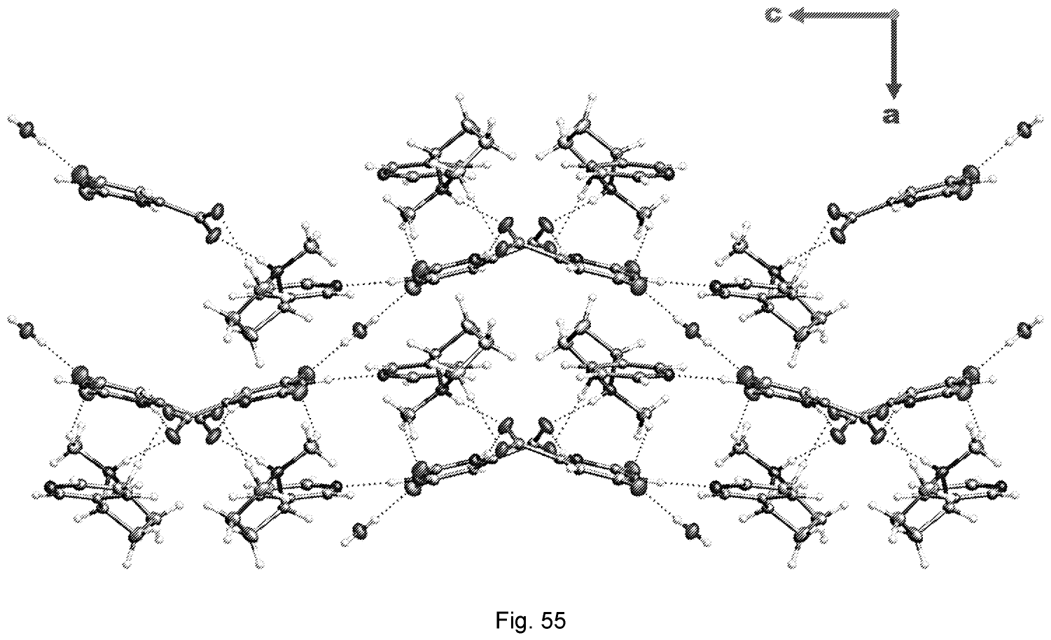

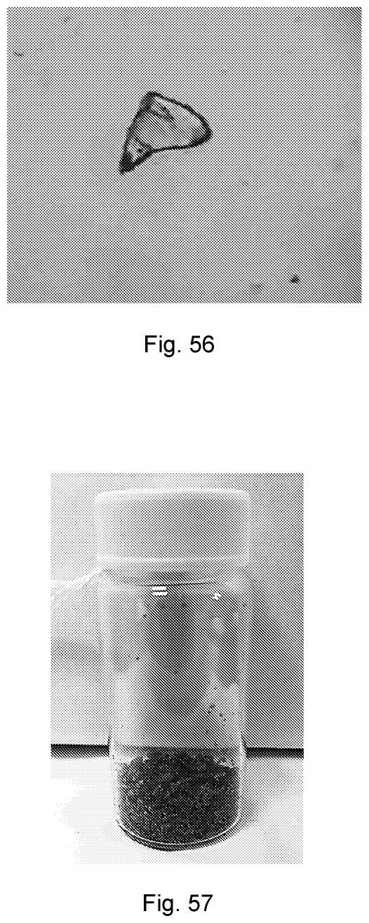

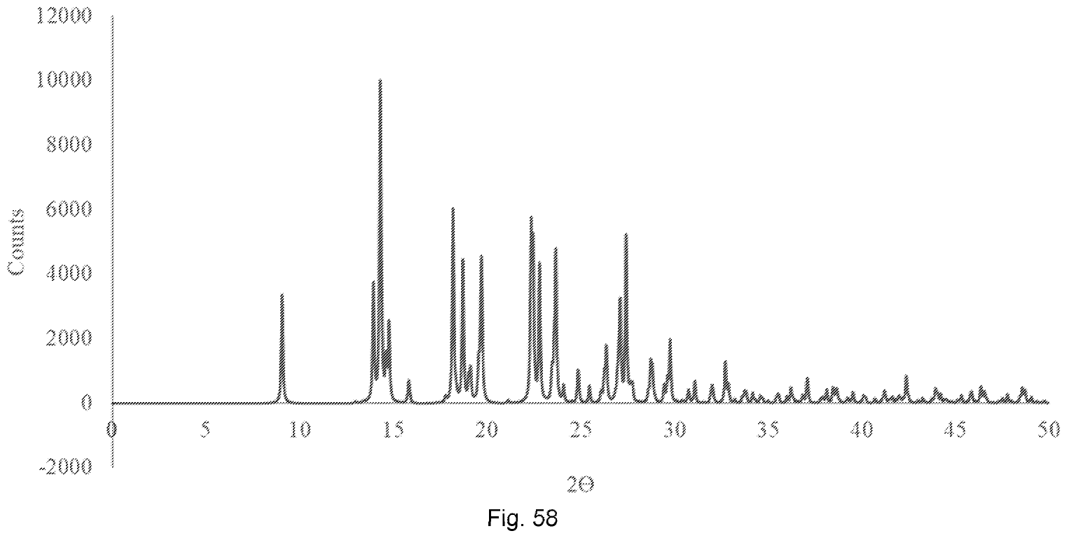

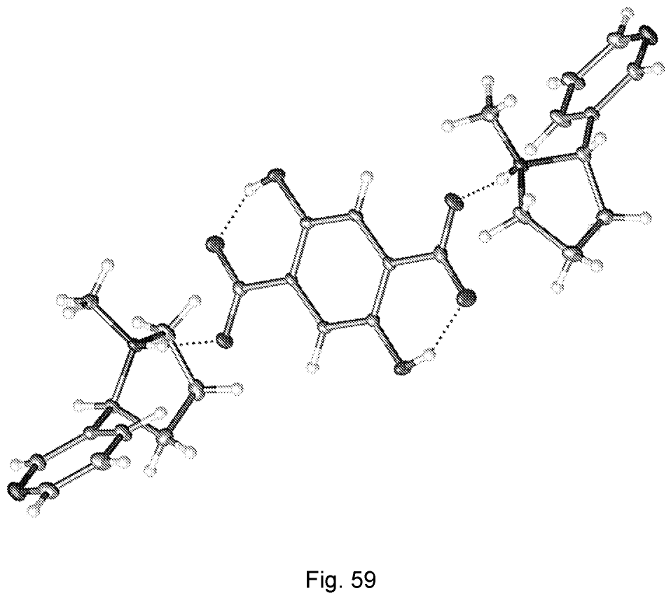

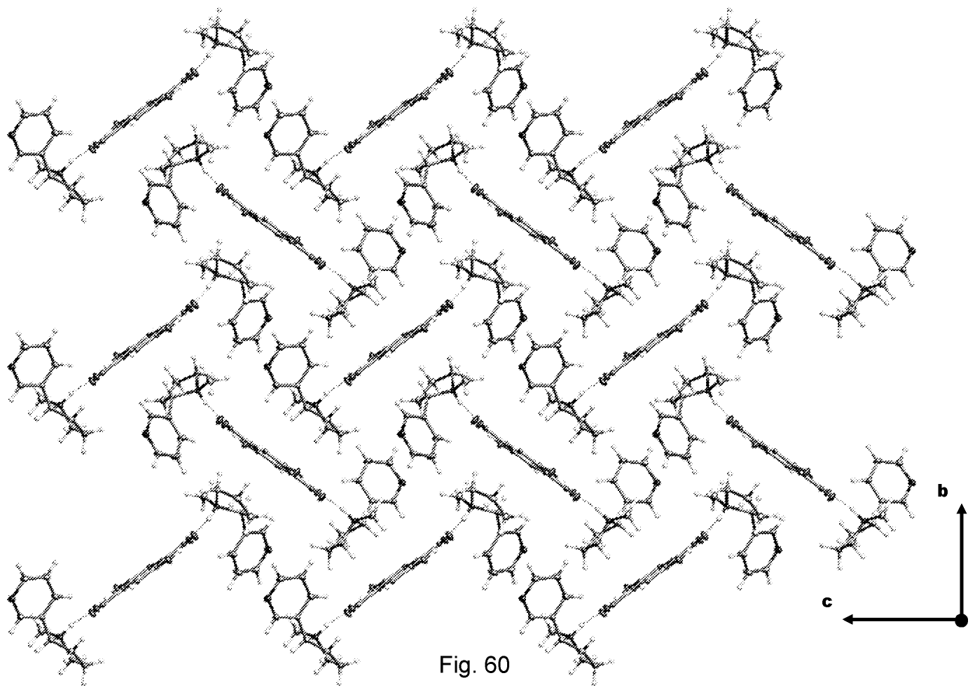

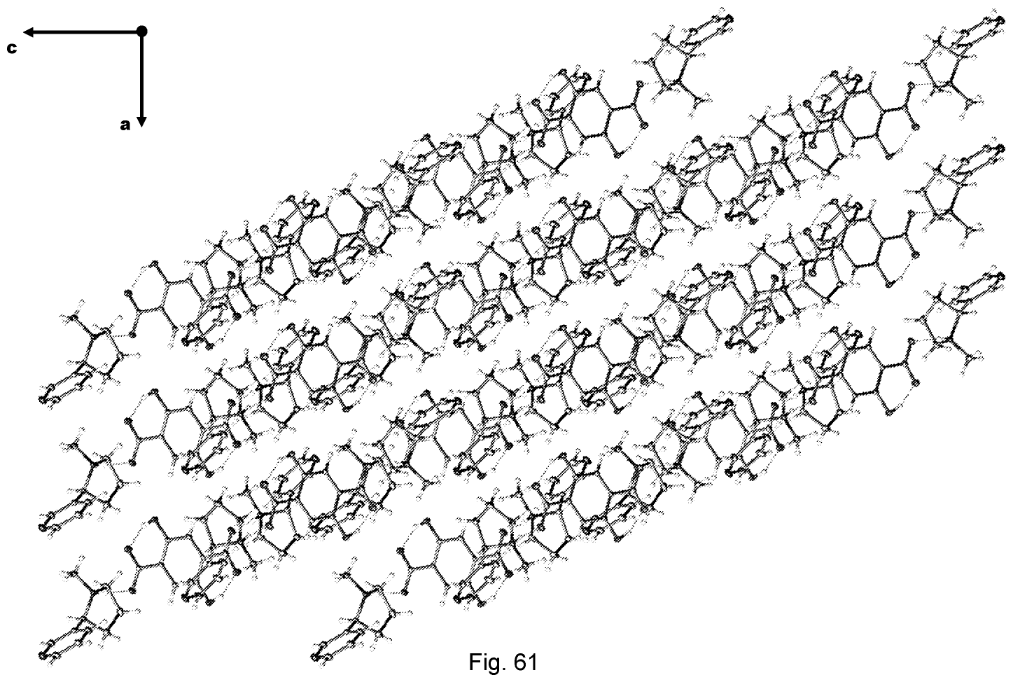

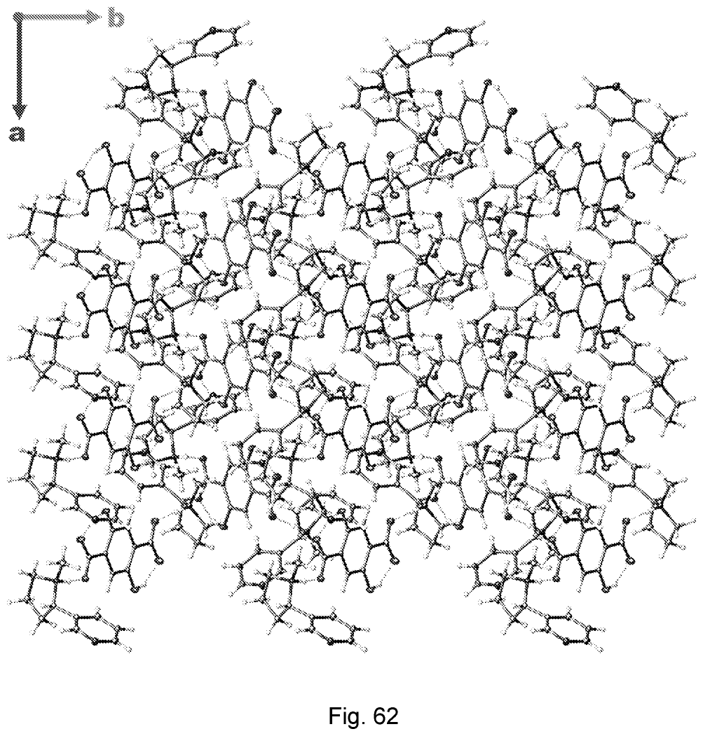

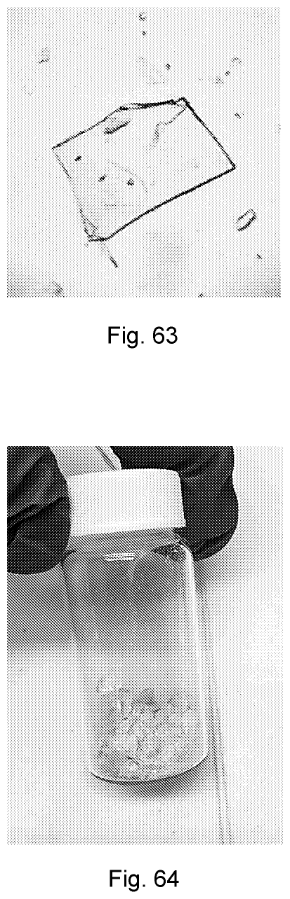

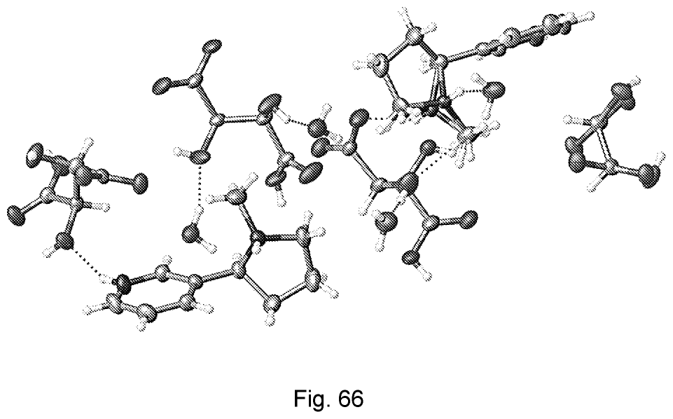

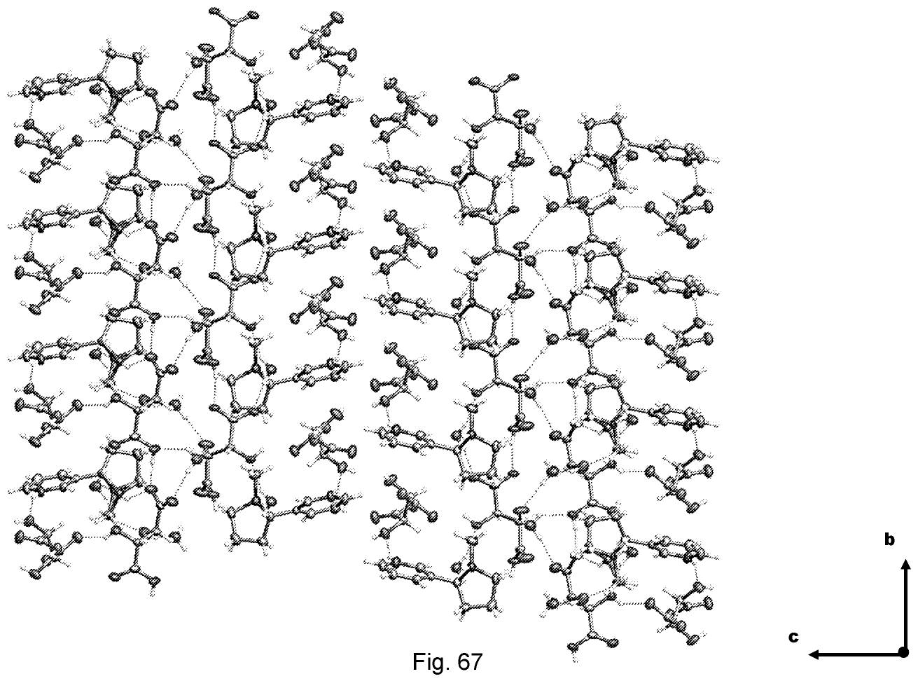

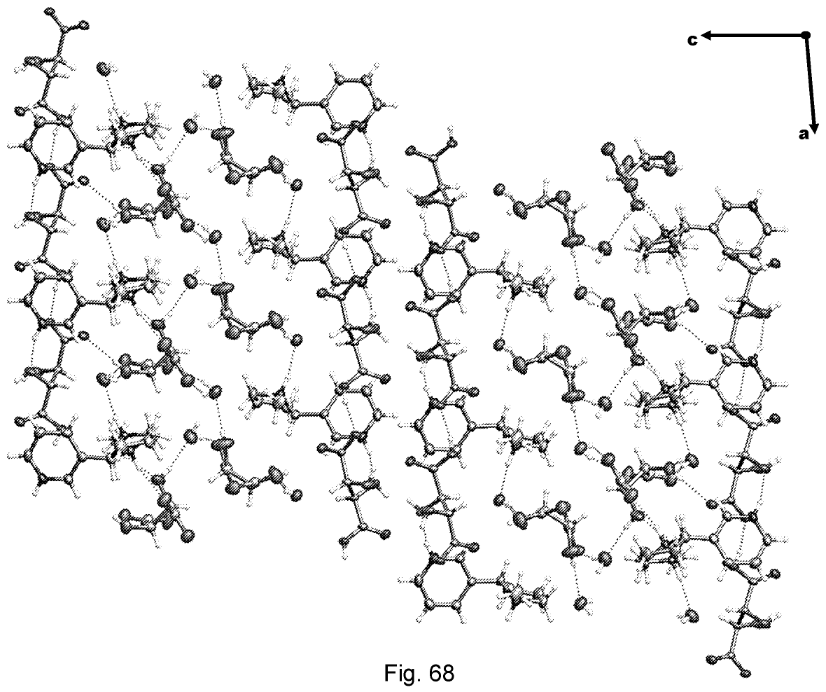

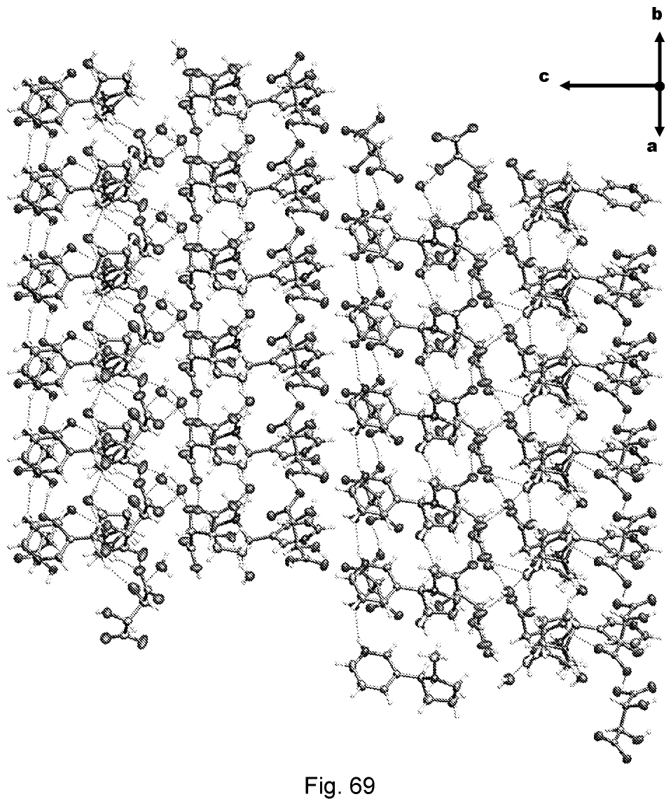

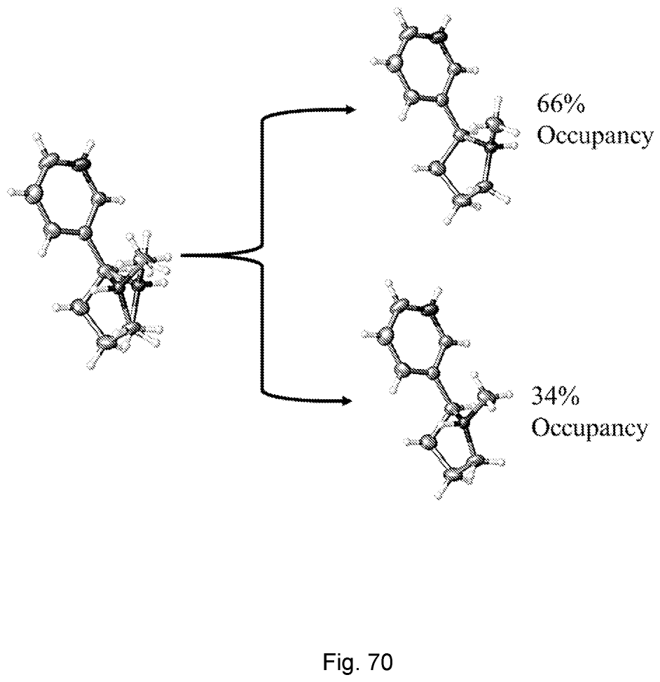

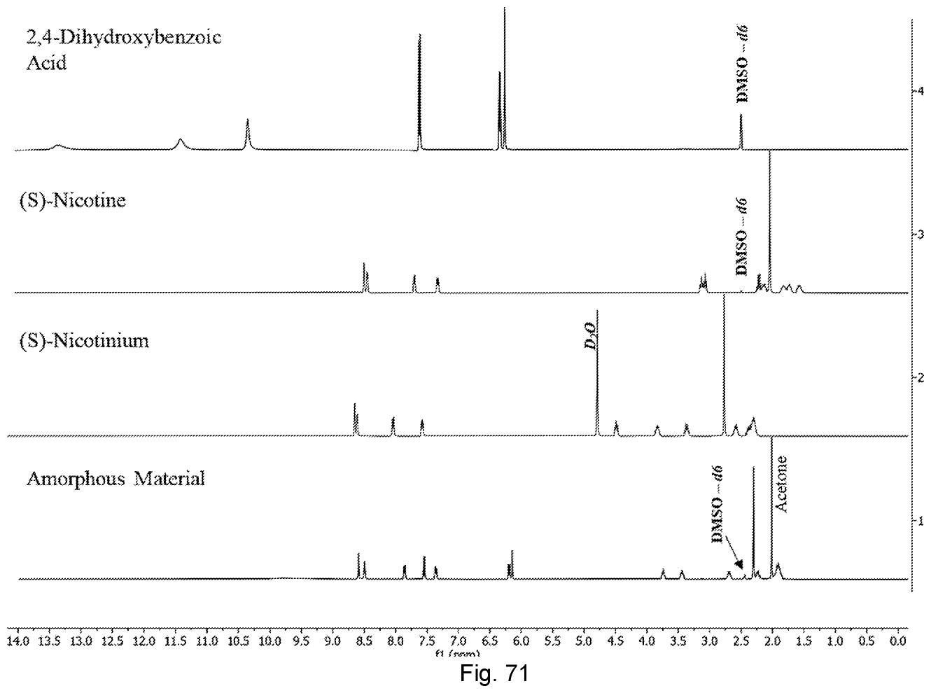

An orthorhombic P2₁2₁2₁ (S)-nicotinium L-malate (FIG. 1-6) was synthesized. Another polymorph, monoclinic P2₁ (S)-nicotinium L-malate (FIG. 7-12), along with orthorhombic P2₁2₁2₁ (S)-nicotinium D-malate (FIG. 13-19), orthorhombic P2₁2₁2₁ (S)-nicotinium DL-malate (FIG. 20-25), monoclinic P2₁ (S)-nicotinium salicylate (FIG. 26-32), a room temperature polymorph monoclinic P2₁ (S)-nicotinium 2,6-dihydroxybenzoate (FIG. 33-39), a 90K phase transition/temperature dependent polymorph of monoclinic P2₁ (S)-nicotinium 2,6-dihydroxybenzoate (FIG. 40-48), orthorhombic P222₁ (S)-nicotinium orotate monohydrate (FIG. 49-55), monoclinic P2₁ (S)-nicotinium 2,5-dihydroxyterephthalate (FIGS. 56-62), and monoclinic P2₁ (S)-nicotinium bi-L-(+)-tartrate dihydrate (FIG. 63-70) were synthesized. An amorphous nicotine salt was also synthesized, (S)-nicotinium 2,4-dihydroxybenzoate (FIG. 71). Monoclinic P2₁ (S)-nicotinium L-malate crystals suitable for single crystal X-ray diffraction were grown and characterized as detailed below. Monoclinic P2₁ (S)-nicotinium L-malate was found to be thermodynamically more stable than the orthorhombic P2₁2₁2₁ (S)-nicotinium L-malate. This could potentially be a favorable trait as this polymorph may not degrade or melt until higher temperatures than the orthorhombic polymorph of (S)-nicotinium L-malate, based upon the differential scanning calorimetry thermal analysis.

Synthesized single crystals of orthorhombic P2₁2₁2₁ (S)-nicotinium D-malate were also grown and found to be suitable for single crystal X-ray diffraction. This nicotine salt was found to be thermodynamically less stable than either of the (S)-nicotinium L-malate salts. This could also be a more favorable trait as the atomizer in a nicotine vaporizer device may not have to heat to as high of a temperature to deliver the nicotine salt to the user.

Synthesized single crystals of monoclinic P2₁ (S)-nicotinium salicylate were also characterized. The monoclinic structure has previously been reported, however, the instant synthesis provides an extremely high yield and high purity salt. The instant monoclinic P2₁ (S)-nicotinium 2,6-dihydroxybenzoate salt and (S)-nicotinium 2,5-dihydroxyterephthalate had the highest observed thermal stability of the single crystals prepared. Orthorhombic P222₁ (S)-nicotinium orotate monohydrate, was formed via slow evaporation.

The synthesized nicotine salts demonstrate desirable tunable thermal stability and photostability in comparison with pure liquid (S)-nicotine, while also, in certain cases, incorporating an engineered proton transfer with safety through designed degradation.

Engineering the Desired Proton Transfer. An important discovery was made from SC-XRD analysis of a successfully synthesized single crystalline salt. The hydroxyl group had undergone a hydrogen bonding interaction to the carboxylic acid group, which aided in the proton transfer from the acid to the nicotine. These details were then utilized in the design and co-crystal engineering of most other nicotine salts created thus far. For further nicotine salts, a hydroxyl group was typically desired within an acceptable distance for intramolecular bonding with the carboxyl group. Typically, these types of O—H—O hydrogen bonds have distances of around 1.8 to 2.6 Å. For designing such proton transfer salts, mostly alpha hydroxy acids and beta hydroxy acids were selected. This is because alpha hydroxy acids have a hydroxyl group, located on the carbon next to the carboxyl group, while beta hydroxy acids have a hydroxyl group located two carbons away from the carboxyl group. These hydroxyl groups lie within an acceptable distance for this intramolecular bond and as such as are capable of undergoing an intramolecular hydrogen bond to an oxygen in the carboxyl group. This intramolecular hydrogen bond causes the acidic proton in the carboxyl group to be held less strongly, due to the hydrogen bond drawing the oxygens' electron density away from the acidic proton of the carboxyl group. This made alpha and beta hydroxy acids desirable candidates for inducing a proton transfer for creating the desired nicotine salts. This intramolecular O—H—O bond also makes the hydroxyl O—H bond lengthen, causing the proton on the hydroxyl group to become further deshielded. This deshielding leads to the hydroxyl groups in the nicotine salts shifting further downfield in ¹H NMR spectra, beyond the detection capabilities of the instruments used. This phenomenon is frequently observed in molecules that possess a hydroxyl group that is engaged in a strong intramolecular hydrogen bond, sometimes shifting the hydroxyl ¹H NMR peaks downfield as far as 19 ppm.

Degradation by Design. With the shortfalls known to exist within nicotine salts and the liquid carrier fluid that is utilized in the formulation cocktail, the synthesized salts herein were engineered to address these issues. GRAS listed coformers were selected for safety reasons. Often selected is benzoic acid, a GRAS substance, with known degradation issues at excessively high temperatures. As such coformers were chosen that, for the most part, degrade into either another GRAS substance or a food safe substance upon the expected decarboxylation reaction(s). This approach eliminates the possibility of benzene formation from the salt coformer.

Also eliminated through this approach is the possibility that benzene or other carcinogens may be formed from any liquid carrier agents. Through co-crystal engineering, nicotine salts can be isolated as a stable crystalline solid that does not require a carrier fluid for delivery. This eliminates the formation of benzene from the cyclization of the propylene glycol and glycerin carrier fluids.

Salts described herein were specifically designed around the sole purpose of degradation, keeping safety in mind. Malic acid was chosen as it is a GRAS listed compound found in many foods. Upon a single decarboxylation, nicotinium malate salts do not degrade into a known GRAS listed compound. However, upon a double decarboxylation, ethanol, A GRAS substance would be formed. As such, nicotinium malate salts would need to either not degrade or degrade fully through a double decarboxylation to remain safe.

Salicylic acid was chosen next as a coformer. Though a structure was previously reported for a nicotinium salicylate salt, it has not been previously studied as a method of nicotine delivery for humans, instead only being studied as a pesticide. Salicylic acid decarboxylates into phenol, which is a compound often used in medicines and found naturally in numerous foods, that has an established history of being safe for human consumption. This makes monoclinic P2₁ (S)-nicotinium salicylate a prime example of a nicotine salt that can be designed around degradation. Likewise, monoclinic P2₁ (S)-nicotinium 2,6-dihydroxybenzoate was created using 2,6-dihydroxybenzoic acid. Though 2,6-dihydroxybenzoic acid is not GRAS listed, that is because it has simply never been subjected to the required FDA and FEMA GRAS testing requirements. It is a naturally occurring molecule that is found among several foods people consume, making it a safe molecule. When this coformer undergoes decarboxylation, it degrades into resorcinol, a safe GRAS listed molecule. Monoclinic P2₁ (S)-nicotinium 2,5-dihydroxyterephthalate was designed with multiple degradation steps in mind. The coformer 2,5-dihydroxyterephthalic acid is currently being used in edible and biocompatible metal organic frameworks (MOFs), among other things, demonstrating that it does have potential as a safe molecule for human consumption. Monoclinic P2₁ (S)-nicotinium 2,5-dihydroxyterephthalate was designed to interact with two nicotine molecules (one on each carboxyl group). It was also designed with multiple degradation products in mind. If monoclinic P2₁ (S)-nicotinium 2,5-dihydroxyterephthalate undergoes a single decarboxylation, the coformer would degrade into gentisic acid, an established GRAS listed compound. If monoclinic P2₁ (S)-nicotinium 2,5-dihydroxyterephthalate undergoes two decarboxylation reactions, the coformer would then degrade into hydroquinone, a far safer compound than benzene.

Another recent concern that has arisen around vaping is flavoring agents. Many flavoring agents are safe for human consumption at ambient conditions, however, when exposed to the vacuum, elevated wattage and elevated heat of the vaporization process, flavoring agents have been shown to react and degrade into a menagerie of substances. This toxic cocktail of flavoring agent degradation products is another issue that crystal engineering could solve. (S)-nicotinium 2,4-dihydroxybenzoate is the result of a successful attempt to create nicotine salts that contain a GRAS flavoring agent as the coformer. This coformer was also selected based upon its degradation products. (S)-nicotinium 2,4-dihydroxybenzoate was designed with the GRAS listed coformer 2,4-dihydroxybenzoic acid. This coformer also degrades into the GRAS listed compound resorcinol upon decarboxylation. (S)-nicotinium 2,4-dihydroxybenzoate was designed with flavor degradation in mind, however, only formed amorphous materials.

Developing a Unique and Simplified Synthetic Approach. The methods developed herein take advantage of a unique spontaneous homogeneous nucleation process. This first in class, developed method requires no seed crystals, and yet has been developed to allow for control of polymorphic phases, as detailed herein. The controllable polymorphism is attainable simply by varying the concentration of solvent and (S)-nicotine within each system. No other large-scale nicotine salt study utilizes simplistic and yet tunable methods such as the ones detailed herein.

GRAS coformers were chosen for each nicotine salt attempt. With the exception of orotic acid, the main focus was drawn to alpha and beta hydroxy acids so as to incorporate a hydroxyl group within intramolecular hydrogen bonding distance of at least one acid group. While not a hydroxy acid, orotic acid's highly acidic nature, as indicated by the pKa1 value, also allowed it to be a favorable coformer candidate.

Instrumentation.

Differential Scanning calorimetry (DSC). A differential scanning calorimeter, model DSC Q200 (TA Instrument, USA) was used to measure the thermal transitions of the samples. About 10 mg of each synthesized compound was placed into an aluminum pan and sealed. Each sample, except (S)-nicotinium 2,6-Dihydroxybenzoate, was scanned from 0 to 150° C. at 20° C./min under nitrogen flow for at least 2 full cycles. (S)-nicotinium 2,6-Dihydroxybenzoate was scanned from 0 to 175° C. at 20° C./min under nitrogen flow for at least 2 full cycles.

Melting Point (MP): A Stuart SMP10 melting point apparatus was utilized to measure the melting point of each of the synthesized compounds. 4 replicates were run for each compound. The plateau temperature was set to 100° C. for each of the salts, except (S)-nicotinium D-malate, and then ramped at 2° C./minute. The plateau temperature was set to 80° C. for the (S)-nicotinium D-malate salt and ramped at 2° C./minute.

X-Ray Diffraction (XRD). X-ray diffraction data on all synthesized nicotine salts was collected using a Bruker SMART APEX2 CCD diffractometer installed at a rotating anode source (MoKα radiation, λ=0.71073 Å). Data collection was achieved at 90 K, unless otherwise specified, by the rotation method with 0.5° frame-width (ω scan) with unique exposure times for each single crystalline sample. Five sets of data (360 frames each) were collected. Using Olex2, the structures were solved with intrinsic phasing via the ShelXT structure solution program and refined with the ShelXL software suite using least squares minimization. Powder patterns were simulated using the obtained single crystal data with the CSD: Mercury Visualization and Analysis of Crystal Structures software suite. Powder patterns were simulated for a CuKα radiation source and normalized to a maximum intensity of 10,000 counts. PXRD data tables were computed using the Diamond 4 software suite from Crystal Impact.

Nuclear Magnetic Resonance (NMR). NMR analysis was run on a Varian Inova-500 broadband spectrometer (500 MHz) and a Varian Inova-400 broadband spectrometer (400 MHz), using an appropriate NMR solvent (e.g., d₄-methanol as the deuterated reference for each sample), except for DL-malic acid. Spectra of DL-malic acid were run in d₃-methanol.

UV Photodegradation. NMR analysis was done on a sample of each coformer (e.g., (S)-nicotine and each of the synthesized (5)-nicotinium salts). Each sample was then irradiated with ultraviolet (UV) light in a home built vented box with air flow for 24 hours using four Southern New England Ultraviolet Company RPR—3000A UV bulbs (λ=300 nm). NMR analysis was then carried out on each sample to screen for any UV photodegradation of products.

Aerosolization/Vaporization Analysis. An iStick Pico with Sai Top Air Flow Atomizer was connected to a two-neck flask, via a reverse banger, and a syringe was used to simulate “puffs”. The device was used with either titanium or quartz bucket coils, using the appropriate temperature and wattage settings for each. A cold bath was used as necessary to collect the vapor. Analysis on the collected vapor and any material that was aerosolized can be carried out via X-ray diffraction, NMR, mass spectrometry or other analytical methods as necessary.

Alternatively, an iStick Pico equipped with a Sai Top Air Flow Atomizer was connected to a syringe that was used to simulate “puffs”. The device was used with either titanium or quartz bucket coils, using the appropriate temperature and wattage settings for each. Analysis on the collected vapor and any material that was aerosolized can be carried out via X-ray diffraction, NMR, mass spectrometry or other analytical methods as necessary.

Future instrumentation and characterization on currently synthesized salts and co-crystals, as well as any salts or co-crystals synthesized in the future, may include but is not limited to: differential scanning calorimetry (DSC), thermogravimetric analysis (TGA), melting point (MP), X-Ray diffraction (XRD), powder X-Ray diffraction (PXRD) (simulated or otherwise), nuclear magnetic resonance (NMR), UV photodegradation, Fourier transform infrared (FTIR) spectroscopy, mass spectrometry (MS) of various methodologies, aerosolization/vaporization analysis, polarized light microscopy, time resolved absorbance microscopy, and Hirschfeld analysis.

Methods

Orthorhombic P2₁2₁2₁ (S)-Nicotinium L-Malate. Small Scale: L-(−)-malic acid (134.1 mg, 1.0 mmol) was placed into a 20 mL scintillation vial. MeOH (3.0 mL) was added with vigorous agitation. (S)-Nicotine (0.24 mL, 1.5 mmol) was added via micropipette in the dark to avoid degradation. The resulting solution was vortexed for 30 seconds at 3,000 rpm on a VWR Mini Vortexer MV I. The solution was then stored in the dark uncapped to allow for crystal formation while the solvent slowly evaporated. Once the solvent was evaporated, the crystalline product was collected via vacuum filtration, washing with n-heptane (3×5 mL) (157.4 mg, 53.12%). The yield was computed relative to the asymmetric unit. The resulting crystalline product was characterized by single crystal X-ray diffraction, simulated powder X-ray diffraction, melting point, differential scanning calorimetry, and a UV irradiation degradation test monitored by ¹H NMR.

Small Scale w/HCl: L-(−)-malic acid (134.1 mg, 1.0 mmol) was placed into a 20 mL scintillation vial. MeOH (3.0 mL) was added with vigorous agitation. (S)-Nicotine (0.16 mL, 1.0 mmol) was added via micropipette in the dark to avoid degradation. The resulting solution was vortexed for 30 seconds at 3,000 rpm on a VWR Mini Vortexer MV I. 5 drops of HCl were added via Pasteur pipette and the solution was vigorously agitated. The solution was then stored in the dark uncapped to allow for crystal formation while the solvent slowly evaporated. Once the solvent was evaporated, the crystalline product was collected via vacuum filtration, washing with n-heptane (3×5 mL) (218.8 mg, 73.84%). The yield was computed relative to the asymmetric unit. The resulting crystalline product was characterized by single crystal X-ray diffraction, simulated powder X-ray diffraction, melting point, differential scanning calorimetry, and a UV irradiation degradation test monitored by ¹H NMR.

Large Scale: L-(−)-malic acid (1.3409 g, 10.0 mmol) was placed into a 250 mL beaker. MeOH (32 mL) was added while the solution was gently heated and stirred vigorously for 30 minutes. (S)-Nicotine (1.606 mL, 10.0 mmol) was added via micropipette in the dark to avoid degradation. The resulting solution was stored in the dark, uncovered to allow for crystal formation while the solvent slowly evaporated. The solution was monitored for single crystal growth via polarized light imaging microscopy. Once the solvent was evaporated, the crystalline product was collected via vacuum filtration, washing with n-heptane (3×15 mL) (2.7748 g, 93.64%). The yield was computed relative to the asymmetric unit. The resulting crystalline product was characterized by single crystal X-ray diffraction, simulated powder X-ray diffraction, melting point, differential scanning calorimetry, and a UV irradiation degradation test monitored by ¹H NMR.

TABLE 2
X-ray powder pattern peak data for orthorhombic
P2₁2₁2₁ (S)-nicotinium L-malate.

| Peak | 2Θ | d-spacing | | | | | | |
|---|---|---|---|---|---|---|---|---|
| No. | [°] | [Å] | Intensity | F | h | k | l | Mult. |
| 1 | 7.39 | 11.95 | 1851.31 | 27.88 | 0 | 0 | 2 | 2 |
| 2 | 11.67 | 7.58 | 7.74 | 2.02 | 0 | 1 | 1 | 4 |
| 3 | 12.18 | 7.26 | 2472.44 | 37.68 | 1 | 0 | 1 | 4 |
| 4 | 13.32 | 6.64 | 292.55 | 14.20 | 0 | 1 | 2 | 4 |
| 5 | 13.77 | 6.43 | 52.11 | 6.20 | 1 | 0 | 2 | 4 |
| 6 | 14.81 | 5.98 | 5069.52 | 93.12 | 0 | 0 | 4 | 2 |
| 7 | 15.70 | 5.64 | 3556.20 | 58.51 | 0 | 1 | 3 | 4 |
| 8 | 16.06 | 5.52 | 1196.67 | 34.74 | 1 | 1 | 0 | 4 |
| 9 | 16.08 | 5.51 | 2559.09 | 50.87 | 1 | 0 | 3 | 4 |
| 10 | 16.48 | 5.37 | 2631.76 | 37.41 | 1 | 1 | 1 | 8 |
| 11 | 17.70 | 5.01 | 4629.69 | 53.38 | 1 | 1 | 2 | 8 |
| 12 | 18.53 | 4.79 | 5810.70 | 88.65 | 0 | 1 | 4 | 4 |
| 13 | 18.85 | 4.70 | 1534.01 | 46.38 | 1 | 0 | 4 | 4 |
| 14 | 19.56 | 4.53 | 8441.24 | 79.90 | 1 | 1 | 3 | 8 |
| 15 | 21.65 | 4.10 | 367.31 | 26.18 | 0 | 1 | 5 | 4 |
| 16 | 21.91 | 4.05 | 4043.91 | 62.21 | 1 | 1 | 4 | 8 |
| 17 | 21.93 | 4.05 | 653.18 | 35.38 | 1 | 0 | 5 | 4 |
| 18 | 22.24 | 3.99 | 1674.23 | 81.29 | 0 | 2 | 0 | 2 |
| 19 | 22.30 | 3.98 | 2788.87 | 105.21 | 0 | 0 | 6 | 2 |
| 20 | 22.55 | 3.94 | 10000.00 | 142.54 | 0 | 2 | 1 | 4 |
| 21 | 23.32 | 3.81 | 894.92 | 62.45 | 2 | 0 | 0 | 2 |
| 22 | 23.46 | 3.79 | 149.99 | 18.20 | 0 | 2 | 2 | 4 |
| 23 | 23.62 | 3.76 | 152.45 | 18.47 | 2 | 0 | 1 | 4 |
| 24 | 24.49 | 3.63 | 75.62 | 13.51 | 2 | 0 | 2 | 4 |
| 25 | 24.63 | 3.61 | 1228.67 | 38.74 | 1 | 1 | 5 | 8 |
| 26 | 24.92 | 3.57 | 1916.19 | 69.26 | 0 | 2 | 3 | 4 |
| 27 | 24.96 | 3.57 | 3727.04 | 96.76 | 0 | 1 | 6 | 4 |
| 28 | 25.15 | 3.54 | 52.49 | 11.58 | 1 | 2 | 0 | 4 |
| 29 | 25.20 | 3.53 | 4183.15 | 103.58 | 1 | 0 | 6 | 4 |
| 30 | 25.43 | 3.50 | 1102.27 | 37.95 | 1 | 2 | 1 | 8 |
| 31 | 25.88 | 3.44 | 6.63 | 4.24 | 2 | 1 | 0 | 4 |
| 32 | 25.89 | 3.44 | 1330.13 | 60.09 | 2 | 0 | 3 | 4 |
| 33 | 26.15 | 3.40 | 293.16 | 20.16 | 2 | 1 | 1 | 8 |
| 34 | 26.25 | 3.39 | 3217.37 | 67.04 | 1 | 2 | 2 | 8 |
| 35 | 26.83 | 3.32 | 2405.10 | 83.88 | 0 | 2 | 4 | 4 |
| 36 | 26.95 | 3.31 | 358.19 | 23.00 | 2 | 1 | 2 | 8 |
| 37 | 27.56 | 3.23 | 78.37 | 11.02 | 1 | 2 | 3 | 8 |
| 38 | 27.60 | 3.23 | 440.29 | 26.16 | 1 | 1 | 6 | 8 |
| 39 | 27.74 | 3.21 | 465.54 | 38.24 | 2 | 0 | 4 | 4 |
| 40 | 28.23 | 3.16 | 27.17 | 6.66 | 2 | 1 | 3 | 8 |
| 41 | 28.40 | 3.14 | 194.89 | 25.37 | 0 | 1 | 7 | 4 |
| 42 | 28.62 | 3.12 | 34.35 | 10.74 | 1 | 0 | 7 | 4 |
| 43 | 29.11 | 3.07 | 1343.10 | 68.38 | 0 | 2 | 5 | 4 |
| 44 | 29.31 | 3.04 | 2089.39 | 60.75 | 1 | 2 | 4 | 8 |
| 45 | 29.88 | 2.99 | 186.61 | 37.07 | 0 | 0 | 8 | 2 |
| 46 | 29.95 | 2.98 | 1164.61 | 46.41 | 2 | 1 | 4 | 8 |
| 47 | 29.96 | 2.98 | 288.73 | 32.69 | 2 | 0 | 5 | 4 |
| 48 | 30.77 | 2.90 | 2817.21 | 74.32 | 1 | 1 | 7 | 8 |
| 49 | 31.43 | 2.84 | 1080.43 | 47.08 | 1 | 2 | 5 | 8 |
| 50 | 31.70 | 2.82 | 597.32 | 49.96 | 0 | 2 | 6 | 4 |
| 51 | 31.96 | 2.80 | 23.23 | 9.94 | 0 | 1 | 8 | 4 |
| 52 | 32.03 | 2.79 | 1.10 | 1.53 | 2 | 1 | 5 | 8 |
| 53 | 32.15 | 2.78 | 392.92 | 41.15 | 1 | 0 | 8 | 4 |
| 54 | 32.44 | 2.76 | 195.57 | 29.31 | 2 | 2 | 0 | 4 |
| 55 | 32.49 | 2.75 | 190.70 | 28.99 | 2 | 0 | 6 | 4 |
| 56 | 32.66 | 2.74 | 244.09 | 23.33 | 2 | 2 | 1 | 8 |
| 57 | 33.32 | 2.69 | 510.98 | 34.49 | 2 | 2 | 2 | 8 |
| 58 | 33.84 | 2.65 | 5.33 | 5.06 | 0 | 3 | 1 | 4 |
| 59 | 33.86 | 2.65 | 981.78 | 48.65 | 1 | 2 | 6 | 8 |
| 60 | 34.10 | 2.63 | 1026.65 | 50.14 | 1 | 1 | 8 | 8 |
| 61 | 34.39 | 2.61 | 172.81 | 20.76 | 2 | 2 | 3 | 8 |
| 62 | 34.42 | 2.60 | 581.98 | 38.13 | 2 | 1 | 6 | 8 |
| 63 | 34.48 | 2.60 | 13.39 | 8.20 | 0 | 3 | 2 | 4 |
| 64 | 34.53 | 2.60 | 234.08 | 34.32 | 0 | 2 | 7 | 4 |
| 65 | 35.26 | 2.54 | 85.92 | 21.27 | 2 | 0 | 7 | 4 |
| 66 | 35.50 | 2.53 | 1537.35 | 90.65 | 3 | 0 | 1 | 4 |
| 67 | 35.52 | 2.53 | 251.72 | 36.70 | 0 | 3 | 3 | 4 |
| 68 | 35.60 | 2.52 | 109.81 | 24.30 | 0 | 1 | 9 | 4 |
| 69 | 35.69 | 2.51 | 711.48 | 62.02 | 1 | 3 | 0 | 4 |
| 70 | 35.77 | 2.51 | 649.19 | 59.41 | 1 | 0 | 9 | 4 |
| 71 | 35.84 | 2.50 | 186.84 | 22.58 | 2 | 2 | 4 | 8 |
| 72 | 35.89 | 2.50 | 274.70 | 27.42 | 1 | 3 | 1 | 8 |
| 73 | 36.11 | 2.49 | 30.94 | 13.10 | 3 | 0 | 2 | 4 |
| 74 | 36.49 | 2.46 | 401.05 | 33.75 | 1 | 3 | 2 | 8 |
| 75 | 36.54 | 2.46 | 141.50 | 20.07 | 1 | 2 | 7 | 8 |
| 76 | 36.93 | 2.43 | 65.80 | 19.58 | 0 | 3 | 4 | 4 |
| 77 | 37.07 | 2.42 | 684.95 | 44.86 | 2 | 1 | 7 | 8 |
| 78 | 37.10 | 2.42 | 136.40 | 28.34 | 3 | 1 | 0 | 4 |
| 79 | 37.11 | 2.42 | 0.91 | 2.31 | 3 | 0 | 3 | 4 |
| 80 | 37.30 | 2.41 | 650.03 | 44.00 | 3 | 1 | 1 | 8 |
| 81 | 37.48 | 2.40 | 192.10 | 24.05 | 1 | 3 | 3 | 8 |
| 82 | 37.56 | 2.39 | 595.32 | 42.44 | 1 | 1 | 9 | 8 |
| 83 | 37.56 | 2.39 | 81.72 | 22.24 | 0 | 2 | 8 | 4 |
| 84 | 37.60 | 2.39 | 52.99 | 25.35 | 0 | 0 | 10 | 2 |
| 85 | 37.63 | 2.39 | 943.06 | 53.52 | 2 | 2 | 5 | 8 |
| 86 | 37.88 | 2.37 | 1454.90 | 66.97 | 3 | 1 | 2 | 8 |
| 87 | 38.25 | 2.35 | 686.59 | 65.75 | 2 | 0 | 8 | 4 |
| 88 | 38.47 | 2.34 | 151.42 | 31.08 | 3 | 0 | 4 | 4 |
| 89 | 38.67 | 2.33 | 366.79 | 48.66 | 0 | 3 | 5 | 4 |
| 90 | 38.83 | 2.32 | 73.76 | 15.50 | 1 | 3 | 4 | 8 |
| 91 | 38.84 | 2.32 | 1008.35 | 57.31 | 3 | 1 | 3 | 8 |
| 92 | 39.31 | 2.29 | 141.34 | 30.76 | 0 | 1 | 10 | 4 |
| 93 | 39.44 | 2.28 | 93.47 | 17.75 | 1 | 2 | 8 | 8 |
| 94 | 39.48 | 2.28 | 1.47 | 3.15 | 1 | 0 | 10 | 4 |
| 95 | 39.72 | 2.27 | 171.67 | 24.25 | 2 | 2 | 6 | 8 |
| 96 | 39.94 | 2.26 | 536.82 | 43.13 | 2 | 1 | 8 | 8 |
| 97 | 40.15 | 2.24 | 219.98 | 27.77 | 3 | 1 | 4 | 8 |
| 98 | 40.16 | 2.24 | 9.19 | 8.03 | 3 | 0 | 5 | 4 |
| 99 | 40.51 | 2.23 | 62.87 | 15.00 | 1 | 3 | 5 | 8 |
| 100 | 40.72 | 2.21 | 177.88 | 35.89 | 0 | 3 | 6 | 4 |
| 101 | 40.77 | 2.21 | 79.66 | 24.04 | 0 | 2 | 9 | 4 |
| 102 | 41.13 | 2.19 | 8.63 | 5.65 | 1 | 1 | 10 | 8 |
| 103 | 41.33 | 2.18 | 9.54 | 8.45 | 2 | 3 | 0 | 4 |
| 104 | 41.41 | 2.18 | 167.18 | 35.44 | 2 | 0 | 9 | 4 |
| 105 | 41.51 | 2.17 | 383.54 | 38.06 | 2 | 3 | 1 | 8 |
| 106 | 41.78 | 2.16 | 82.18 | 17.75 | 3 | 1 | 5 | 8 |
| 107 | 42.04 | 2.15 | 67.77 | 16.23 | 2 | 3 | 2 | 8 |
| 108 | 42.09 | 2.15 | 310.31 | 34.77 | 2 | 2 | 7 | 8 |
| 109 | 42.11 | 2.14 | 65.60 | 22.62 | 3 | 2 | 0 | 4 |
| 110 | 42.15 | 2.14 | 409.98 | 56.61 | 3 | 0 | 6 | 4 |
| 111 | 42.29 | 2.14 | 46.42 | 13.52 | 3 | 2 | 1 | 8 |
| 112 | 42.49 | 2.13 | 40.27 | 12.66 | 1 | 3 | 6 | 8 |
| 113 | 42.53 | 2.12 | 274.60 | 33.09 | 1 | 2 | 9 | 8 |
| 114 | 42.82 | 2.11 | 457.75 | 43.05 | 3 | 2 | 2 | 8 |
| 115 | 42.92 | 2.11 | 106.13 | 20.79 | 2 | 3 | 3 | 8 |
| 116 | 42.99 | 2.10 | 340.34 | 37.29 | 2 | 1 | 9 | 8 |
| 117 | 43.04 | 2.10 | 457.84 | 61.25 | 0 | 3 | 7 | 4 |
| 118 | 43.11 | 2.10 | 38.77 | 17.85 | 0 | 1 | 11 | 4 |
| 119 | 43.26 | 2.09 | 95.01 | 28.06 | 1 | 0 | 11 | 4 |
| 120 | 43.69 | 2.07 | 660.25 | 52.89 | 3 | 2 | 3 | 8 |
| 121 | 43.71 | 2.07 | 73.49 | 17.66 | 3 | 1 | 6 | 8 |
| 122 | 44.12 | 2.05 | 37.87 | 18.11 | 0 | 2 | 10 | 4 |
| 123 | 44.13 | 2.05 | 70.50 | 17.48 | 2 | 3 | 4 | 8 |
| 124 | 44.40 | 2.04 | 1.94 | 4.13 | 3 | 0 | 7 | 4 |
| 125 | 44.68 | 2.03 | 25.56 | 10.67 | 2 | 2 | 8 | 8 |
| 126 | 44.72 | 2.03 | 127.04 | 33.68 | 2 | 0 | 10 | 4 |
| 127 | 44.73 | 2.02 | 220.92 | 31.42 | 1 | 3 | 7 | 8 |
| 128 | 44.79 | 2.02 | 121.71 | 23.36 | 1 | 1 | 11 | 8 |
| 129 | 44.88 | 2.02 | 49.03 | 14.86 | 3 | 2 | 4 | 8 |

TABLE 3
Unit cell parameters for orthorhombic
P2₁2₁2₁ (S)-nicotinium L-malate.

| | |
|---|---|
| Orthorhombic P2₁2₁2₁ | R₁ = 3.01% |

| | | |
|---|---|---|
| a = 7.6153 (4) | α = 90° | d min (Mo) = 0.62 |
| b = 7.9772 (4) | β = 90° | Flack = −0.24 (15) |
| c = 23.9107 (11) | γ = 90° | Volume = 1452.55 (12) |

Monoclinic P2₁ (S)-Nicotinium L-Malate. Small Scale: L-(−)-malic acid (134.8 mg, 1.0 mmol) was added into a 20 mL scintillation vial. MeOH (1.0 mL) was added with vigorous agitation. (S)-Nicotine (0.40 mL, 2.5 mmol) was added via micropipette in the dark to avoid degradation. The resulting solution was vortexed for 30 seconds at 3,000 rpm on a VWR Mini Vortexer MV I. The solution was then stored in the dark uncapped to allow for crystal formation while the solvent slowly evaporated. Once the solvent evaporated, the crystalline product was collected via vacuum filtration, washing with n-heptane (3×5 mL) (229.0 mg, 77.28%). The resulting crystalline product was characterized by single crystal X-ray diffraction, simulated powder X-ray diffraction, melting point, differential scanning calorimetry, and a UV irradiation degradation test monitored by ¹H NMR.

Large Scale: L-(−)-malic acid (1.3410 g, 10.0 mmol) was weighed into a 20 mL scintillation vial. MeOH (32 mL) was added with vigorous agitation. (S)-Nicotine (4.00 mL, 25.0 mmol) was added via micropipette in the dark to avoid degradation. The resulting solution was vortexed for 30 seconds at 3,000 rpm on a VWR Mini Vortexer MV I. The solution was then stored in the dark with the cap on tightly until the solution turned deep orange. The vial was then uncovered to allow for crystal formation while the solvent slowly evaporated. Once the solvent was evaporated, the crystalline product was collected via vacuum filtration, washing with n-heptane (3×15 mL) (2.6932 g, 90.89%). The yield was computed relative to the asymmetric unit. The resulting crystalline product was characterized by single crystal X-ray diffraction, simulated powder X-ray diffraction, melting point, differential scanning calorimetry, and a UV irradiation degradation test monitored by ¹H NMR.

TABLE 4
X-ray powder pattern peak data for monoclinic
P2₁ (S)-nicotinium L-malate.

| Peak | 2Θ | d-spacing | | | | | | |
|---|---|---|---|---|---|---|---|---|
| No. | [°] | [Å] | Intensity | F | h | k | l | Mult. |
| 1 | 7.20 | 12.26 | 2721.60 | 19.39 | 0 | 0 | 1 | 2 |
| 2 | 11.72 | 7.55 | 1996.16 | 27.11 | 1 | 0 | 0 | 2 |
| 3 | 13.17 | 6.72 | 51.84 | 4.92 | −1 | 0 | 1 | 2 |
| 4 | 13.34 | 6.63 | 638.30 | 12.37 | 0 | −1 | 1 | 4 |
| 5 | 14.34 | 6.17 | 387.43 | 14.66 | 1 | 0 | 1 | 2 |
| 6 | 14.43 | 6.13 | 3861.29 | 46.59 | 0 | 0 | 2 | 2 |
| 7 | 16.25 | 5.45 | 1320.25 | 21.74 | −1 | 1 | 0 | 4 |
| 8 | 17.34 | 5.11 | 123.70 | 7.11 | −1 | 1 | 1 | 4 |
| 9 | 17.75 | 4.99 | 72.93 | 7.91 | −1 | 0 | 2 | 2 |
| 10 | 18.24 | 4.86 | 10000.00 | 67.36 | 1 | −1 | 1 | 4 |
| 11 | 18.32 | 4.84 | 6548.26 | 54.75 | 0 | −1 | 2 | 4 |
| 12 | 19.48 | 4.55 | 2581.30 | 51.78 | 1 | 0 | 2 | 2 |
| 13 | 21.04 | 4.22 | 3250.82 | 44.51 | −1 | −1 | 2 | 4 |
| 14 | 21.72 | 4.09 | 2132.31 | 52.70 | 0 | 0 | 3 | 2 |
| 15 | 22.53 | 3.94 | 636.07 | 21.14 | 1 | −1 | 2 | 4 |
| 16 | 22.55 | 3.94 | 7494.10 | 102.70 | 0 | 2 | 0 | 2 |
| 17 | 23.56 | 3.77 | 734.63 | 33.66 | 2 | 0 | 0 | 2 |
| 18 | 23.70 | 3.75 | 402.31 | 17.72 | 0 | −2 | 1 | 4 |
| 19 | 23.74 | 3.74 | 1279.10 | 44.78 | −1 | 0 | 3 | 2 |
| 20 | 23.99 | 3.71 | 34.87 | 7.48 | −2 | 0 | 1 | 2 |
| 21 | 24.51 | 3.63 | 2579.07 | 46.49 | 0 | −1 | 3 | 4 |
| 22 | 25.32 | 3.51 | 1160.78 | 45.64 | 2 | 0 | 1 | 2 |
| 23 | 25.48 | 3.49 | 60.63 | 7.43 | −1 | 2 | 0 | 4 |
| 24 | 25.72 | 3.46 | 2323.38 | 65.65 | 1 | 0 | 3 | 2 |
| 25 | 26.16 | 3.40 | 23.38 | 4.74 | −2 | 1 | 0 | 4 |
| 26 | 26.20 | 3.40 | 2511.82 | 49.22 | −1 | 2 | 1 | 4 |
| 27 | 26.33 | 3.38 | 1269.67 | 35.17 | −1 | −1 | 3 | 4 |
| 28 | 26.53 | 3.36 | 15.49 | 5.54 | −2 | 0 | 2 | 2 |
| 29 | 26.56 | 3.35 | 417.00 | 20.34 | −2 | 1 | 1 | 4 |
| 30 | 26.82 | 3.32 | 887.21 | 29.98 | 1 | −2 | 1 | 4 |
| 31 | 26.87 | 3.32 | 4466.81 | 67.42 | 0 | −2 | 2 | 4 |
| 32 | 27.77 | 3.21 | 56.89 | 7.88 | 2 | −1 | 1 | 4 |
| 33 | 28.14 | 3.17 | 816.24 | 30.26 | 1 | −1 | 3 | 4 |
| 34 | 28.84 | 3.09 | 1421.61 | 40.99 | −1 | −2 | 2 | 4 |
| 35 | 28.88 | 3.09 | 272.41 | 17.97 | −2 | 1 | 2 | 4 |
| 36 | 28.90 | 3.09 | 0.87 | 1.44 | 2 | 0 | 2 | 2 |
| 37 | 29.10 | 3.07 | 260.47 | 25.06 | 0 | 0 | 4 | 2 |
| 38 | 29.96 | 2.98 | 438.25 | 23.71 | 1 | −2 | 2 | 4 |
| 39 | 30.40 | 2.94 | 1.18 | 1.77 | −1 | 0 | 4 | 2 |
| 40 | 30.67 | 2.91 | 193.29 | 22.84 | −2 | 0 | 3 | 2 |
| 41 | 31.09 | 2.87 | 339.55 | 21.72 | 2 | −1 | 2 | 4 |
| 42 | 31.28 | 2.86 | 9.24 | 3.61 | 0 | −1 | 4 | 4 |
| 43 | 31.51 | 2.84 | 554.80 | 28.16 | 0 | −2 | 3 | 4 |
| 44 | 32.49 | 2.75 | 969.58 | 38.49 | −1 | −1 | 4 | 4 |
| 45 | 32.51 | 2.75 | 1300.01 | 63.07 | 1 | 0 | 4 | 2 |
| 46 | 32.75 | 2.73 | 49.46 | 8.77 | −2 | 1 | 3 | 4 |
| 47 | 32.84 | 2.73 | 182.70 | 16.90 | −2 | 2 | 0 | 4 |
| 48 | 32.97 | 2.71 | 898.71 | 37.64 | −1 | −2 | 3 | 4 |
| 49 | 33.16 | 2.70 | 456.69 | 27.00 | −2 | 2 | 1 | 4 |
| 50 | 33.78 | 2.65 | 220.98 | 27.10 | 2 | 0 | 3 | 2 |
| 51 | 34.16 | 2.62 | 194.64 | 18.20 | 2 | −2 | 1 | 4 |
| 52 | 34.46 | 2.60 | 706.78 | 35.03 | 1 | −2 | 3 | 4 |
| 53 | 34.50 | 2.60 | 94.15 | 12.80 | 1 | −1 | 4 | 4 |
| 54 | 34.90 | 2.57 | 44.60 | 8.92 | 0 | −3 | 1 | 4 |
| 55 | 35.08 | 2.56 | 119.72 | 14.70 | −2 | 2 | 2 | 4 |
| 56 | 35.66 | 2.52 | 331.34 | 35.21 | 3 | 0 | 0 | 2 |
| 57 | 35.70 | 2.51 | 1098.93 | 45.40 | 2 | −1 | 3 | 4 |
| 58 | 35.73 | 2.51 | 946.04 | 59.61 | −3 | 0 | 1 | 2 |
| 59 | 35.93 | 2.50 | 184.28 | 26.48 | −2 | 0 | 4 | 2 |
| 60 | 36.18 | 2.48 | 638.12 | 35.10 | −1 | 3 | 0 | 4 |
| 61 | 36.61 | 2.45 | 41.76 | 12.86 | 0 | 0 | 5 | 2 |
| 62 | 36.71 | 2.45 | 75.58 | 12.27 | −1 | 3 | 1 | 4 |
| 63 | 36.96 | 2.43 | 323.80 | 25.60 | 2 | −2 | 2 | 4 |
| 64 | 37.12 | 2.42 | 1.00 | 2.02 | 3 | 0 | 1 | 2 |
| 65 | 37.13 | 2.42 | 272.77 | 23.61 | 0 | −2 | 4 | 4 |
| 66 | 37.17 | 2.42 | 188.74 | 19.66 | 1 | −3 | 1 | 4 |
| 67 | 37.21 | 2.41 | 367.29 | 27.46 | 0 | −3 | 2 | 4 |
| 68 | 37.31 | 2.41 | 2.50 | 3.21 | −3 | 0 | 2 | 2 |
| 69 | 37.44 | 2.40 | 15.55 | 8.05 | −1 | 0 | 5 | 2 |
| 70 | 37.50 | 2.40 | 80.02 | 12.93 | −3 | 1 | 0 | 4 |
| 71 | 37.56 | 2.39 | 597.90 | 35.41 | −3 | 1 | 1 | 4 |
| 72 | 37.76 | 2.38 | 656.07 | 37.30 | −2 | −1 | 4 | 4 |
| 73 | 38.17 | 2.36 | 49.10 | 10.33 | −1 | −2 | 4 | 4 |
| 74 | 38.40 | 2.34 | 193.48 | 20.64 | −2 | 2 | 3 | 4 |
| 75 | 38.41 | 2.34 | 169.50 | 19.32 | 0 | −1 | 5 | 4 |
| 76 | 38.70 | 2.33 | 75.71 | 13.02 | −1 | −3 | 2 | 4 |
| 77 | 38.90 | 2.31 | 1116.30 | 50.29 | 3 | −1 | 1 | 4 |
| 78 | 39.08 | 2.30 | 428.21 | 31.31 | −3 | 1 | 2 | 4 |
| 79 | 39.21 | 2.30 | 356.75 | 28.68 | −1 | −1 | 5 | 4 |
| 80 | 39.55 | 2.28 | 2.57 | 3.47 | 2 | 0 | 4 | 2 |
| 81 | 39.57 | 2.28 | 58.12 | 11.69 | 1 | −3 | 2 | 4 |
| 82 | 39.66 | 2.27 | 5.30 | 5.01 | 1 | 0 | 5 | 2 |
| 83 | 39.93 | 2.26 | 18.69 | 6.70 | 1 | −2 | 4 | 4 |
| 84 | 39.97 | 2.25 | 184.31 | 29.78 | 3 | 0 | 2 | 2 |
| 85 | 40.26 | 2.24 | 276.97 | 36.80 | −3 | 0 | 3 | 2 |
| 86 | 40.80 | 2.21 | 352.96 | 29.81 | 0 | −3 | 3 | 4 |
| 87 | 41.00 | 2.20 | 427.35 | 32.99 | 2 | −2 | 3 | 4 |
| 88 | 41.24 | 2.19 | 130.29 | 18.33 | 2 | −1 | 4 | 4 |
| 89 | 41.35 | 2.18 | 177.16 | 21.44 | 1 | −1 | 5 | 4 |
| 90 | 41.64 | 2.17 | 83.99 | 14.88 | 3 | −1 | 2 | 4 |
| 91 | 41.87 | 2.16 | 213.61 | 23.87 | −2 | 3 | 0 | 4 |
| 92 | 41.92 | 2.15 | 15.11 | 6.36 | −3 | 1 | 3 | 4 |
| 93 | 41.97 | 2.15 | 150.51 | 28.42 | −2 | 0 | 5 | 2 |
| 94 | 41.98 | 2.15 | 56.66 | 12.33 | −1 | −3 | 3 | 4 |
| 95 | 42.13 | 2.14 | 60.61 | 12.81 | −2 | 3 | 1 | 4 |
| 96 | 42.61 | 2.12 | 188.35 | 22.86 | −3 | 2 | 0 | 4 |
| 97 | 42.66 | 2.12 | 86.74 | 15.54 | −3 | 2 | 1 | 4 |
| 98 | 42.84 | 2.11 | 12.01 | 5.81 | −2 | −2 | 4 | 4 |
| 99 | 42.95 | 2.10 | 42.81 | 11.00 | 2 | −3 | 1 | 4 |
| 100 | 43.20 | 2.09 | 44.24 | 11.25 | 1 | −3 | 3 | 4 |
| 101 | 43.42 | 2.08 | 22.36 | 8.05 | 0 | −2 | 5 | 4 |
| 102 | 43.58 | 2.08 | 156.47 | 21.37 | −2 | −1 | 5 | 4 |
| 103 | 43.72 | 2.07 | 75.55 | 14.90 | −2 | 3 | 2 | 4 |
| 104 | 43.87 | 2.06 | 421.67 | 35.35 | 3 | −2 | 1 | 4 |
| 105 | 43.97 | 2.06 | 12.40 | 8.59 | 3 | 0 | 3 | 2 |
| 106 | 44.03 | 2.05 | 129.60 | 19.68 | −3 | 2 | 2 | 4 |
| 107 | 44.15 | 2.05 | 50.00 | 12.26 | −1 | −2 | 5 | 4 |
| 108 | 44.28 | 2.04 | 38.97 | 15.36 | 0 | 0 | 6 | 2 |
| 109 | 44.34 | 2.04 | 38.18 | 15.23 | −3 | 0 | 4 | 2 |
| 110 | 44.80 | 2.02 | 5.04 | 5.60 | −1 | 0 | 6 | 2 |

TABLE 5
Unit cell parameters for monoclinic P2₁ (S)-nicotinium L-malate.

| | |
|---|---|
| Monoclinic P2₁ | R₁ = 3.76% |

| | | |
|---|---|---|
| a = 7.5803 (6) | α = 90° | d min (Mo) = 0.67 |
| b = 7.8810 (6) | β = 95.396 (2)° | Flack = −0.3 (4) |
| c = 12.3182 (9) | γ = 90° | Volume = 732.63 (10) |

Orthorhombic P2₁2₁2₁ (S)-Nicotinium D-Malate. 1^{st} Small Scale: D-(+)-malic acid (134.6 mg, 1.0 mmol) was added into a 20 mL scintillation vial. MeOH (3.0 mL) was added with vigorous agitation. (S)-Nicotine (0.16 mL, 1.0 mmol) was added via micropipette in the dark to avoid degradation. The resulting solution was vortexed for 30 seconds at 3,000 rpm on a VWR Mini Vortexer MV I. The solution was then stored in the dark, uncapped to allow for crystal formation while the solvent slowly evaporated. Once the solvent evaporated, the crystalline product was collected via vacuum filtration, washing with n-heptane (3×5 mL) (184.3 mg, 62.20%). The resulting crystalline product was characterized by single crystal X-ray diffraction, simulated powder X-ray diffraction, melting point, differential scanning calorimetry, and a UV irradiation degradation test monitored by ¹H NMR.

2nd Small Scale: D-(+)-malic acid (135.1 mg, 1.0 mmol) was added into a 20 mL scintillation vial. MeOH (3.0 mL) was added with vigorous agitation. (S)-Nicotine (0.38 mL, 2.38 mmol) was added via micropipette in the dark to avoid degradation. The resulting solution was vortexed for 30 seconds at 3,000 rpm on a VWR Mini Vortexer MV I. The solution was then stored in the dark, uncapped to allow for crystal formation while the solvent slowly evaporated. Once the solvent evaporated, the crystalline product was collected via vacuum filtration, washing with n-heptane (3×5 mL) (198.1 mg, 66.86%). The resulting crystalline product was characterized by single crystal X-ray diffraction, simulated powder X-ray diffraction, melting point, differential scanning calorimetry, and a UV irradiation degradation test monitored by ¹H NMR.

TABLE 6
X-ray powder pattern peak data for orthorhombic
P2₁2₁2₁ (S)-nicotinium D-malate.

| Peak | 2Θ | d-spacing | | | | | | |
|---|---|---|---|---|---|---|---|---|
| No. | [°] | [Å] | Intensity | F | h | k | l | Mult. |
| 1 | 7.76 | 11.39 | 438.38 | 14.43 | 0 | 0 | 2 | 2 |
| 2 | 11.13 | 7.94 | 167.33 | 9.07 | 0 | 1 | 1 | 4 |
| 3 | 12.29 | 7.20 | 147.64 | 9.42 | 1 | 0 | 1 | 4 |
| 4 | 13.01 | 6.80 | 57.62 | 6.24 | 0 | 1 | 2 | 4 |
| 5 | 14.02 | 6.31 | 5228.62 | 64.05 | 1 | 0 | 2 | 4 |
| 6 | 15.55 | 5.69 | 3313.96 | 80.17 | 0 | 0 | 4 | 2 |
| 7 | 15.66 | 5.65 | 25.09 | 4.97 | 0 | 1 | 3 | 4 |
| 8 | 15.67 | 5.65 | 557.66 | 23.43 | 1 | 1 | 0 | 4 |
| 9 | 16.14 | 5.49 | 3823.66 | 44.74 | 1 | 1 | 1 | 8 |
| 10 | 16.51 | 5.37 | 2742.79 | 54.81 | 1 | 0 | 3 | 4 |
| 11 | 17.50 | 5.06 | 5090.30 | 56.07 | 1 | 1 | 2 | 8 |
| 12 | 18.76 | 4.73 | 9048.99 | 113.56 | 0 | 1 | 4 | 4 |
| 13 | 19.48 | 4.55 | 3577.19 | 74.20 | 1 | 0 | 4 | 4 |
| 14 | 19.57 | 4.53 | 2472.39 | 43.83 | 1 | 1 | 3 | 8 |
| 15 | 20.95 | 4.24 | 10000.00 | 189.23 | 0 | 2 | 0 | 2 |
| 16 | 21.32 | 4.16 | 1929.32 | 59.83 | 0 | 2 | 1 | 4 |
| 17 | 22.14 | 4.01 | 39.11 | 8.86 | 0 | 1 | 5 | 4 |
| 18 | 22.14 | 4.01 | 8803.62 | 94.02 | 1 | 1 | 4 | 8 |
| 19 | 22.37 | 3.97 | 78.21 | 12.67 | 0 | 2 | 2 | 4 |
| 20 | 22.75 | 3.91 | 286.17 | 24.66 | 1 | 0 | 5 | 4 |
| 21 | 23.42 | 3.80 | 4251.39 | 138.53 | 0 | 0 | 6 | 2 |
| 22 | 23.43 | 3.79 | 230.95 | 32.31 | 2 | 0 | 0 | 2 |
| 23 | 23.76 | 3.74 | 38.60 | 9.48 | 2 | 0 | 1 | 4 |
| 24 | 24.04 | 3.70 | 38.20 | 9.54 | 0 | 2 | 3 | 4 |
| 25 | 24.04 | 3.70 | 47.12 | 10.60 | 1 | 2 | 0 | 4 |
| 26 | 24.36 | 3.65 | 1757.37 | 46.41 | 1 | 2 | 1 | 8 |
| 27 | 24.72 | 3.60 | 1063.28 | 51.84 | 2 | 0 | 2 | 4 |
| 28 | 25.09 | 3.55 | 1009.49 | 36.28 | 1 | 1 | 5 | 8 |
| 29 | 25.30 | 3.52 | 567.85 | 27.45 | 1 | 2 | 2 | 8 |
| 30 | 25.70 | 3.46 | 1533.36 | 64.86 | 0 | 1 | 6 | 4 |
| 31 | 25.71 | 3.46 | 11.86 | 5.71 | 2 | 1 | 0 | 4 |
| 32 | 26.01 | 3.42 | 617.19 | 29.47 | 2 | 1 | 1 | 8 |
| 33 | 26.20 | 3.40 | 4177.25 | 109.25 | 0 | 2 | 4 | 4 |
| 34 | 26.23 | 3.39 | 37.46 | 10.36 | 1 | 0 | 6 | 4 |
| 35 | 26.24 | 3.39 | 1380.27 | 62.91 | 2 | 0 | 3 | 4 |
| 36 | 26.79 | 3.33 | 4295.98 | 80.21 | 1 | 2 | 3 | 8 |
| 37 | 26.89 | 3.31 | 831.64 | 35.44 | 2 | 1 | 2 | 8 |
| 38 | 28.25 | 3.16 | 264.96 | 29.80 | 2 | 0 | 4 | 4 |
| 39 | 28.30 | 3.15 | 2382.94 | 63.32 | 1 | 1 | 6 | 8 |
| 40 | 28.31 | 3.15 | 820.57 | 37.17 | 2 | 1 | 3 | 8 |
| 41 | 28.76 | 3.10 | 112.57 | 19.80 | 0 | 2 | 5 | 4 |
| 42 | 28.76 | 3.10 | 177.30 | 17.57 | 1 | 2 | 4 | 8 |
| 43 | 29.38 | 3.04 | 101.16 | 19.21 | 0 | 1 | 7 | 4 |
| 44 | 29.86 | 2.99 | 497.64 | 43.33 | 1 | 0 | 7 | 4 |
| 45 | 30.19 | 2.96 | 93.76 | 13.46 | 2 | 1 | 4 | 8 |
| 46 | 30.65 | 2.92 | 123.98 | 22.24 | 2 | 0 | 5 | 4 |
| 47 | 31.12 | 2.87 | 756.06 | 39.49 | 1 | 2 | 5 | 8 |
| 48 | 31.40 | 2.85 | 242.62 | 45.17 | 0 | 0 | 8 | 2 |
| 49 | 31.62 | 2.83 | 204.60 | 29.56 | 0 | 2 | 6 | 4 |
| 50 | 31.63 | 2.83 | 192.63 | 28.69 | 2 | 2 | 0 | 4 |
| 51 | 31.71 | 2.82 | 1992.28 | 65.40 | 1 | 1 | 7 | 8 |
| 52 | 31.88 | 2.80 | 802.03 | 41.74 | 2 | 2 | 1 | 8 |
| 53 | 31.90 | 2.80 | 1084.18 | 68.69 | 0 | 3 | 1 | 4 |
| 54 | 32.46 | 2.76 | 276.77 | 25.00 | 2 | 1 | 5 | 8 |
| 55 | 32.62 | 2.74 | 91.17 | 14.43 | 2 | 2 | 2 | 8 |
| 56 | 32.64 | 2.74 | 16.12 | 8.59 | 0 | 3 | 2 | 4 |
| 57 | 33.17 | 2.70 | 241.66 | 33.82 | 0 | 1 | 8 | 4 |
| 58 | 33.37 | 2.68 | 837.43 | 63.37 | 2 | 0 | 6 | 4 |
| 59 | 33.60 | 2.67 | 98.00 | 21.84 | 1 | 0 | 8 | 4 |
| 60 | 33.81 | 2.65 | 439.99 | 32.95 | 1 | 2 | 6 | 8 |
| 61 | 33.82 | 2.65 | 96.22 | 15.41 | 2 | 2 | 3 | 8 |
| 62 | 33.84 | 2.65 | 1150.90 | 75.42 | 0 | 3 | 3 | 4 |
| 63 | 33.84 | 2.65 | 1037.31 | 71.61 | 1 | 3 | 0 | 4 |
| 64 | 34.07 | 2.63 | 174.10 | 20.90 | 1 | 3 | 1 | 8 |
| 65 | 34.74 | 2.58 | 42.11 | 14.85 | 0 | 2 | 7 | 4 |
| 66 | 34.77 | 2.58 | 537.87 | 37.56 | 1 | 3 | 2 | 8 |
| 67 | 35.05 | 2.56 | 559.81 | 38.65 | 2 | 1 | 6 | 8 |
| 68 | 35.27 | 2.54 | 382.77 | 32.18 | 1 | 1 | 8 | 8 |
| 69 | 35.43 | 2.53 | 657.19 | 42.38 | 2 | 2 | 4 | 8 |
| 70 | 35.45 | 2.53 | 359.04 | 44.33 | 0 | 3 | 4 | 4 |
| 71 | 35.69 | 2.51 | 125.16 | 26.36 | 3 | 0 | 1 | 4 |
| 72 | 35.90 | 2.50 | 248.33 | 26.43 | 1 | 3 | 3 | 8 |
| 73 | 36.35 | 2.47 | 1273.86 | 85.81 | 2 | 0 | 7 | 4 |
| 74 | 36.36 | 2.47 | 904.47 | 72.33 | 3 | 0 | 2 | 4 |
| 75 | 36.76 | 2.44 | 360.67 | 32.69 | 1 | 2 | 7 | 8 |
| 76 | 37.05 | 2.42 | 125.80 | 27.54 | 0 | 1 | 9 | 4 |
| 77 | 37.07 | 2.42 | 273.60 | 40.63 | 3 | 1 | 0 | 4 |
| 78 | 37.28 | 2.41 | 579.45 | 42.08 | 3 | 1 | 1 | 8 |
| 79 | 37.42 | 2.40 | 276.80 | 29.20 | 2 | 2 | 5 | 8 |
| 80 | 37.43 | 2.40 | 9.20 | 7.53 | 1 | 0 | 9 | 4 |
| 81 | 37.44 | 2.40 | 220.92 | 36.91 | 0 | 3 | 5 | 4 |
| 82 | 37.44 | 2.40 | 243.44 | 27.40 | 1 | 3 | 4 | 8 |
| 83 | 37.45 | 2.40 | 359.35 | 47.10 | 3 | 0 | 3 | 4 |
| 84 | 37.92 | 2.37 | 999.31 | 56.30 | 2 | 1 | 7 | 8 |
| 85 | 37.93 | 2.37 | 510.11 | 40.24 | 3 | 1 | 2 | 8 |
| 86 | 38.05 | 2.36 | 224.49 | 37.89 | 0 | 2 | 8 | 4 |
| 87 | 38.94 | 2.31 | 1136.65 | 87.44 | 3 | 0 | 4 | 4 |
| 88 | 38.97 | 2.31 | 160.87 | 23.28 | 1 | 1 | 9 | 8 |
| 89 | 38.98 | 2.31 | 45.92 | 12.44 | 3 | 1 | 3 | 8 |
| 90 | 39.34 | 2.29 | 16.30 | 7.49 | 1 | 3 | 5 | 8 |
| 91 | 39.54 | 2.28 | 62.15 | 29.41 | 0 | 0 | 10 | 2 |
| 92 | 39.55 | 2.28 | 33.63 | 15.30 | 2 | 0 | 8 | 4 |
| 93 | 39.73 | 2.27 | 228.62 | 28.36 | 2 | 2 | 6 | 8 |
| 94 | 39.75 | 2.27 | 265.96 | 43.28 | 0 | 3 | 6 | 4 |
| 95 | 39.76 | 2.27 | 145.68 | 32.04 | 2 | 3 | 0 | 4 |
| 96 | 39.93 | 2.26 | 207.93 | 27.20 | 1 | 2 | 8 | 8 |
| 97 | 39.96 | 2.25 | 99.71 | 18.85 | 2 | 3 | 1 | 8 |
| 98 | 40.42 | 2.23 | 908.65 | 57.63 | 3 | 1 | 4 | 8 |
| 99 | 40.57 | 2.22 | 127.88 | 21.71 | 2 | 3 | 2 | 8 |
| 100 | 40.78 | 2.21 | 475.89 | 59.56 | 3 | 0 | 5 | 4 |
| 101 | 41.00 | 2.20 | 206.94 | 39.52 | 0 | 1 | 10 | 4 |
| 102 | 41.01 | 2.20 | 54.19 | 14.30 | 2 | 1 | 8 | 8 |
| 103 | 41.36 | 2.18 | 658.76 | 71.19 | 1 | 0 | 10 | 4 |
| 104 | 41.54 | 2.17 | 98.34 | 27.64 | 0 | 2 | 9 | 4 |
| 105 | 41.55 | 2.17 | 113.69 | 29.73 | 3 | 2 | 0 | 4 |
| 106 | 41.56 | 2.17 | 11.20 | 6.60 | 1 | 3 | 6 | 8 |
| 107 | 41.57 | 2.17 | 78.78 | 17.51 | 2 | 3 | 3 | 8 |
| 108 | 41.75 | 2.16 | 14.40 | 7.52 | 3 | 2 | 1 | 8 |
| 109 | 42.21 | 2.14 | 40.47 | 12.76 | 3 | 1 | 5 | 8 |
| 110 | 42.33 | 2.13 | 369.31 | 38.68 | 2 | 2 | 7 | 8 |
| 111 | 42.34 | 2.13 | 88.17 | 18.91 | 3 | 2 | 2 | 8 |
| 112 | 42.34 | 2.13 | 0.97 | 2.81 | 0 | 3 | 7 | 4 |
| 113 | 42.65 | 2.12 | 53.23 | 29.62 | 0 | 4 | 0 | 2 |
| 114 | 42.77 | 2.11 | 299.40 | 35.24 | 1 | 1 | 10 | 8 |
| 115 | 42.84 | 2.11 | 10.01 | 9.13 | 0 | 4 | 1 | 4 |
| 116 | 42.93 | 2.11 | 152.97 | 35.77 | 2 | 0 | 9 | 4 |
| 117 | 42.93 | 2.10 | 338.45 | 37.63 | 2 | 3 | 4 | 8 |
| 118 | 42.94 | 2.10 | 18.33 | 12.38 | 3 | 0 | 6 | 4 |
| 119 | 43.29 | 2.09 | 305.07 | 36.05 | 1 | 2 | 9 | 8 |
| 120 | 43.30 | 2.09 | 189.44 | 28.42 | 3 | 2 | 3 | 8 |
| 121 | 43.42 | 2.08 | 50.42 | 20.80 | 0 | 4 | 2 | 4 |
| 122 | 44.07 | 2.05 | 145.14 | 25.37 | 1 | 3 | 7 | 8 |
| 123 | 44.30 | 2.04 | 128.45 | 24.01 | 2 | 1 | 9 | 8 |
| 124 | 44.31 | 2.04 | 325.72 | 38.25 | 3 | 1 | 6 | 8 |
| 125 | 44.36 | 2.04 | 72.39 | 25.53 | 0 | 4 | 3 | 4 |
| 126 | 44.37 | 2.04 | 7.54 | 8.24 | 1 | 4 | 0 | 4 |
| 127 | 44.55 | 2.03 | 58.08 | 16.25 | 1 | 4 | 1 | 8 |
| 128 | 44.62 | 2.03 | 16.29 | 8.62 | 3 | 2 | 4 | 8 |
| 129 | 44.64 | 2.03 | 10.17 | 6.82 | 2 | 3 | 5 | 8 |

TABLE 7
Unit cell parameters for orthorhombic
P2₁2₁2₁ (S)-nicotinium D-malate.

| | |
|---|---|
| Orthorhombic P2₁2₁2₁ | R₁ = 3.28% |

| | | |
|---|---|---|
| a = 7.5902 (7) | α = 90° | d min (Mo) = 0.62 |
| b = 8.4613 (8) | β = 90° | Flack = −0.37 (19) |
| c = 22.813 (2) | γ = 90° | Volume = 1465.1 (2) |

Orthorhombic P2₁2₁2₁ (S)-Nicotinium DL-Malate. DL-malic acid (269.8 mg, 2.0 mmol) was added into a 20 mL scintillation vial. MeOH (5.0 mL) was added with vigorous agitation. (S)-Nicotine (0.48 mL, 3.0 mmol) was added via micropipette in the dark to avoid degradation. The resulting solution was vortexed for 30 seconds at 3,000 rpm on a VWR Mini Vortexer MV I. The solution was then stored in the dark, uncapped to allow for crystal formation while the solvent slowly evaporated. Once the solvent evaporated, the crystalline product was collected via vacuum filtration, washing with n-heptane (3×5 mL) (336.3 mg, 56.75%). The resulting crystalline product was characterized by single crystal X-ray diffraction, simulated powder X-ray diffraction, melting point, differential scanning calorimetry, and a UV irradiation degradation test monitored by ¹H NMR.

TABLE 8
X-ray powder pattern peak data for orthorhombic
P2₁2₁2₁ (S)-nicotinium DL-malate.

| Peak | 2Θ | d-spacing | | | | | | |
|---|---|---|---|---|---|---|---|---|
| No. | [°] | [Å] | Intensity | F | h | k | l | Mult. |
| 1 | 7.41 | 11.93 | 1793.80 | 27.20 | 0 | 0 | 2 | 2 |
| 2 | 11.58 | 7.64 | 14.14 | 2.68 | 0 | 1 | 1 | 4 |
| 3 | 12.22 | 7.24 | 2676.70 | 38.92 | 1 | 0 | 1 | 4 |
| 4 | 13.24 | 6.68 | 281.49 | 13.70 | 0 | 1 | 2 | 4 |
| 5 | 13.81 | 6.41 | 44.94 | 5.71 | 1 | 0 | 2 | 4 |
| 6 | 14.84 | 5.96 | 5345.90 | 94.80 | 0 | 0 | 4 | 2 |
| 7 | 15.64 | 5.66 | 3758.53 | 59.28 | 0 | 1 | 3 | 4 |
| 8 | 16.02 | 5.53 | 1296.99 | 35.68 | 1 | 1 | 0 | 4 |
| 9 | 16.12 | 5.49 | 2555.31 | 50.42 | 1 | 0 | 3 | 4 |
| 10 | 16.44 | 5.39 | 3010.45 | 39.49 | 1 | 1 | 1 | 8 |
| 11 | 17.67 | 5.02 | 4787.44 | 53.59 | 1 | 1 | 2 | 8 |
| 12 | 18.49 | 4.79 | 5745.07 | 87.02 | 0 | 1 | 4 | 4 |
| 13 | 18.90 | 4.69 | 1629.96 | 47.41 | 1 | 0 | 4 | 4 |
| 14 | 19.54 | 4.54 | 8573.97 | 79.56 | 1 | 1 | 3 | 8 |
| 15 | 21.63 | 4.11 | 348.78 | 25.21 | 0 | 1 | 5 | 4 |
| 16 | 21.90 | 4.05 | 4302.84 | 63.45 | 1 | 1 | 4 | 8 |
| 17 | 21.98 | 4.04 | 438.31 | 28.75 | 1 | 0 | 5 | 4 |
| 18 | 22.03 | 4.03 | 1867.28 | 84.10 | 0 | 2 | 0 | 2 |
| 19 | 22.35 | 3.98 | 3319.24 | 113.79 | 0 | 0 | 6 | 2 |
| 20 | 22.35 | 3.98 | 10000.00 | 139.67 | 0 | 2 | 1 | 4 |
| 21 | 23.27 | 3.82 | 207.00 | 20.96 | 0 | 2 | 2 | 4 |
| 22 | 23.40 | 3.80 | 850.45 | 60.44 | 2 | 0 | 0 | 2 |
| 23 | 23.70 | 3.75 | 112.98 | 15.79 | 2 | 0 | 1 | 4 |
| 24 | 24.58 | 3.62 | 134.20 | 17.87 | 2 | 0 | 2 | 4 |
| 25 | 24.63 | 3.61 | 1401.11 | 40.92 | 1 | 1 | 5 | 8 |
| 26 | 24.74 | 3.60 | 2004.49 | 69.56 | 0 | 2 | 3 | 4 |
| 27 | 24.95 | 3.57 | 3935.82 | 98.35 | 0 | 1 | 6 | 4 |
| 28 | 24.98 | 3.56 | 103.11 | 15.94 | 1 | 2 | 0 | 4 |
| 29 | 25.26 | 3.52 | 3902.02 | 99.21 | 1 | 0 | 6 | 4 |
| 30 | 25.27 | 3.52 | 1054.05 | 36.46 | 1 | 2 | 1 | 8 |
| 31 | 25.91 | 3.44 | 10.65 | 5.32 | 2 | 1 | 0 | 4 |
| 32 | 25.98 | 3.43 | 1460.72 | 62.50 | 2 | 0 | 3 | 4 |
| 33 | 26.09 | 3.41 | 3625.70 | 69.96 | 1 | 2 | 2 | 8 |
| 34 | 26.18 | 3.40 | 257.96 | 18.73 | 2 | 1 | 1 | 8 |
| 35 | 26.67 | 3.34 | 2441.83 | 83.10 | 0 | 2 | 4 | 4 |
| 36 | 26.98 | 3.30 | 364.86 | 22.99 | 2 | 1 | 2 | 8 |
| 37 | 27.42 | 3.25 | 111.82 | 12.95 | 1 | 2 | 3 | 8 |
| 38 | 27.61 | 3.23 | 497.38 | 27.51 | 1 | 1 | 6 | 8 |
| 39 | 27.82 | 3.20 | 387.79 | 34.64 | 2 | 0 | 4 | 4 |
| 40 | 28.27 | 3.15 | 15.57 | 4.99 | 2 | 1 | 3 | 8 |
| 41 | 28.41 | 3.14 | 190.41 | 24.82 | 0 | 1 | 7 | 4 |
| 42 | 28.69 | 3.11 | 39.05 | 11.36 | 1 | 0 | 7 | 4 |
| 43 | 28.97 | 3.08 | 1433.35 | 69.53 | 0 | 2 | 5 | 4 |
| 44 | 29.19 | 3.06 | 2335.18 | 63.24 | 1 | 2 | 4 | 8 |
| 45 | 29.94 | 2.98 | 175.69 | 35.66 | 0 | 0 | 8 | 2 |
| 46 | 29.99 | 2.98 | 1140.64 | 45.50 | 2 | 1 | 4 | 8 |
| 47 | 30.05 | 2.97 | 281.32 | 32.02 | 2 | 0 | 5 | 4 |
| 48 | 30.80 | 2.90 | 2691.04 | 71.91 | 1 | 1 | 7 | 8 |
| 49 | 31.32 | 2.85 | 1101.39 | 46.85 | 1 | 2 | 5 | 8 |
| 50 | 31.58 | 2.83 | 482.98 | 44.27 | 0 | 2 | 6 | 4 |
| 51 | 31.98 | 2.80 | 38.77 | 12.71 | 0 | 1 | 8 | 4 |
| 52 | 32.08 | 2.79 | 30.76 | 8.03 | 2 | 1 | 5 | 8 |
| 53 | 32.23 | 2.78 | 425.99 | 42.49 | 1 | 0 | 8 | 4 |
| 54 | 32.36 | 2.76 | 202.87 | 29.45 | 2 | 2 | 0 | 4 |
| 55 | 32.58 | 2.75 | 111.54 | 22.00 | 2 | 0 | 6 | 4 |
| 56 | 32.58 | 2.75 | 182.95 | 19.92 | 2 | 2 | 1 | 8 |
| 57 | 33.24 | 2.69 | 527.89 | 34.59 | 2 | 2 | 2 | 8 |
| 58 | 33.53 | 2.67 | 0.61 | 1.68 | 0 | 3 | 1 | 4 |
| 59 | 33.76 | 2.65 | 926.48 | 46.61 | 1 | 2 | 6 | 8 |
| 60 | 34.14 | 2.62 | 1002.07 | 49.05 | 1 | 1 | 8 | 8 |
| 61 | 34.17 | 2.62 | 12.41 | 7.73 | 0 | 3 | 2 | 4 |
| 62 | 34.31 | 2.61 | 170.85 | 20.37 | 2 | 2 | 3 | 8 |
| 63 | 34.43 | 2.60 | 221.73 | 32.94 | 0 | 2 | 7 | 4 |
| 64 | 34.47 | 2.60 | 612.87 | 38.77 | 2 | 1 | 6 | 8 |
| 65 | 35.22 | 2.55 | 369.45 | 43.58 | 0 | 3 | 3 | 4 |
| 66 | 35.36 | 2.54 | 109.63 | 23.84 | 2 | 0 | 7 | 4 |
| 67 | 35.40 | 2.53 | 655.33 | 58.36 | 1 | 3 | 0 | 4 |
| 68 | 35.60 | 2.52 | 248.51 | 25.58 | 1 | 3 | 1 | 8 |
| 69 | 35.62 | 2.52 | 1639.15 | 92.95 | 3 | 0 | 1 | 4 |
| 70 | 35.63 | 2.52 | 85.67 | 21.25 | 0 | 1 | 9 | 4 |
| 71 | 35.77 | 2.51 | 180.24 | 21.89 | 2 | 2 | 4 | 8 |
| 72 | 35.86 | 2.50 | 624.43 | 57.78 | 1 | 0 | 9 | 4 |
| 73 | 36.22 | 2.48 | 349.35 | 30.89 | 1 | 3 | 2 | 8 |
| 74 | 36.24 | 2.48 | 114.02 | 24.98 | 3 | 0 | 2 | 4 |
| 75 | 36.47 | 2.46 | 124.80 | 18.61 | 1 | 2 | 7 | 8 |
| 76 | 36.65 | 2.45 | 73.60 | 20.32 | 0 | 3 | 4 | 4 |
| 77 | 37.13 | 2.42 | 751.36 | 46.56 | 2 | 1 | 7 | 8 |
| 78 | 37.19 | 2.42 | 88.97 | 22.70 | 3 | 1 | 0 | 4 |
| 79 | 37.21 | 2.41 | 187.87 | 23.34 | 1 | 3 | 3 | 8 |
| 80 | 37.23 | 2.41 | 0.01 | 0.25 | 3 | 0 | 3 | 4 |
| 81 | 37.38 | 2.40 | 530.32 | 39.42 | 3 | 1 | 1 | 8 |
| 82 | 37.49 | 2.40 | 110.53 | 25.53 | 0 | 2 | 8 | 4 |
| 83 | 37.57 | 2.39 | 932.61 | 52.56 | 2 | 2 | 5 | 8 |
| 84 | 37.61 | 2.39 | 722.18 | 46.30 | 1 | 1 | 9 | 8 |
| 85 | 37.68 | 2.39 | 54.92 | 25.59 | 0 | 0 | 10 | 2 |
| 86 | 37.97 | 2.37 | 1612.17 | 69.91 | 3 | 1 | 2 | 8 |
| 87 | 38.35 | 2.35 | 549.90 | 58.38 | 2 | 0 | 8 | 4 |
| 88 | 38.41 | 2.34 | 480.78 | 54.69 | 0 | 3 | 5 | 4 |
| 89 | 38.58 | 2.33 | 56.10 | 13.27 | 1 | 3 | 4 | 8 |
| 90 | 38.60 | 2.33 | 193.60 | 34.89 | 3 | 0 | 4 | 4 |
| 91 | 38.93 | 2.31 | 1002.25 | 56.67 | 3 | 1 | 3 | 8 |
| 92 | 39.36 | 2.29 | 145.22 | 30.88 | 0 | 1 | 10 | 4 |
| 93 | 39.38 | 2.29 | 95.74 | 17.74 | 1 | 2 | 8 | 8 |
| 94 | 39.57 | 2.28 | 0.01 | 0.28 | 1 | 0 | 10 | 4 |
| 95 | 39.68 | 2.27 | 174.27 | 24.14 | 2 | 2 | 6 | 8 |
| 96 | 40.00 | 2.25 | 394.13 | 36.63 | 2 | 1 | 8 | 8 |
| 97 | 40.24 | 2.24 | 300.68 | 32.20 | 3 | 1 | 4 | 8 |
| 98 | 40.27 | 2.24 | 42.06 | 12.05 | 1 | 3 | 5 | 8 |
| 99 | 40.29 | 2.24 | 2.97 | 4.53 | 3 | 0 | 5 | 4 |
| 100 | 40.48 | 2.23 | 171.26 | 34.60 | 0 | 3 | 6 | 4 |
| 101 | 40.71 | 2.21 | 100.54 | 26.68 | 0 | 2 | 9 | 4 |
| 102 | 41.11 | 2.19 | 9.95 | 8.48 | 2 | 3 | 0 | 4 |
| 103 | 41.18 | 2.19 | 10.30 | 6.12 | 1 | 1 | 10 | 8 |
| 104 | 41.29 | 2.18 | 438.40 | 40.02 | 2 | 3 | 1 | 8 |
| 105 | 41.51 | 2.17 | 238.73 | 42.01 | 2 | 0 | 9 | 4 |
| 106 | 41.83 | 2.16 | 35.94 | 11.63 | 2 | 3 | 2 | 8 |
| 107 | 41.88 | 2.16 | 65.81 | 15.75 | 3 | 1 | 5 | 8 |
| 108 | 42.05 | 2.15 | 306.06 | 34.13 | 2 | 2 | 7 | 8 |
| 109 | 42.10 | 2.14 | 67.64 | 22.72 | 3 | 2 | 0 | 4 |
| 110 | 42.26 | 2.14 | 47.19 | 13.48 | 1 | 3 | 6 | 8 |
| 111 | 42.28 | 2.14 | 344.34 | 51.50 | 3 | 0 | 6 | 4 |
| 112 | 42.28 | 2.14 | 79.38 | 17.49 | 3 | 2 | 1 | 8 |
| 113 | 42.48 | 2.13 | 266.56 | 32.21 | 1 | 2 | 9 | 8 |
| 114 | 42.72 | 2.12 | 67.53 | 16.31 | 2 | 3 | 3 | 8 |
| 115 | 42.81 | 2.11 | 403.01 | 39.96 | 3 | 2 | 2 | 8 |
| 116 | 42.82 | 2.11 | 501.01 | 63.01 | 0 | 3 | 7 | 4 |
| 117 | 43.07 | 2.10 | 291.78 | 34.22 | 2 | 1 | 9 | 8 |
| 118 | 43.17 | 2.09 | 42.60 | 18.54 | 0 | 1 | 11 | 4 |
| 119 | 43.36 | 2.09 | 65.05 | 23.03 | 1 | 0 | 11 | 4 |
| 120 | 43.68 | 2.07 | 628.80 | 51.05 | 3 | 2 | 3 | 8 |
| 121 | 43.81 | 2.06 | 70.34 | 17.13 | 3 | 1 | 6 | 8 |
| 122 | 43.94 | 2.06 | 48.73 | 14.30 | 2 | 3 | 4 | 8 |
| 123 | 44.08 | 2.05 | 39.54 | 18.29 | 0 | 2 | 10 | 4 |
| 124 | 44.52 | 2.03 | 212.58 | 30.33 | 1 | 3 | 7 | 8 |
| 125 | 44.54 | 2.03 | 1.58 | 3.70 | 3 | 0 | 7 | 4 |
| 126 | 44.66 | 2.03 | 9.92 | 6.57 | 2 | 2 | 8 | 8 |
| 127 | 44.83 | 2.02 | 102.49 | 30.01 | 2 | 0 | 10 | 4 |
| 128 | 44.86 | 2.02 | 113.60 | 22.36 | 1 | 1 | 11 | 8 |
| 129 | 44.88 | 2.02 | 18.40 | 9.00 | 3 | 2 | 4 | 8 |
| 130 | 44.93 | 2.02 | 21.12 | 19.32 | 0 | 4 | 0 | 2 |

TABLE 9
Unit cell parameters for orthorhombic
P2₁2₁2₁ (S)-nicotinium DL-malate.

| | |
|---|---|
| Orthorhombic P2₁2₁2₁ | R₁ = 4.64% |

| | | |
|---|---|---|
| a = 7.5971 (5) | α = 90° | d min (Mo) = 0.70 |
| b = 8.0630 (5) | β = 90° | Flack = 1.1 (10) |
| c = 23.8530 (16) | γ = 90° | Volume = 1461.13 (16) |

Monoclinic P2₁ (S)-Nicotinium Salicylate. Small Scale: Salicylic acid (321.8 mg, 2.3 mmol) was added into a 20 mL scintillation vial. MeOH (3.0 mL) was added with vigorous agitation. (S)-Nicotine (0.32 mL, 2.0 mmol) was added via micropipette in the dark to avoid degradation. The resulting solution was vortexed for 30 seconds at 3,000 rpm on a VWR Mini Vortexer MV I. The solution was then stored in the dark, uncapped to allow for crystal formation while the solvent slowly evaporated. Once the solvent evaporated, the crystalline product was collected via vacuum filtration, washing with n-heptane (3×5 mL) (545.8 mg, 90.86%). The resulting crystalline product was characterized by single crystal X-ray diffraction, simulated powder X-ray diffraction, melting point, differential scanning calorimetry, and a UV irradiation degradation test monitored by ¹H NMR.

Small Scale w/HCl: Salicylic acid (276.2 mg, 2.0 mmol) was added into a 20 mL scintillation vial. MeOH (3.0 mL) was added with vigorous agitation. (S)-Nicotine (0.32 mL, 2.0 mmol) was added via micropipette in the dark to avoid degradation. The resulting solution was vortexed for 30 seconds at 3,000 rpm on a VWR Mini Vortexer MV I. 5 drops of HCl were added via Pasteur pipette and the solution was vigorously agitated. The solution was then stored in the dark, uncapped to allow for crystal formation while the solvent slowly evaporated. Once the solvent evaporated, the crystalline product was collected via vacuum filtration, washing with n-heptane (3×5 mL) (512.7 mg, 85.35%). The resulting crystalline product was characterized by single crystal X-ray diffraction, simulated powder X-ray diffraction, melting point, differential scanning calorimetry, and a UV irradiation degradation test monitored by ¹H NMR.

Large Scale: Salicylic acid (3.4530 g, 25.0 mmol) was added into a 150 mL beaker. EtOH (15.0 mL) was added with vigorous agitation. (S)-Nicotine (4.00 mL, 25.0 mmol) was added via micropipette in the dark to avoid degradation. The resulting solution was vortexed for 30 seconds at 3,000 rpm on a VWR Mini Vortexer MV I. The solution was then stored unsealed in the dark to allow for crystal formation while the solvent slowly evaporated. Once the solvent evaporated, the crystalline product was collected via vacuum filtration, washing with n-heptane (3×15 mL) (5.4095 g, 72.04%). The resulting crystalline product was characterized by single crystal X-ray diffraction, simulated powder X-ray diffraction, melting point, differential scanning calorimetry, and a UV irradiation degradation test monitored by ¹H NMR.

TABLE 10
X-ray powder pattern peak data for monoclinic
P2₁ (S)-nicotinium salicylate.

| Peak | 2Θ | d-spacing | | | | | | |
|---|---|---|---|---|---|---|---|---|
| No. | [°] | [Å] | Intensity | F | h | k | l | Mult. |
| 1 | 9.91 | 8.92 | 16.60 | 2.16 | 0 | 0 | 1 | 2 |
| 2 | 12.34 | 7.17 | 3447.39 | 38.79 | 1 | 0 | 0 | 2 |
| 3 | 12.65 | 6.99 | 1374.95 | 17.76 | 0 | −1 | 1 | 4 |
| 4 | 13.04 | 6.79 | 1606.91 | 27.99 | −1 | 0 | 1 | 2 |
| 5 | 14.64 | 6.05 | 833.91 | 16.04 | −1 | 1 | 0 | 4 |
| 6 | 15.23 | 5.81 | 3751.07 | 35.43 | −1 | 1 | 1 | 4 |
| 7 | 15.73 | 5.63 | 564.24 | 20.07 | 0 | 2 | 0 | 2 |
| 8 | 18.25 | 4.86 | 3134.87 | 55.11 | 1 | 0 | 1 | 2 |
| 9 | 18.62 | 4.76 | 10000.00 | 71.06 | 0 | −2 | 1 | 4 |
| 10 | 19.65 | 4.51 | 1004.79 | 33.68 | −1 | 0 | 2 | 2 |
| 11 | 19.89 | 4.46 | 2297.19 | 36.46 | 1 | −1 | 1 | 4 |
| 12 | 19.89 | 4.46 | 1176.35 | 36.90 | 0 | 0 | 2 | 2 |
| 13 | 20.04 | 4.43 | 500.30 | 17.15 | −1 | 2 | 0 | 4 |
| 14 | 20.48 | 4.33 | 7161.06 | 66.34 | −1 | 2 | 1 | 4 |
| 15 | 21.19 | 4.19 | 4628.27 | 55.26 | −1 | −1 | 2 | 4 |
| 16 | 21.41 | 4.15 | 860.57 | 24.09 | 0 | −1 | 2 | 4 |
| 17 | 23.49 | 3.78 | 94.30 | 12.42 | −2 | 0 | 1 | 2 |
| 18 | 24.18 | 3.68 | 699.12 | 24.64 | 1 | −2 | 1 | 4 |
| 19 | 24.80 | 3.59 | 51.47 | 6.87 | −2 | 1 | 1 | 4 |
| 20 | 24.83 | 3.58 | 542.79 | 31.58 | 2 | 0 | 0 | 2 |
| 21 | 25.27 | 3.52 | 4062.33 | 62.22 | −1 | −2 | 2 | 4 |
| 22 | 25.46 | 3.50 | 3165.07 | 55.35 | 0 | −2 | 2 | 4 |
| 23 | 25.73 | 3.46 | 5637.81 | 74.70 | 0 | −3 | 1 | 4 |
| 24 | 26.08 | 3.41 | 68.00 | 8.32 | −2 | 1 | 0 | 4 |
| 25 | 26.24 | 3.39 | 2219.76 | 67.70 | −2 | 0 | 2 | 2 |
| 26 | 26.78 | 3.33 | 87.56 | 13.74 | 1 | 0 | 2 | 2 |
| 27 | 26.79 | 3.33 | 2785.16 | 54.80 | −1 | 3 | 0 | 4 |
| 28 | 27.12 | 3.28 | 75.83 | 9.16 | −1 | 3 | 1 | 4 |
| 29 | 27.43 | 3.25 | 512.94 | 24.12 | −2 | 1 | 2 | 4 |
| 30 | 27.95 | 3.19 | 119.84 | 11.89 | 1 | −1 | 2 | 4 |
| 31 | 28.39 | 3.14 | 306.07 | 19.32 | −2 | 2 | 1 | 4 |
| 32 | 28.43 | 3.14 | 45.82 | 10.59 | −1 | 0 | 3 | 2 |
| 33 | 29.53 | 3.02 | 283.03 | 19.38 | −2 | 2 | 0 | 4 |
| 34 | 29.53 | 3.02 | 333.26 | 21.03 | −1 | −1 | 3 | 4 |
| 35 | 29.76 | 3.00 | 32.02 | 9.29 | 2 | 0 | 1 | 2 |
| 36 | 30.03 | 2.97 | 212.65 | 24.18 | 0 | 0 | 3 | 2 |
| 37 | 30.06 | 2.97 | 1342.89 | 43.02 | 1 | −3 | 1 | 4 |
| 38 | 30.74 | 2.91 | 439.41 | 25.21 | −2 | 2 | 2 | 4 |
| 39 | 30.82 | 2.90 | 19.01 | 5.26 | 2 | −1 | 1 | 4 |
| 40 | 30.96 | 2.89 | 675.59 | 31.49 | −1 | −3 | 2 | 4 |
| 41 | 31.08 | 2.88 | 213.05 | 17.76 | 0 | −1 | 3 | 4 |
| 42 | 31.11 | 2.87 | 1968.10 | 54.04 | 0 | −3 | 2 | 4 |
| 43 | 31.21 | 2.86 | 86.15 | 11.34 | 1 | −2 | 2 | 4 |
| 44 | 31.76 | 2.82 | 433.55 | 36.67 | 0 | 4 | 0 | 2 |
| 45 | 32.13 | 2.78 | 811.21 | 50.79 | −2 | 0 | 3 | 2 |
| 46 | 32.65 | 2.74 | 409.65 | 25.97 | −1 | −2 | 3 | 4 |
| 47 | 33.12 | 2.70 | 217.40 | 19.22 | −2 | −1 | 3 | 4 |
| 48 | 33.34 | 2.69 | 273.75 | 21.72 | 0 | −4 | 1 | 4 |
| 49 | 33.60 | 2.67 | 1.14 | 1.41 | −2 | 3 | 1 | 4 |
| 50 | 33.83 | 2.65 | 144.94 | 16.06 | 2 | −2 | 1 | 4 |
| 51 | 34.07 | 2.63 | 684.30 | 35.16 | 0 | −2 | 3 | 4 |
| 52 | 34.19 | 2.62 | 178.22 | 18.01 | −1 | 4 | 0 | 4 |
| 53 | 34.46 | 2.60 | 6.04 | 3.34 | −1 | 4 | 1 | 4 |
| 54 | 34.58 | 2.59 | 104.38 | 13.95 | −2 | 3 | 0 | 4 |
| 55 | 35.53 | 2.52 | 49.17 | 13.95 | −3 | 0 | 1 | 2 |
| 56 | 35.64 | 2.52 | 265.22 | 22.99 | −2 | 3 | 2 | 4 |
| 57 | 35.96 | 2.50 | 656.53 | 36.53 | −2 | −2 | 3 | 4 |
| 58 | 36.05 | 2.49 | 276.64 | 23.78 | 1 | −3 | 2 | 4 |
| 59 | 36.30 | 2.47 | 1.05 | 2.09 | 1 | 0 | 3 | 2 |
| 60 | 36.31 | 2.47 | 4.31 | 4.23 | −3 | 0 | 2 | 2 |
| 61 | 36.44 | 2.46 | 216.18 | 21.27 | −3 | 1 | 1 | 4 |
| 62 | 36.87 | 2.44 | 5.80 | 3.53 | 1 | −4 | 1 | 4 |
| 63 | 36.98 | 2.43 | 180.41 | 27.92 | 2 | 0 | 2 | 2 |
| 64 | 37.20 | 2.42 | 5.98 | 3.62 | 1 | −1 | 3 | 4 |
| 65 | 37.20 | 2.41 | 37.72 | 9.09 | −3 | 1 | 2 | 4 |
| 66 | 37.32 | 2.41 | 259.23 | 23.91 | −1 | −3 | 3 | 4 |
| 67 | 37.62 | 2.39 | 1085.19 | 49.35 | −1 | −4 | 2 | 4 |
| 68 | 37.63 | 2.39 | 27.84 | 11.18 | 3 | 0 | 0 | 2 |
| 69 | 37.75 | 2.38 | 77.74 | 13.26 | 0 | −4 | 2 | 4 |
| 70 | 37.86 | 2.37 | 58.03 | 11.49 | 2 | −1 | 2 | 4 |
| 71 | 38.07 | 2.36 | 271.29 | 35.35 | −1 | 0 | 4 | 2 |
| 72 | 38.38 | 2.34 | 9.99 | 4.84 | 2 | −3 | 1 | 4 |
| 73 | 38.50 | 2.34 | 74.62 | 13.28 | −3 | 1 | 0 | 4 |
| 74 | 38.60 | 2.33 | 379.97 | 30.04 | 0 | −3 | 3 | 4 |
| 75 | 38.93 | 2.31 | 183.04 | 21.05 | −1 | −1 | 4 | 4 |
| 76 | 39.07 | 2.30 | 148.58 | 19.04 | −3 | 2 | 1 | 4 |
| 77 | 39.78 | 2.26 | 303.01 | 27.74 | 1 | −2 | 3 | 4 |
| 78 | 39.79 | 2.26 | 40.69 | 10.17 | −3 | 2 | 2 | 4 |
| 79 | 39.82 | 2.26 | 122.04 | 24.92 | −3 | 0 | 3 | 2 |
| 80 | 39.88 | 2.26 | 103.59 | 16.26 | −2 | 4 | 1 | 4 |
| 81 | 39.92 | 2.26 | 6.35 | 5.70 | −2 | 0 | 4 | 2 |
| 82 | 40.30 | 2.24 | 174.45 | 21.36 | −2 | −3 | 3 | 4 |
| 83 | 40.41 | 2.23 | 313.64 | 28.72 | 2 | −2 | 2 | 4 |
| 84 | 40.41 | 2.23 | 279.00 | 38.31 | 0 | 0 | 4 | 2 |
| 85 | 40.65 | 2.22 | 27.99 | 8.64 | −3 | 1 | 3 | 4 |
| 86 | 40.73 | 2.21 | 2.48 | 2.58 | −2 | 4 | 0 | 4 |
| 87 | 40.74 | 2.21 | 155.76 | 20.43 | −2 | −1 | 4 | 4 |
| 88 | 41.01 | 2.20 | 151.28 | 20.28 | −3 | 2 | 0 | 4 |
| 89 | 41.23 | 2.19 | 201.89 | 23.57 | 0 | −1 | 4 | 4 |
| 90 | 41.31 | 2.18 | 42.51 | 10.84 | 0 | −5 | 1 | 4 |
| 91 | 41.42 | 2.18 | 8.97 | 4.99 | −1 | −2 | 4 | 4 |
| 92 | 41.65 | 2.17 | 137.33 | 19.66 | −2 | 4 | 2 | 4 |
| 93 | 42.01 | 2.15 | 281.62 | 28.42 | 1 | −4 | 2 | 4 |
| 94 | 42.01 | 2.15 | 168.68 | 22.00 | −1 | 5 | 0 | 4 |
| 95 | 42.24 | 2.14 | 182.02 | 22.99 | −1 | 5 | 1 | 4 |
| 96 | 42.24 | 2.14 | 497.65 | 53.76 | 3 | 0 | 1 | 2 |
| 97 | 43.04 | 2.10 | 212.59 | 25.37 | 3 | −1 | 1 | 4 |
| 98 | 43.06 | 2.10 | 355.17 | 32.81 | −3 | 2 | 3 | 4 |
| 99 | 43.14 | 2.10 | 153.48 | 21.61 | −1 | −4 | 3 | 4 |
| 100 | 43.15 | 2.10 | 31.19 | 9.75 | −3 | 3 | 1 | 4 |
| 101 | 43.15 | 2.09 | 151.98 | 21.52 | −2 | −2 | 4 | 4 |
| 102 | 43.62 | 2.07 | 15.16 | 6.88 | 0 | −2 | 4 | 4 |
| 103 | 43.81 | 2.07 | 158.73 | 22.36 | 1 | −3 | 3 | 4 |
| 104 | 43.81 | 2.06 | 99.03 | 17.67 | −3 | 3 | 2 | 4 |
| 105 | 44.08 | 2.05 | 26.24 | 9.16 | 2 | −4 | 1 | 4 |
| 106 | 44.27 | 2.04 | 25.72 | 9.11 | 0 | −4 | 3 | 4 |
| 107 | 44.29 | 2.04 | 423.88 | 37.00 | 1 | −5 | 1 | 4 |
| 108 | 44.39 | 2.04 | 31.92 | 10.18 | 2 | −3 | 2 | 4 |
| 109 | 44.94 | 2.02 | 217.86 | 26.97 | −1 | −5 | 2 | 4 |
| 110 | 44.95 | 2.02 | 88.99 | 17.24 | −3 | 3 | 0 | 4 |

TABLE 11
Unit cell parameters for monoclinic P2₁ (S)-nicotinium salicylate.

| | |
|---|---|
| Monoclinic P2₁ | R₁ = 4.34% |

| | | |
|---|---|---|
| a = 7.5908 (12) | α = 90° | d min (Mo) = 0.83 |
| b = 11.2623 (18) | β = 109.274 (5)° | Flack = −0.3 (4) |
| c = 9.4502 (15) | γ = 90° | Volume = 762.6 (2) |

Monoclinic P2₁ (S)-Nicotinium 2,6-Dihydroxybenzoate. 2,6-Dihydroxybenzoic acid (310.5 mg, 2.0 mmol) was added into a 20 mL scintillation vial. MeOH (3.5 mL) was added with vigorous agitation. (S)-Nicotine (0.32 mL, 2.0 mmol) was added via micropipette in the dark to avoid degradation. The resulting solution was vortexed for 30 seconds at 3,000 rpm on a VWR Mini Vortexer MV I. The solution was then stored in the dark, uncapped to allow for crystal formation while the solvent slowly evaporated. Once the solvent evaporated, the crystalline product was collected via vacuum filtration, washing with n-heptane (3×5 mL) (464.5 mg, 73.42%). The resulting crystalline product was characterized by single crystal X-ray diffraction, simulated powder X-ray diffraction, melting point, differential scanning calorimetry, and a UV irradiation degradation test monitored by ¹H NMR.

TABLE 12
X-ray powder pattern peak data for monoclinic P2₁ (S)-
nicotinium 2,6-dihydroxybenzoate at room temperature.

| Peak | 2Θ | d-spacing | | | | | | |
|---|---|---|---|---|---|---|---|---|
| No. | [°] | [Å] | Intensity | F | h | k | l | Mult. |
| 1 | 9.91 | 8.92 | 20.10 | 2.48 | 0 | 0 | 1 | 2 |
| 2 | 12.10 | 7.31 | 3631.21 | 44.65 | 1 | 0 | 0 | 2 |
| 3 | 12.47 | 7.09 | 2715.72 | 27.84 | 0 | −1 | 1 | 4 |
| 4 | 12.92 | 6.85 | 456.79 | 15.45 | −1 | 0 | 1 | 2 |
| 5 | 14.28 | 6.20 | 517.33 | 12.87 | −1 | 1 | 0 | 4 |
| 6 | 14.98 | 5.91 | 4086.04 | 41.21 | −1 | 1 | 1 | 4 |
| 7 | 15.11 | 5.86 | 413.24 | 17.24 | 0 | 2 | 0 | 2 |
| 8 | 18.01 | 4.92 | 2200.66 | 47.60 | 1 | 0 | 1 | 2 |
| 9 | 18.11 | 4.90 | 8754.31 | 72.14 | 0 | −2 | 1 | 4 |
| 10 | 19.41 | 4.57 | 452.09 | 16.47 | −1 | 2 | 0 | 4 |
| 11 | 19.55 | 4.54 | 1650.82 | 31.72 | 1 | −1 | 1 | 4 |
| 12 | 19.66 | 4.51 | 969.01 | 34.56 | −1 | 0 | 2 | 2 |
| 13 | 19.90 | 4.46 | 305.38 | 19.65 | 0 | 0 | 2 | 2 |
| 14 | 19.93 | 4.45 | 6066.63 | 69.11 | −1 | 2 | 1 | 4 |
| 15 | 21.08 | 4.21 | 4882.46 | 64.94 | −1 | −1 | 2 | 4 |
| 16 | 21.31 | 4.17 | 576.37 | 20.49 | 0 | −1 | 2 | 4 |
| 17 | 23.10 | 3.85 | 123.41 | 14.59 | −2 | 0 | 1 | 2 |
| 18 | 23.59 | 3.77 | 1011.80 | 30.19 | 1 | −2 | 1 | 4 |
| 19 | 24.33 | 3.66 | 116.80 | 10.59 | −2 | 1 | 1 | 4 |
| 20 | 24.35 | 3.65 | 1175.46 | 47.56 | 2 | 0 | 0 | 2 |
| 21 | 24.87 | 3.58 | 10000 | 87.86 | 0 | −3 | 1 | 4 |
| 22 | 24.89 | 3.57 | 5751.72 | 76.12 | −1 | −2 | 2 | 4 |
| 23 | 25.08 | 3.55 | 3593.85 | 60.67 | 0 | −2 | 2 | 4 |
| 24 | 25.52 | 3.49 | 66.14 | 8.38 | −2 | 1 | 0 | 4 |
| 25 | 25.85 | 3.44 | 1739.97 | 43.58 | −1 | 3 | 0 | 4 |
| 26 | 26.01 | 3.42 | 1178.63 | 51.05 | −2 | 0 | 2 | 2 |
| 27 | 26.25 | 3.39 | 229.17 | 16.07 | −1 | 3 | 1 | 4 |
| 28 | 26.57 | 3.35 | 43.15 | 9.99 | 1 | 0 | 2 | 2 |
| 29 | 27.12 | 3.29 | 650.21 | 28.02 | −2 | 1 | 2 | 4 |
| 30 | 27.66 | 3.22 | 471.27 | 24.36 | 1 | −1 | 2 | 4 |
| 31 | 27.72 | 3.22 | 391.31 | 22.25 | −2 | 2 | 1 | 4 |
| 32 | 28.49 | 3.13 | 183.98 | 22.21 | −1 | 0 | 3 | 2 |
| 33 | 28.78 | 3.10 | 418.64 | 23.95 | −2 | 2 | 0 | 4 |
| 34 | 29.17 | 3.06 | 1190.55 | 40.98 | 1 | −3 | 1 | 4 |
| 35 | 29.26 | 3.05 | 15.81 | 6.70 | 2 | 0 | 1 | 2 |
| 36 | 29.51 | 3.02 | 428.88 | 24.90 | −1 | −1 | 3 | 4 |
| 37 | 30.05 | 2.97 | 62.00 | 13.65 | 0 | 0 | 3 | 2 |
| 38 | 30.22 | 2.96 | 790.32 | 34.67 | −2 | 2 | 2 | 4 |
| 39 | 30.24 | 2.95 | 862.55 | 36.25 | −1 | −3 | 2 | 4 |
| 40 | 30.25 | 2.95 | 110.40 | 12.98 | 2 | −1 | 1 | 4 |
| 41 | 30.41 | 2.94 | 1382.93 | 46.17 | 0 | −3 | 2 | 4 |
| 42 | 30.50 | 2.93 | 158.30 | 22.16 | 0 | 4 | 0 | 2 |
| 43 | 30.71 | 2.91 | 114.31 | 13.42 | 1 | −2 | 2 | 4 |
| 44 | 31.02 | 2.88 | 360.68 | 24.09 | 0 | −1 | 3 | 4 |
| 45 | 32.03 | 2.79 | 442.29 | 39.06 | −2 | 0 | 3 | 2 |
| 46 | 32.14 | 2.78 | 343.42 | 24.43 | 0 | −4 | 1 | 4 |
| 47 | 32.40 | 2.76 | 171.61 | 17.42 | −1 | −2 | 3 | 4 |
| 48 | 32.65 | 2.74 | 82.85 | 12.20 | −2 | 3 | 1 | 4 |
| 49 | 32.92 | 2.72 | 107.28 | 14.01 | −1 | 4 | 0 | 4 |
| 50 | 32.96 | 2.72 | 421.07 | 27.79 | −2 | −1 | 3 | 4 |
| 51 | 33.09 | 2.71 | 157.81 | 17.08 | 2 | −2 | 1 | 4 |
| 52 | 33.25 | 2.69 | 113.65 | 14.57 | −1 | 4 | 1 | 4 |
| 53 | 33.57 | 2.67 | 80.30 | 12.38 | −2 | 3 | 0 | 4 |
| 54 | 33.80 | 2.65 | 597.34 | 34.01 | 0 | −2 | 3 | 4 |
| 55 | 34.83 | 2.57 | 268.18 | 23.55 | −2 | 3 | 2 | 4 |
| 56 | 34.87 | 2.57 | 210.83 | 29.57 | −3 | 0 | 1 | 2 |
| 57 | 35.26 | 2.54 | 330.52 | 26.50 | 1 | −3 | 2 | 4 |
| 58 | 35.59 | 2.52 | 563.61 | 34.96 | −2 | −2 | 3 | 4 |
| 59 | 35.65 | 2.52 | 47.07 | 10.12 | 1 | −4 | 1 | 4 |
| 60 | 35.72 | 2.51 | 130.06 | 16.86 | −3 | 1 | 1 | 4 |
| 61 | 35.78 | 2.51 | 0.76 | 1.83 | −3 | 0 | 2 | 2 |
| 62 | 36.11 | 2.49 | 8.25 | 6.08 | 1 | 0 | 3 | 2 |
| 63 | 36.49 | 2.46 | 71.74 | 18.13 | 2 | 0 | 2 | 2 |
| 64 | 36.55 | 2.46 | 1053.97 | 49.22 | −1 | −4 | 2 | 4 |
| 65 | 36.62 | 2.45 | 139.30 | 17.93 | −3 | 1 | 2 | 4 |
| 66 | 36.69 | 2.45 | 148.47 | 18.55 | 0 | −4 | 2 | 4 |
| 67 | 36.77 | 2.44 | 319.41 | 27.27 | −1 | −3 | 3 | 4 |
| 68 | 36.88 | 2.44 | 86.91 | 20.19 | 3 | 0 | 0 | 2 |
| 69 | 36.95 | 2.43 | 25.70 | 7.78 | 1 | −1 | 3 | 4 |
| 70 | 37.31 | 2.41 | 44.36 | 10.33 | 2 | −1 | 2 | 4 |
| 71 | 37.38 | 2.40 | 44.41 | 10.36 | 2 | −3 | 1 | 4 |
| 72 | 37.70 | 2.38 | 52.87 | 11.41 | −3 | 1 | 0 | 4 |
| 73 | 38.02 | 2.36 | 117.56 | 17.17 | 0 | −3 | 3 | 4 |
| 74 | 38.16 | 2.36 | 304.48 | 39.24 | −1 | 0 | 4 | 2 |
| 75 | 38.20 | 2.35 | 202.26 | 22.63 | −3 | 2 | 1 | 4 |
| 76 | 38.60 | 2.33 | 68.68 | 13.35 | −2 | 4 | 1 | 4 |
| 77 | 38.96 | 2.31 | 120.39 | 17.85 | −1 | −1 | 4 | 4 |
| 78 | 39.05 | 2.31 | 89.77 | 15.45 | −3 | 2 | 2 | 4 |
| 79 | 39.35 | 2.29 | 349.93 | 30.77 | 1 | −2 | 3 | 4 |
| 80 | 39.40 | 2.29 | 8.07 | 4.68 | −2 | 4 | 0 | 4 |
| 81 | 39.46 | 2.28 | 304.31 | 40.70 | −3 | 0 | 3 | 2 |
| 82 | 39.65 | 2.27 | 138.09 | 19.50 | −2 | −3 | 3 | 4 |
| 83 | 39.70 | 2.27 | 182.69 | 22.45 | 2 | −2 | 2 | 4 |
| 84 | 39.74 | 2.27 | 92.54 | 16.00 | 0 | −5 | 1 | 4 |
| 85 | 39.92 | 2.26 | 3.63 | 4.51 | −2 | 0 | 4 | 2 |
| 86 | 40.07 | 2.25 | 186.62 | 22.93 | −3 | 2 | 0 | 4 |
| 87 | 40.23 | 2.24 | 64.15 | 13.50 | −3 | 1 | 3 | 4 |
| 88 | 40.39 | 2.23 | 223.72 | 25.33 | −1 | 5 | 0 | 4 |
| 89 | 40.44 | 2.23 | 129.04 | 27.24 | 0 | 0 | 4 | 2 |
| 90 | 40.50 | 2.23 | 228.99 | 25.70 | −2 | 4 | 2 | 4 |
| 91 | 40.67 | 2.22 | 157.65 | 21.42 | −1 | 5 | 1 | 4 |
| 92 | 40.69 | 2.22 | 125.04 | 19.09 | −2 | −1 | 4 | 4 |
| 93 | 40.89 | 2.21 | 373.45 | 33.17 | 1 | −4 | 2 | 4 |
| 94 | 41.20 | 2.19 | 206.39 | 24.87 | 0 | −1 | 4 | 4 |
| 95 | 41.27 | 2.19 | 2.26 | 2.61 | −1 | −2 | 4 | 4 |
| 96 | 41.46 | 2.18 | 503.97 | 55.35 | 3 | 0 | 1 | 2 |
| 97 | 42.04 | 2.15 | 44.93 | 11.87 | −3 | 3 | 1 | 4 |
| 98 | 42.20 | 2.14 | 138.37 | 20.92 | 3 | −1 | 1 | 4 |
| 99 | 42.22 | 2.14 | 332.47 | 32.44 | −1 | −4 | 3 | 4 |
| 100 | 42.48 | 2.13 | 371.96 | 34.55 | −3 | 2 | 3 | 4 |
| 101 | 42.71 | 2.12 | 465.87 | 38.90 | 1 | −5 | 1 | 4 |
| 102 | 42.77 | 2.11 | 41.43 | 11.62 | 2 | −4 | 1 | 4 |
| 103 | 42.83 | 2.11 | 181.52 | 24.36 | −3 | 3 | 2 | 4 |
| 104 | 42.92 | 2.11 | 44.38 | 12.07 | −2 | −2 | 4 | 4 |
| 105 | 43.11 | 2.10 | 199.16 | 25.70 | 1 | −3 | 3 | 4 |
| 106 | 43.34 | 2.09 | 4.16 | 3.74 | 0 | −4 | 3 | 4 |
| 107 | 43.41 | 2.08 | 62.59 | 14.52 | 0 | −2 | 4 | 4 |
| 108 | 43.44 | 2.08 | 40.28 | 11.66 | 2 | −3 | 2 | 4 |
| 109 | 43.48 | 2.08 | 116.99 | 19.89 | −1 | −5 | 2 | 4 |
| 110 | 43.61 | 2.07 | 334.45 | 33.74 | 0 | −5 | 2 | 4 |
| 111 | 43.77 | 2.07 | 167.76 | 24.00 | −3 | 3 | 0 | 4 |
| 112 | 44.37 | 2.04 | 11.87 | 6.48 | 3 | −2 | 1 | 4 |
| 113 | 44.81 | 2.02 | 41.98 | 12.33 | −2 | −4 | 3 | 4 |
| 114 | 44.89 | 2.02 | 160.82 | 24.18 | −1 | −3 | 4 | 4 |

TABLE 13
Unit cell parameters for monoclinic P2₁ (S)-nicotinium
2,6-dihydroxybenzoate at room temperature.

| | |
|---|---|
| Monoclinic P2₁ | R₁ = 3.84% |

| | | |
|---|---|---|
| a = 7.7196 (5) | α = 90° | d min (Mo) = 0.75 |
| b = 11.7045 (7) | β = 108.980 (2)° | Flack = −0.4 (4) |
| c = 9.4241 (6) | γ = 90° | Volume = 805.21 (9) |

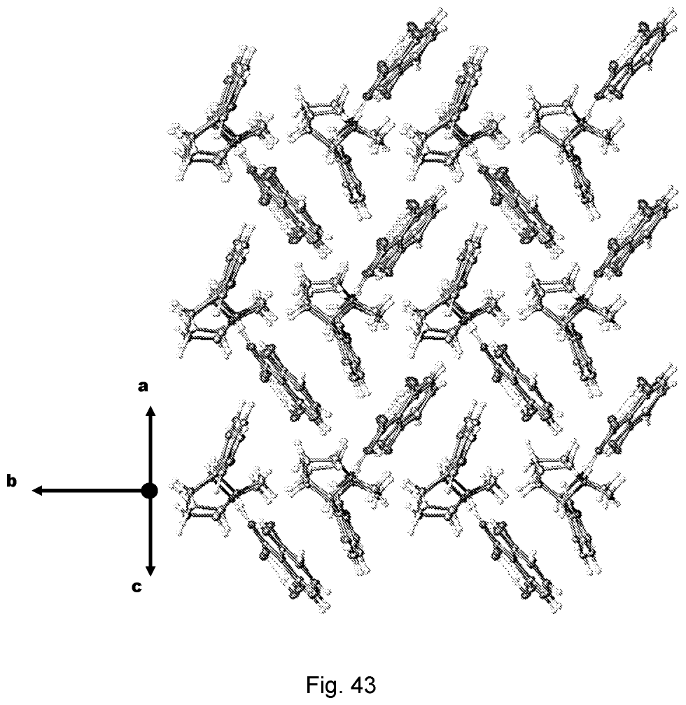

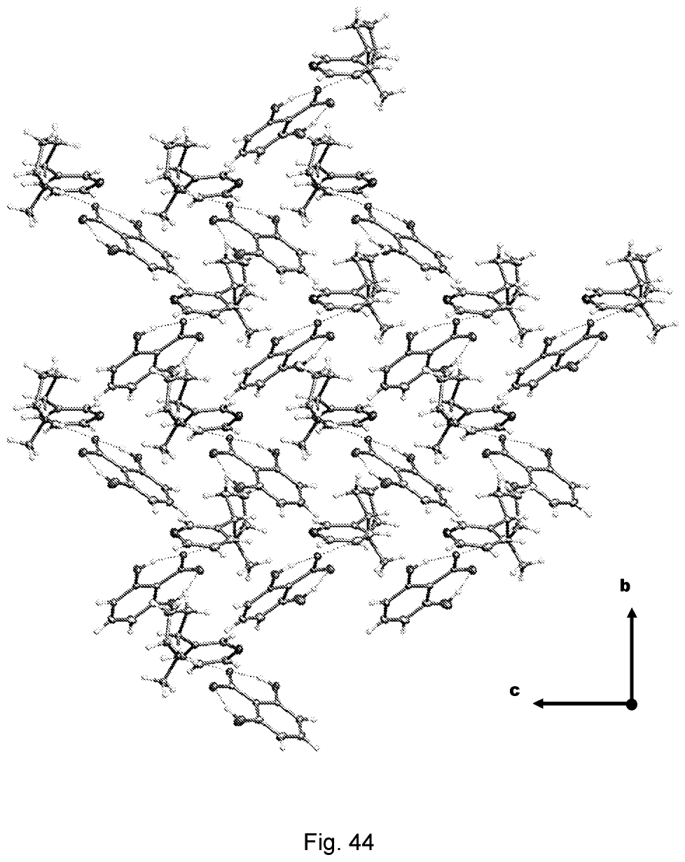

Monoclinic P2₁ (S)-icotinium 2,6-Dihydroxybenzoate Phase Transition. The same crystal used for the room temperature single crystal x-ray diffraction experiment was ramped from 110 K to 100 K and back to 110 K to observe the temperature dependent polymorphic change known as a phase transition. The crystal underwent first order transitions, which can be changes with regards to the unit cell volume, the lattice, the packing, or the enthalpy or entropy of the crystalline system. In this instance, there was a perturbation of the monoclinic P2₁ (S)-nicotinium 2,6-dihydroxybenzoate packing as the crystal was cooled to 90 K. As seen in FIG. 42 and FIG. 43 the nicotinium 2,6-dihydroxybenzoate are no longer uniformly aligned as every other row has slipped slightly. This caused the unit cell parameters to change. While the Bravais lattice and space group of the phase transition structure remained the same, the most notable difference is the change in the unit cell volume. The volume nearly doubled as a result of every other salt row slipping in the phase transition structure. Further SC-XRD experiments were run to track the unit cell parameters as the temperature was ramped from 110 K to 100 K to isolate the exact temperature of the observed phase transition. The unit cell was observed as changing drastically from 107 K to 106 K as well as reversibly transitioning from 106 K to 107 K.

TABLE 14
X-ray powder pattern peak data for monoclinic
P2₁ (S)-nicotinium 2,6-dihydroxybenzoate at 90 K.

| Peak | 2Θ | d-spacing | | | | | | |
|---|---|---|---|---|---|---|---|---|
| No. | [°] | [Å] | Intensity | F | h | k | l | Mult. |
| 1 | 9.91 | 8.92 | 20.10 | 2.48 | 0 | 0 | 1 | 2 |
| 2 | 12.22 | 7.31 | 3026.97 | 44.65 | 1 | 0 | 0 | 2 |
| 3 | 12.47 | 7.09 | 2456.03 | 27.84 | 0 | −1 | 1 | 4 |
| 4 | 13.10 | 6.85 | 317.55 | 15.45 | −1 | 0 | 1 | 2 |
| 5 | 14.42 | 6.20 | 439.36 | 12.87 | −1 | 1 | 0 | 4 |
| 6 | 15.16 | 5.91 | 3310.84 | 41.21 | −1 | 1 | 1 | 4 |
| 7 | 15.18 | 5.86 | 3555.69 | 17.24 | 0 | 2 | 0 | 2 |
| 8 | 18.28 | 4.92 | 3085.87 | 47.60 | 1 | 0 | 1 | 2 |
| 9 | 18.34 | 4.90 | 7288.20 | 72.14 | 0 | −2 | 1 | 4 |
| 10 | 19.41 | 4.57 | 452.09 | 16.47 | −1 | 2 | 0 | 4 |
| 11 | 19.66 | 4.51 | 969.01 | 34.56 | −1 | 0 | 2 | 2 |
| 12 | 19.90 | 4.46 | 305.38 | 19.65 | 0 | 0 | 2 | 2 |
| 13 | 20.12 | 4.54 | 1515.42 | 31.72 | 1 | −1 | 1 | 4 |
| 14 | 20.20 | 4.45 | 5510.22 | 69.11 | −1 | 2 | 1 | 4 |
| 15 | 21.31 | 4.17 | 576.37 | 20.49 | 0 | −1 | 2 | 4 |
| 16 | 21.34 | 4.21 | 4332.39 | 64.94 | −1 | −1 | 2 | 4 |
| 17 | 23.10 | 3.85 | 123.41 | 14.59 | −2 | 0 | 1 | 2 |
| 18 | 23.90 | 3.77 | 560.06 | 30.19 | 1 | −2 | 1 | 4 |
| 19 | 24.33 | 3.66 | 116.80 | 10.59 | −2 | 1 | 1 | 4 |
| 20 | 24.64 | 3.65 | 762.72 | 47.56 | 2 | 0 | 0 | 2 |
| 21 | 25.22 | 3.58 | 10000 | 87.86 | 0 | −3 | 1 | 4 |
| 22 | 25.28 | 3.57 | 5751.72 | 76.12 | −1 | −2 | 2 | 4 |
| 23 | 25.36 | 3.55 | 3211.62 | 60.67 | 0 | −2 | 2 | 4 |
| 24 | 25.52 | 3.49 | 66.14 | 8.38 | −2 | 1 | 0 | 4 |
| 25 | 25.18 | 3.44 | 1218.14 | 43.58 | −1 | 3 | 0 | 4 |
| 26 | 26.26 | 3.42 | 740.09 | 51.05 | −2 | 0 | 2 | 2 |
| 27 | 26.34 | 3.39 | 229.17 | 16.07 | −1 | 3 | 1 | 4 |
| 28 | 26.57 | 3.35 | 43.15 | 9.99 | 1 | 0 | 2 | 2 |
| 29 | 27.12 | 3.29 | 650.21 | 28.02 | −2 | 1 | 2 | 4 |
| 30 | 27.66 | 3.22 | 471.27 | 24.36 | 1 | −1 | 2 | 4 |
| 31 | 27.72 | 3.22 | 391.31 | 22.25 | −2 | 2 | 1 | 4 |
| 32 | 28.49 | 3.13 | 183.98 | 22.21 | −1 | 0 | 3 | 2 |
| 33 | 28.78 | 3.10 | 418.64 | 23.95 | −2 | 2 | 0 | 4 |
| 34 | 29.17 | 3.06 | 1190.55 | 40.98 | 1 | −3 | 1 | 4 |
| 35 | 29.26 | 3.05 | 15.81 | 6.70 | 2 | 0 | 1 | 2 |
| 36 | 29.51 | 3.02 | 428.88 | 24.90 | −1 | −1 | 3 | 4 |
| 37 | 30.05 | 2.97 | 62.00 | 13.65 | 0 | 0 | 3 | 2 |
| 38 | 30.22 | 2.96 | 790.32 | 34.67 | −2 | 2 | 2 | 4 |
| 39 | 30.24 | 2.95 | 862.55 | 36.25 | −1 | −3 | 2 | 4 |
| 40 | 30.25 | 2.95 | 110.40 | 12.98 | 2 | −1 | 1 | 4 |
| 41 | 30.62 | 2.94 | 1273.43 | 46.17 | 0 | −3 | 2 | 4 |
| 42 | 30.66 | 2.93 | 1234.48 | 22.16 | 0 | 4 | 0 | 2 |
| 43 | 30.71 | 2.91 | 114.31 | 13.42 | 1 | −2 | 2 | 4 |
| 44 | 31.02 | 2.88 | 360.68 | 24.09 | 0 | −1 | 3 | 4 |
| 45 | 32.03 | 2.79 | 442.29 | 39.06 | −2 | 0 | 3 | 2 |
| 46 | 32.14 | 2.78 | 343.42 | 24.43 | 0 | −4 | 1 | 4 |
| 47 | 32.40 | 2.76 | 171.61 | 17.42 | −1 | −2 | 3 | 4 |
| 48 | 32.65 | 2.74 | 82.85 | 12.20 | −2 | 3 | 1 | 4 |
| 49 | 32.92 | 2.72 | 107.28 | 14.01 | −1 | 4 | 0 | 4 |
| 50 | 32.96 | 2.72 | 421.07 | 27.79 | −2 | −1 | 3 | 4 |
| 51 | 33.09 | 2.71 | 157.81 | 17.08 | 2 | −2 | 1 | 4 |
| 52 | 33.25 | 2.69 | 113.65 | 14.57 | −1 | 4 | 1 | 4 |
| 53 | 33.57 | 2.67 | 80.30 | 12.38 | −2 | 3 | 0 | 4 |
| 54 | 33.80 | 2.65 | 597.34 | 34.01 | 0 | −2 | 3 | 4 |
| 55 | 34.83 | 2.57 | 268.18 | 23.55 | −2 | 3 | 2 | 4 |
| 56 | 34.87 | 2.57 | 210.83 | 29.57 | −3 | 0 | 1 | 2 |
| 57 | 35.26 | 2.54 | 330.52 | 26.50 | 1 | −3 | 2 | 4 |
| 58 | 35.59 | 2.52 | 563.61 | 34.96 | −2 | −2 | 3 | 4 |
| 59 | 35.65 | 2.52 | 47.07 | 10.12 | 1 | −4 | 1 | 4 |
| 60 | 35.72 | 2.51 | 130.06 | 16.86 | −3 | 1 | 1 | 4 |
| 61 | 35.78 | 2.51 | 0.76 | 1.83 | −3 | 0 | 2 | 2 |
| 62 | 36.11 | 2.49 | 8.25 | 6.08 | 1 | 0 | 3 | 2 |
| 63 | 36.49 | 2.46 | 71.74 | 18.13 | 2 | 0 | 2 | 2 |
| 64 | 36.62 | 2.45 | 139.30 | 17.93 | −3 | 1 | 2 | 4 |
| 65 | 36.69 | 2.45 | 148.47 | 18.55 | 0 | −4 | 2 | 4 |
| 66 | 36.77 | 2.44 | 319.41 | 27.27 | −1 | −3 | 3 | 4 |
| 67 | 36.88 | 2.44 | 86.91 | 20.19 | 3 | 0 | 0 | 2 |
| 68 | 36.95 | 2.43 | 25.70 | 7.78 | 1 | −1 | 3 | 4 |
| 69 | 37.08 | 2.46 | 774.53 | 49.22 | −1 | −4 | 2 | 4 |
| 70 | 37.31 | 2.41 | 44.36 | 10.33 | 2 | −1 | 2 | 4 |
| 71 | 37.38 | 2.40 | 44.41 | 10.36 | 2 | −3 | 1 | 4 |
| 72 | 37.70 | 2.38 | 52.87 | 11.41 | −3 | 1 | 0 | 4 |
| 73 | 38.02 | 2.36 | 117.56 | 17.17 | 0 | −3 | 3 | 4 |
| 74 | 38.16 | 2.36 | 304.48 | 39.24 | −1 | 0 | 4 | 2 |
| 75 | 38.20 | 2.35 | 202.26 | 22.63 | −3 | 2 | 1 | 4 |
| 76 | 38.60 | 2.33 | 68.68 | 13.35 | −2 | 4 | 1 | 4 |
| 77 | 38.96 | 2.31 | 120.39 | 17.85 | −1 | −1 | 4 | 4 |
| 78 | 39.05 | 2.31 | 89.77 | 15.45 | −3 | 2 | 2 | 4 |
| 79 | 39.35 | 2.29 | 349.93 | 30.77 | 1 | −2 | 3 | 4 |
| 80 | 39.40 | 2.29 | 8.07 | 4.68 | −2 | 4 | 0 | 4 |
| 81 | 39.46 | 2.28 | 304.31 | 40.70 | −3 | 0 | 3 | 2 |
| 82 | 39.65 | 2.27 | 138.09 | 19.50 | −2 | −3 | 3 | 4 |
| 83 | 39.70 | 2.27 | 182.69 | 22.45 | 2 | −2 | 2 | 4 |
| 84 | 39.74 | 2.27 | 92.54 | 16.00 | 0 | −5 | 1 | 4 |
| 85 | 39.92 | 2.26 | 3.63 | 4.51 | −2 | 0 | 4 | 2 |
| 86 | 40.07 | 2.25 | 186.62 | 22.93 | −3 | 2 | 0 | 4 |
| 87 | 40.23 | 2.24 | 64.15 | 13.50 | −3 | 1 | 3 | 4 |
| 88 | 40.39 | 2.23 | 223.72 | 25.33 | −1 | 5 | 0 | 4 |
| 89 | 40.44 | 2.23 | 129.04 | 27.24 | 0 | 0 | 4 | 2 |
| 90 | 40.50 | 2.23 | 228.99 | 25.70 | −2 | 4 | 2 | 4 |
| 91 | 40.67 | 2.22 | 157.65 | 21.42 | −1 | 5 | 1 | 4 |
| 92 | 40.69 | 2.22 | 125.04 | 19.09 | −2 | −1 | 4 | 4 |
| 93 | 40.89 | 2.21 | 373.45 | 33.17 | 1 | −4 | 2 | 4 |
| 94 | 41.20 | 2.19 | 206.39 | 24.87 | 0 | −1 | 4 | 4 |
| 95 | 41.27 | 2.19 | 2.26 | 2.61 | −1 | −2 | 4 | 4 |
| 96 | 41.46 | 2.18 | 503.97 | 55.35 | 3 | 0 | 1 | 2 |
| 97 | 42.04 | 2.15 | 44.93 | 11.87 | −3 | 3 | 1 | 4 |
| 98 | 42.20 | 2.14 | 138.37 | 20.92 | 3 | −1 | 1 | 4 |
| 99 | 42.22 | 2.14 | 332.47 | 32.44 | −1 | −4 | 3 | 4 |
| 100 | 42.48 | 2.13 | 371.96 | 34.55 | −3 | 2 | 3 | 4 |
| 101 | 42.71 | 2.12 | 465.87 | 38.90 | 1 | −5 | 1 | 4 |
| 102 | 42.77 | 2.11 | 41.43 | 11.62 | 2 | −4 | 1 | 4 |
| 103 | 42.83 | 2.11 | 181.52 | 24.36 | −3 | 3 | 2 | 4 |
| 104 | 42.92 | 2.11 | 44.38 | 12.07 | −2 | −2 | 4 | 4 |
| 105 | 43.11 | 2.10 | 199.16 | 25.70 | 1 | −3 | 3 | 4 |
| 106 | 43.34 | 2.09 | 4.16 | 3.74 | 0 | −4 | 3 | 4 |
| 107 | 43.41 | 2.08 | 62.59 | 14.52 | 0 | −2 | 4 | 4 |
| 108 | 43.44 | 2.08 | 40.28 | 11.66 | 2 | −3 | 2 | 4 |
| 109 | 43.48 | 2.08 | 116.99 | 19.89 | −1 | −5 | 2 | 4 |
| 110 | 43.61 | 2.07 | 334.45 | 33.74 | 0 | −5 | 2 | 4 |
| 111 | 43.77 | 2.07 | 167.76 | 24.00 | −3 | 3 | 0 | 4 |
| 112 | 44.37 | 2.04 | 11.87 | 6.48 | 3 | −2 | 1 | 4 |
| 113 | 44.81 | 2.02 | 41.98 | 12.33 | −2 | −4 | 3 | 4 |
| 114 | 44.89 | 2.02 | 160.82 | 24.18 | −1 | −3 | 4 | 4 |

TABLE 15
Unit cell parameters of monoclinic P2₁ (S)-
nicotinium 2,6-dihydroxybenzoate at 90 K.

| | |
|---|---|
| Monoclinic P2₁ | R₁ = 3.27% |

| | | |
|---|---|---|
| a = 9.9691(4) | α = 90° | d min (Mo) = 0.75 |
| b = 11.5520(4) | β = 101.995(1)° | Flack = −0.3(3) |
| c = 13.8196(5) | γ = 90° | Volume = 1556.76(10) |

TABLE 16
Unit cell data for monoclinic P2₁ (S)-nicotinium
2,6-dihydroxybenzoate temperature ramp study.

| Temperature (K) | a (Å) | b (Å) | c (Å) | β (°) | Volume (Å³) |
|---|---|---|---|---|---|
| 110 | 7.63 | 11.61 | 9.33 | 108.69 | 782 |
| 109 | 7.57 | 11.47 | 9.21 | 108.49 | 788 |
| 108 | 7.63 | 11.63 | 9.34 | 108.7 | 786 |
| 107 | 7.63 | 11.61 | 9.33 | 108.7 | 783 |
| 106 | 9.97 | 11.59 | 13.82 | 102.02 | 1562 |
| 105 | 9.99 | 11.59 | 13.86 | 102.1 | 1569 |
| 104 | 10.01 | 11.6 | 13.85 | 102.17 | 1573 |
| 103 | 10.00 | 11.59 | 13.87 | 102.2 | 1571 |
| 102 | 9.97 | 11.58 | 13.81 | 102.01 | 1560 |
| 101 | 9.98 | 11.55 | 13.81 | 102.09 | 1558 |
| 100 | 10.05 | 11.58 | 13.84 | 102.03 | 1569 |
| 101 | 9.98 | 11.55 | 13.79 | 102.08 | 1553 |
| 102 | 9.99 | 11.57 | 13.84 | 102.08 | 1564 |
| 103 | 9.99 | 11.59 | 13.85 | 102.07 | 1568 |
| 104 | 9.98 | 11.57 | 13.83 | 102.02 | 1561 |
| 105 | 9.98 | 11.57 | 13.82 | 102.1 | 1561 |
| 106 | 9.97 | 11.59 | 13.79 | 102.02 | 1559 |
| 107 | 7.63 | 11.61 | 9.32 | 108.79 | 782 |
| 108 | 7.63 | 11.61 | 9.33 | 108.76 | 782 |
| 109 | 7.64 | 11.63 | 9.35 | 108.77 | 786 |
| 110 | 7.66 | 11.65 | 9.35 | 108.79 | 790 |

Orthorhombic P222₁ (S)-Nicotinium Orotate Monohydrate. DMF Method: Orotic acid (101.6 mg, 0.7 mmol) was added into a 20 mL scintillation vial. DMF (5.0 mL) was added with vigorous agitation. (S)-Nicotine (0.1 mL, 0.6 mmol) was added via micropipette in the dark to avoid degradation. The resulting solution was sonicated for 30 minutes at 45° C. Ethyl acetate was diffused into the solution. The solution was then stored in the dark, uncapped to allow for crystal formation while the solvent slowly evaporated. Crystal formation was noted to occur on the walls of the vial, while the solvent slowly evaporated. Individuals crystals were pulled from the solution and walls of the vial (3.2 mg, 1.68%). The resulting crystalline product was characterized by single crystal X-ray diffraction and simulated powder X-ray diffraction.

H₂O Method: Orotic acid (1.00 g, 6.41 mmol) was added into a 1 L beaker. DI water (700.0 mL) was added with vigorous stirring. The solution was then stirred at 850 rpm while the heat was maintained at 90° C. (S)-Nicotine (1.10 mL, 6.88 mmol) was added via micropipette in the dark to avoid degradation. The resulting solution was stirred in the dark for 30 additional minutes without heat. The solution was then stored in the dark, unsealed to allow for crystal formation while air was blown into the top of the beaker to aid in the solvent evaporation process. Once the solvent evaporated, the crystalline product was collected via vacuum filtration, washing with n-heptane (3×15 mL) (1.4404 g, 70.59%). The resulting crystalline product was characterized by single crystal X-ray diffraction, simulated powder X-ray diffraction, melting point, differential scanning calorimetry, and a UV irradiation degradation test monitored by ¹H NMR.

TABLE 17
X-ray powder pattern peak data for orthorhombic
P222₁ (S)-nicotinium orotate monohydrate.

| | 2Θ | d-spacing | | | | | | |
|---|---|---|---|---|---|---|---|---|
| No. | [°] | [Å] | Intensity | F | h | k | l | Mult. |
| 1 | 7.67 | 11.52 | 1113.00 | 27.53 | 0 | 0 | 2 | 2 |
| 2 | 9.05 | 9.77 | 5491.16 | 72.21 | 0 | 1 | 0 | 2 |
| 3 | 9.83 | 8.99 | 1314.07 | 27.15 | 0 | 1 | 1 | 4 |
| 4 | 11.87 | 7.45 | 413.23 | 18.42 | 0 | 1 | 2 | 4 |
| 5 | 12.49 | 7.08 | 555.26 | 31.81 | 1 | 0 | 0 | 2 |
| 6 | 13.07 | 6.77 | 117.69 | 10.84 | 1 | 0 | 1 | 4 |
| 7 | 14.66 | 6.04 | 751.69 | 30.79 | 0 | 1 | 3 | 4 |
| 8 | 14.67 | 6.03 | 4530.72 | 75.66 | 1 | 0 | 2 | 4 |
| 9 | 15.37 | 5.76 | 8029.14 | 149.35 | 0 | 0 | 4 | 2 |
| 10 | 15.44 | 5.73 | 165.48 | 15.23 | 1 | 1 | 0 | 4 |
| 11 | 15.92 | 5.56 | 682.87 | 22.57 | 1 | 1 | 1 | 8 |
| 12 | 17.02 | 5.21 | 1389.26 | 48.75 | 1 | 0 | 3 | 4 |
| 13 | 17.26 | 5.13 | 10000.00 | 93.85 | 1 | 1 | 2 | 8 |
| 14 | 17.86 | 4.96 | 1015.81 | 43.81 | 0 | 1 | 4 | 4 |
| 15 | 18.15 | 4.88 | 2509.70 | 99.00 | 0 | 2 | 0 | 2 |
| 16 | 18.55 | 4.78 | 1487.87 | 55.14 | 0 | 2 | 1 | 4 |
| 17 | 19.30 | 4.60 | 1869.54 | 45.52 | 1 | 1 | 3 | 8 |
| 18 | 19.73 | 4.50 | 1688.44 | 62.57 | 0 | 2 | 2 | 4 |
| 19 | 19.85 | 4.47 | 831.57 | 44.20 | 1 | 0 | 4 | 4 |
| 20 | 21.30 | 4.17 | 1039.71 | 53.16 | 0 | 1 | 5 | 4 |
| 21 | 21.54 | 4.12 | 92.89 | 16.08 | 0 | 2 | 3 | 4 |
| 22 | 21.85 | 4.06 | 458.83 | 25.65 | 1 | 1 | 4 | 8 |
| 23 | 22.09 | 4.02 | 864.20 | 50.34 | 1 | 2 | 0 | 4 |
| 24 | 22.43 | 3.96 | 90.22 | 11.68 | 1 | 2 | 1 | 8 |
| 25 | 23.00 | 3.86 | 229.73 | 27.08 | 1 | 0 | 5 | 4 |
| 26 | 23.14 | 3.84 | 306.28 | 44.48 | 0 | 0 | 6 | 2 |
| 27 | 23.41 | 3.80 | 1400.86 | 48.16 | 1 | 2 | 2 | 8 |
| 28 | 23.87 | 3.73 | 15.25 | 7.25 | 0 | 2 | 4 | 4 |
| 29 | 24.76 | 3.59 | 819.29 | 39.06 | 1 | 1 | 5 | 8 |
| 30 | 24.89 | 3.57 | 153.62 | 24.05 | 0 | 1 | 6 | 4 |
| 31 | 24.98 | 3.56 | 2424.93 | 67.80 | 1 | 2 | 3 | 8 |
| 32 | 25.13 | 3.54 | 2725.92 | 144.69 | 2 | 0 | 0 | 2 |
| 33 | 25.43 | 3.50 | 5306.64 | 144.54 | 2 | 0 | 1 | 4 |
| 34 | 26.31 | 3.38 | 2097.99 | 94.21 | 2 | 0 | 2 | 4 |
| 35 | 26.38 | 3.38 | 1196.21 | 71.33 | 1 | 0 | 6 | 4 |
| 36 | 26.57 | 3.35 | 832.88 | 59.98 | 0 | 2 | 5 | 4 |
| 37 | 26.76 | 3.33 | 344.69 | 38.87 | 2 | 1 | 0 | 4 |
| 38 | 27.02 | 3.30 | 832.47 | 43.17 | 1 | 2 | 4 | 8 |
| 39 | 27.04 | 3.30 | 6511.46 | 120.81 | 2 | 1 | 1 | 8 |
| 40 | 27.37 | 3.26 | 63.21 | 24.11 | 0 | 3 | 0 | 2 |
| 41 | 27.64 | 3.22 | 82.80 | 19.72 | 0 | 3 | 1 | 4 |
| 42 | 27.72 | 3.22 | 30.76 | 12.06 | 2 | 0 | 3 | 4 |
| 43 | 27.87 | 3.20 | 1673.20 | 63.25 | 2 | 1 | 2 | 8 |
| 44 | 27.94 | 3.19 | 934.28 | 47.38 | 1 | 1 | 6 | 8 |
| 45 | 28.46 | 3.13 | 6.66 | 5.77 | 0 | 3 | 2 | 4 |
| 46 | 28.59 | 3.12 | 12.24 | 7.86 | 0 | 1 | 7 | 4 |
| 47 | 29.21 | 3.05 | 3171.39 | 91.54 | 2 | 1 | 3 | 8 |
| 48 | 29.46 | 3.03 | 506.77 | 36.92 | 1 | 2 | 5 | 8 |
| 49 | 29.56 | 3.02 | 8.44 | 6.76 | 0 | 2 | 6 | 4 |
| 50 | 29.59 | 3.02 | 4317.99 | 153.13 | 2 | 0 | 4 | 4 |
| 51 | 29.78 | 3.00 | 123.65 | 26.09 | 0 | 3 | 3 | 4 |
| 52 | 29.91 | 2.99 | 171.70 | 30.89 | 1 | 0 | 7 | 4 |
| 53 | 30.18 | 2.96 | 25.31 | 11.98 | 1 | 3 | 0 | 4 |
| 54 | 30.44 | 2.93 | 254.68 | 27.11 | 1 | 3 | 1 | 8 |
| 55 | 31.00 | 2.88 | 10.91 | 5.72 | 2 | 1 | 4 | 8 |
| 56 | 31.02 | 2.88 | 82.96 | 31.58 | 0 | 0 | 8 | 2 |
| 57 | 31.17 | 2.87 | 114.42 | 26.36 | 2 | 2 | 0 | 4 |
| 58 | 31.19 | 2.87 | 321.75 | 31.28 | 1 | 3 | 2 | 8 |
| 59 | 31.31 | 2.86 | 1202.25 | 60.70 | 1 | 1 | 7 | 8 |
| 60 | 31.42 | 2.85 | 39.92 | 11.10 | 2 | 2 | 1 | 8 |
| 61 | 31.53 | 2.83 | 68.15 | 20.60 | 0 | 3 | 4 | 4 |
| 62 | 31.84 | 2.81 | 118.00 | 27.39 | 2 | 0 | 5 | 4 |
| 63 | 32.15 | 2.78 | 625.26 | 45.05 | 2 | 2 | 2 | 8 |
| 64 | 32.20 | 2.78 | 350.43 | 33.79 | 1 | 2 | 6 | 8 |
| 65 | 32.38 | 2.76 | 18.17 | 10.95 | 0 | 1 | 8 | 4 |
| 66 | 32.40 | 2.76 | 67.16 | 14.89 | 1 | 3 | 3 | 8 |
| 67 | 32.78 | 2.73 | 151.33 | 32.01 | 0 | 2 | 7 | 4 |
| 68 | 33.17 | 2.70 | 42.89 | 12.20 | 2 | 1 | 5 | 8 |
| 69 | 33.33 | 2.69 | 1071.09 | 61.32 | 2 | 2 | 3 | 8 |
| 70 | 33.56 | 2.67 | 33.21 | 15.38 | 1 | 0 | 8 | 4 |
| 71 | 33.67 | 2.66 | 149.63 | 32.77 | 0 | 3 | 5 | 4 |
| 72 | 34.04 | 2.63 | 194.03 | 26.70 | 1 | 3 | 4 | 8 |
| 73 | 34.42 | 2.60 | 290.50 | 46.77 | 2 | 0 | 6 | 4 |
| 74 | 34.83 | 2.57 | 252.27 | 31.21 | 1 | 1 | 8 | 8 |
| 75 | 34.93 | 2.57 | 562.67 | 46.77 | 2 | 2 | 4 | 8 |
| 76 | 35.20 | 2.55 | 280.02 | 33.28 | 1 | 2 | 7 | 8 |
| 77 | 35.66 | 2.52 | 12.42 | 7.11 | 2 | 1 | 6 | 8 |
| 78 | 36.05 | 2.49 | 37.93 | 12.57 | 1 | 3 | 5 | 8 |
| 79 | 36.14 | 2.48 | 12.18 | 10.10 | 0 | 3 | 6 | 4 |
| 80 | 36.17 | 2.48 | 0.59 | 2.22 | 0 | 2 | 8 | 4 |
| 81 | 36.24 | 2.48 | 420.63 | 59.53 | 0 | 1 | 9 | 4 |
| 82 | 36.77 | 2.44 | 2.11 | 6.06 | 0 | 4 | 0 | 2 |
| 83 | 36.89 | 2.43 | 35.56 | 12.48 | 2 | 2 | 5 | 8 |
| 84 | 36.99 | 2.43 | 22.74 | 14.16 | 0 | 4 | 1 | 4 |
| 85 | 37.26 | 2.41 | 12.45 | 10.56 | 2 | 0 | 7 | 4 |
| 86 | 37.31 | 2.41 | 1474.09 | 115.09 | 1 | 0 | 9 | 4 |
| 87 | 37.49 | 2.40 | 70.72 | 25.34 | 2 | 3 | 0 | 4 |
| 88 | 37.62 | 2.39 | 282.47 | 50.83 | 0 | 4 | 2 | 4 |
| 89 | 37.70 | 2.38 | 340.44 | 39.56 | 2 | 3 | 1 | 8 |
| 90 | 38.09 | 2.36 | 8.40 | 12.57 | 3 | 0 | 0 | 2 |
| 91 | 38.29 | 2.35 | 22.22 | 14.54 | 3 | 0 | 1 | 4 |
| 92 | 38.33 | 2.35 | 204.51 | 31.22 | 2 | 3 | 2 | 8 |
| 93 | 38.37 | 2.34 | 93.20 | 21.11 | 1 | 3 | 6 | 8 |
| 94 | 38.41 | 2.34 | 609.48 | 54.03 | 1 | 2 | 8 | 8 |
| 95 | 38.42 | 2.34 | 778.55 | 61.09 | 2 | 1 | 7 | 8 |
| 96 | 38.47 | 2.34 | 308.20 | 38.49 | 1 | 1 | 9 | 8 |
| 97 | 38.66 | 2.33 | 162.31 | 39.71 | 0 | 4 | 3 | 4 |
| 98 | 38.87 | 2.32 | 47.21 | 21.55 | 0 | 3 | 7 | 4 |
| 99 | 38.91 | 2.31 | 60.01 | 24.32 | 3 | 0 | 2 | 4 |
| 100 | 38.98 | 2.31 | 1.58 | 3.96 | 1 | 4 | 0 | 4 |
| 101 | 39.06 | 2.30 | 101.01 | 44.81 | 0 | 0 | 10 | 2 |
| 102 | 39.18 | 2.30 | 391.75 | 44.28 | 2 | 2 | 6 | 8 |
| 103 | 39.18 | 2.30 | 210.27 | 32.44 | 1 | 4 | 1 | 8 |
| 104 | 39.23 | 2.29 | 73.26 | 27.12 | 3 | 1 | 0 | 4 |
| 105 | 39.35 | 2.29 | 49.87 | 15.87 | 2 | 3 | 3 | 8 |
| 106 | 39.43 | 2.28 | 38.54 | 13.99 | 3 | 1 | 1 | 8 |
| 107 | 39.71 | 2.27 | 60.34 | 24.95 | 0 | 2 | 9 | 4 |
| 108 | 39.79 | 2.26 | 138.83 | 26.81 | 1 | 4 | 2 | 8 |
| 109 | 39.92 | 2.26 | 311.30 | 56.99 | 3 | 0 | 3 | 4 |
| 110 | 40.03 | 2.25 | 1.58 | 2.88 | 3 | 1 | 2 | 8 |
| 111 | 40.07 | 2.25 | 134.33 | 37.59 | 0 | 4 | 4 | 4 |
| 112 | 40.17 | 2.24 | 4.15 | 6.63 | 0 | 1 | 10 | 4 |
| 113 | 40.33 | 2.23 | 2.14 | 4.78 | 2 | 0 | 8 | 4 |
| 114 | 40.74 | 2.21 | 147.53 | 28.38 | 2 | 3 | 4 | 8 |
| 115 | 40.78 | 2.21 | 98.56 | 23.22 | 1 | 4 | 3 | 8 |
| 116 | 40.98 | 2.20 | 123.13 | 26.10 | 1 | 3 | 7 | 8 |
| 117 | 41.02 | 2.20 | 168.86 | 30.59 | 3 | 1 | 3 | 8 |
| 118 | 41.16 | 2.19 | 10.74 | 10.95 | 1 | 0 | 10 | 4 |
| 119 | 41.30 | 2.18 | 28.88 | 18.03 | 3 | 0 | 4 | 4 |
| 120 | 41.42 | 2.18 | 222.88 | 35.53 | 2 | 1 | 8 | 8 |
| 121 | 41.74 | 2.16 | 68.19 | 19.82 | 2 | 2 | 7 | 8 |
| 122 | 41.79 | 2.16 | 22.97 | 11.52 | 1 | 2 | 9 | 8 |
| 123 | 41.83 | 2.16 | 101.82 | 34.34 | 0 | 4 | 5 | 4 |
| 124 | 41.84 | 2.16 | 14.53 | 12.97 | 0 | 3 | 8 | 4 |
| 125 | 42.13 | 2.14 | 69.01 | 20.15 | 1 | 4 | 4 | 8 |
| 126 | 42.23 | 2.14 | 171.77 | 31.88 | 1 | 1 | 10 | 8 |
| 127 | 42.36 | 2.13 | 187.64 | 33.43 | 3 | 1 | 4 | 8 |
| 128 | 42.47 | 2.13 | 92.06 | 23.49 | 2 | 3 | 5 | 8 |
| 129 | 42.50 | 2.13 | 55.86 | 25.89 | 3 | 2 | 0 | 4 |
| 130 | 42.69 | 2.12 | 80.09 | 22.03 | 3 | 2 | 1 | 8 |
| 131 | 43.01 | 2.10 | 9.60 | 10.88 | 3 | 0 | 5 | 4 |
| 132 | 43.25 | 2.09 | 471.52 | 54.24 | 3 | 2 | 2 | 8 |
| 133 | 43.38 | 2.08 | 11.21 | 11.87 | 0 | 2 | 10 | 4 |
| 134 | 43.58 | 2.07 | 17.13 | 14.75 | 2 | 0 | 9 | 4 |
| 135 | 43.82 | 2.06 | 114.73 | 27.16 | 1 | 4 | 5 | 8 |
| 136 | 43.83 | 2.06 | 4.37 | 5.30 | 1 | 3 | 8 | 8 |
| 137 | 43.90 | 2.06 | 502.22 | 80.51 | 0 | 4 | 6 | 4 |
| 138 | 44.05 | 2.05 | 20.46 | 11.53 | 3 | 1 | 5 | 8 |
| 139 | 44.18 | 2.05 | 9.06 | 7.70 | 3 | 2 | 3 | 8 |
| 140 | 44.18 | 2.05 | 143.16 | 43.29 | 0 | 1 | 11 | 4 |
| 141 | 44.52 | 2.03 | 1.66 | 3.32 | 2 | 3 | 6 | 8 |
| 142 | 44.55 | 2.03 | 103.31 | 26.25 | 2 | 2 | 8 | 8 |
| 143 | 44.61 | 2.03 | 7.76 | 7.20 | 2 | 1 | 9 | 8 |

TABLE 18
Unit cell parameters for orthorhombic P222₁ (S)-nicotinium.

| | |
|---|---|
| Orthorhombic P222₁ | R₁ = 4.81% |

| | | |
|---|---|---|
| a = 6.9489 (7) | α = 90° | d min (Mo) = 0.77 |
| b = 9.7256 (10) | β = 90° | Flack = 0.0 (6) |
| c = 23.026 (2) | γ = 90° | Volume = 1556.1 (3) |

Monoclinic P2₁ (S)-Nicotinium 2,5-Dihydroxyterephthalate. Small Scale: 2,5-Dihydroxyterephthalic acid (198.1 mg, 1.0 mmol) was added into a 20 mL scintillation vial. MeOH (14.0 mL) was added with vigorous agitation. (S)-Nicotine (0.32 mL, 2.0 mmol) was added via micropipette in the dark to avoid degradation. The resulting solution was vortexed for 60 seconds at 3,000 rpm on a VWR Mini Vortexer MV I. The solution was then stored in the dark, uncapped to allow for crystal formation while the solvent slowly evaporated. Once the solvent evaporated, the crystalline product was collected via vacuum filtration, washing with n-heptane (3×5 mL) (514.7 mg, 98.49%). The resulting crystalline product was characterized by single crystal X-ray diffraction, simulated powder X-ray diffraction, melting point, differential scanning calorimetry, and a UV irradiation degradation test monitored by ¹H NMR.

Large Scale: 2,5-Dihydroxyterephthalic acid (990.7 mg, 5.0 mmol) was added into a 150 mL beaker. MeOH (100.0 mL) was added with vigorous stirring on a hot plate. (S)-Nicotine (1.60 mL, 10.0 mmol) was added via micropipette in the dark to avoid degradation. The resulting solution was stirred for 180 seconds on a hot plate. The solution was then stored in the dark, uncapped to allow for crystal formation while the solvent slowly evaporated. Once the solvent evaporated, the crystalline product was collected via vacuum filtration, washing with n-heptane (3×20 mL) (2.6013 g, 99.55%). The resulting crystalline product was characterized by single crystal X-ray diffraction, simulated powder X-ray diffraction, melting point, differential scanning calorimetry, and a UV irradiation degradation test monitored by ¹H NMR.

TABLE 19
X-ray powder pattern peak data for monoclinic
P2₁ (S)-nicotinium 2,5-dihydroxyterephthalate.

| Peak | 2Θ | d-spacing | | | | | | |
|---|---|---|---|---|---|---|---|---|
| No. | [°] | [Å] | Intensity | F | h | k | l | Mult. |
| 1 | 6.09 | 14.50 | 0.43 | 0.32 | 0 | 0 | 1 | 2 |
| 2 | 9.06 | 9.75 | 3573.49 | 30.77 | 0 | −1 | 1 | 4 |
| 3 | 12.20 | 7.25 | 6.29 | 2.46 | 0 | 0 | 2 | 2 |
| 4 | 12.96 | 6.82 | 47.41 | 7.19 | 1 | 0 | 0 | 2 |
| 5 | 13.44 | 6.59 | 48.97 | 7.58 | 0 | 2 | 0 | 2 |
| 6 | 13.93 | 6.35 | 3907.92 | 49.71 | 0 | −1 | 2 | 4 |
| 7 | 14.30 | 6.19 | 10000.00 | 115.47 | −1 | 0 | 1 | 2 |
| 8 | 14.36 | 6.16 | 1342.35 | 42.50 | 1 | 0 | 1 | 2 |
| 9 | 14.61 | 6.06 | 1265.56 | 29.68 | −1 | 1 | 0 | 4 |
| 10 | 14.76 | 6.00 | 2504.89 | 42.21 | 0 | −2 | 1 | 4 |
| 11 | 15.81 | 5.60 | 650.02 | 23.06 | −1 | 1 | 1 | 4 |
| 12 | 15.87 | 5.58 | 284.72 | 15.32 | 1 | −1 | 1 | 4 |
| 13 | 17.78 | 4.98 | 178.14 | 19.26 | −1 | 0 | 2 | 2 |
| 14 | 17.89 | 4.96 | 0.42 | 0.94 | 1 | 0 | 2 | 2 |
| 15 | 18.19 | 4.87 | 6516.95 | 84.28 | 0 | −2 | 2 | 4 |
| 16 | 18.34 | 4.83 | 255.83 | 23.83 | 0 | 0 | 3 | 2 |
| 17 | 18.71 | 4.74 | 4854.08 | 74.89 | −1 | 2 | 0 | 4 |
| 18 | 19.03 | 4.66 | 756.28 | 30.08 | −1 | 1 | 2 | 4 |
| 19 | 19.12 | 4.64 | 998.77 | 34.74 | 1 | −1 | 2 | 4 |
| 20 | 19.55 | 4.54 | 1182.95 | 38.69 | 0 | −1 | 3 | 4 |
| 21 | 19.67 | 4.51 | 2034.20 | 51.05 | −1 | 2 | 1 | 4 |
| 22 | 19.72 | 4.50 | 3634.24 | 68.40 | 1 | −2 | 1 | 4 |
| 23 | 21.13 | 4.20 | 99.52 | 12.16 | 0 | −3 | 1 | 4 |
| 24 | 22.36 | 3.97 | 5348.62 | 94.53 | −1 | 2 | 2 | 4 |
| 25 | 22.44 | 3.96 | 1282.78 | 46.47 | 1 | −2 | 2 | 4 |
| 26 | 22.46 | 3.95 | 3819.12 | 113.54 | −1 | 0 | 3 | 2 |
| 27 | 22.59 | 3.93 | 20.28 | 8.32 | 1 | 0 | 3 | 2 |
| 28 | 22.81 | 3.90 | 4648.35 | 89.98 | 0 | −2 | 3 | 4 |
| 29 | 23.47 | 3.79 | 1007.39 | 43.16 | −1 | −1 | 3 | 4 |
| 30 | 23.59 | 3.77 | 1284.08 | 48.98 | 1 | −1 | 3 | 4 |
| 31 | 23.67 | 3.76 | 5045.99 | 97.48 | 0 | −3 | 2 | 4 |
| 32 | 24.08 | 3.69 | 578.49 | 33.61 | −1 | 3 | 0 | 4 |
| 33 | 24.54 | 3.62 | 46.28 | 13.71 | 0 | 0 | 4 | 2 |
| 34 | 24.85 | 3.58 | 558.32 | 34.11 | −1 | 3 | 1 | 4 |
| 35 | 24.88 | 3.58 | 716.58 | 38.71 | 1 | −3 | 1 | 4 |
| 36 | 25.47 | 3.49 | 626.06 | 37.07 | 0 | −1 | 4 | 4 |
| 37 | 26.09 | 3.41 | 361.93 | 40.90 | 2 | 0 | 0 | 2 |
| 38 | 26.27 | 3.39 | 933.56 | 46.77 | −1 | −2 | 3 | 4 |
| 39 | 26.37 | 3.38 | 1839.22 | 65.93 | 1 | −2 | 3 | 4 |
| 40 | 26.78 | 3.33 | 55.97 | 16.53 | −2 | 0 | 1 | 2 |
| 41 | 26.85 | 3.32 | 164.16 | 28.39 | 2 | 0 | 1 | 2 |
| 42 | 26.97 | 3.30 | 723.12 | 42.33 | −2 | 1 | 0 | 4 |
| 43 | 27.05 | 3.29 | 852.92 | 46.11 | −1 | 3 | 2 | 4 |
| 44 | 27.06 | 3.29 | 141.54 | 26.58 | 0 | 4 | 0 | 2 |
| 45 | 27.12 | 3.29 | 3113.93 | 88.34 | 1 | −3 | 2 | 4 |
| 46 | 27.43 | 3.25 | 5749.99 | 121.50 | 0 | −3 | 3 | 4 |
| 47 | 27.64 | 3.22 | 361.01 | 30.70 | −2 | 1 | 1 | 4 |
| 48 | 27.71 | 3.22 | 152.48 | 20.00 | 2 | −1 | 1 | 4 |
| 49 | 27.76 | 3.21 | 512.62 | 36.75 | 0 | −4 | 1 | 4 |
| 50 | 27.78 | 3.21 | 0.69 | 1.92 | −1 | 0 | 4 | 2 |
| 51 | 27.91 | 3.19 | 3.54 | 4.34 | 1 | 0 | 4 | 2 |
| 52 | 28.08 | 3.18 | 17.54 | 6.88 | 0 | −2 | 4 | 4 |
| 53 | 28.61 | 3.12 | 336.09 | 30.73 | −1 | −1 | 4 | 4 |
| 54 | 28.74 | 3.10 | 1220.35 | 58.84 | 1 | −1 | 4 | 4 |
| 55 | 28.83 | 3.09 | 1117.13 | 79.87 | −2 | 0 | 2 | 2 |
| 56 | 28.96 | 3.08 | 130.17 | 27.40 | 2 | 0 | 2 | 2 |
| 57 | 29.46 | 3.03 | 564.43 | 41.08 | −2 | 2 | 0 | 4 |
| 58 | 29.63 | 3.01 | 779.55 | 48.59 | −2 | 1 | 2 | 4 |
| 59 | 29.76 | 3.00 | 25.35 | 8.80 | 2 | −1 | 2 | 4 |
| 60 | 29.78 | 3.00 | 2145.33 | 81.02 | 0 | −4 | 2 | 4 |
| 61 | 30.08 | 2.97 | 1.30 | 2.01 | −2 | 2 | 1 | 4 |
| 62 | 30.11 | 2.97 | 49.21 | 12.42 | −1 | 4 | 0 | 4 |
| 63 | 30.14 | 2.96 | 13.44 | 6.50 | 2 | −2 | 1 | 4 |
| 64 | 30.40 | 2.94 | 70.22 | 14.98 | −1 | −3 | 3 | 4 |
| 65 | 30.49 | 2.93 | 71.86 | 15.21 | 1 | −3 | 3 | 4 |
| 66 | 30.73 | 2.91 | 169.19 | 23.54 | −1 | 4 | 1 | 4 |
| 67 | 30.77 | 2.90 | 296.89 | 31.21 | 1 | −4 | 1 | 4 |
| 68 | 30.81 | 2.90 | 45.97 | 17.40 | 0 | 0 | 5 | 2 |
| 69 | 30.98 | 2.88 | 41.76 | 11.79 | −1 | −2 | 4 | 4 |
| 70 | 31.10 | 2.87 | 766.11 | 50.73 | 1 | −2 | 4 | 4 |
| 71 | 31.57 | 2.83 | 34.81 | 10.99 | 0 | −1 | 5 | 4 |
| 72 | 31.93 | 2.80 | 140.57 | 22.36 | −2 | 2 | 2 | 4 |
| 73 | 32.00 | 2.80 | 0.26 | 1.35 | −2 | 0 | 3 | 2 |
| 74 | 32.00 | 2.80 | 379.58 | 36.82 | 0 | −3 | 4 | 4 |
| 75 | 32.05 | 2.79 | 392.60 | 37.51 | 2 | −2 | 2 | 4 |
| 76 | 32.17 | 2.78 | 37.56 | 16.48 | 2 | 0 | 3 | 2 |
| 77 | 32.57 | 2.75 | 5.90 | 4.68 | −1 | 4 | 2 | 4 |
| 78 | 32.63 | 2.74 | 54.16 | 14.20 | 1 | −4 | 2 | 4 |
| 79 | 32.73 | 2.73 | 1514.56 | 75.36 | −2 | 1 | 3 | 4 |
| 80 | 32.89 | 2.72 | 632.14 | 48.95 | 0 | −4 | 3 | 4 |
| 81 | 32.90 | 2.72 | 13.97 | 7.28 | 2 | −1 | 3 | 4 |
| 82 | 33.23 | 2.69 | 122.30 | 21.77 | −2 | 3 | 0 | 4 |
| 83 | 33.48 | 2.67 | 0.06 | 0.70 | −1 | 0 | 5 | 2 |
| 84 | 33.62 | 2.66 | 186.56 | 38.52 | 1 | 0 | 5 | 2 |
| 85 | 33.75 | 2.65 | 315.79 | 35.58 | 0 | −2 | 5 | 4 |
| 86 | 33.78 | 2.65 | 87.69 | 18.77 | −2 | 3 | 1 | 4 |
| 87 | 33.84 | 2.65 | 328.23 | 36.38 | 2 | −3 | 1 | 4 |
| 88 | 34.19 | 2.62 | 379.69 | 39.56 | −1 | −1 | 5 | 4 |
| 89 | 34.32 | 2.61 | 103.89 | 20.79 | 1 | −1 | 5 | 4 |
| 90 | 34.58 | 2.59 | 4.35 | 4.29 | 0 | −5 | 1 | 4 |
| 91 | 34.60 | 2.59 | 253.21 | 32.73 | −1 | −3 | 4 | 4 |
| 92 | 34.71 | 2.58 | 167.08 | 26.68 | 1 | −3 | 4 | 4 |
| 93 | 34.84 | 2.57 | 12.51 | 7.33 | −2 | 2 | 3 | 4 |
| 94 | 35.01 | 2.56 | 107.83 | 21.64 | 2 | −2 | 3 | 4 |
| 95 | 35.45 | 2.53 | 151.45 | 25.99 | −1 | −4 | 3 | 4 |
| 96 | 35.46 | 2.53 | 25.17 | 10.60 | −2 | 3 | 2 | 4 |
| 97 | 35.53 | 2.52 | 164.86 | 27.19 | 1 | −4 | 3 | 4 |
| 98 | 35.57 | 2.52 | 235.75 | 32.56 | 2 | −3 | 2 | 4 |
| 99 | 36.02 | 2.49 | 242.05 | 47.29 | −2 | 0 | 4 | 2 |
| 100 | 36.23 | 2.48 | 57.00 | 16.33 | −1 | −2 | 5 | 4 |
| 101 | 36.23 | 2.48 | 455.63 | 65.31 | 2 | 0 | 4 | 2 |
| 102 | 36.26 | 2.48 | 53.40 | 15.82 | 0 | −5 | 2 | 4 |
| 103 | 36.36 | 2.47 | 153.74 | 26.93 | 1 | −2 | 5 | 4 |
| 104 | 36.54 | 2.46 | 61.98 | 17.19 | −1 | 5 | 0 | 4 |
| 105 | 36.68 | 2.45 | 26.54 | 11.30 | −2 | 1 | 4 | 4 |
| 106 | 36.85 | 2.44 | 187.17 | 30.16 | 0 | −4 | 4 | 4 |
| 107 | 36.89 | 2.43 | 89.77 | 20.91 | 2 | −1 | 4 | 4 |
| 108 | 37.06 | 2.42 | 30.32 | 12.21 | −1 | 5 | 1 | 4 |
| 109 | 37.09 | 2.42 | 570.93 | 53.04 | 1 | −5 | 1 | 4 |
| 110 | 37.13 | 2.42 | 450.20 | 47.16 | 0 | −3 | 5 | 4 |
| 111 | 37.18 | 2.42 | 11.13 | 10.50 | 0 | 0 | 6 | 2 |
| 112 | 37.82 | 2.38 | 163.25 | 28.98 | 0 | −1 | 6 | 4 |
| 113 | 37.94 | 2.37 | 188.84 | 31.28 | −2 | 4 | 0 | 4 |
| 114 | 38.14 | 2.36 | 508.84 | 51.64 | −2 | 3 | 3 | 4 |
| 115 | 38.29 | 2.35 | 1.96 | 3.22 | 2 | −3 | 3 | 4 |
| 116 | 38.44 | 2.34 | 206.15 | 33.16 | −2 | 4 | 1 | 4 |
| 117 | 38.49 | 2.34 | 429.02 | 47.90 | 2 | −4 | 1 | 4 |
| 118 | 38.60 | 2.33 | 153.87 | 28.78 | −2 | 2 | 4 | 4 |
| 119 | 38.63 | 2.33 | 14.53 | 8.85 | −1 | 5 | 2 | 4 |
| 120 | 38.68 | 2.33 | 467.02 | 50.25 | 1 | −5 | 2 | 4 |
| 121 | 38.80 | 2.32 | 81.15 | 21.02 | 2 | −2 | 4 | 4 |
| 122 | 38.91 | 2.31 | 16.04 | 9.37 | 0 | −5 | 3 | 4 |
| 123 | 39.17 | 2.30 | 69.16 | 19.61 | −1 | −4 | 4 | 4 |
| 124 | 39.27 | 2.29 | 183.74 | 32.05 | 1 | −4 | 4 | 4 |
| 125 | 39.42 | 2.28 | 62.73 | 18.81 | −1 | −3 | 5 | 4 |
| 126 | 39.46 | 2.28 | 0.52 | 2.43 | −1 | 0 | 6 | 2 |
| 127 | 39.55 | 2.28 | 370.75 | 45.88 | 1 | −3 | 5 | 4 |
| 128 | 39.58 | 2.28 | 8.52 | 9.85 | 3 | 0 | 0 | 2 |
| 129 | 39.60 | 2.27 | 0.02 | 0.44 | 1 | 0 | 6 | 2 |
| 130 | 39.70 | 2.27 | 17.86 | 10.12 | 0 | −2 | 6 | 4 |
| 131 | 39.95 | 2.25 | 29.60 | 13.11 | −2 | 4 | 2 | 4 |
| 132 | 40.05 | 2.25 | 6.38 | 6.10 | 2 | −4 | 2 | 4 |
| 133 | 40.05 | 2.25 | 36.48 | 20.65 | −3 | 0 | 1 | 2 |
| 134 | 40.07 | 2.25 | 63.61 | 19.29 | −1 | −1 | 6 | 4 |
| 135 | 40.12 | 2.25 | 215.29 | 50.25 | 3 | 0 | 1 | 2 |
| 136 | 40.19 | 2.24 | 16.58 | 9.88 | −3 | 1 | 0 | 4 |
| 137 | 40.22 | 2.24 | 170.33 | 31.69 | 1 | −1 | 6 | 4 |
| 138 | 40.66 | 2.22 | 15.29 | 9.61 | −3 | 1 | 1 | 4 |
| 139 | 40.68 | 2.22 | 66.28 | 28.32 | −2 | 0 | 5 | 2 |
| 140 | 40.73 | 2.21 | 124.69 | 27.50 | 3 | −1 | 1 | 4 |
| 141 | 40.92 | 2.20 | 0.78 | 3.09 | 2 | 0 | 5 | 2 |
| 142 | 41.09 | 2.20 | 96.18 | 34.49 | 0 | 6 | 0 | 2 |
| 143 | 41.14 | 2.19 | 25.52 | 12.58 | −1 | −5 | 3 | 4 |
| 144 | 41.21 | 2.19 | 397.26 | 49.73 | 1 | −5 | 3 | 4 |
| 145 | 41.28 | 2.19 | 176.96 | 33.25 | −2 | −1 | 5 | 4 |
| 146 | 41.46 | 2.18 | 68.00 | 20.71 | 0 | −4 | 5 | 4 |
| 147 | 41.50 | 2.17 | 1.83 | 4.81 | −3 | 0 | 2 | 2 |
| 148 | 41.52 | 2.17 | 79.14 | 22.38 | 2 | −1 | 5 | 4 |
| 149 | 41.58 | 2.17 | 28.22 | 13.39 | 0 | −6 | 1 | 4 |
| 150 | 41.64 | 2.17 | 20.28 | 16.08 | 3 | 0 | 2 | 2 |
| 151 | 41.65 | 2.17 | 189.06 | 34.71 | −2 | 3 | 4 | 4 |
| 152 | 41.83 | 2.16 | 68.45 | 20.99 | 2 | −3 | 4 | 4 |
| 153 | 41.86 | 2.16 | 56.12 | 19.02 | −1 | −2 | 6 | 4 |
| 154 | 41.98 | 2.15 | 91.45 | 24.36 | −3 | 2 | 0 | 4 |
| 155 | 42.00 | 2.15 | 155.15 | 31.75 | 1 | −2 | 6 | 4 |
| 156 | 42.09 | 2.15 | 121.26 | 28.13 | −3 | 1 | 2 | 4 |
| 157 | 42.23 | 2.14 | 104.44 | 26.20 | 3 | −1 | 2 | 4 |
| 158 | 42.39 | 2.13 | 5.04 | 5.78 | −2 | 4 | 3 | 4 |
| 159 | 42.39 | 2.13 | 984.35 | 80.78 | 0 | −5 | 4 | 4 |
| 160 | 42.43 | 2.13 | 77.17 | 22.65 | −3 | 2 | 1 | 4 |
| 161 | 42.50 | 2.13 | 96.00 | 25.30 | 3 | −2 | 1 | 4 |
| 162 | 42.52 | 2.12 | 71.68 | 21.88 | 2 | −4 | 3 | 4 |
| 163 | 42.68 | 2.12 | 27.20 | 13.53 | 0 | −3 | 6 | 4 |
| 164 | 43.02 | 2.10 | 107.07 | 27.09 | 0 | −6 | 2 | 4 |
| 165 | 43.03 | 2.10 | 6.22 | 6.53 | −2 | −2 | 5 | 4 |
| 166 | 43.26 | 2.09 | 138.22 | 30.97 | −1 | 6 | 0 | 4 |
| 167 | 43.26 | 2.09 | 48.75 | 18.40 | 2 | −2 | 5 | 4 |
| 168 | 43.36 | 2.09 | 43.79 | 17.48 | −2 | 5 | 0 | 4 |
| 169 | 43.57 | 2.08 | 13.42 | 9.73 | −1 | −4 | 5 | 4 |
| 170 | 43.67 | 2.07 | 0.20 | 1.68 | 0 | 0 | 7 | 2 |
| 171 | 43.68 | 2.07 | 3.37 | 4.89 | 1 | −4 | 5 | 4 |
| 172 | 43.72 | 2.07 | 6.91 | 7.01 | −1 | 6 | 1 | 4 |
| 173 | 43.74 | 2.07 | 143.26 | 31.93 | 1 | −6 | 1 | 4 |
| 174 | 43.81 | 2.06 | 3.88 | 5.26 | −2 | 5 | 1 | 4 |
| 175 | 43.82 | 2.06 | 2.50 | 4.23 | −3 | 2 | 2 | 4 |
| 176 | 43.85 | 2.06 | 30.16 | 14.69 | 2 | −5 | 1 | 4 |
| 177 | 43.85 | 2.06 | 26.09 | 19.32 | −3 | 0 | 3 | 2 |
| 178 | 43.95 | 2.06 | 509.39 | 60.53 | 3 | −2 | 2 | 4 |
| 179 | 44.06 | 2.05 | 300.56 | 65.93 | 3 | 0 | 3 | 2 |
| 180 | 44.23 | 2.05 | 338.42 | 49.69 | 0 | −1 | 7 | 4 |
| 181 | 44.42 | 2.04 | 18.16 | 11.56 | −3 | 1 | 3 | 4 |
| 182 | 44.46 | 2.04 | 115.37 | 29.18 | −1 | −5 | 4 | 4 |
| 183 | 44.55 | 2.03 | 102.94 | 27.63 | 1 | −5 | 4 | 4 |
| 184 | 44.62 | 2.03 | 9.35 | 8.34 | 3 | −1 | 3 | 4 |
| 185 | 44.72 | 2.02 | 42.61 | 17.85 | −1 | −3 | 6 | 4 |
| 186 | 44.84 | 2.02 | 32.97 | 15.75 | −3 | 3 | 0 | 4 |
| 187 | 44.86 | 2.02 | 21.15 | 12.62 | 1 | −3 | 6 | 4 |
| 188 | 45.10 | 2.01 | 41.49 | 17.78 | −1 | 6 | 2 | 4 |
| 189 | 45.14 | 2.01 | 72.05 | 23.46 | 1 | −6 | 2 | 4 |
| 190 | 45.17 | 2.01 | 18.21 | 11.80 | −2 | 5 | 2 | 4 |
| 191 | 45.26 | 2.00 | 74.05 | 23.85 | 2 | −5 | 2 | 4 |
| 192 | 45.26 | 2.00 | 4.50 | 5.88 | −3 | 3 | 1 | 4 |
| 193 | 45.33 | 2.00 | 2.66 | 4.52 | 3 | −3 | 1 | 4 |
| 194 | 45.34 | 2.00 | 254.90 | 44.35 | 0 | −6 | 3 | 4 |
| 195 | 45.62 | 1.99 | 16.11 | 11.23 | −2 | 4 | 4 | 4 |
| 196 | 45.67 | 1.99 | 34.84 | 23.37 | −1 | 0 | 7 | 2 |
| 197 | 45.80 | 1.98 | 136.99 | 32.88 | 2 | −4 | 4 | 4 |
| 198 | 45.82 | 1.98 | 24.83 | 19.81 | 1 | 0 | 7 | 2 |
| 199 | 45.83 | 1.98 | 27.97 | 14.87 | −2 | −3 | 5 | 4 |
| 200 | 45.85 | 1.98 | 167.09 | 51.43 | −2 | 0 | 6 | 2 |
| 201 | 45.89 | 1.98 | 261.38 | 45.52 | 0 | −2 | 7 | 4 |
| 202 | 46.05 | 1.97 | 1.37 | 3.31 | 2 | −3 | 5 | 4 |
| 203 | 46.07 | 1.97 | 42.38 | 18.41 | −3 | 2 | 3 | 4 |
| 204 | 46.12 | 1.97 | 4.04 | 8.05 | 2 | 0 | 6 | 2 |
| 205 | 46.21 | 1.96 | 29.50 | 15.41 | −1 | −1 | 7 | 4 |
| 206 | 46.27 | 1.96 | 6.11 | 7.03 | 3 | −2 | 3 | 4 |
| 207 | 46.36 | 1.96 | 460.45 | 61.12 | 1 | −1 | 7 | 4 |
| 208 | 46.40 | 1.96 | 249.54 | 45.04 | −2 | −1 | 6 | 4 |
| 209 | 46.54 | 1.95 | 288.91 | 48.63 | 0 | −5 | 5 | 4 |
| 210 | 46.58 | 1.95 | 12.50 | 10.12 | −3 | 3 | 2 | 4 |
| 211 | 46.58 | 1.95 | 190.59 | 39.54 | 0 | −4 | 6 | 4 |
| 212 | 46.66 | 1.95 | 65.56 | 23.23 | 2 | −1 | 6 | 4 |
| 213 | 46.71 | 1.94 | 59.40 | 22.14 | 3 | −3 | 2 | 4 |
| 214 | 47.00 | 1.93 | 37.13 | 24.93 | −3 | 0 | 4 | 2 |
| 215 | 47.26 | 1.92 | 7.08 | 10.95 | 3 | 0 | 4 | 2 |
| 216 | 47.33 | 1.92 | 42.34 | 18.97 | −1 | −6 | 3 | 4 |
| 217 | 47.39 | 1.92 | 24.52 | 14.46 | −2 | 5 | 3 | 4 |
| 218 | 47.39 | 1.92 | 26.91 | 15.15 | 1 | −6 | 3 | 4 |
| 219 | 47.51 | 1.91 | 178.64 | 39.14 | 2 | −5 | 3 | 4 |
| 220 | 47.53 | 1.91 | 22.51 | 13.90 | −3 | 1 | 4 | 4 |
| 221 | 47.79 | 1.90 | 303.61 | 51.36 | 3 | −1 | 4 | 4 |
| 222 | 47.82 | 1.90 | 53.46 | 21.57 | −1 | −2 | 7 | 4 |
| 223 | 47.97 | 1.90 | 22.02 | 13.89 | 1 | −2 | 7 | 4 |
| 224 | 48.00 | 1.89 | 65.74 | 24.02 | −2 | −2 | 6 | 4 |
| 225 | 48.25 | 1.88 | 23.03 | 14.30 | 2 | −2 | 6 | 4 |
| 226 | 48.44 | 1.88 | 82.31 | 27.16 | 0 | −6 | 4 | 4 |
| 227 | 48.47 | 1.88 | 98.59 | 29.74 | −1 | −5 | 5 | 4 |
| 228 | 48.50 | 1.88 | 29.85 | 16.38 | −1 | −4 | 6 | 4 |
| 229 | 48.56 | 1.87 | 22.67 | 14.30 | 0 | −3 | 7 | 4 |
| 230 | 48.57 | 1.87 | 328.07 | 54.39 | 1 | −5 | 5 | 4 |
| 231 | 48.61 | 1.87 | 197.59 | 42.24 | −3 | 4 | 0 | 4 |
| 232 | 48.62 | 1.87 | 66.30 | 24.48 | 1 | −4 | 6 | 4 |
| 233 | 48.74 | 1.87 | 446.71 | 63.71 | −3 | 3 | 3 | 4 |
| 234 | 48.77 | 1.87 | 22.14 | 14.19 | 0 | −7 | 1 | 4 |
| 235 | 48.92 | 1.86 | 32.36 | 17.22 | 3 | −3 | 3 | 4 |
| 236 | 49.01 | 1.86 | 18.38 | 13.01 | −3 | 4 | 1 | 4 |
| 237 | 49.07 | 1.86 | 220.17 | 45.07 | 3 | −4 | 1 | 4 |
| 238 | 49.11 | 1.85 | 9.59 | 9.42 | −3 | 2 | 4 | 4 |
| 239 | 49.32 | 1.85 | 0.32 | 1.73 | −2 | 6 | 0 | 4 |
| 240 | 49.36 | 1.85 | 69.39 | 25.47 | 3 | −2 | 4 | 4 |
| 241 | 49.55 | 1.84 | 47.55 | 21.18 | −2 | −4 | 5 | 4 |
| 242 | 49.73 | 1.83 | 19.76 | 13.71 | −2 | 6 | 1 | 4 |
| 243 | 49.75 | 1.83 | 22.64 | 14.68 | 2 | −4 | 5 | 4 |
| 244 | 49.77 | 1.83 | 66.64 | 25.20 | 2 | −6 | 1 | 4 |

TABLE 20
Unit cell parameters for monoclinic P2₁ (S)-
nicotinium 2,5-dihydroxyterephthalate.

| | |
|---|---|
| Monoclinic P2₁ | R₁ = 4.33% |

| | | |
|---|---|---|
| a = 6.8250 (3) | α = 90° | d min (Mo) = 0.68 |
| b = 13.1702 (6) | β = 90.326 (1)° | Flack = −0.6 (5) |
| c = 14.4987 (6) | γ = 90° | Volume = 1303.22 (10) |

Monoclinic P2₁ (S)-Nicotinium Bi-L-(+)-Tartrate Dihydrate. (S)-nicotine (0.80 mL, 5.0 mmol) was added into a 30 mL beaker via micropipette in the dark to avoid degradation. EtOH (10.0 mL) was added with vigorous agitation to achieve a homogeneous room temperature solution. In a separate vessel, L-tartaric (1.50 g, 10.0 mmol) was dissolved into boiling water (10 mL). The solutions were then combined and the resulting solution was stirred at 1000 rpm at a simmer for 15 minutes. The solution was then slowly evaporated to yield a powdered product. This material was then recrystallized from 50 mL of a 50:50 EtOH: water mixture. The resulting material was analyzed via single crystal X-ray diffraction, simulated powder X-ray diffraction, and melting point analysis.

TABLE 21
X-ray powder pattern peak data for monoclinic
P2₁ (S)-nicotinium bi-L-(+)-tartrate dihydrate.

| Peak | 2Θ | d-spacing | | | | | | |
|---|---|---|---|---|---|---|---|---|
| No. | [°] | [Å] | Intensity | F | h | k | l | Mult. |
| 1 | 2.24 | 39.48 | 1706.26 | 8.23 | 0 | 0 | 1 | 2 |
| 2 | 4.47 | 19.74 | 324.70 | 7.19 | 0 | 0 | 2 | 2 |
| 3 | 6.71 | 13.16 | 262.84 | 9.71 | 0 | 0 | 3 | 2 |
| 4 | 8.95 | 9.87 | 219.06 | 11.84 | 0 | 0 | 4 | 2 |
| 5 | 11.20 | 7.90 | 31.08 | 5.59 | 0 | 0 | 5 | 2 |
| 6 | 11.71 | 7.55 | 1307.94 | 37.94 | 1 | 0 | 0 | 2 |
| 7 | 11.73 | 7.54 | 196.34 | 14.73 | −1 | 0 | 1 | 2 |
| 8 | 12.11 | 7.30 | 946.28 | 33.39 | 1 | 0 | 1 | 2 |
| 9 | 12.17 | 7.27 | 4439.21 | 72.69 | −1 | 0 | 2 | 2 |
| 10 | 12.27 | 7.21 | 769.94 | 21.59 | 0 | −1 | 1 | 4 |
| 11 | 12.87 | 6.87 | 47.99 | 5.66 | 0 | −1 | 2 | 4 |
| 12 | 12.90 | 6.86 | 8.80 | 3.43 | 1 | 0 | 2 | 2 |
| 13 | 12.99 | 6.81 | 51.38 | 8.36 | −1 | 0 | 3 | 2 |
| 14 | 13.45 | 6.58 | 124.14 | 13.45 | 0 | 0 | 6 | 2 |
| 15 | 13.82 | 6.40 | 1353.61 | 32.29 | 0 | −1 | 3 | 4 |
| 16 | 14.00 | 6.32 | 386.81 | 24.74 | 1 | 0 | 3 | 2 |
| 17 | 14.12 | 6.27 | 1468.92 | 48.64 | −1 | 0 | 4 | 2 |
| 18 | 15.04 | 5.88 | 1158.60 | 32.57 | 0 | −1 | 4 | 4 |
| 19 | 15.36 | 5.76 | 116.67 | 14.94 | 1 | 0 | 4 | 2 |
| 20 | 15.51 | 5.71 | 1755.94 | 58.49 | −1 | 0 | 5 | 2 |
| 21 | 15.70 | 5.64 | 116.46 | 15.26 | 0 | 0 | 7 | 2 |
| 22 | 16.49 | 5.37 | 2310.22 | 50.51 | 0 | −1 | 5 | 4 |
| 23 | 16.84 | 5.26 | 2313.89 | 51.66 | −1 | 1 | 0 | 4 |
| 24 | 16.86 | 5.26 | 3448.67 | 63.12 | −1 | 1 | 1 | 4 |
| 25 | 16.92 | 5.24 | 4017.27 | 96.71 | 1 | 0 | 5 | 2 |
| 26 | 17.08 | 5.19 | 935.64 | 47.12 | −1 | 0 | 6 | 2 |
| 27 | 17.12 | 5.17 | 2358.28 | 53.05 | 1 | −1 | 1 | 4 |
| 28 | 17.17 | 5.16 | 2393.45 | 53.58 | −1 | 1 | 2 | 4 |
| 29 | 17.69 | 5.01 | 7038.59 | 94.78 | 1 | −1 | 2 | 4 |
| 30 | 17.76 | 4.99 | 3433.31 | 66.46 | −1 | 1 | 3 | 4 |
| 31 | 17.96 | 4.93 | 10000.00 | 162.25 | 0 | 0 | 8 | 2 |
| 32 | 18.10 | 4.90 | 6398.20 | 92.51 | 0 | −1 | 6 | 4 |
| 33 | 18.52 | 4.79 | 4933.10 | 83.17 | 1 | −1 | 3 | 4 |
| 34 | 18.62 | 4.76 | 4503.20 | 79.87 | −1 | 1 | 4 | 4 |
| 35 | 18.62 | 4.76 | 1437.56 | 63.84 | 1 | 0 | 6 | 2 |
| 36 | 18.79 | 4.72 | 492.59 | 37.72 | −1 | 0 | 7 | 2 |
| 37 | 19.58 | 4.53 | 638.77 | 31.69 | 1 | −1 | 4 | 4 |
| 38 | 19.69 | 4.50 | 746.20 | 34.45 | −1 | 1 | 5 | 4 |
| 39 | 19.85 | 4.47 | 2725.54 | 66.39 | 0 | −1 | 7 | 4 |
| 40 | 20.23 | 4.39 | 79.78 | 16.38 | 0 | 0 | 9 | 2 |
| 41 | 20.44 | 4.34 | 450.19 | 39.33 | 1 | 0 | 7 | 2 |
| 42 | 20.62 | 4.31 | 419.84 | 38.32 | −1 | 0 | 8 | 2 |
| 43 | 20.83 | 4.26 | 45.65 | 9.03 | 1 | −1 | 5 | 4 |
| 44 | 20.96 | 4.24 | 621.69 | 33.55 | −1 | −1 | 6 | 4 |
| 45 | 21.69 | 4.09 | 424.42 | 28.73 | 0 | −1 | 8 | 4 |
| 46 | 22.25 | 3.99 | 3090.80 | 79.57 | 1 | −1 | 6 | 4 |
| 47 | 22.34 | 3.98 | 4705.84 | 139.46 | 1 | 0 | 8 | 2 |
| 48 | 22.39 | 3.97 | 1269.38 | 51.34 | −1 | −1 | 7 | 4 |
| 49 | 22.51 | 3.95 | 3114.89 | 114.35 | 0 | 0 | 10 | 2 |
| 50 | 22.53 | 3.94 | 57.18 | 15.51 | −1 | 0 | 9 | 2 |
| 51 | 23.45 | 3.79 | 69.69 | 17.85 | −2 | 0 | 1 | 2 |
| 52 | 23.54 | 3.78 | 7.98 | 6.07 | 2 | 0 | 0 | 2 |
| 53 | 23.58 | 3.77 | 37.19 | 13.12 | −2 | 0 | 2 | 2 |
| 54 | 23.62 | 3.76 | 2065.03 | 69.24 | 0 | −1 | 9 | 4 |
| 55 | 23.80 | 3.74 | 1645.25 | 62.29 | 1 | −1 | 7 | 4 |
| 56 | 23.84 | 3.73 | 7136.44 | 183.84 | 2 | 0 | 1 | 2 |
| 57 | 23.93 | 3.72 | 6125.41 | 170.94 | −2 | 0 | 3 | 2 |
| 58 | 23.95 | 3.71 | 4245.18 | 100.75 | −1 | −1 | 8 | 4 |
| 59 | 24.26 | 3.67 | 439.86 | 46.49 | 0 | 2 | 0 | 2 |
| 60 | 24.31 | 3.66 | 513.94 | 50.35 | 1 | 0 | 9 | 2 |
| 61 | 24.36 | 3.65 | 0.59 | 1.72 | 2 | 0 | 2 | 2 |
| 62 | 24.37 | 3.65 | 7383.18 | 135.29 | 0 | −2 | 1 | 4 |
| 63 | 24.48 | 3.63 | 82.82 | 20.36 | −2 | 0 | 4 | 2 |
| 64 | 24.50 | 3.63 | 237.27 | 34.50 | −1 | 0 | 10 | 2 |
| 65 | 24.68 | 3.60 | 1239.98 | 56.20 | 0 | −2 | 2 | 4 |
| 66 | 24.79 | 3.59 | 272.49 | 37.42 | 0 | 0 | 11 | 2 |
| 67 | 25.07 | 3.55 | 4730.49 | 157.76 | 2 | 0 | 3 | 2 |
| 68 | 25.20 | 3.53 | 703.68 | 43.27 | 0 | −2 | 3 | 4 |
| 69 | 25.23 | 3.53 | 4606.15 | 156.70 | −2 | 0 | 5 | 2 |
| 70 | 25.46 | 3.50 | 936.20 | 50.45 | 1 | −1 | 8 | 4 |
| 71 | 25.61 | 3.48 | 764.52 | 45.87 | 0 | −1 | 10 | 4 |
| 72 | 25.63 | 3.47 | 2744.20 | 86.96 | −1 | −1 | 9 | 4 |
| 73 | 25.91 | 3.44 | 2.14 | 2.46 | 0 | −2 | 4 | 4 |
| 74 | 25.96 | 3.43 | 444.94 | 50.20 | 2 | 0 | 4 | 2 |
| 75 | 26.15 | 3.41 | 4.40 | 5.03 | −2 | 0 | 6 | 2 |
| 76 | 26.34 | 3.38 | 1264.73 | 85.95 | 1 | 0 | 10 | 2 |
| 77 | 26.45 | 3.37 | 1304.03 | 61.98 | −2 | 1 | 1 | 4 |
| 78 | 26.53 | 3.36 | 803.14 | 48.79 | −2 | 1 | 0 | 4 |
| 79 | 26.54 | 3.36 | 45.63 | 16.46 | −1 | 0 | 11 | 2 |
| 80 | 26.57 | 3.35 | 862.91 | 50.65 | −2 | 1 | 2 | 4 |
| 81 | 26.79 | 3.32 | 2196.03 | 81.54 | 0 | −2 | 5 | 4 |
| 82 | 26.80 | 3.32 | 624.59 | 43.50 | 2 | −1 | 1 | 4 |
| 83 | 26.88 | 3.31 | 607.90 | 43.04 | −2 | 1 | 3 | 4 |
| 84 | 27.02 | 3.30 | 173.98 | 32.74 | 2 | 0 | 5 | 2 |
| 85 | 27.02 | 3.30 | 2733.98 | 91.79 | −1 | 2 | 0 | 4 |
| 86 | 27.03 | 3.30 | 1513.12 | 68.31 | −1 | 2 | 1 | 4 |
| 87 | 27.08 | 3.29 | 1463.84 | 95.23 | 0 | 0 | 12 | 2 |
| 88 | 27.20 | 3.28 | 2089.19 | 80.81 | 1 | −2 | 1 | 4 |
| 89 | 27.22 | 3.27 | 1278.09 | 63.26 | 1 | −1 | 9 | 4 |
| 90 | 27.23 | 3.27 | 803.42 | 50.17 | −1 | 2 | 2 | 4 |
| 91 | 27.24 | 3.27 | 81.07 | 22.55 | −2 | 0 | 7 | 2 |
| 92 | 27.26 | 3.27 | 21.37 | 8.19 | 2 | −1 | 2 | 4 |
| 93 | 27.37 | 3.26 | 178.38 | 23.77 | −2 | 1 | 4 | 4 |
| 94 | 27.40 | 3.25 | 436.53 | 37.22 | −1 | −1 | 10 | 4 |
| 95 | 27.57 | 3.23 | 283.47 | 30.20 | 1 | −2 | 2 | 4 |
| 96 | 27.62 | 3.23 | 2010.65 | 80.56 | −1 | 2 | 3 | 4 |
| 97 | 27.65 | 3.22 | 2212.79 | 84.64 | 0 | −1 | 11 | 4 |
| 98 | 27.84 | 3.20 | 1386.83 | 67.49 | 0 | −2 | 6 | 4 |
| 99 | 27.90 | 3.19 | 593.25 | 44.25 | 2 | −1 | 3 | 4 |
| 100 | 28.05 | 3.18 | 1453.44 | 69.63 | −2 | 1 | 5 | 4 |
| 101 | 28.12 | 3.17 | 2146.12 | 84.85 | 1 | −2 | 3 | 4 |
| 102 | 28.18 | 3.16 | 374.81 | 35.54 | −1 | 2 | 4 | 4 |
| 103 | 28.22 | 3.16 | 1596.64 | 103.90 | 2 | 0 | 6 | 2 |
| 104 | 28.42 | 3.14 | 1368.55 | 96.91 | 1 | 0 | 11 | 2 |
| 105 | 28.47 | 3.13 | 225.59 | 39.42 | −2 | 0 | 8 | 2 |
| 106 | 28.62 | 3.12 | 111.11 | 27.82 | −1 | 0 | 12 | 2 |
| 107 | 28.71 | 3.11 | 225.52 | 28.12 | 2 | −1 | 4 | 4 |
| 108 | 28.85 | 3.09 | 918.10 | 57.02 | 1 | −2 | 4 | 4 |
| 109 | 28.89 | 3.09 | 169.75 | 24.56 | −2 | 1 | 6 | 4 |
| 110 | 28.92 | 3.08 | 2123.60 | 86.97 | −1 | 2 | 5 | 4 |
| 111 | 29.03 | 3.07 | 3144.72 | 106.26 | 0 | −2 | 7 | 4 |
| 112 | 29.06 | 3.07 | 1676.86 | 77.69 | 1 | −1 | 10 | 4 |
| 113 | 29.24 | 3.05 | 469.63 | 41.39 | −1 | −1 | 11 | 4 |
| 114 | 29.39 | 3.04 | 356.06 | 51.23 | 0 | 0 | 13 | 2 |
| 115 | 29.56 | 3.02 | 723.05 | 73.46 | 2 | 0 | 7 | 2 |
| 116 | 29.68 | 3.01 | 436.23 | 40.52 | 2 | −1 | 5 | 4 |
| 117 | 29.73 | 3.00 | 2091.05 | 88.87 | 1 | −2 | 5 | 4 |
| 118 | 29.74 | 3.00 | 407.94 | 39.28 | 0 | −1 | 12 | 4 |
| 119 | 29.82 | 2.99 | 1427.18 | 73.67 | −1 | −2 | 6 | 4 |
| 120 | 29.83 | 2.99 | 2354.70 | 133.86 | −2 | 0 | 9 | 2 |
| 121 | 29.88 | 2.99 | 675.34 | 50.79 | −2 | 1 | 7 | 4 |
| 122 | 30.35 | 2.94 | 1201.69 | 68.89 | 0 | −2 | 8 | 4 |
| 123 | 30.54 | 2.92 | 594.97 | 69.01 | 1 | 0 | 12 | 2 |
| 124 | 30.75 | 2.91 | 197.57 | 40.05 | −1 | 0 | 13 | 2 |
| 125 | 30.76 | 2.90 | 1568.17 | 79.83 | 1 | −2 | 6 | 4 |
| 126 | 30.79 | 2.90 | 691.26 | 53.06 | 2 | −1 | 6 | 4 |
| 127 | 30.87 | 2.89 | 1699.18 | 83.41 | −1 | −2 | 7 | 4 |
| 128 | 30.97 | 2.88 | 372.34 | 39.19 | 1 | −1 | 11 | 4 |
| 129 | 31.01 | 2.88 | 84.26 | 26.40 | 2 | 0 | 8 | 2 |
| 130 | 31.02 | 2.88 | 1030.43 | 65.30 | −2 | 1 | 8 | 4 |
| 131 | 31.16 | 2.87 | 472.68 | 44.44 | −1 | −1 | 12 | 4 |
| 132 | 31.31 | 2.86 | 32.39 | 16.54 | −2 | 0 | 10 | 2 |
| 133 | 31.71 | 2.82 | 682.32 | 76.94 | 0 | 0 | 14 | 2 |
| 134 | 31.79 | 2.81 | 38.16 | 12.90 | 0 | −2 | 9 | 4 |
| 135 | 31.87 | 2.81 | 16.99 | 8.63 | 0 | −1 | 13 | 4 |
| 136 | 31.93 | 2.80 | 1105.02 | 69.75 | 1 | −2 | 7 | 4 |
| 137 | 32.03 | 2.79 | 367.12 | 40.35 | 2 | −1 | 7 | 4 |
| 138 | 32.04 | 2.79 | 935.01 | 64.42 | −1 | −2 | 8 | 4 |
| 139 | 32.28 | 2.77 | 717.76 | 56.90 | −2 | 1 | 9 | 4 |
| 140 | 32.57 | 2.75 | 579.67 | 73.01 | 2 | 0 | 9 | 2 |
| 141 | 32.70 | 2.74 | 11.31 | 10.24 | 1 | 0 | 13 | 2 |
| 142 | 32.88 | 2.72 | 29.22 | 16.56 | −2 | 0 | 11 | 2 |
| 143 | 32.90 | 2.72 | 112.12 | 32.47 | −1 | 0 | 14 | 2 |
| 144 | 32.94 | 2.72 | 850.74 | 63.32 | 1 | −1 | 12 | 4 |
| 145 | 33.14 | 2.70 | 120.36 | 23.97 | −1 | −1 | 13 | 4 |
| 146 | 33.22 | 2.70 | 154.15 | 27.19 | 1 | −2 | 8 | 4 |
| 147 | 33.33 | 2.69 | 331.57 | 40.03 | 0 | −2 | 10 | 4 |
| 148 | 33.35 | 2.68 | 2077.12 | 100.24 | −1 | −2 | 9 | 4 |
| 149 | 33.39 | 2.68 | 796.38 | 62.16 | 2 | −1 | 8 | 4 |
| 150 | 33.66 | 2.66 | 480.96 | 48.73 | −2 | 1 | 10 | 4 |
| 151 | 34.00 | 2.64 | 1158.90 | 76.47 | −2 | 2 | 1 | 4 |
| 152 | 34.04 | 2.63 | 1385.69 | 83.73 | 0 | −1 | 14 | 4 |
| 153 | 34.04 | 2.63 | 155.96 | 39.73 | 0 | 0 | 15 | 2 |
| 154 | 34.06 | 2.63 | 917.93 | 68.19 | −2 | 2 | 0 | 4 |
| 155 | 34.09 | 2.63 | 1100.24 | 74.73 | −2 | 2 | 2 | 4 |
| 156 | 34.22 | 2.62 | 305.79 | 55.95 | 2 | 0 | 10 | 2 |
| 157 | 34.28 | 2.61 | 509.44 | 51.16 | 2 | −2 | 1 | 4 |
| 158 | 34.34 | 2.61 | 437.98 | 47.52 | −2 | 2 | 3 | 4 |
| 159 | 34.55 | 2.59 | 337.71 | 59.41 | −2 | 0 | 12 | 2 |
| 160 | 34.62 | 2.59 | 1474.50 | 87.97 | 1 | −2 | 9 | 4 |
| 161 | 34.65 | 2.59 | 106.74 | 23.69 | 2 | −2 | 2 | 4 |
| 162 | 34.74 | 2.58 | 88.74 | 21.66 | −2 | 2 | 4 | 4 |
| 163 | 34.76 | 2.58 | 499.16 | 51.41 | −1 | −2 | 10 | 4 |
| 164 | 34.85 | 2.57 | 572.18 | 55.20 | 2 | −1 | 9 | 4 |
| 165 | 34.88 | 2.57 | 107.14 | 33.82 | 1 | 0 | 14 | 2 |
| 166 | 34.96 | 2.56 | 521.71 | 52.90 | 0 | −2 | 11 | 4 |
| 167 | 34.97 | 2.56 | 194.47 | 32.30 | 1 | −1 | 13 | 4 |
| 168 | 35.10 | 2.55 | 79.90 | 29.40 | −1 | 0 | 15 | 2 |
| 169 | 35.14 | 2.55 | 876.73 | 68.96 | −2 | −1 | 11 | 4 |
| 170 | 35.17 | 2.55 | 481.14 | 51.12 | −1 | −1 | 14 | 4 |
| 171 | 35.17 | 2.55 | 875.27 | 68.95 | 2 | −2 | 3 | 4 |
| 172 | 35.28 | 2.54 | 656.51 | 59.93 | −2 | 2 | 5 | 4 |
| 173 | 35.50 | 2.53 | 1198.33 | 115.29 | −3 | 0 | 1 | 2 |
| 174 | 35.52 | 2.53 | 236.89 | 51.29 | −3 | 0 | 2 | 2 |
| 175 | 35.63 | 2.52 | 813.15 | 95.35 | 3 | 0 | 0 | 2 |
| 176 | 35.69 | 2.51 | 4.63 | 7.21 | −3 | 0 | 3 | 2 |
| 177 | 35.83 | 2.50 | 37.07 | 14.48 | 2 | −2 | 4 | 4 |
| 178 | 35.91 | 2.50 | 148.32 | 41.07 | 3 | 0 | 1 | 2 |
| 179 | 35.95 | 2.50 | 392.66 | 66.91 | 2 | 0 | 11 | 2 |
| 180 | 35.97 | 2.49 | 98.55 | 23.72 | −2 | 2 | 6 | 4 |
| 181 | 36.01 | 2.49 | 803.50 | 95.90 | −3 | 0 | 4 | 2 |
| 182 | 36.12 | 2.49 | 8.49 | 6.99 | 1 | −2 | 10 | 4 |
| 183 | 36.24 | 2.48 | 467.57 | 52.08 | 0 | −1 | 15 | 4 |
| 184 | 36.27 | 2.48 | 173.83 | 31.78 | −1 | −2 | 11 | 4 |
| 185 | 36.29 | 2.47 | 5.85 | 8.25 | −2 | 0 | 13 | 2 |
| 186 | 36.33 | 2.47 | 252.28 | 54.25 | 3 | 0 | 2 | 2 |
| 187 | 36.39 | 2.47 | 426.22 | 70.64 | 0 | 0 | 16 | 2 |
| 188 | 36.41 | 2.47 | 748.17 | 66.22 | 2 | −1 | 10 | 4 |
| 189 | 36.48 | 2.46 | 13.73 | 12.71 | −3 | 0 | 5 | 2 |
| 190 | 36.63 | 2.45 | 157.70 | 30.60 | 2 | −2 | 5 | 4 |
| 191 | 36.68 | 2.45 | 236.32 | 37.52 | 0 | −2 | 12 | 4 |
| 192 | 36.72 | 2.45 | 31.57 | 13.73 | −2 | −1 | 12 | 4 |
| 193 | 36.80 | 2.44 | 467.11 | 52.94 | −2 | 2 | 7 | 4 |
| 194 | 36.82 | 2.44 | 3209.42 | 138.87 | 0 | −3 | 1 | 4 |
| 195 | 36.89 | 2.43 | 10.81 | 11.42 | 3 | 0 | 3 | 2 |
| 196 | 37.04 | 2.43 | 71.20 | 20.82 | 1 | −1 | 14 | 4 |
| 197 | 37.04 | 2.43 | 2331.26 | 119.13 | 0 | −3 | 2 | 4 |
| 198 | 37.08 | 2.42 | 504.55 | 78.47 | −3 | 0 | 6 | 2 |
| 199 | 37.11 | 2.42 | 12.66 | 12.44 | 1 | 0 | 15 | 2 |
| 200 | 37.24 | 2.41 | 28.59 | 13.27 | −1 | −1 | 15 | 4 |
| 201 | 37.32 | 2.41 | 0.03 | 0.58 | −1 | 0 | 16 | 2 |
| 202 | 37.40 | 2.40 | 764.83 | 68.97 | 0 | −3 | 3 | 4 |
| 203 | 37.55 | 2.39 | 616.40 | 62.19 | 2 | −2 | 6 | 4 |
| 204 | 37.59 | 2.39 | 1688.07 | 145.72 | 3 | 0 | 4 | 2 |
| 205 | 37.63 | 2.39 | 875.35 | 74.27 | −3 | 1 | 1 | 4 |
| 206 | 37.65 | 2.39 | 298.51 | 43.40 | −3 | 1 | 2 | 4 |
| 207 | 37.71 | 2.38 | 29.65 | 13.70 | 1 | −2 | 11 | 4 |
| 208 | 37.75 | 2.38 | 205.04 | 36.07 | −2 | 2 | 8 | 4 |
| 209 | 37.75 | 2.38 | 836.64 | 72.87 | −3 | 1 | 0 | 4 |
| 210 | 37.76 | 2.38 | 233.80 | 54.49 | 2 | 0 | 12 | 2 |
| 211 | 37.81 | 2.38 | 321.70 | 45.27 | −3 | 1 | 3 | 4 |
| 212 | 37.82 | 2.38 | 886.74 | 106.32 | −3 | 0 | 7 | 2 |
| 213 | 37.86 | 2.37 | 191.77 | 35.01 | −1 | −2 | 12 | 4 |
| 214 | 37.90 | 2.37 | 86.08 | 23.48 | 0 | −3 | 4 | 4 |
| 215 | 38.01 | 2.37 | 19.40 | 11.18 | 3 | −1 | 1 | 4 |
| 216 | 38.05 | 2.36 | 15.77 | 10.09 | 2 | −1 | 11 | 4 |
| 217 | 38.11 | 2.36 | 435.48 | 75.14 | −2 | 0 | 14 | 2 |
| 218 | 38.11 | 2.36 | 5.16 | 5.78 | −3 | 1 | 4 | 4 |
| 219 | 38.38 | 2.34 | 388.77 | 50.59 | −2 | −1 | 13 | 4 |
| 220 | 38.41 | 2.34 | 25.68 | 13.01 | 3 | −1 | 2 | 4 |
| 221 | 38.42 | 2.34 | 446.64 | 76.79 | 3 | 0 | 5 | 2 |
| 222 | 38.47 | 2.34 | 8.59 | 7.54 | 0 | −2 | 13 | 4 |
| 223 | 38.47 | 2.34 | 194.81 | 35.90 | 0 | −1 | 16 | 4 |
| 224 | 38.54 | 2.33 | 19.92 | 11.50 | 0 | −3 | 5 | 4 |
| 225 | 38.56 | 2.33 | 73.15 | 22.06 | −3 | 1 | 5 | 4 |
| 226 | 38.60 | 2.33 | 591.45 | 62.80 | 2 | −2 | 7 | 4 |
| 227 | 38.69 | 2.33 | 1801.04 | 155.36 | −3 | 0 | 8 | 2 |
| 228 | 38.70 | 2.32 | 28.55 | 13.84 | −1 | 3 | 0 | 4 |
| 229 | 38.71 | 2.32 | 410.35 | 52.46 | −1 | 3 | 1 | 4 |
| 230 | 38.75 | 2.32 | 6.11 | 9.07 | 0 | 0 | 17 | 2 |
| 231 | 38.82 | 2.32 | 275.15 | 43.10 | −2 | 2 | 9 | 4 |
| 232 | 38.83 | 2.32 | 255.64 | 41.56 | 1 | −3 | 1 | 4 |
| 233 | 38.85 | 2.32 | 350.31 | 48.68 | −1 | 3 | 2 | 4 |
| 234 | 38.95 | 2.31 | 176.99 | 34.70 | 3 | −1 | 3 | 4 |
| 235 | 39.10 | 2.30 | 245.09 | 41.01 | 1 | −3 | 2 | 4 |
| 236 | 39.13 | 2.30 | 411.67 | 53.19 | −3 | 1 | 6 | 4 |
| 237 | 39.14 | 2.30 | 1252.44 | 92.79 | −1 | 3 | 3 | 4 |
| 238 | 39.15 | 2.30 | 26.08 | 13.40 | 1 | −1 | 15 | 4 |
| 239 | 39.30 | 2.29 | 32.47 | 15.01 | 0 | −3 | 6 | 4 |
| 240 | 39.35 | 2.29 | 252.64 | 59.30 | 1 | 0 | 16 | 2 |
| 241 | 39.36 | 2.29 | 147.14 | 32.01 | −1 | −1 | 16 | 4 |
| 242 | 39.38 | 2.29 | 392.50 | 73.96 | 3 | 0 | 6 | 2 |
| 243 | 39.38 | 2.29 | 131.93 | 30.32 | 1 | −2 | 12 | 4 |
| 244 | 39.51 | 2.28 | 327.57 | 47.96 | 1 | −3 | 3 | 4 |
| 245 | 39.55 | 2.28 | 1.85 | 3.61 | −1 | −2 | 13 | 4 |
| 246 | 39.55 | 2.28 | 294.79 | 45.55 | −1 | 3 | 4 | 4 |
| 247 | 39.57 | 2.28 | 69.20 | 31.23 | −1 | 0 | 17 | 2 |
| 248 | 39.62 | 2.27 | 351.48 | 49.83 | 3 | −1 | 4 | 4 |
| 249 | 39.63 | 2.27 | 201.12 | 53.32 | 2 | 0 | 13 | 2 |
| 250 | 39.67 | 2.27 | 59.48 | 29.03 | −3 | 0 | 9 | 2 |
| 251 | 39.76 | 2.27 | 299.42 | 46.17 | 2 | −2 | 8 | 4 |
| 252 | 39.78 | 2.26 | 42.14 | 17.33 | 2 | −1 | 12 | 4 |
| 253 | 39.84 | 2.26 | 319.30 | 47.79 | −3 | 1 | 7 | 4 |
| 254 | 40.00 | 2.25 | 52.91 | 27.63 | −2 | 0 | 15 | 2 |
| 255 | 40.00 | 2.25 | 388.94 | 52.97 | −2 | 2 | 10 | 4 |
| 256 | 40.05 | 2.25 | 308.44 | 47.24 | 1 | −3 | 4 | 4 |
| 257 | 40.11 | 2.25 | 328.17 | 48.81 | −1 | 3 | 5 | 4 |
| 258 | 40.12 | 2.25 | 105.32 | 27.66 | −2 | −1 | 14 | 4 |
| 259 | 40.19 | 2.24 | 161.05 | 34.27 | 0 | −3 | 7 | 4 |
| 260 | 40.32 | 2.23 | 268.05 | 44.38 | 0 | −2 | 14 | 4 |
| 261 | 40.42 | 2.23 | 70.90 | 22.88 | 3 | −1 | 5 | 4 |
| 262 | 40.45 | 2.23 | 96.37 | 37.76 | 3 | 0 | 7 | 2 |
| 263 | 40.67 | 2.22 | 230.61 | 41.55 | −3 | 1 | 8 | 4 |
| 264 | 40.71 | 2.21 | 58.34 | 20.93 | 1 | −3 | 5 | 4 |
| 265 | 40.73 | 2.21 | 136.32 | 32.00 | 0 | −1 | 17 | 4 |
| 266 | 40.77 | 2.21 | 34.59 | 22.83 | −3 | 0 | 10 | 2 |
| 267 | 40.79 | 2.21 | 208.66 | 39.65 | −1 | −3 | 6 | 4 |
| 268 | 41.03 | 2.20 | 35.16 | 16.38 | 2 | −2 | 9 | 4 |
| 269 | 41.13 | 2.19 | 185.89 | 53.42 | 0 | 0 | 18 | 2 |
| 270 | 41.13 | 2.19 | 20.30 | 12.49 | 1 | −2 | 13 | 4 |
| 271 | 41.19 | 2.19 | 1239.71 | 97.73 | 0 | −3 | 8 | 4 |
| 272 | 41.28 | 2.19 | 90.38 | 26.45 | −2 | −2 | 11 | 4 |
| 273 | 41.30 | 2.18 | 86.52 | 25.89 | −1 | −2 | 14 | 4 |
| 274 | 41.31 | 2.18 | 227.15 | 41.97 | 1 | −1 | 16 | 4 |
| 275 | 41.33 | 2.18 | 589.09 | 67.62 | 3 | −1 | 6 | 4 |
| 276 | 41.50 | 2.17 | 41.93 | 18.13 | 1 | −3 | 6 | 4 |
| 277 | 41.52 | 2.17 | 177.37 | 37.29 | −1 | −1 | 17 | 4 |
| 278 | 41.56 | 2.17 | 7.53 | 10.88 | 2 | 0 | 14 | 2 |
| 279 | 41.57 | 2.17 | 203.61 | 40.01 | 2 | −1 | 13 | 4 |
| 280 | 41.59 | 2.17 | 198.33 | 39.51 | −1 | −3 | 7 | 4 |
| 281 | 41.62 | 2.17 | 574.97 | 67.32 | −3 | 1 | 9 | 4 |
| 282 | 41.62 | 2.17 | 116.37 | 42.84 | 3 | 0 | 8 | 2 |
| 283 | 41.63 | 2.17 | 13.07 | 14.36 | 1 | 0 | 17 | 2 |
| 284 | 41.85 | 2.16 | 502.72 | 89.59 | −1 | 0 | 18 | 2 |
| 285 | 41.93 | 2.15 | 343.38 | 52.46 | −2 | −1 | 15 | 4 |
| 286 | 41.94 | 2.15 | 22.88 | 19.16 | −2 | 0 | 16 | 2 |
| 287 | 41.98 | 2.15 | 2.71 | 6.60 | −3 | 0 | 11 | 2 |
| 288 | 42.24 | 2.14 | 279.70 | 47.75 | 0 | −2 | 15 | 4 |
| 289 | 42.31 | 2.13 | 69.61 | 23.86 | 0 | −3 | 9 | 4 |
| 290 | 42.36 | 2.13 | 304.01 | 49.93 | 3 | −1 | 7 | 4 |
| 291 | 42.39 | 2.13 | 122.32 | 31.70 | 2 | −2 | 10 | 4 |
| 292 | 42.41 | 2.13 | 584.46 | 69.33 | 1 | −3 | 7 | 4 |
| 293 | 42.51 | 2.13 | 59.30 | 22.14 | −1 | −3 | 8 | 4 |
| 294 | 42.67 | 2.12 | 125.38 | 32.33 | −2 | −2 | 12 | 4 |
| 295 | 42.68 | 2.12 | 155.29 | 35.99 | −3 | 1 | 10 | 4 |
| 296 | 42.90 | 2.11 | 1.74 | 5.43 | 3 | 0 | 9 | 2 |
| 297 | 42.95 | 2.10 | 233.61 | 44.45 | 1 | −2 | 14 | 4 |
| 298 | 43.01 | 2.10 | 73.90 | 25.05 | 0 | −1 | 18 | 4 |
| 299 | 43.13 | 2.10 | 52.66 | 21.20 | −1 | −2 | 15 | 4 |
| 300 | 43.29 | 2.09 | 13.49 | 15.24 | −3 | 0 | 12 | 2 |
| 301 | 43.43 | 2.08 | 127.76 | 33.29 | 1 | −3 | 8 | 4 |
| 302 | 43.43 | 2.08 | 241.19 | 45.74 | 2 | −1 | 14 | 4 |
| 303 | 43.47 | 2.08 | 147.05 | 35.75 | −3 | 2 | 1 | 4 |
| 304 | 43.49 | 2.08 | 427.68 | 60.99 | −3 | 2 | 2 | 4 |
| 305 | 43.49 | 2.08 | 133.28 | 34.06 | 3 | −1 | 8 | 4 |
| 306 | 43.50 | 2.08 | 99.50 | 29.43 | 1 | −1 | 17 | 4 |
| 307 | 43.52 | 2.08 | 318.97 | 52.73 | 0 | −3 | 10 | 4 |
| 308 | 43.52 | 2.08 | 13.39 | 15.28 | 0 | 0 | 19 | 2 |
| 309 | 43.53 | 2.08 | 143.18 | 35.34 | −1 | −3 | 9 | 4 |
| 310 | 43.55 | 2.08 | 310.81 | 73.65 | 2 | 0 | 15 | 2 |
| 311 | 43.58 | 2.08 | 168.61 | 38.39 | −3 | 2 | 0 | 4 |
| 312 | 43.63 | 2.07 | 35.77 | 17.71 | −3 | 2 | 3 | 4 |
| 313 | 43.72 | 2.07 | 157.80 | 37.27 | −1 | −1 | 18 | 4 |
| 314 | 43.80 | 2.07 | 122.87 | 32.96 | −2 | −1 | 16 | 4 |
| 315 | 43.81 | 2.06 | 85.95 | 27.57 | 3 | −2 | 1 | 4 |
| 316 | 43.84 | 2.06 | 89.35 | 28.13 | −3 | 1 | 11 | 4 |
| 317 | 43.85 | 2.06 | 224.42 | 44.60 | 2 | −2 | 11 | 4 |
| 318 | 43.90 | 2.06 | 404.90 | 59.99 | −3 | 2 | 4 | 4 |
| 319 | 43.94 | 2.06 | 76.41 | 36.89 | −2 | 0 | 17 | 2 |
| 320 | 43.94 | 2.06 | 327.10 | 76.32 | 1 | 0 | 18 | 2 |
| 321 | 44.06 | 2.05 | 912.50 | 90.41 | −2 | 3 | 1 | 4 |
| 322 | 44.11 | 2.05 | 118.90 | 32.68 | −2 | 3 | 0 | 4 |
| 323 | 44.13 | 2.05 | 76.64 | 26.25 | −2 | 3 | 2 | 4 |
| 324 | 44.14 | 2.05 | 409.19 | 60.67 | −2 | −2 | 13 | 4 |
| 325 | 44.16 | 2.05 | 1.16 | 4.57 | −1 | 0 | 19 | 2 |
| 326 | 44.17 | 2.05 | 210.34 | 43.53 | 3 | −2 | 2 | 4 |
| 327 | 44.22 | 2.05 | 164.19 | 38.51 | 0 | −2 | 16 | 4 |
| 328 | 44.28 | 2.04 | 42.50 | 27.75 | 3 | 0 | 10 | 2 |
| 329 | 44.28 | 2.04 | 70.34 | 25.25 | 2 | −3 | 1 | 4 |
| 330 | 44.30 | 2.04 | 10.57 | 9.79 | −3 | 2 | 5 | 4 |
| 331 | 44.33 | 2.04 | 6.93 | 7.93 | −2 | 3 | 3 | 4 |
| 332 | 44.56 | 2.03 | 619.46 | 75.44 | 1 | −3 | 9 | 4 |
| 333 | 44.58 | 2.03 | 1342.07 | 111.12 | 2 | −3 | 2 | 4 |
| 334 | 44.65 | 2.03 | 120.97 | 33.42 | 3 | −2 | 3 | 4 |
| 335 | 44.65 | 2.03 | 794.28 | 85.64 | −2 | 3 | 4 | 4 |
| 336 | 44.67 | 2.03 | 66.43 | 24.78 | −1 | −3 | 10 | 4 |
| 337 | 44.69 | 2.03 | 1.38 | 5.05 | −3 | 0 | 13 | 2 |
| 338 | 44.73 | 2.02 | 2.90 | 5.18 | 3 | −1 | 9 | 4 |
| 339 | 44.81 | 2.02 | 156.61 | 38.18 | −3 | 2 | 6 | 4 |
| 340 | 44.83 | 2.02 | 140.86 | 36.23 | 1 | −2 | 15 | 4 |
| 341 | 44.84 | 2.02 | 470.05 | 66.19 | 0 | −3 | 11 | 4 |
| 342 | 45.00 | 2.01 | 20.82 | 13.99 | 2 | −3 | 3 | 4 |
| 343 | 45.01 | 2.01 | 737.25 | 83.26 | −1 | −2 | 16 | 4 |
| 344 | 45.10 | 2.01 | 70.07 | 25.72 | −2 | 3 | 5 | 4 |
| 345 | 45.10 | 2.01 | 12.78 | 10.98 | −3 | 1 | 12 | 4 |
| 346 | 45.25 | 2.00 | 135.79 | 35.94 | 3 | −2 | 4 | 4 |
| 347 | 45.33 | 2.00 | 158.95 | 38.97 | 0 | −1 | 19 | 4 |
| 348 | 45.35 | 2.00 | 125.05 | 34.58 | 2 | −1 | 15 | 4 |
| 349 | 45.39 | 2.00 | 44.54 | 20.66 | 2 | −2 | 12 | 4 |
| 350 | 45.44 | 1.99 | 10.18 | 9.89 | −3 | 2 | 7 | 4 |
| 351 | 45.54 | 1.99 | 140.50 | 36.83 | 2 | −3 | 4 | 4 |
| 352 | 45.59 | 1.99 | 10.80 | 14.46 | 2 | 0 | 16 | 2 |
| 353 | 45.66 | 1.99 | 565.22 | 74.09 | −2 | 3 | 6 | 4 |
| 354 | 45.70 | 1.98 | 28.12 | 16.54 | −2 | −2 | 14 | 4 |
| 355 | 45.73 | 1.98 | 9.28 | 9.51 | −2 | −1 | 17 | 4 |
| 356 | 45.73 | 1.98 | 286.45 | 52.84 | 1 | −1 | 18 | 4 |
| 357 | 45.74 | 1.98 | 20.71 | 20.09 | 3 | 0 | 11 | 2 |
| 358 | 45.78 | 1.98 | 93.81 | 30.27 | 1 | −3 | 10 | 4 |
| 359 | 45.90 | 1.98 | 115.18 | 33.64 | −1 | −3 | 11 | 4 |
| 360 | 45.94 | 1.97 | 1.07 | 4.59 | 0 | 0 | 20 | 2 |
| 361 | 45.95 | 1.97 | 80.66 | 28.19 | −1 | −1 | 19 | 4 |
| 362 | 45.96 | 1.97 | 34.19 | 18.36 | 3 | −2 | 5 | 4 |
| 363 | 45.99 | 1.97 | 1.33 | 5.12 | −2 | 0 | 18 | 2 |
| 364 | 46.06 | 1.97 | 75.10 | 27.27 | 3 | −1 | 10 | 4 |
| 365 | 46.18 | 1.96 | 7.89 | 12.53 | −3 | 0 | 14 | 2 |
| 366 | 46.19 | 1.96 | 266.36 | 51.53 | −3 | 2 | 8 | 4 |
| 367 | 46.20 | 1.96 | 60.31 | 24.52 | 2 | −3 | 5 | 4 |
| 368 | 46.24 | 1.96 | 284.31 | 53.30 | 0 | −3 | 12 | 4 |
| 369 | 46.24 | 1.96 | 317.88 | 56.36 | 0 | −2 | 17 | 4 |
| 370 | 46.27 | 1.96 | 60.97 | 34.93 | 1 | 0 | 19 | 2 |
| 371 | 46.34 | 1.96 | 529.08 | 72.89 | −2 | 3 | 7 | 4 |
| 372 | 46.46 | 1.95 | 99.44 | 31.69 | −3 | 1 | 13 | 4 |
| 373 | 46.50 | 1.95 | 0.04 | 0.91 | −1 | 0 | 20 | 2 |
| 374 | 46.77 | 1.94 | 348.86 | 59.81 | 1 | −2 | 16 | 4 |
| 375 | 46.79 | 1.94 | 382.35 | 62.64 | 3 | −2 | 6 | 4 |
| 376 | 46.96 | 1.93 | 216.49 | 47.33 | −1 | −2 | 17 | 4 |
| 377 | 46.97 | 1.93 | 72.68 | 27.43 | 2 | −3 | 6 | 4 |
| 378 | 47.01 | 1.93 | 80.04 | 28.81 | 2 | −2 | 13 | 4 |
| 379 | 47.05 | 1.93 | 1.26 | 3.62 | −3 | 2 | 9 | 4 |
| 380 | 47.10 | 1.93 | 78.64 | 28.62 | 1 | −3 | 11 | 4 |
| 381 | 47.13 | 1.93 | 158.22 | 40.63 | −2 | 3 | 8 | 4 |
| 382 | 47.23 | 1.92 | 46.75 | 22.14 | −1 | −3 | 12 | 4 |
| 383 | 47.28 | 1.92 | 6.14 | 11.36 | 3 | 0 | 12 | 2 |
| 384 | 47.33 | 1.92 | 84.57 | 29.85 | −2 | −2 | 15 | 4 |
| 385 | 47.33 | 1.92 | 114.36 | 34.71 | 2 | −1 | 16 | 4 |
| 386 | 47.48 | 1.91 | 38.66 | 20.25 | 3 | −1 | 11 | 4 |
| 387 | 47.68 | 1.91 | 102.09 | 46.76 | 2 | 0 | 17 | 2 |
| 388 | 47.68 | 1.91 | 194.33 | 45.62 | 0 | −1 | 20 | 4 |
| 389 | 47.72 | 1.90 | 1.06 | 3.37 | −2 | −1 | 18 | 4 |
| 390 | 47.72 | 1.90 | 26.00 | 16.71 | 3 | −2 | 7 | 4 |
| 391 | 47.74 | 1.90 | 12.77 | 11.71 | 0 | −3 | 13 | 4 |
| 392 | 47.75 | 1.90 | 31.24 | 25.91 | −3 | 0 | 15 | 2 |
| 393 | 47.85 | 1.90 | 186.24 | 44.84 | 2 | −3 | 7 | 4 |
| 394 | 47.91 | 1.90 | 146.72 | 39.86 | −3 | 1 | 14 | 4 |
| 395 | 47.96 | 1.90 | 305.79 | 81.47 | −4 | 0 | 2 | 2 |
| 396 | 48.00 | 1.89 | 2.10 | 6.76 | −4 | 0 | 1 | 2 |
| 397 | 48.00 | 1.89 | 77.91 | 29.11 | 1 | −1 | 19 | 4 |
| 398 | 48.01 | 1.89 | 75.90 | 28.74 | −3 | 2 | 10 | 4 |
| 399 | 48.03 | 1.89 | 21.60 | 15.34 | −2 | 3 | 9 | 4 |
| 400 | 48.04 | 1.89 | 20.82 | 21.30 | −4 | 0 | 3 | 2 |
| 401 | 48.08 | 1.89 | 17.64 | 19.62 | −2 | 0 | 19 | 2 |
| 402 | 48.15 | 1.89 | 84.57 | 43.04 | 4 | 0 | 0 | 2 |
| 403 | 48.22 | 1.89 | 99.24 | 33.02 | −1 | −1 | 20 | 4 |
| 404 | 48.24 | 1.89 | 165.50 | 60.34 | −4 | 0 | 4 | 2 |
| 405 | 48.32 | 1.88 | 101.42 | 33.46 | 0 | −2 | 18 | 4 |
| 406 | 48.38 | 1.88 | 46.67 | 32.15 | 0 | 0 | 21 | 2 |
| 407 | 48.42 | 1.88 | 132.26 | 54.17 | 4 | 0 | 1 | 2 |
| 408 | 48.51 | 1.88 | 568.72 | 79.59 | 1 | −3 | 12 | 4 |
| 409 | 48.55 | 1.87 | 59.80 | 36.54 | −4 | 0 | 5 | 2 |
| 410 | 48.64 | 1.87 | 86.99 | 44.15 | 1 | 0 | 20 | 2 |
| 411 | 48.65 | 1.87 | 19.32 | 14.72 | −1 | −3 | 13 | 4 |
| 412 | 48.71 | 1.87 | 327.02 | 60.63 | 2 | −2 | 14 | 4 |
| 413 | 48.76 | 1.87 | 142.02 | 40.01 | 3 | −2 | 8 | 4 |
| 414 | 48.77 | 1.87 | 35.71 | 20.07 | 1 | −2 | 17 | 4 |
| 415 | 48.80 | 1.86 | 0.09 | 1.45 | 4 | 0 | 2 | 2 |
| 416 | 48.83 | 1.86 | 20.05 | 15.06 | 2 | −3 | 8 | 4 |
| 417 | 48.87 | 1.86 | 50.19 | 33.72 | −1 | 0 | 21 | 2 |
| 418 | 48.91 | 1.86 | 0.92 | 4.58 | 3 | 0 | 13 | 2 |
| 419 | 48.97 | 1.86 | 505.23 | 75.82 | −1 | −2 | 18 | 4 |
| 420 | 48.98 | 1.86 | 4.57 | 10.20 | −4 | 0 | 6 | 2 |
| 421 | 48.98 | 1.86 | 43.08 | 22.15 | 3 | −1 | 12 | 4 |
| 422 | 49.03 | 1.86 | 80.32 | 30.28 | −2 | 3 | 10 | 4 |
| 423 | 49.04 | 1.86 | 299.29 | 58.46 | −2 | −2 | 16 | 4 |
| 424 | 49.08 | 1.85 | 17.93 | 14.32 | −3 | 2 | 11 | 4 |
| 425 | 49.30 | 1.85 | 2.52 | 7.63 | 4 | 0 | 3 | 2 |
| 426 | 49.31 | 1.85 | 27.59 | 17.86 | 0 | −3 | 14 | 4 |
| 427 | 49.37 | 1.84 | 190.14 | 46.94 | 2 | −1 | 17 | 4 |
| 428 | 49.39 | 1.84 | 106.26 | 49.66 | −3 | 0 | 16 | 2 |
| 429 | 49.43 | 1.84 | 41.50 | 21.97 | −3 | 1 | 15 | 4 |
| 430 | 49.52 | 1.84 | 0.52 | 3.49 | −4 | 0 | 7 | 2 |
| 431 | 49.64 | 1.84 | 51.87 | 24.67 | −4 | 1 | 2 | 4 |
| 432 | 49.68 | 1.83 | 316.67 | 61.02 | −4 | 1 | 1 | 4 |
| 433 | 49.71 | 1.83 | 91.37 | 46.39 | 0 | 4 | 0 | 2 |
| 434 | 49.72 | 1.83 | 265.81 | 55.96 | −4 | 1 | 3 | 4 |
| 435 | 49.76 | 1.83 | 177.51 | 45.77 | −2 | −1 | 19 | 4 |
| 436 | 49.77 | 1.83 | 60.02 | 26.62 | 0 | −4 | 1 | 4 |
| 437 | 49.81 | 1.83 | 15.08 | 18.89 | 2 | 0 | 18 | 2 |
| 438 | 49.83 | 1.83 | 12.27 | 12.05 | −4 | 1 | 0 | 4 |
| 439 | 49.89 | 1.83 | 333.28 | 62.91 | 3 | −2 | 9 | 4 |
| 440 | 49.91 | 1.83 | 13.61 | 17.98 | 4 | 0 | 4 | 2 |
| 441 | 49.91 | 1.83 | 332.55 | 62.87 | 2 | −3 | 9 | 4 |
| 442 | 49.91 | 1.83 | 91.16 | 32.92 | −4 | 1 | 4 | 4 |
| 443 | 49.94 | 1.82 | 5.09 | 7.78 | 0 | −4 | 2 | 4 |
| 444 | 50.00 | 1.82 | 46.32 | 23.51 | 1 | −3 | 13 | 4 |

TABLE 22
Unit cell parameters for monoclinic P2₁ (S)-
nicotinium bi-L-(+)-tartrate dihydrate.

| | |
|---|---|
| Monoclinic P2₁ | R₁ = 6.72% |

| | | |
|---|---|---|
| a = 7.5818 (4) | α = 90° | d min (Mo) = 0.69 |
| b = 7.3307 (4) | β = 94.960 (1)° | Flack = 0.1 (3) |
| c = 39.6246 (19) | γ = 90° | Volume = 2194.1 (2) |

Amorphous Nicotinium 2,4-Dihydroxybenzoate. 2,4-Dihydroxybenzoic acid (154.1 mg, 1.0 mmol) was added into a 20 mL scintillation vial. Acetone (4.0 mL) was added with vigorous agitation. (S)-Nicotine (0.16 mL, 1.0 mmol) was added via micropipette in the dark to avoid degradation. The resulting solution was vortexed for 60 seconds at 3,000 rpm on a VWR Mini Vortexer MV I. The solution was then stored in the dark, uncapped to allow for amorphous material formation while the solvent slowly evaporated. The resulting amorphous material was rinsed with n-heptane (2×3 mL) and dried with air overnight. The amorphous material (227.9 mg) was then characterized via ¹H NMR.

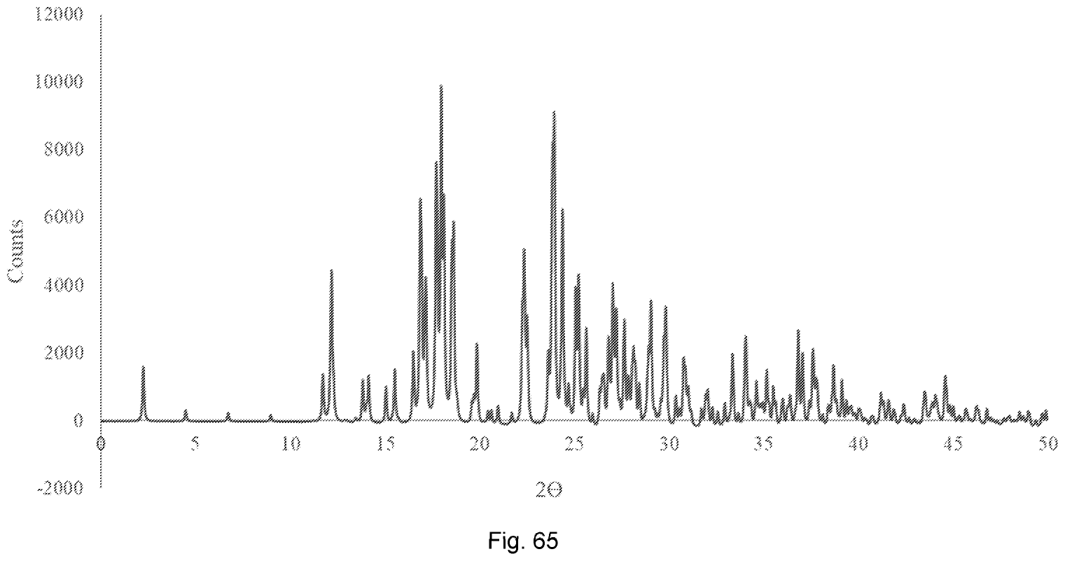

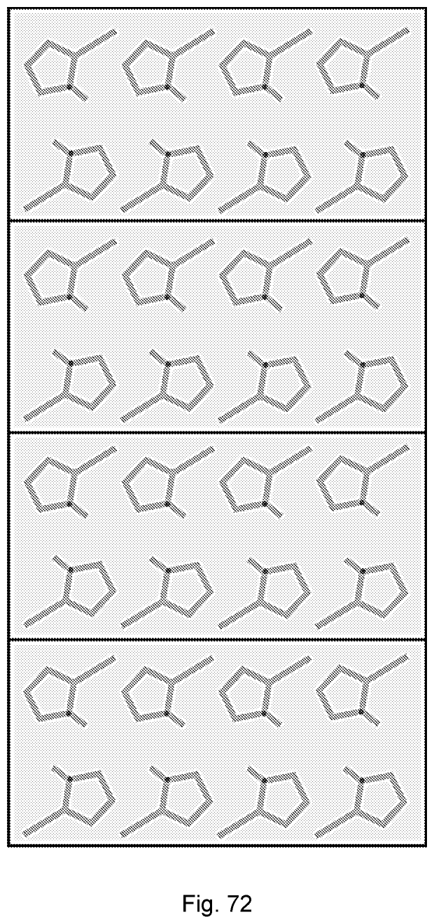

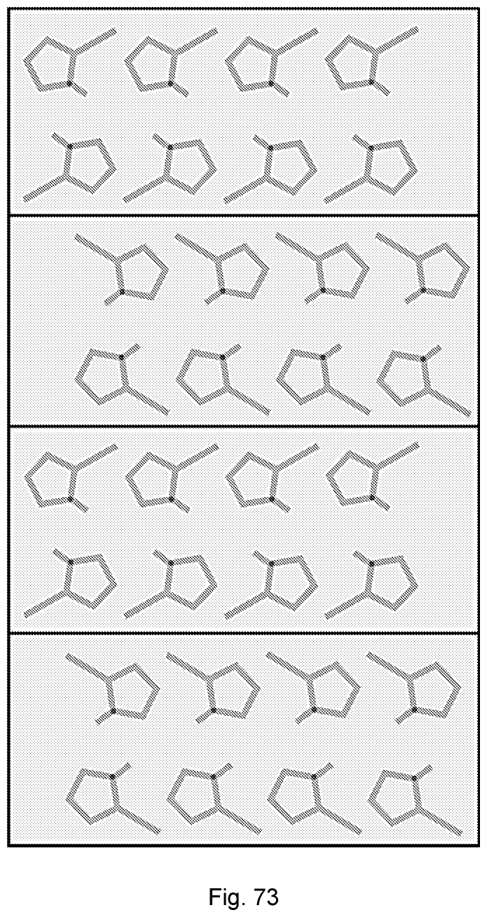

Structural Analysis. By varying the concentration of nicotine in the system, two different polymorphs of (S)-nicotinium L-malate were isolated. Identical asymmetric units are observed in orthorhombic P2₁2₁2₁ (S)-nicotinium L-malate and monoclinic P2₁ (S)-nicotinium L-malate. While the asymmetric units are identical, when the packing of each crystal system is expanded, one can see distinct differences between the orthorhombic P2₁2₁2₁ (S)-nicotinium L-malate lattice and the monoclinic P2₁ (S)-nicotinium L-malate lattice. The difference is most notably distinct when each is viewed down the crystallographic b-axis, while comparing the arrangement of the nicotine molecules. As depicted in FIGS. 72 and 73, orthorhombic P2₁2₁2₁ (S)-nicotinium L-malate and monoclinic P2₁ (S)-nicotinium L-malate each have sets of two opposing columns of methylated pyrrolidines that form a pillar. In the monoclinic P2₁ (S)-nicotinium L-malate system, the lattice down the crystallographic b-axis depicts a regular repeating translational pattern of these pillars (FIG. 63). In the orthorhombic system, however, every other pillar appears to be rotated 180° about a horizontal axis. (FIG. 64). At the interface of each pillar in the monoclinic P2₁ (S)-nicotinium L-malate crystal system, the pyridine ring portion of the larger nicotine structure point in opposing directions on either side of the pillar interface. Meanwhile, in the orthorhombic P2₁2₁2₁ (S)-nicotinium L-malate crystal system, a herringbone pattern is observed at the interface of each pillar. As these crystalline systems differ in the packing arrangement, they are an example of packing polymorphs.

Orthorhombic P2₁2₁2₁ (S)-nicotinium DL-malate was created using DL-malic acid. This racemic form of malic acid consists of an equal ratio of D and L malic acid. The crystal structure of orthorhombic P2₁2₁2₁ (S)-nicotinium DL-malate did indeed possess a mixture of the two forms, though the ratio was no longer 50:50. The solved crystal structure of orthorhombic P2₁2₁2₁ (S)-nicotinium DL-malate was determined to have a roughly 80:20 ratio of L-malic acid to D-malic acid, meaning that within the crystal lattice, there is an 80% occupancy of the L enantiomer and a 20% occupancy of the D enantiomer. This demonstrates nicotine's preference toward interacting with the natural L enantiomer of malic acid over the D enantiomer.

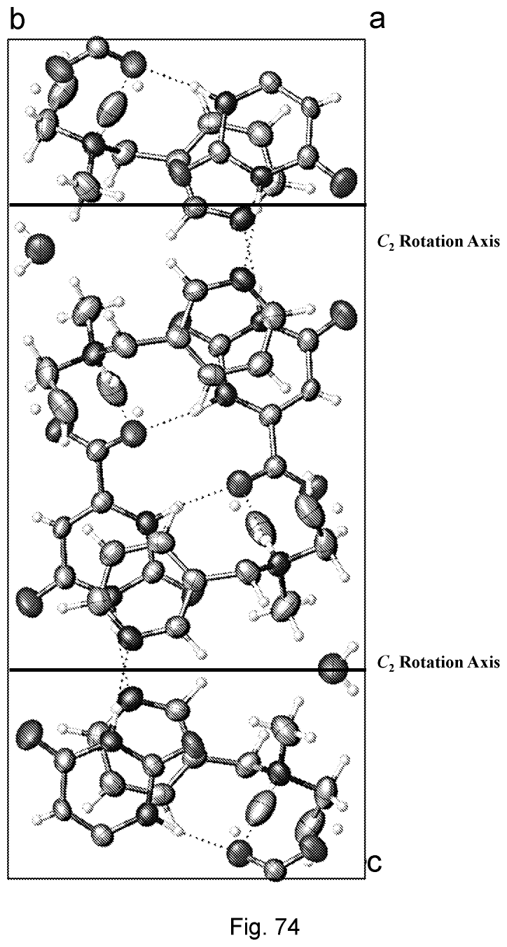

In orthorhombic P222₁ (S)-nicotinium orotate monohydrate, it was noted that the water molecules sit on crystallographically special positions along the edge of the unit cell. Crystallographic special positions are places in the unit cell where atoms can sit directly on a symmetry operator point, line, or plane. These points, lines, or planes correspond to inversion centers, rotational axes, or mirror planes respectively. Atoms on a crystallographic special position possess less symmetry related locations in the unit cell, in contrast to an atom in a more general/non-special position. The water molecules in the unit cell of orthorhombic P222₁ (S)-nicotinium orotate monohydrate, each reside on a C2 rotation axis, which go down the crystallographic b-axis (FIG. 74).

In monoclinic P2₁ (S)-nicotinium 2,6-dihydroxybenzoate, both hydroxyl functional groups undergo hydrogen bonding interactions to the carboxyl group. This further acidifies the carboxyl group proton, as each hydroxyl group draws the electron density away from the carboxyl group proton. This leads to the two observed intramolecular hydrogen bonds, that further acidify the 2,6-dihydroxybenzoic acid, aiding in the proton transfer that leads to the nicotine salt formation.

Monoclinic P2₁ (S)-nicotinium 2,6-dihydroxybenzoate also exhibited a special type of polymorphism. By running a SC-XRD experiment on the same crystal at room temperature and then again at 90 K, a temperature dependent polymorph, known as a low temperature crystalline phase transition, was observed. The unit cell parameters drastically changed between the two once the crystal was cooled to 90 K. In this instance, as with most solid to solid phase transitions, the crystal underwent first order transitions, which can be changes with regards to the unit cell volume, the lattice, the packing, or the enthalpy or entropy of the crystalline system. In this instance, there was a perturbation of the salt packing as the crystal was cooled to 90 K. The nicotinium 2,6-dihydroxybenzoate s no longer uniformly aligned as every other row has slipped slightly. This caused the unit cell parameters to change. While the Bravais lattice and space group of the phase transition structure remained the same, the most notable difference is the change in the unit cell volume. The volume nearly doubled as a result of every other salt row slipping in the phase transition structure.

Further SC-XRD experiments were run to track the unit cell parameters as the temperature was ramped from 110 K to 100 K to isolate the exact temperature of the observed phase transition. The unit cell was observed as changing drastically from 107 K to 106 K as well as reversibly transitioning from 106 K to 107 K.

Salicylic acid and 2,6-dihydroxybenzoic acid have shown to selectively crystallize (S)-nicotine in a less favorable conformation, known as the “cis” conformation of nicotine. In the monoclinic P2₁ (S)-nicotinium bi-L-(+)-tartrate dihydrate salt, an N-methylpyrrolidine ring puckering conformation was observed on one of the nicotine molecules' 5 membered N-methylpyrrolidine ring.

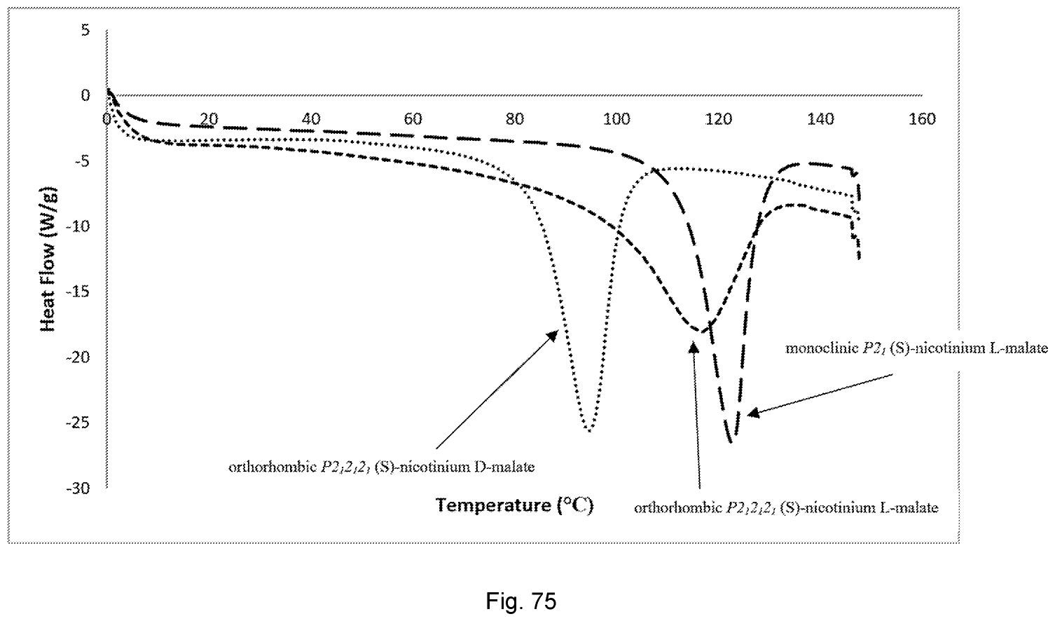

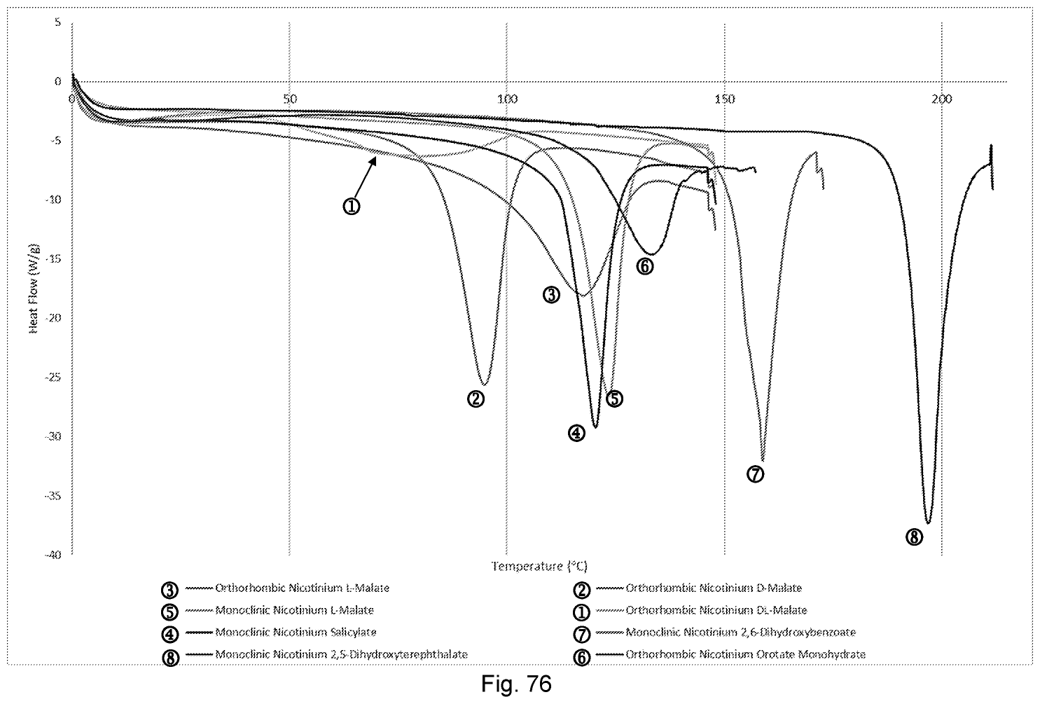

Thermal Analysis. As detailed in FIGS. 75 and 76, the orthorhombic P2₁2₁2₁ (S)-nicotinium L-malate was initially observed as having a broad melting point of 112-121° C. This was confirmed via differential scanning calorimetry (DSC) by a very broad endotherm 116.4° C. The monoclinic P2₁ (S)-nicotinium L-malate had an observed melting point between 120-123° C. This was confirmed by DSC where in the sharp endotherm at 123.5° C. indicated the melting of this (S)-nicotinium L-malate salt. The orthorhombic P2₁2₁2₁ (S)-nicotinium D-malate had a melting point range between 90-95° C. This was confirmed via DSC, wherein the sharp endotherm at 95.2° C. The orthorhombic P2₁2₁2₁ (S)-nicotinium DL-malate was initially observed as having a broad melting point of 76-78° C. The was confirmed via DSC wherein a melting point of 77.8° C., though with a very broad curve. The monoclinic P2₁ (S)-nicotinium L-malate salt was found to be the most thermodynamically favored and stable malate salt, with the orthorhombic P2₁2₁2₁ (S)-nicotinium L-malate more thermodynamically stable than the orthorhombic P2₁2₁2₁ (S)-nicotinium D-malate and a bit less thermodynamically stable than the monoclinic P2₁ (S)-nicotinium L-malate salt. The orthorhombic P2₁2₁2₁ (S)-nicotinium DL-malate exhibited a very broad and low temperature endotherm, due to the disorder arising from partial occupancy of each enantiomer within the crystal lattice. The monoclinic P2₁ (S)-nicotinium salicylate salt had a sharp endotherm at 120.5° C., confirming the observed melting point. The orthorhombic P222₁ (S)-nicotinium orotate monohydrate had an initial observed melting point range between 131 and 134° C. This was confirmed via DSC wherein a peak was observed at 133.50° C. Monoclinic P2₁ (S)-nicotinium 2,6-dihydroxybenzoate salt had the highest melting observed thus far. The sharp endotherm at 158.5° C. confirmed the observed melting point. Monoclinic P2₁ (S)-nicotinium 2,5-dihydroxyterephthalate was observed initially as having a tight melting point range of 196-198° C. This was confirmed via DSC wherein a melting point of 198.0° C. was observed, making it the synthesized salt possessing the highest melting point thus far. The monoclinic P2₁ (S)-nicotinium bi-L-(+)-tartrate dihydrate salt had an observed melting point range of 88-95° C.

TABLE 23
Observed melting point ranges on a Stuart SMP 10 melting point apparatus.

| | | | Stuart SMP10 | DSC |
|---|---|---|---|---|
| | | Coformer | Observed Salt | Peak Salt |
| Salt Number | | Melting Point | Melting Point | Melting Point |
| in FIG. 75 | Acid Coformer | (° C.) | (° C.) | (° C.) |
| 3 | L-malic acid | 130 | 115-118 | 116.4 |
| 5 | L-malic acid | 130 | 119-124 | 123.5 |
| 2 | D-malic acid | 130 | 93-97 | 95.2 |
| 1 | DL-malic acid | 130 | 76-78 | 77.8 |
| 6 | Orotic acid | 345 | 131-134 | 133.5 |
| 4 | Salicylic acid | 158 | 119-120 | 120.5 |
| 7 | 2,6-Dihydroxybenzoic acid | 165 | 157-160 | 158.5 |
| 8 | 2,5-Dihydroxyterephthalic | 339 | 196-198 | 198.0 |
| | acid | | | |
| N/A | L-Tartaric Acid | 171 | 88-95 | N/A |

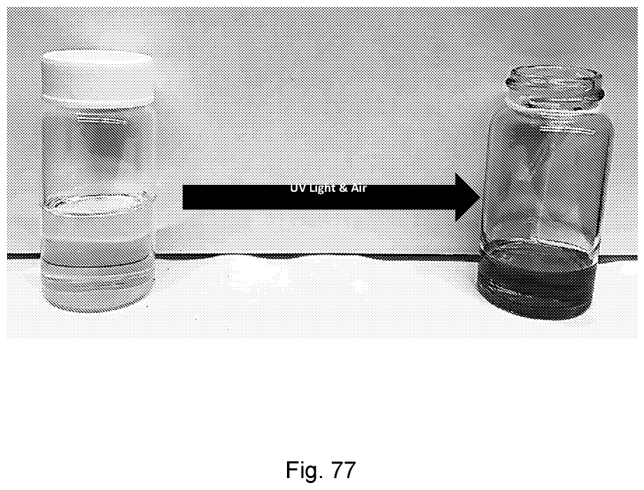

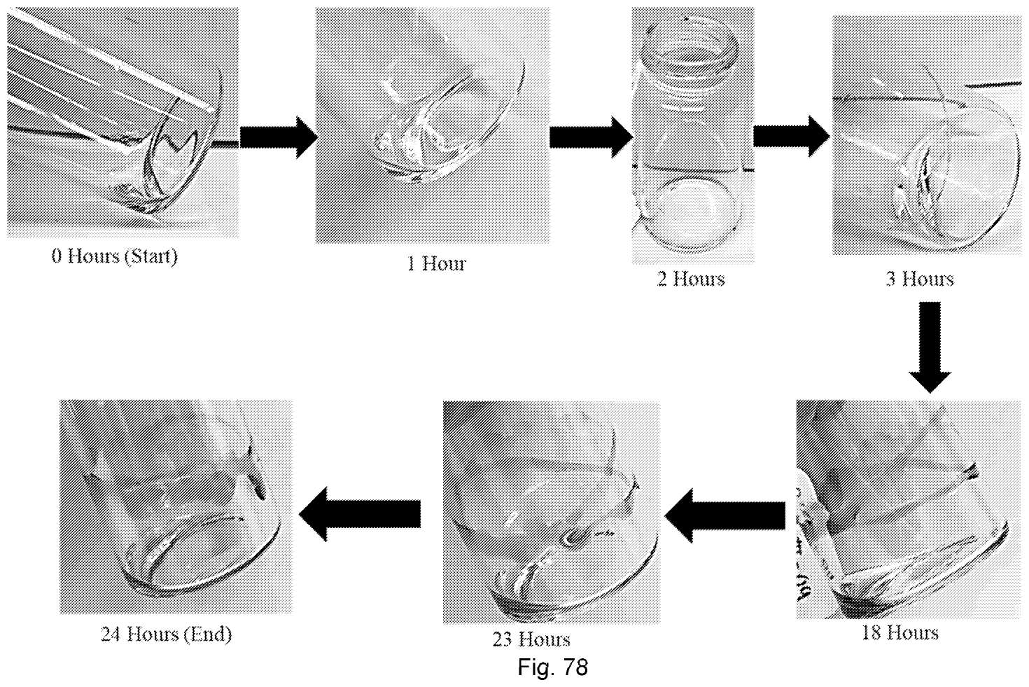

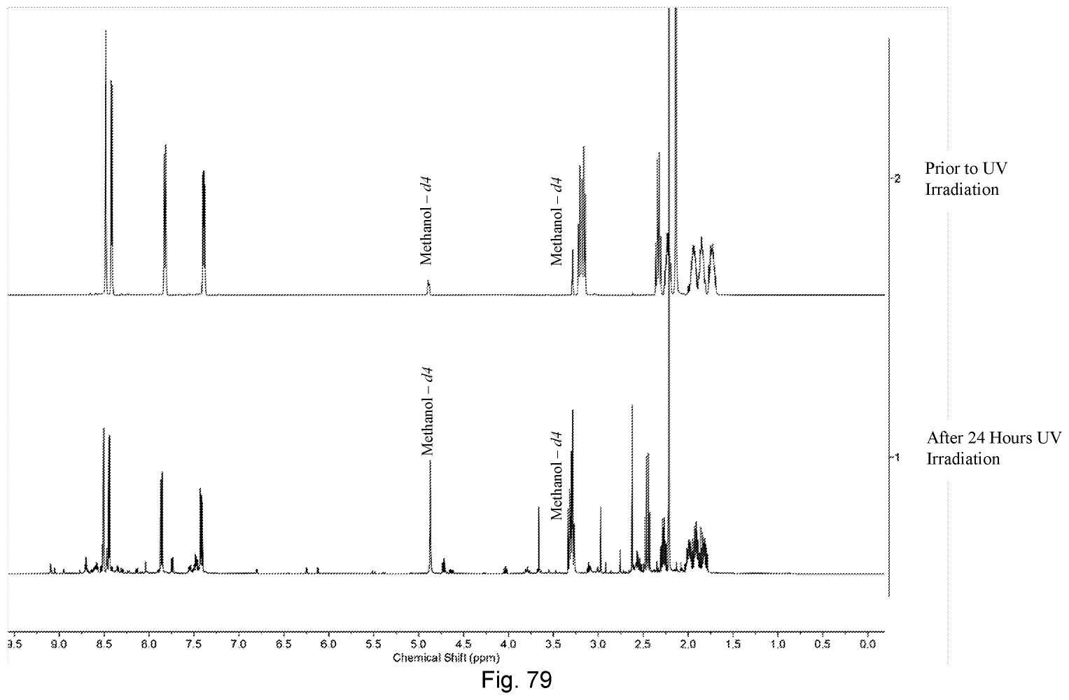

UV Photodegradation Analysis. Nicotine is also a highly sensitive naturally occurring molecule. Described as a clear to light yellow oily liquid, nicotine is hygroscopic, meaning that it will absorb atmospheric moisture. It is also has been shown to oxidize at ambient conditions due to atmospheric oxygen and has been shown to undergo degradation pathways when exposed to ambient ultraviolet (UV) radiation. The UV degradation of nicotine is known to produce oxidized nicotine (nicotine N-oxide), nicotinic acid (vitamin B3), and methylamine. As such the stability of the produced crystalline nicotine salts were studied by placing them in a homemade UV irradiation box for 24 hours while airflow into the box was maintained. The pure coformers, as well as pure nicotine (FIG. 79), were also each tested as a standard for their respective nicotine salt. This test was designed to attempt to forcefully stress test and degrade the crystalline nicotine materials. NMR was selected as the most readily available means to quantify any degradation that may occur. As observed in the ¹H NMR spectra, many new peaks were starting to grow in as the nicotine is exposed to UV irradiation under air flow. This shows that pure nicotine is indeed incredibly unstable. The most obvious sign of nicotine degradation comes from the color change that occurs to the liquid (FIGS. 77 and 78).

Neither pure enantiomer of malic acid undergoes photodegradation when irradiated with UV light over 24 hours. However, (S)-nicotine does decompose when exposed to UV light over 24 hours, as chronicled in FIGS. 77 and 78, wherein the liquid (S)-nicotine gradually gets darker the longer the sample is irradiated with UV light with air exposure. This visual combined with the NMR data from FIG. 79, indicate that pure (S)-nicotine decomposes when exposed to UV light. Each of the synthesized nicotine salts did not decompose after 24 hours of UV irradiation. This indicates that each of the synthesized nicotine salts possess a greater photostability over pure (S)-nicotine, and as such they will not degrade when exposed to ultraviolet (UV) light despite each salt having nicotine in the crystal.

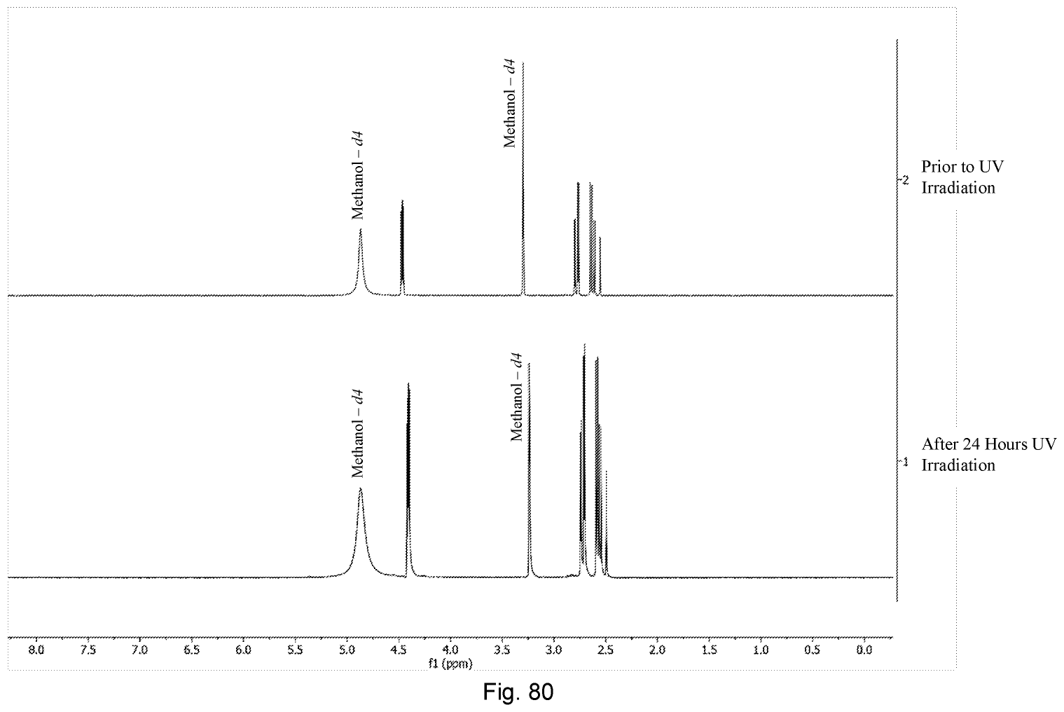

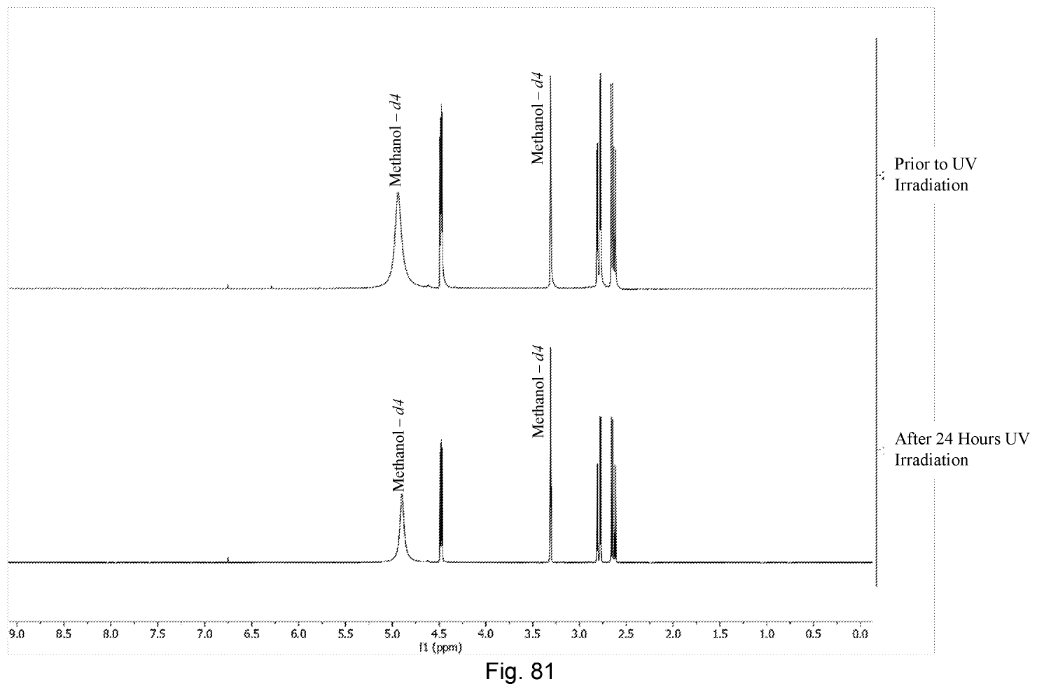

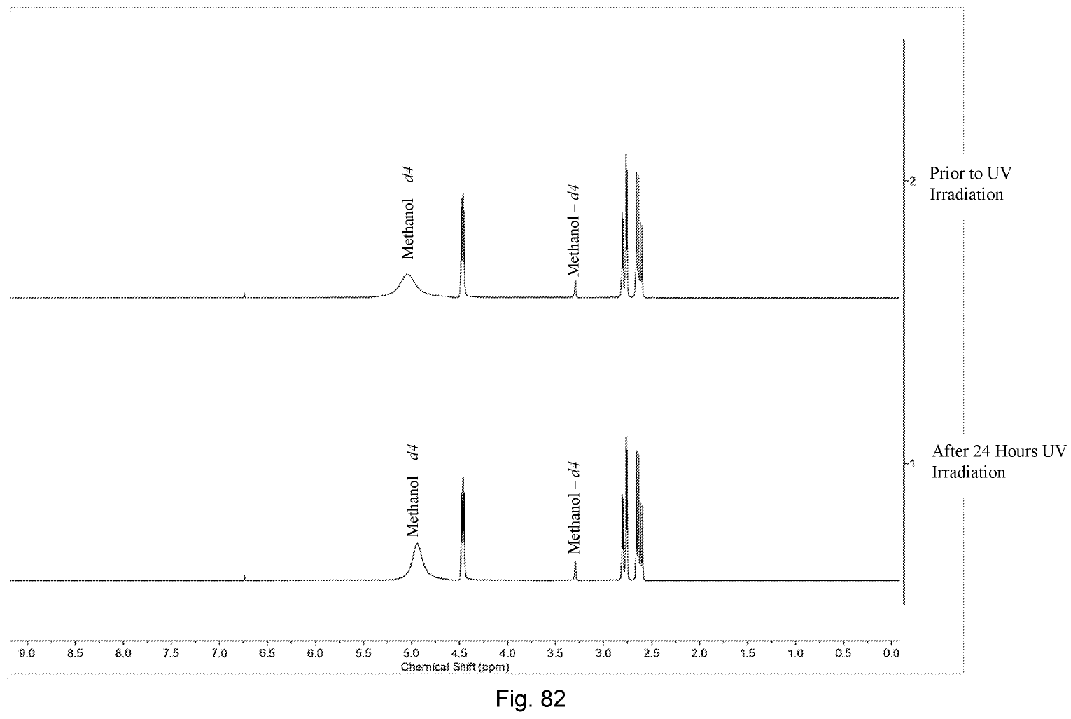

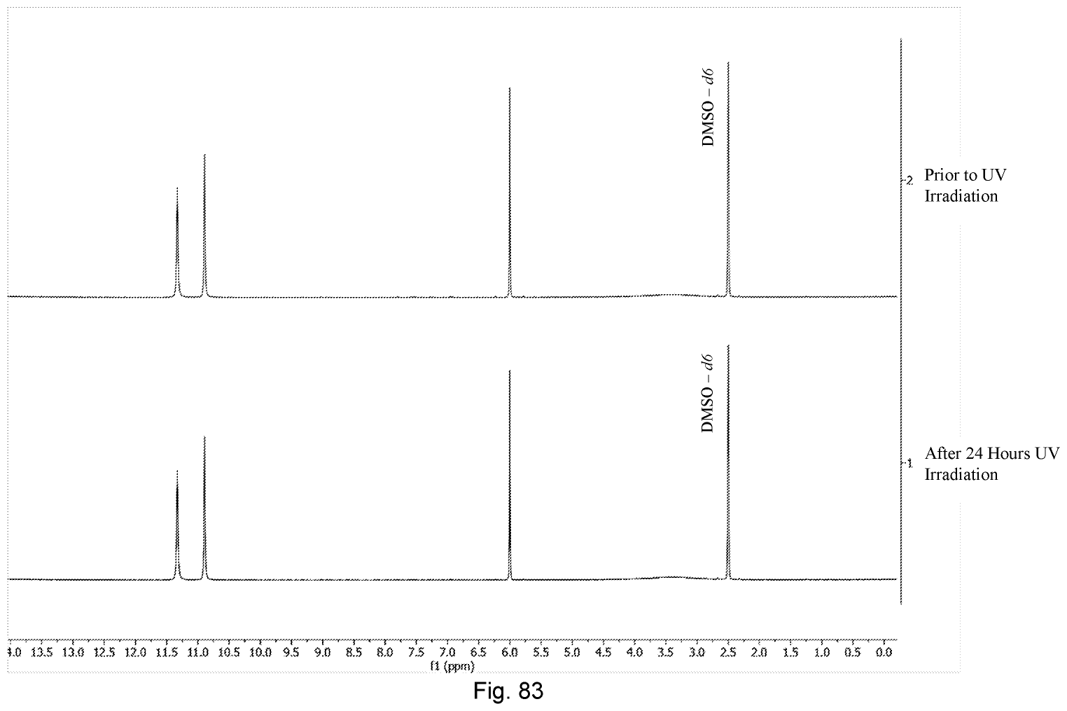

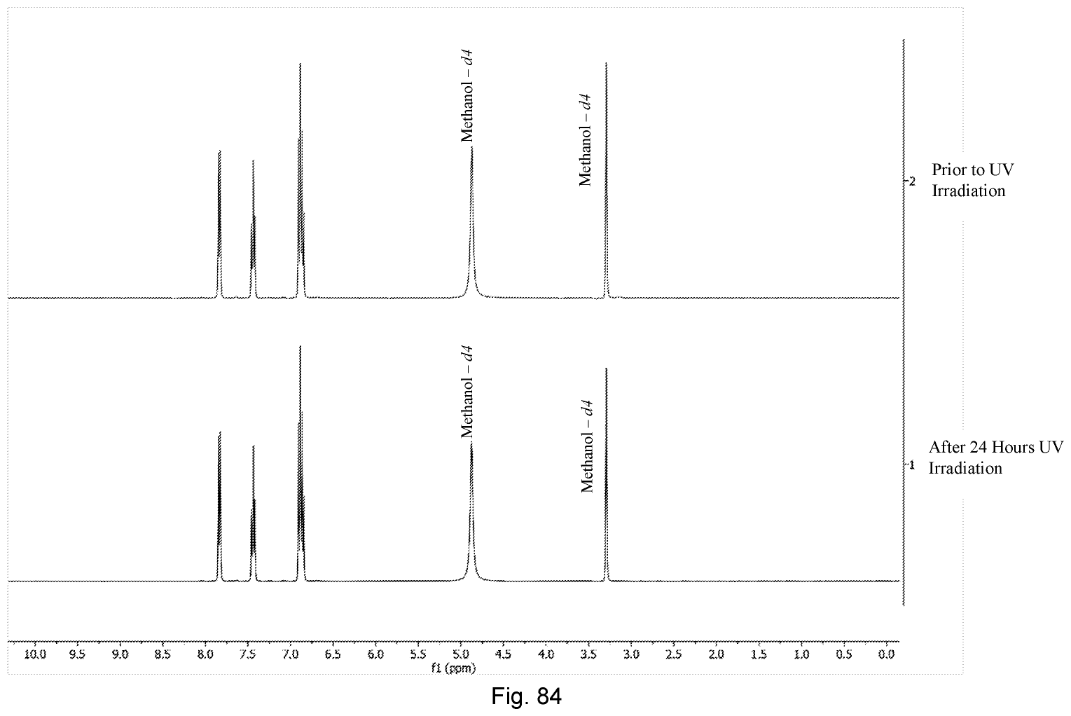

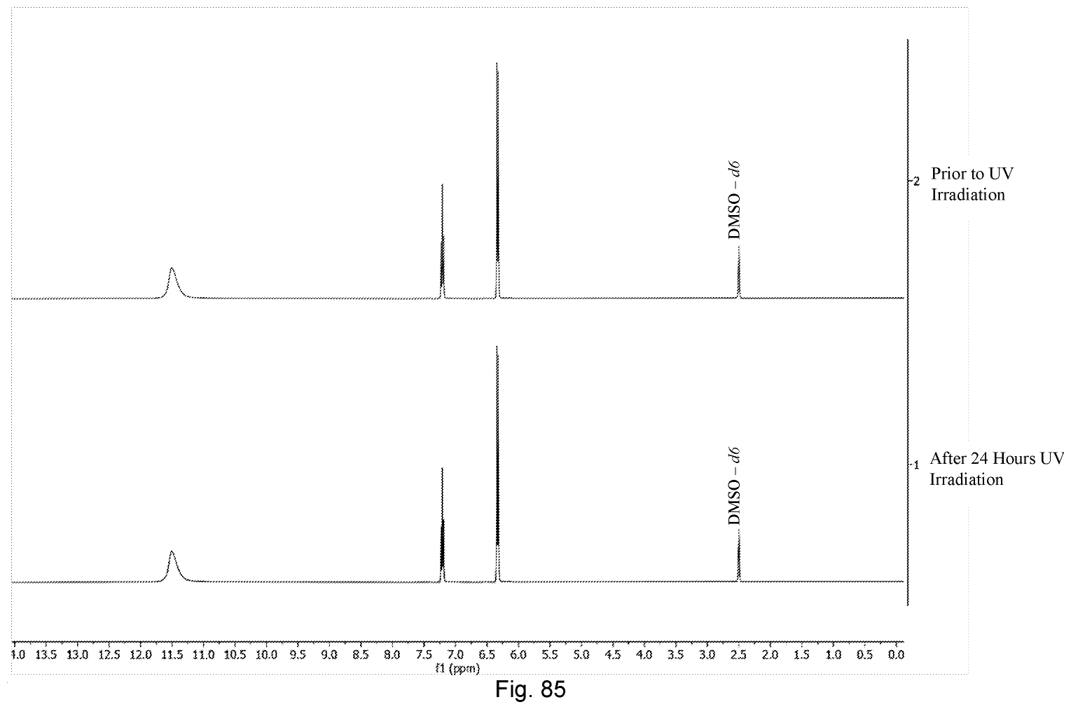

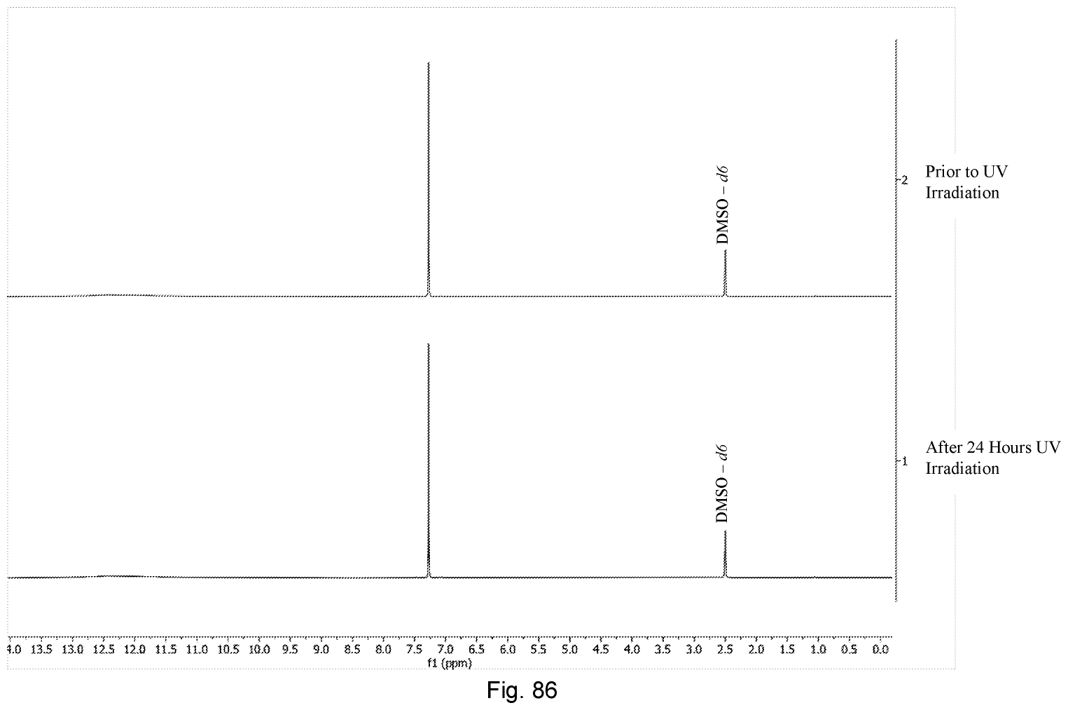

Results have shown that each coformer selected for the crystalline salts thus far do not degrade under the UV and air exposure (FIGS. 80-86). This shows that the coformers may potentially be good candidates for enhancing the photostability and air sensitivity of pure nicotine, as they themselves exhibit photostability under these conditions.

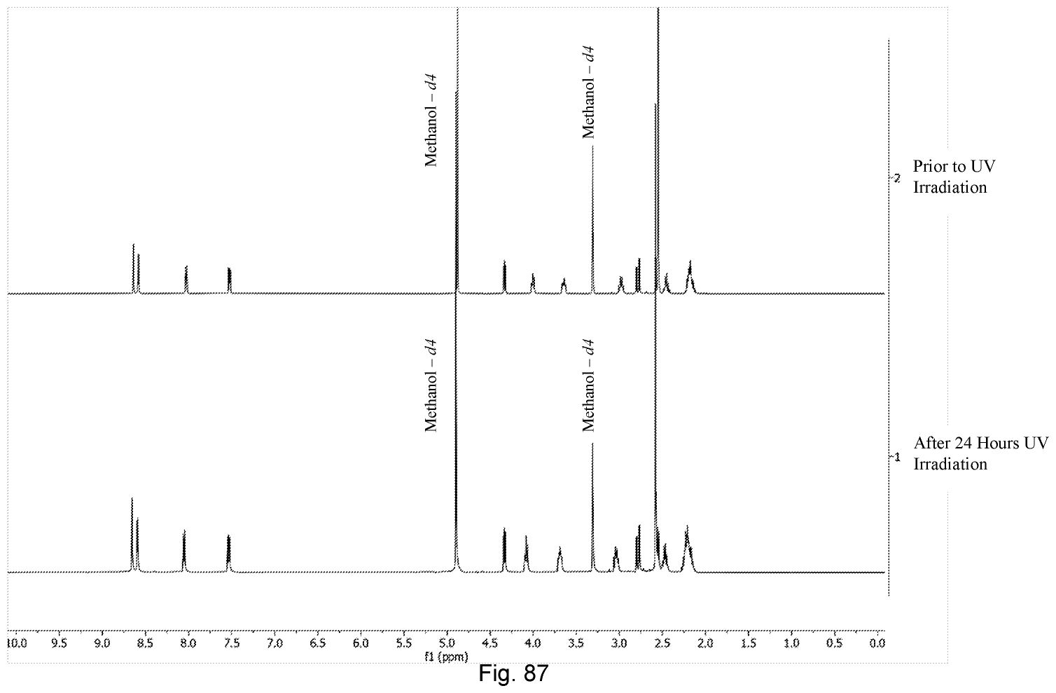

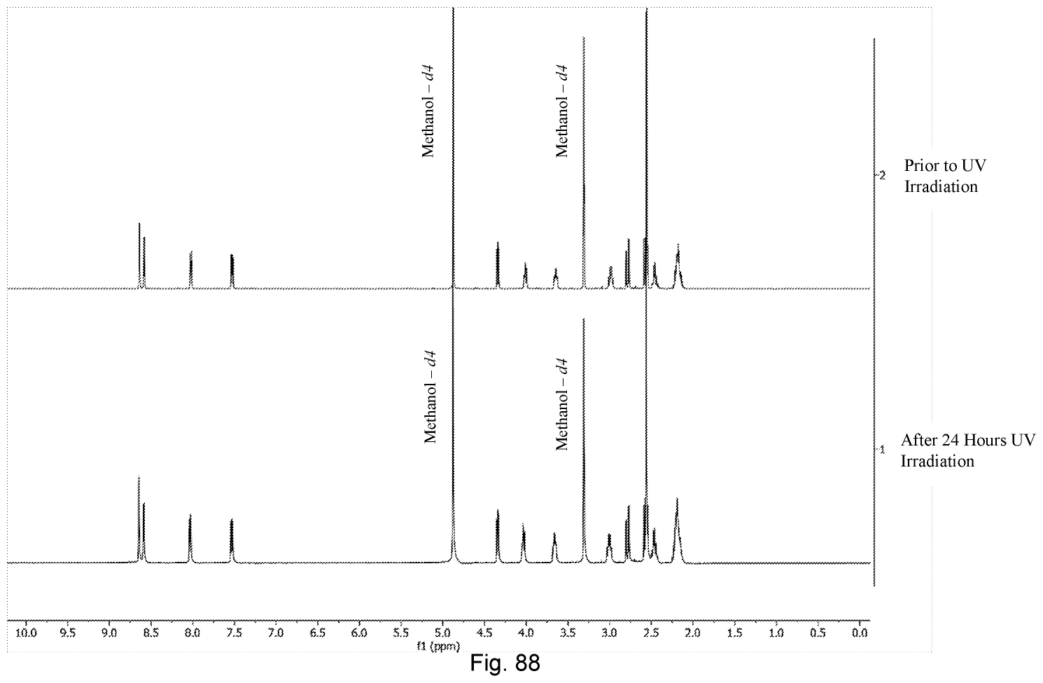

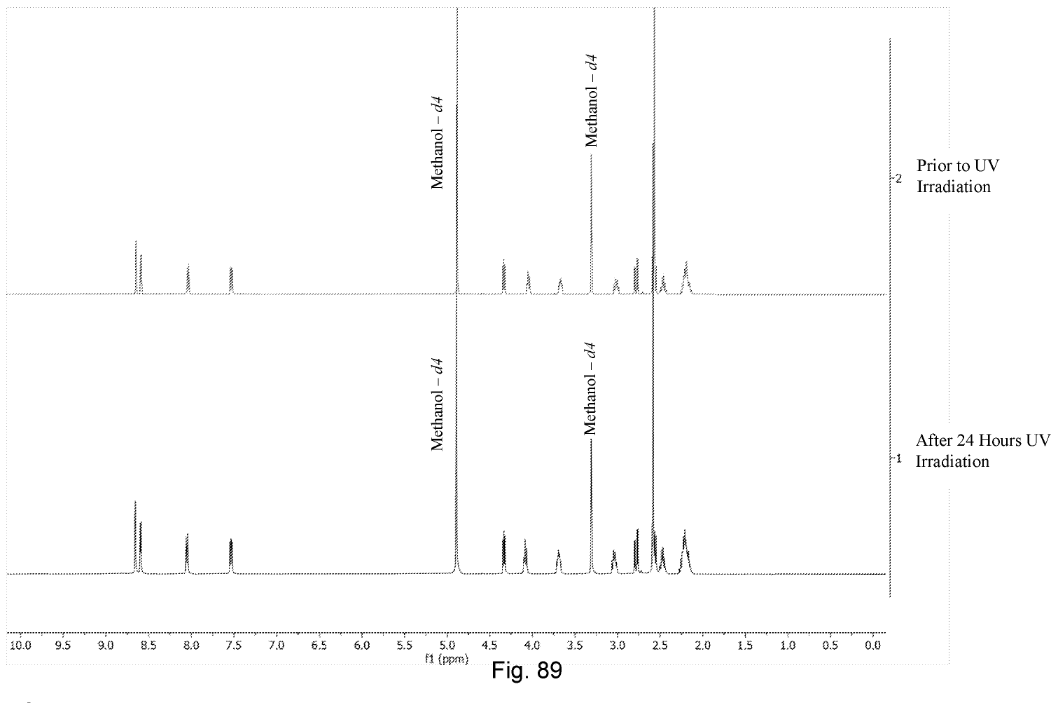

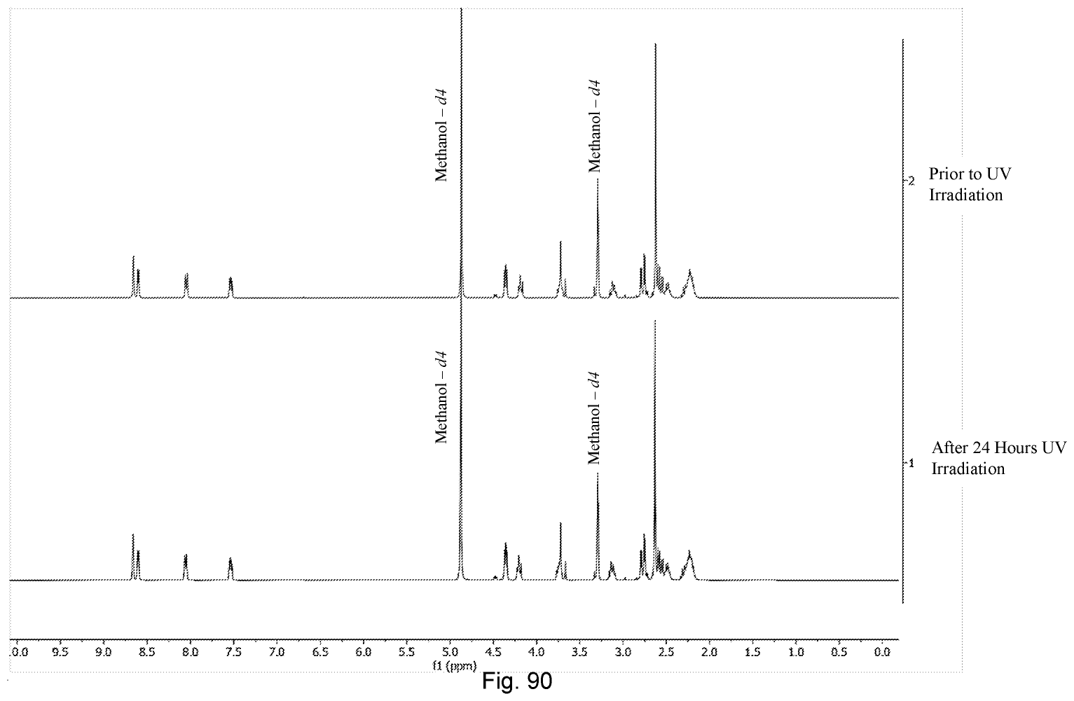

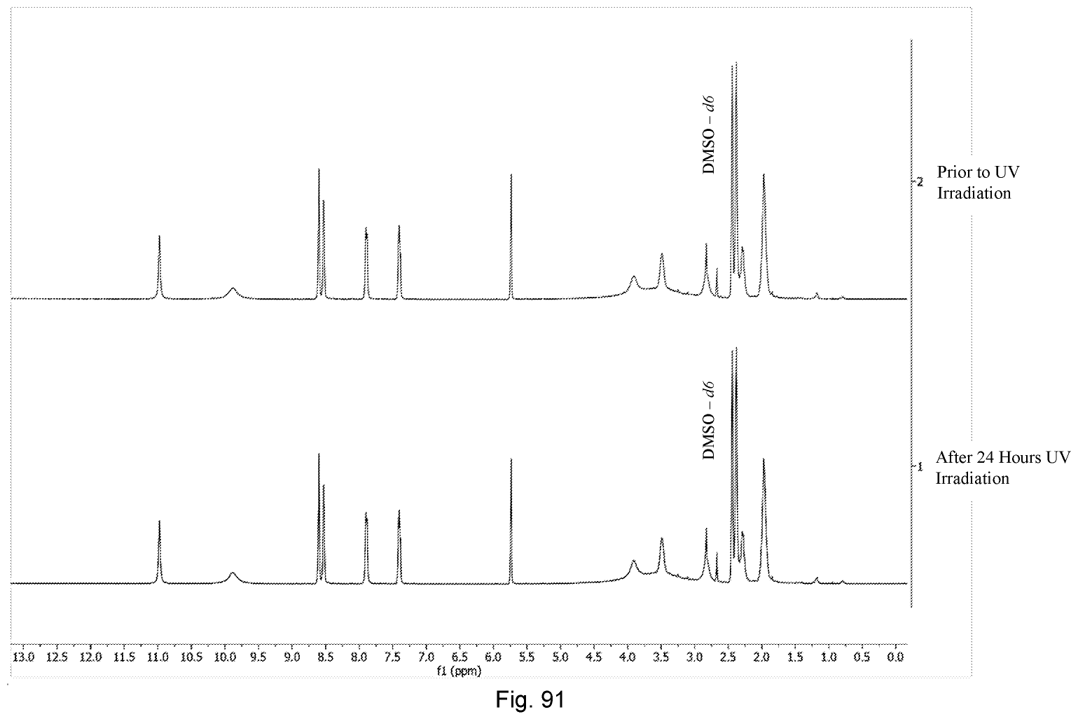

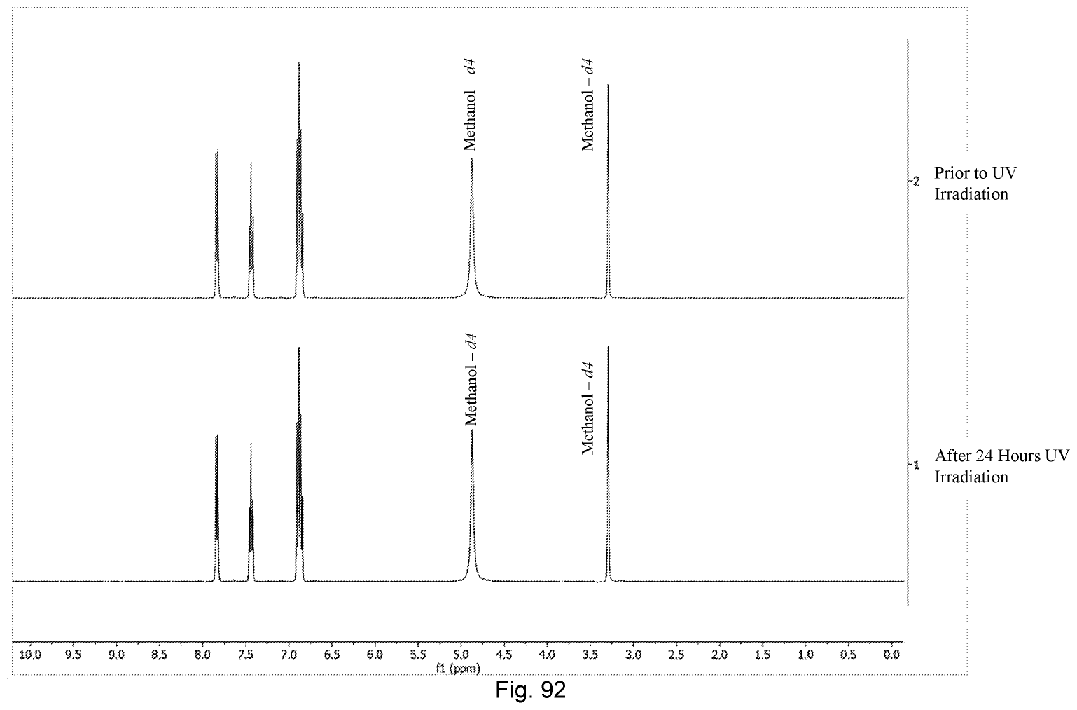

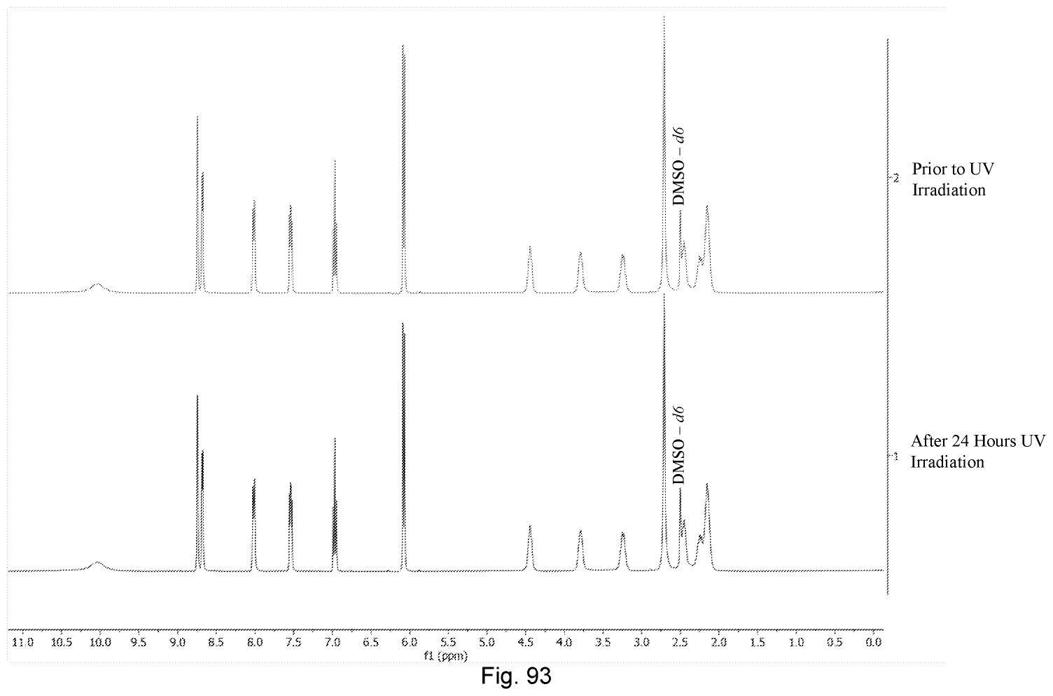

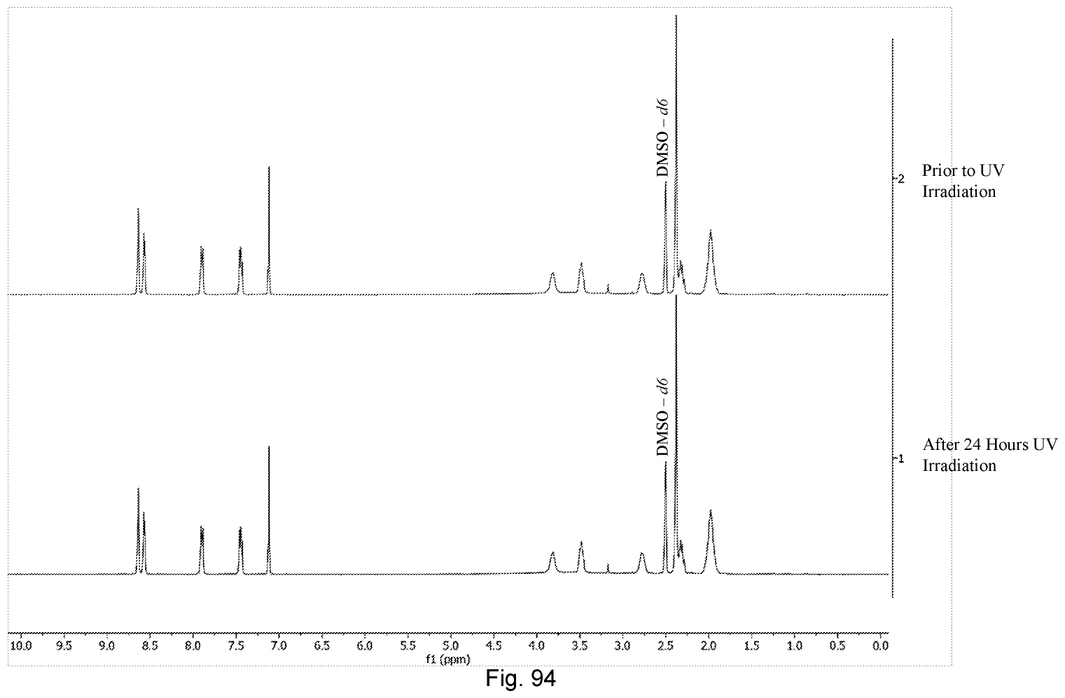

A sample of each crystalline nicotine salt was dissolved in an appropriate NMR solvent and the resulting solution spectra were used for comparison of any detectable degradation upon UV irradiation. Results have thus far shown that each crystalline nicotine salt that has been synthesized has exhibited no detectable degradation once an irradiated crystalline sample is dissolved in NMR solution after 24 hours of UV irradiation (FIGS. 87-94). Each synthesized crystalline nicotine salt offers vastly improved photostability in comparison to pure nicotine, with no detectable degradation observed in the irradiated sample spectra. Correlation can be drawn successfully between the selected coformers' photostability and the respective nicotine salt. Each crystalline salt that was shown to be photostable also had a coformer that was also shown to be photostable.

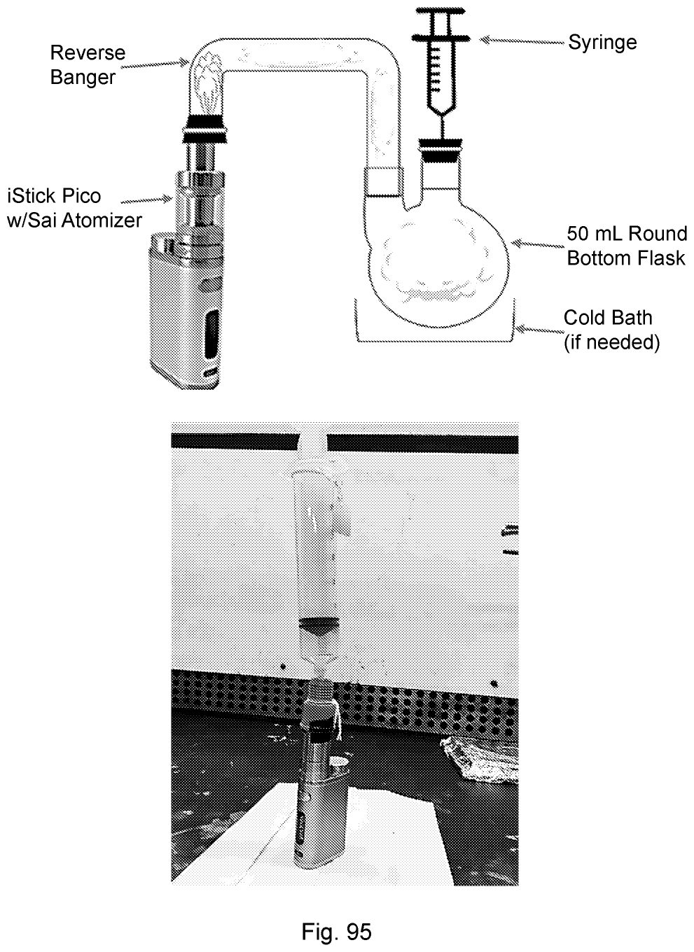

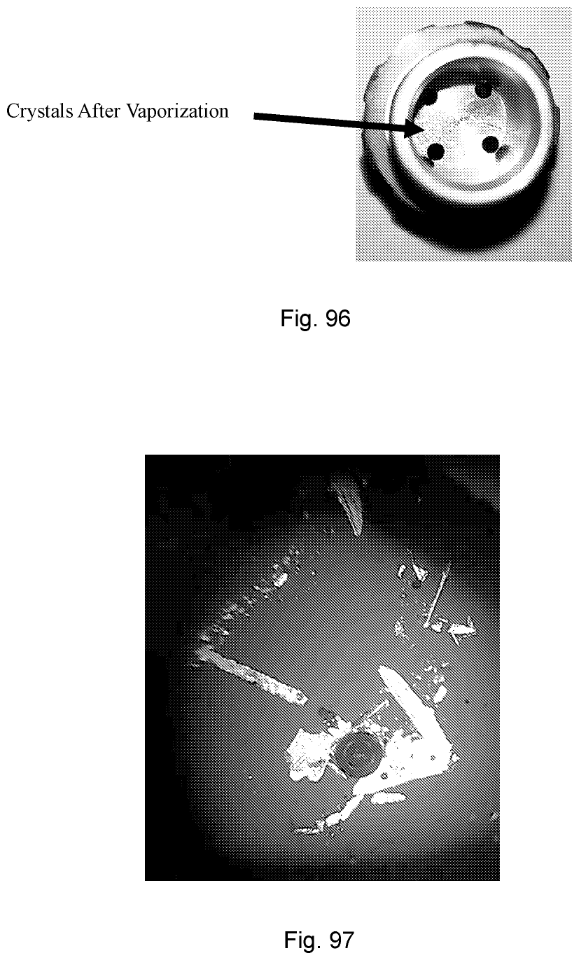

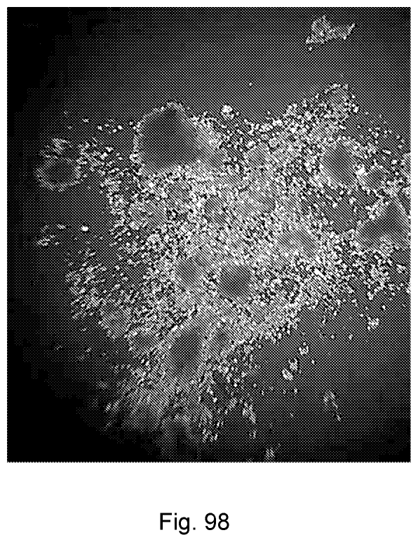

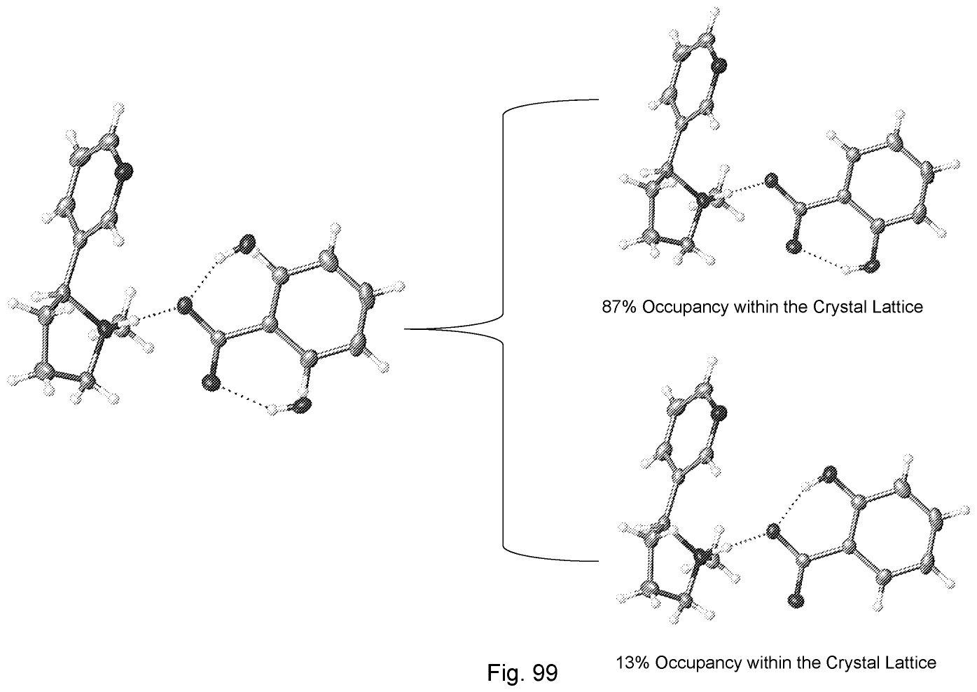

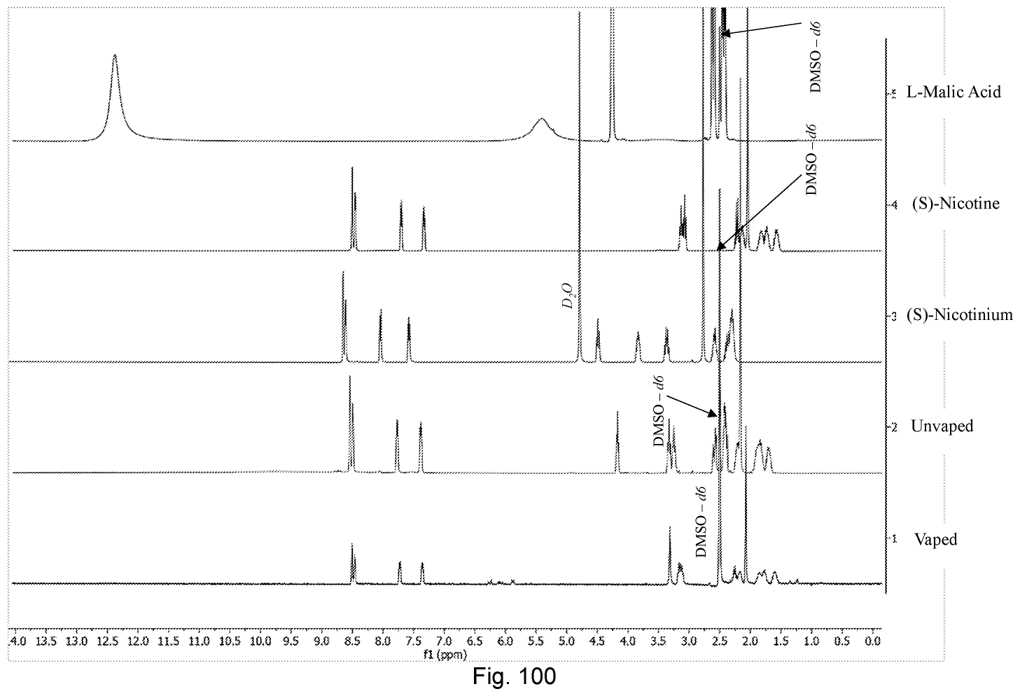

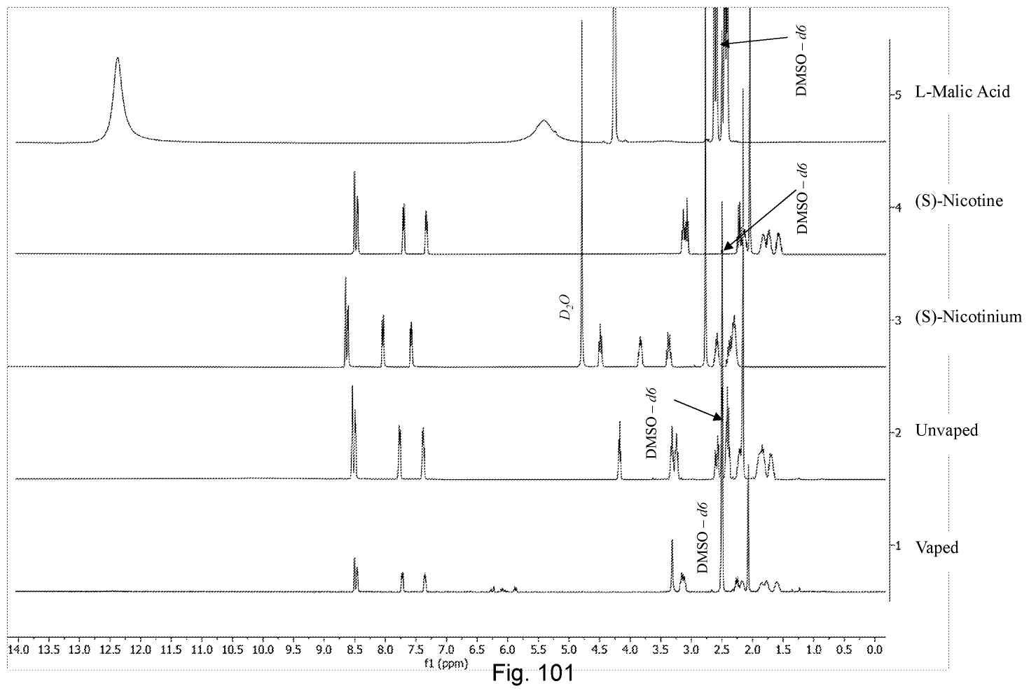

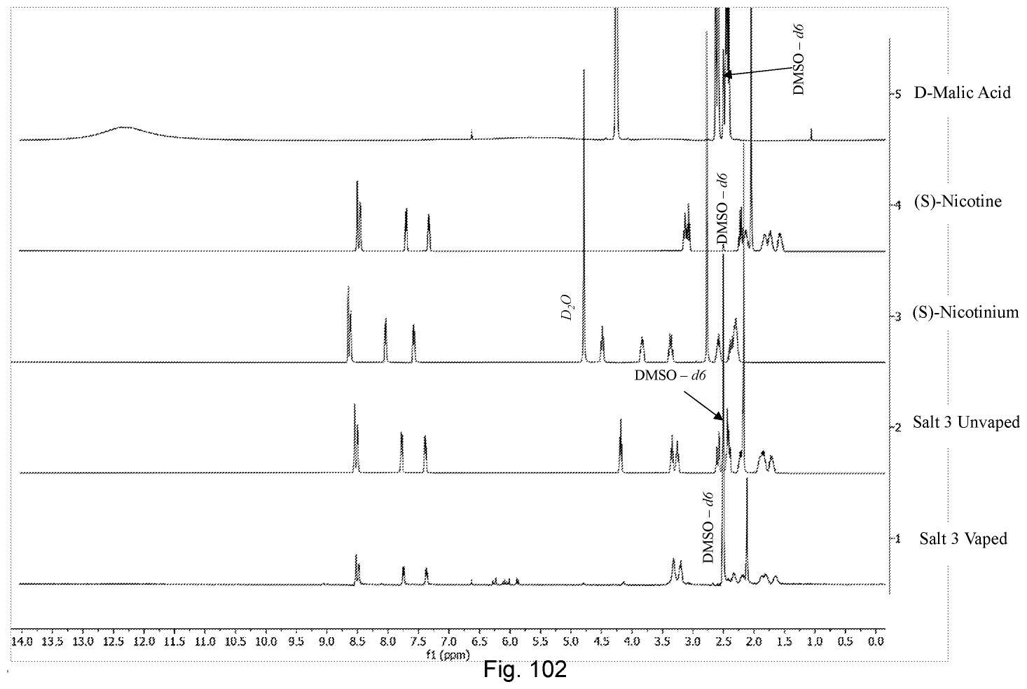

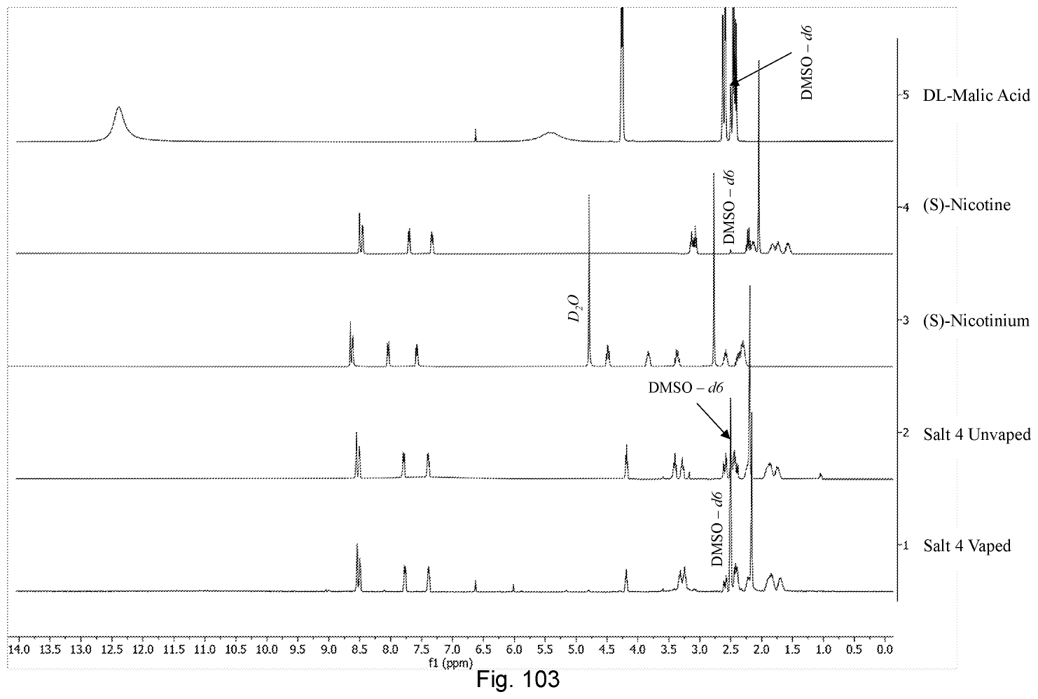

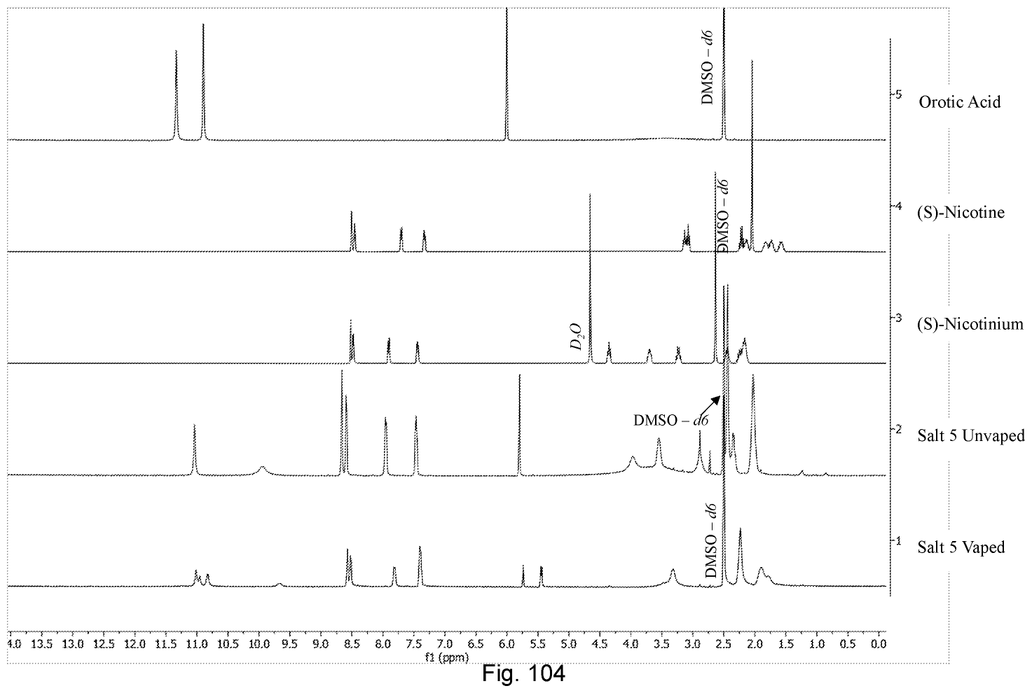

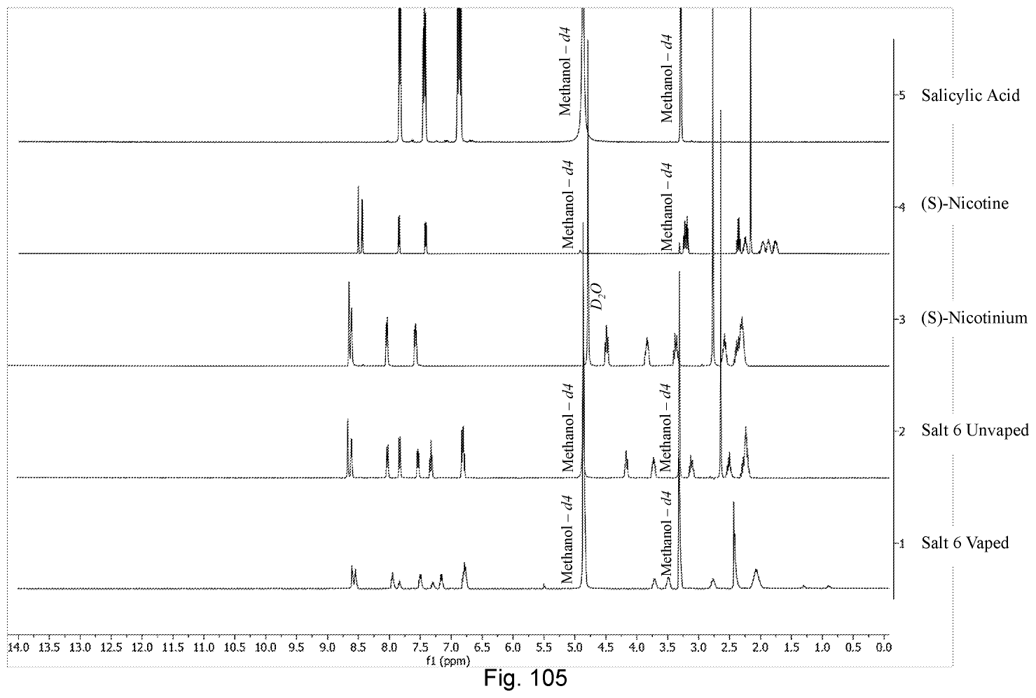

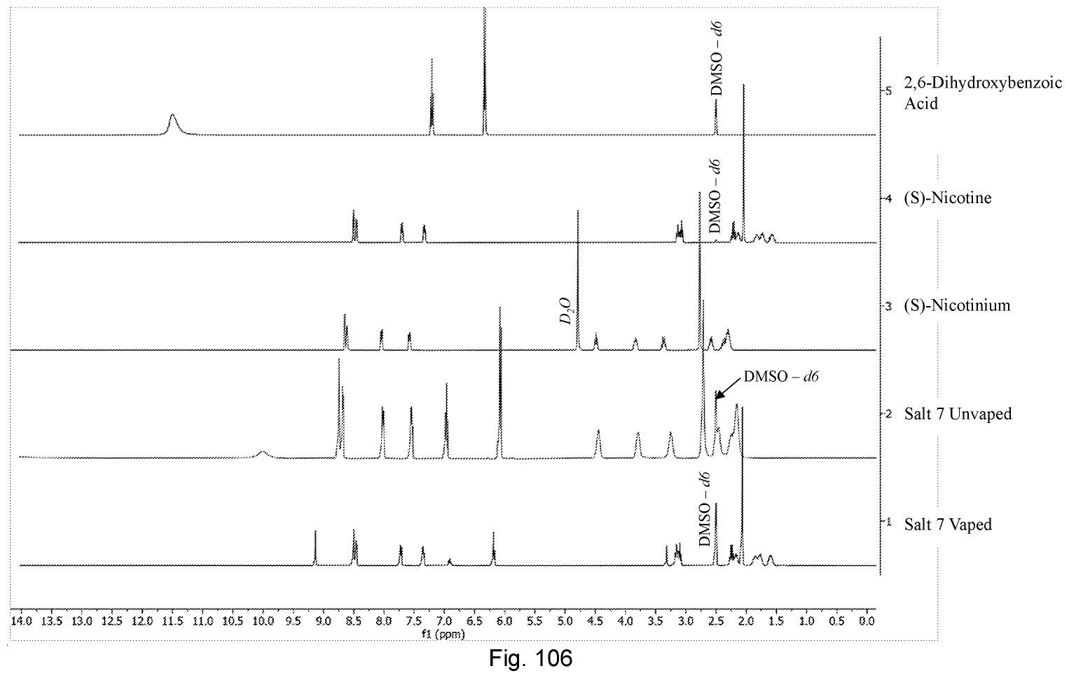

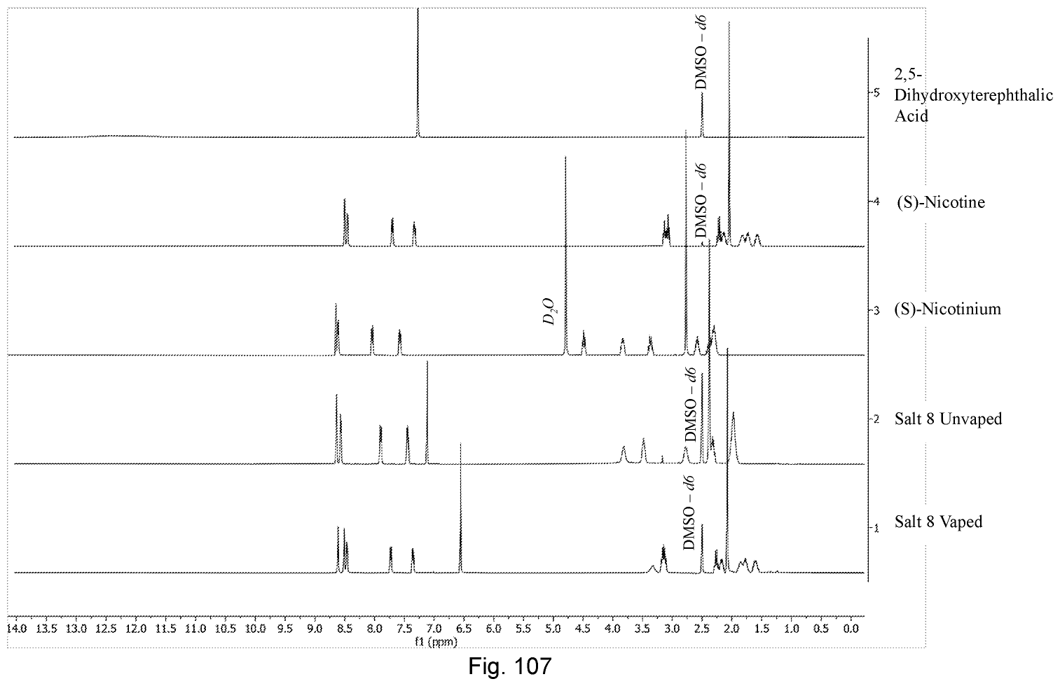

Aerosolization/Vaporization Analysis. Using the aforementioned aerosolization/vaporization analytical setup, seen in FIG. 95, monoclinic P2₁ (S)-nicotinium salicylate was weighed into the bucket coil, attached to the i Stick Pico mod and connected to the system. Amounts up to 50 mg of the salt were loaded and vaporized. Numerous simulated “puffs” were done using the syringe as a scaled down analog of a lung breathing the vapor. Vapor was noticed settling and condensing within the mouthpiece, where it was recrystallizing (FIGS. 96 and 97). Similarly, nanocrystalline material was collected from the inside of the lung analog syringe (FIG. 98). The amorphous material that was collected in the flask was analyzed via ¹H NMR, confirming that the salt had been delivered to the lung analog from the solid-state nicotine salt. This crude setup will be refined in the future, but yielded a proof of concept in that the instant synthesized solid-state nicotine salts can be delivered via aerosolization without additives. It was worth noting that phenol was likely also formed due to a decarboxylation of the acid coformer during heating. Single crystals suitable for analysis by X-ray diffraction were recovered from inside the mouthpiece, as well as in the syringe, which acts as a simulated lung. The recovered crystalline material, from both the mouthpiece and inner syringe, exhibited a new structure, different from the nicotine material prior to vaporization. The material underwent an order-disorder phase transition, during which the benzyl ring was allowed to rotate about a C2 axis of symmetry within the salicylate molecule, thus allowing carboxylate group to remain bonded to the nicotine. After this transformation, the condensed nicotine material exhibited a structure that had a 13% occupancy of the hydroxyl group pointing up, in the direction of the pyridine portion of the nicotine structure. Meanwhile 87% of the crystalline material exhibited the hydroxyl group pointing down opposite of the pyridine portion of the nicotine (FIG. 99). As such, it can be said that a new kinetically trapped morphology, or polymorph, forms when monoclinic (S)-nicotinium salicylate is vaporized, undergoes an order-disorder phase transition and then re-condenses. In addition, all salts have been vaporized and the resulting aerosols have been characterized via ¹H-NMR, detailing delivery of the nicotine, nicotinium, salt, coformer, decarboxylated coformer, or combination thereof (FIGS. 100-107).

Although the present disclosure has been described with respect to one or more particular embodiment(s) and/or example(s), it will be understood that other embodiment(s) and/or example(s) of the present disclosure may be made without departing from the scope of the present disclosure.

1. A method of making a nicotine material comprising:
providing a nicotine material-forming solution comprising nicotine, one or more coformer(s), and, optionally, one or more solvent(s); and
evaporating at least a portion of the coformer(s), if the coformer(s) are a solvent or solvents, and/or solvent(s), if present, from the nicotine material-forming solution,
wherein the nicotine material is formed.

2. A method of claim 1, further comprising isolating the nicotine material.

3. A method of claim 1, further comprising rinsing the nicotine material.

4. A method of claim 3, wherein the nicotine material is rinsed with a solvent chosen from hydrocarbon solvents, alcohols, ketone solvents, ester solvents, halogenated solvents, and combinations thereof.

5. A method of claim 1, further comprising drying the nicotine material.

6. A method of claim 5, wherein the drying is carried out under vacuum and/or at a temperature below the melting and/or decomposition temperature of the nicotine material.

7. A method of claim 1, wherein the solvent of the nicotine material-forming solution is chosen from organic solvents, water, and combinations thereof.

8. A method of claim 7, wherein the organic solvents are chosen from alcohols, cyclic ethers, polar protic solvents, polar aprotic solvents, halogenated alkanes, halogenated aryl solvents, and combinations thereof.

9. A method of claim 1, wherein the nicotine is S-nicotine, R-nicotine, or a combination thereof.

10. A method of claim 1, wherein the nicotine is present in the nicotine material-forming solution at its solubility limit in the solvent(s) or at 90% or more of its solubility limit in the solvent(s) or at a concentration of at least 0.01 M.

11. A method of claim 1, wherein the coformer(s) is/are chosen from organic compounds, mineral acids, and combinations thereof.

12. A method of claim 11, wherein the organic compounds are chosen from carboxylic acids, hydroxycarboxylic acids, alcohols, polyacids, sweetening agents, amino acids, and combinations thereof.

13. A method of claim 1, wherein the coformer(s) is/are present in the nicotine material-forming solution at 0.01 to 2 M.

14. A method of claim 1, wherein the nicotine material is amorphous, polycrystalline, single crystalline, or a combination thereof.

15. A method of claim 1, wherein at least a portion of or all of the nicotine material exhibits orthorhombic symmetry, monoclinic symmetry, hexagonal symmetry, trigonal symmetry, triclinic symmetry, tetragonal symmetry, or cubic symmetry.

16. A method of claim 1, further comprising forming the nicotine material into a tablet or forming a liquid comprising the nicotine material.

17. A nicotine material comprising nicotine and one or more coformer(s).

18. A nicotine material of claim 17, wherein the nicotine material is amorphous, polycrystalline, single crystalline, or a combination thereof.

19. A nicotine material of claim 17, wherein the coformer(s) is/are chosen from organic compounds, mineral acids, and combinations thereof.

20. A nicotine material of claim 19, wherein the organic compounds are chosen from carboxylic acids, alcohols, polyacids, sweetening agents, amino acids, and combinations thereof.

21. A nicotine material of claim 17, wherein at least a portion of or all of the nicotine material exhibits orthorhombic symmetry, monoclinic symmetry, hexagonal symmetry, trigonal symmetry, triclinic symmetry, tetragonal symmetry, or cubic symmetry.

22. A nicotine material of claim 17, wherein the ratio of nicotine molecules to coformer molecule(s) is 1:5 to 5:1.

23. A nicotine material of claim 17, wherein the coformer is chosen from L-malic acid, D-malic acid, DL-malic acid, and 2,6-dihydroxybenzoic acid, and
at least a portion of or all of the nicotine material is a polymorph chosen from monoclinic P2₁ (S)-nicotinium L-malate, orthorhombic P2₁2₁2₁ (S)-nicotinium D-malate, orthorhombic P2₁2₁2₁ (S)-nicotinium DL-malate, and monoclinic P2₁ (S)-nicotinium 2,6-dihydroxybenzoate.

24. A nicotine material of claim 17, wherein the coformer is chosen from L-malic acid, D-malic acid, DL-malic acid, and 2,6-dihydroxybenzoic acid, and
at least a portion of or all of the nicotine material exhibits an orthorhombic structure, a monoclinic structure, or a combination thereof.

25. A nicotine material of claim 17, wherein the nicotine material exhibits substantially no degradation after 6 hours under UV photodegradation conditions.

26. A nicotine material of claim 17, wherein the nicotine material exhibits a desired melting point, vaporization phase stability, dissolution rate, or a combination thereof dependent upon the coformer(s) present in the material.

27. A composition comprising one or more nicotine material(s) of claim 17 and one or more additive(s).

28. A composition of claim 27, wherein the additive(s) is/are chosen from flavoring agents, excipients, sweeteners, binding agents, and combinations thereof.

29. A composition of claim 27, wherein the composition is in a solid form, a liquid form, a solution, a vapor form, or an aerosol form.

30. An article of manufacture comprising one or more nicotine material(s) of claim 17.

31. An article of manufacture of claim 30, wherein the article of manufacture is a transdermal delivery device, an oral delivery device, a solid state vaporization tablet, a solid state vaporization capsule, or a dissolvable formulation tablet.

32. A method of forming vapor phase or aerosol phase nicotine comprising:
vaporizing or aerosolizing one or more nicotine material claim 17,
such that the vapor phase or aerosol phase of the nicotine is formed.

33. A method of forming vapor phase or aerosol phase nicotine of claim 32, wherein the vaporizing is carried out at a temperature of 100 to 350° C.

34. A method of forming vapor phase or aerosol phase nicotine of claim 32, wherein the vaporizing or aerosolizing is carried out in a device.

35. A method of forming vapor phase or aerosol phase nicotine of claim 32, wherein 90 to 100% is vaporized or aerosolized.
